(19) United States
(12) Patent Application Publication
(10) Pub. No.: US 2016/0222458 A1
(43) Pub. Date: Aug. 4, 2016

(54) Gene Expression Profile Breast Tumour Grading

(71) Applicant: AGENCY FOR SCIENCE, TECHNOLOGY AND RESEARCH, Singapore (SG)

(72) Inventors: Lance D. Miller, Winston-Salem, NC (US); Vladimir Kuznetsov, Singapore (SG); Anna Ivshina, Singapore (SG); Luay Aswad, Singapore (SG); Surya Pavan Yenamandra, Singapore (SG)

(73) Assignee: AGENCY FOR SCIENCE, TECHNOLOGY AND RESEARCH, Singapore (SG)

(21) Appl. No.: 14/737,807

(22) Filed: Jun. 12, 2015

Related U.S. Application Data
(63) Continuation-in-part of application No. 13/954,050, filed on Jul. 30, 2013, now abandoned.
Application No. 13/954,050 is a continuation of application No. 12/446,195, filed on Oct. 12, 2010, now abandoned.
(60) Provisional application No. 60/862,519, filed on Oct. 23, 2006.

(30) Foreign Application Priority Data
Oct. 20, 2006 (SG) 200607354-8

(51) Int. Cl. C12Q 1/68; G06F 19/20

(52) U.S. Cl. CPC C12Q 1/6886; G06F 19/20; C12Q 2600/158; C12Q 2600/112

(57) ABSTRACT

We describe a method of assigning a grade to a breast tumour, which grade is indicative of the aggressiveness of the tumour, the method comprising detecting the expression of a gene selected from the genes set out in Table D0 (6g-TAGs) or Table D1 (SWS Classifier 0). We also describe methods of treating patients having a high aggressiveness tumour or a low aggressiveness tumour, by identifying the aggressiveness tumour by obtaining, from a sample of a histological Grade 2 tumour isolated from the patient, gene expression data of BRRN1, AURKA, MELK, PRR11, CENPW and E2F1; assigning a grade to the tumour by applying a class prediction algorithm to the gene expression data, wherein a Grade 3 tumour is classified as a high aggressiveness tumour and a Grade 1 tumour is classified as a low aggressiveness tumour; and specifically treating the patient accordingly.

### CROSS REFERENCE TO RELATED APPLICATIONS

This application is a continuation-in-part of U.S. patent application Ser. No. 13/954,050 filed Jul. 30, 2013, which is a continuation of U.S. patent application Ser. No. 12/446,195 filed Apr. 17, 2009 (with a 371(c) date of Oct. 12, 2010), which is a 371 of PCT/SG2007/000357 filed Oct. 19, 2007, which claims the benefit of U.S. Patent Application No. 60/862,519 filed Oct. 23, 2007. This application claims priority from Singapore Patent Application No 200607354-8, filed Oct. 20, 2006.

The foregoing applications, and each document cited or referenced in each of the present and foregoing applications, including during the prosecution of each of the foregoing applications (“application and article cited documents”), and any manufacturer's instructions or catalogues for any products cited or mentioned in each of the foregoing applications and articles and in any of the application and article cited documents, are hereby incorporated herein by reference. Furthermore, all documents cited in this text, and all documents cited or reference in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art.

### FIELD

The present invention relates to the fields of medicine, cell biology, molecular biology and genetics. More particularly, the invention relates to a method of assigning a grade to a breast tumour which reflects its aggressiveness.

### BACKGROUND

The effective treatment of cancer depends, to a large extent, on the accuracy with which malignant tissue can be subtyped according to clinicopathological features that reflect disease aggressiveness.

Some clinical subtypes, despite phenotypic homogeneity, are associated with substantial clinical heterogeneity (e.g., refractory response to treatment) confounding their clinical meaning. Recent studies using DNA microarray technology suggest that such clinical heterogeneity may be resolvable at the molecular level (1-4). Indeed, some have demonstrated that gene expression signatures underlying specific biological properties of cancer cells may be superior indicators of clinical subtypes with robust prognostic value (1, 2). Thus, global analysis of gene expression has the potential to uncover molecular determinants of clinical heterogeneity providing a more objective and biologically-rational approach to cancer subtyping.

Accordingly, there is a need in the art for gene markers which are diagnostic or reflective of tumourigenicity.

In breast cancer, histologic grade is an important parameter for classifying tumours into morphological subtypes informative of patient risk. Grading seeks to integrate measurements of cellular differentiation and replicative potential into a composite score that quantifies the aggressive behaviour of the tumour.

The most studied and widely used method of breast tumour grading is the Elston-Ellis modified Scarff, Bloom, Richardson grading system, also known as the Nottingham grading system (NGS) (5, 6, Haybittle et al, 1982). The NGS is based on a phenotypic scoring procedure that involves the microscopic evaluation of morphologic and cytologic features of tumour cells including degree of tubule formation, nuclear pleomorphism and mitotic count (6). The sum of these scores stratifies breast tumours into Grade I (G1) (well-differentiated, slow-growing), Grade II (G2) (moderately differentiated), and Grade III (G3) (poorly-differentiated, highly-proliferative) malignancies.

Multivariate analyses in large patient cohorts have consistently demonstrated that the histologic grade of invasive breast cancer is a powerful prognostic indicator of disease recurrence and patient death independent of lymph node status and tumour size (6-9). Untreated patients with G1 disease have a ˜95% five-year survival rate, whereas those with G2 and G3 malignancies have survival rates at 5 years of ˜75% and ˜50%, respectively.

However, the value of histologic grade in patient prognosis has been questioned by reports of substantial inter-observer variability among pathologists (10-13) leading to debate over the role that grade should play in therapeutic planning (14, 15). Furthermore, where the prognostic significance of G1 and G3 disease is of more obvious clinical relevance, it is less clear what the prognostic value is of the more heterogeneous, moderately differentiated Grade II tumours, which comprise approximately 50% of all breast cancer cases (9, 15, 16).

There is therefore a need for methods which are capable of discriminating between heterogeneous tumour grades, particularly Grade II breast tumours.

### SUMMARY

We have now demonstrated that a gene expression signature comprising one or more of a set of 232 genes, represented by 264 probesets (e.g., Affymetrix probesets), is capable of discriminating between high and low grade tumours. Such a gene expression signature may be used to provide an objective and clinically valuable measure of tumour grade.

We further describe a novel strategy of clinical class discovery that combines gene discovery and class prediction algorithms with patient survival analysis, and between-group statistical analyses of conventional clinical markers and gene ontologies represented by differentially expressed genes.

Our findings show that the genetic reclassification of histologic grade reveals new clinical subtypes of invasive breast cancer and can improve therapeutic planning for patients with moderately differentiated tumours.

Furthermore, our results support the view that tumours of low and high grade, as defined genetically, may reflect independent pathobiological entities rather than a continuum of cancer progression.

According to a 1^{st} aspect of the present invention, we provide a method of assigning a grade to a breast tumour, which grade is indicative of the aggressiveness of the tumour, the method comprising detecting the expression of a gene selected from the genes set out in Table D0 (6g-TAG) or Table D1 (SWS Classifier 0).

The method may comprise detecting the expression of level of 5 or more genes. The 5 or more genes may comprise the genes set out in Table D0 (6g-TAGs).

The method may comprise detecting the expression of BRRN1 (GenBank Accession No. NM_015341), AURKA (GenBank Accession No. NM_003600), MELK (GenBank Accession No. NM_014791), PRR11 (GenBank Accession No. NM_018304), CENPW (GenBank Accession No. NM_001012507) and E2F1 (GenBank Accession No. NM_005225).

There is provided, according to a 2^{nd} aspect of the present invention, a method of classifying a histological Grade 2 tumour into a low aggressiveness tumour or a high aggressiveness tumour, the method comprising assigning a grade to the histological Grade 2 tumour according to the 1^{st} aspect of the invention.

We provide, according to a 3^{rd} aspect of the present invention, a method of predicting a survival rate for an individual with a histological Grade 2 breast tumour, the method comprising assigning a grade to the breast tumour by a method according to any preceding aspect of the invention.

As a 4^{th} aspect of the present invention, there is provided a method of prognosis of an individual with a breast tumour, the method comprising assigning a grade to the breast tumour by a method as described,

We provide, according to a 5^{th} aspect of the present invention, a method of diagnosis of aggressive breast cancer in an individual, the method comprising assigning a grade indicative of high aggressiveness to a breast tumour of the individual by a method as described.

The present invention, in a 6^{th} aspect, provides a method of choosing a therapy for an individual with breast cancer, the method comprising assigning a grade to the breast tumour by a method as described, and choosing an appropriate therapy based on the aggressiveness of the breast tumour.

In a 7^{th} aspect of the present invention, there is provided a method of treatment of an individual with breast cancer, the method comprising assigning a grade to the breast tumour by a method as described, and administering an appropriate therapy to the individual based on the aggressiveness of the breast tumour.

According to an 8^{th} aspect of the present invention, we provide a method of determining the likelihood of success of a particular therapy on an individual with a breast tumour, the method comprising comparing the therapy with the therapy determined by a such a method.

We provide, according to a 9^{th} aspect of the invention, a method of assigning a breast tumour patient into a prognostic group, the method comprising applying the Nottingham Prognostic Index to a breast tumour, in which the histologic grade score of the breast tumour is replaced by a grade obtained by a method as described.

There is provided, in accordance with a 10^{th} aspect of the present invention, a method of assigning a breast tumour patient into a prognostic group, the method comprising deriving a score which is the sum of the following: (a) (0.2× tumour size in cm); (b) tumour grade in which the tumour grade is assigned by a method as described; and (c) lymph node stage; in which the tumour size and the lymph node stage are determined according to the Nottingham Prognostic Index, in which a patient with a score of 2.4 or less is categorised to a EPG (excellent prognostic group), a patient with a score of less than 3.4 is categorised to a GPG (good prognostic group), a patient with a score of between 3.4 and 5.4 is categorised to a MPG (moderate prognostic group), a patient with a score of greater than 5.4 is categorised to a PPG (poor prognostic group).

As an 11^{th} aspect of the invention, we provide a method of determining whether a breast tumour is a metastatic breast tumour, the method comprising assigning a grade to the breast tumour by a method as described.

We provide, according to a 12^{th} aspect of the invention, a method of identifying a molecule capable of treating or preventing breast cancer, the method comprising: (a) grading a breast tumour; (b) exposing the breast tumour to a candidate molecule; and (c) detecting a change in tumour grade; in which the grade or change thereof, or both, is assigned by a method as described.

According to a 13^{th} aspect of the present invention, we provide a molecule identified by such a method.

There is provided, according to a 14^{th} aspect of the present invention, use of such a molecule in a method of treatment or prevention of cancer in an individual.

We provide, according to a 15^{th} aspect of the present invention, a method of treatment of an individual suffering from breast cancer, the method comprising modulating the expression of a gene set out in Table D0 (6g-TAG) or Table D1 (SWS Classifier 0).

According to a 16^{th} aspect of the present invention, we provide a method of determining the proliferative state of a cell, the method comprising detecting the expression of a gene selected from the genes set out in Table D1 (SWS Classifier 0), in which: (a) a high level of expression of a gene which is annotated “3” in Column 7 (“Grade with Higher Expression”) indicates a highly proliferative cell; (b) a high level of expression of a gene which is annotated “1” in Column 7 (“Grade with Higher Expression”) indicates a non-proliferating cell or a slow-growing cell; (c) a low level of expression of a gene which is annotated “3” in Column 8 (“Grade with Lower Expression”) indicates a highly proliferative cell; and (d) a low level of expression of a gene which is annotated “1” in Column 8 (“Grade with Lower Expression”) indicates a non-proliferating cell or a slow-growing cell.

According to a 17^{th} aspect of the present invention, we provide a combination comprising the genes set out in Table D1 (SWS Classifier 0).

We provide, according to an 18^{th} aspect of the present invention, a combination comprising the probesets set out in Table D1 (SWS Classifier 0). According to a 19^{th} aspect of the present invention, we provide a combination comprising the genes set out in the above aspects of the invention. As an 20^{th} aspect of the invention, we provide a combination comprising the probesets set out in the above aspects of the invention. According to a 21^{st} aspect of the present invention, we provide a combination according to any of the above aspects of the invention in the form of an array. According to a 21^{st} aspect of the present invention, we provide a combination according to the above aspects of the invention in the form of a microarray.

There is provided, according to a 22^{nd} aspect of the present invention, a kit comprising such a combination, array or microarray, together with instructions for use in a method as described. We provide, according to a 23^{rd} aspect of the present invention, use of such a combination, array or a microarray or kit in a method as described.

The method may comprise a method of assigning a grade to a breast tumour as described.

As a 24^{th} aspect of the present invention, there is provided a computer implemented method of assigning a grade to a breast tumour, the method comprising processing expression data for one or more genes set out in Table D1 (SWS Classifier 0) and obtaining a grade indicative of aggressiveness of the breast tumour.

We provide, according to a 25^{th} aspect of the present invention, a program storage device readable by a machine, tangibly embodying a program of instructions executable by the machine to perform method of assigning a grade to a breast tumour, the method comprising: processing expression data for one or more genes set out in Table D1 (SWS Classifier 0); and obtaining a grade indicative of aggressiveness of the breast tumour.

According to a 1^{st} aspect of the present invention, we provide a method of assigning a grade to a breast tumour, which grade is indicative of the aggressiveness of the tumour, the method comprising detecting the expression of a gene selected from the genes set out in Table D1 (SWS 0 Classifier).

There is provided, according to a 2^{nd} aspect of the present invention, a method of classifying a histological Grade 2 tumour into a low aggressiveness tumour or a high aggressiveness tumour, the method comprising assigning a grade to the histological Grade 2 tumour according to the 1^{st} aspect of the invention.

We provide, according to a 3^{rd} aspect of the present invention, a method of predicting a survival rate for an individual with a histological Grade 2 breast tumour, the method comprising assigning a grade to the breast tumour by a method according to the 1^{st} or 2^{nd} aspect of the invention.

As a 4^{th} aspect of the present invention, there is provided a method of prognosis of an individual with a breast tumour, the method comprising assigning a grade to the breast tumour by a method according to the 1^{st} aspect of the invention.

We provide, according to a 5^{th} aspect of the present invention, a method of diagnosis of aggressive breast cancer in an individual, the method comprising assigning a grade indicative of high aggressiveness to a breast tumour of the individual by a method according to the 1^{st} aspect of the invention.

The present invention, in a 6^{th} aspect, provides a method of choosing a therapy for an individual with breast cancer, the method comprising assigning a grade to the breast tumour by a method according the 1^{st} aspect of the invention, and choosing an appropriate therapy based on the aggressiveness of the breast tumour.

In a 7^{th} aspect of the present invention, there is provided a method of treatment of an individual with breast cancer, the method comprising assigning a grade to the breast tumour by a method according to the 1^{st} aspect of the invention, and administering an appropriate therapy to the individual based on the aggressiveness of the breast tumour.

According to an 8^{th} aspect of the present invention, we provide a method of determining the likelihood of success of a particular therapy on an individual with a breast tumour, the method comprising comparing the therapy with the therapy determined.

We provide, according to a 9^{th} aspect of the invention, a method of assigning a breast tumour patient into a prognostic group, the method comprising applying the Nottingham Prognostic Index to a breast tumour, in which the histologic grade score of the breast tumour is replaced by a grade obtained by a method according to the 1^{st} aspect of the invention.

There is provided, in accordance with a 10^{th} aspect of the present invention, a method of assigning a breast tumour patient into a prognostic group, the method comprising deriving a score which is the sum of the following: (a) (0.2× tumour size in cm); (b) tumour grade in which the tumour grade is assigned by a method according to the 1^{st} aspect of the invention, and (c) lymph node stage; in which the tumour size and the lymph node stage are determined according to the Nottingham Prognostic Index, in which a patient with a score of 2.4 or less is categorised to a EPG (excellent prognostic group), a patient with a score of less than 3.4 is categorised to a GPG (good prognostic group), a patient with a score of between 3.4 and 5.4 is categorised to a MPG (moderate prognostic group), and a patient with a score of greater than 5.4 is categorised to a PPG (poor prognostic group).

As an 11^{th} aspect of the invention, we provide a method of determining whether a breast tumour is a metastatic breast tumour, the method comprising assigning a grade to the breast tumour by a method according to the 1^{st} aspect of the invention.

According to a 12^{th} aspect of the present invention, we provide a method of identifying a molecule capable of treating or preventing breast cancer, the method comprising (a) grading a breast tumour; (b) exposing the breast tumour to a candidate molecule; and (c) detecting a change in tumour grade, in which the grade is assigned by a method according to the 1^{st} aspect of the invention.

There is provided, according to a 13^{th} aspect of the present invention, a molecule identified by such a method.

We provide, according to a 14th aspect of the present invention, a method of treatment of an individual suffering from breast cancer, the method comprising modulating the expression of a gene set out in Table D1 (SWS 0 Classifier).

According to a 15^{th} aspect of the present invention, we provide a method of determining the proliferative state of a cell, the method comprising detecting the expression of a gene selected from the genes set out in Table D1 (SWS 0 Classifier), in which: (a) a high level of expression of a gene which is annotated “3” in Column 7 indicates a highly proliferative cell; (b) a high level of expression of a gene which is annotated “1” in Column 7 indicates a non-proliferating cell or a slow-growing cell; (c) a low level of expression of a gene which is annotated “3” in Column 8 indicates a highly proliferative cell; and (d) a low level of expression of a gene which is annotated “1” in Column 8 indicates a non-proliferating cell or a slow-growing cell.

According to a 16^{th} aspect of the present invention, we provide an array, preferably a microarray, comprising the genes set out in Table D1 (SWS 0 Classifier).

We provide, according to a 17^{th} aspect of the present invention, an array, preferably a microarray, comprising the probesets set out in Table D1 (SWS 0 Classifier).

According to an 18^{th} aspect of the present invention, we provide use of an array or microarray according to the 16^{th} or 17^{th} aspect of the invention in a method of assigning a grade to a breast tumour.

As a 19^{th} aspect of the invention, we provide such a use, in which the method comprises the 1^{st} aspect of the invention.

According to a 20^{th} aspect of the present invention, we provide a computer implemented method of assigning a grade to a breast tumour, the method comprising processing expression data for one or more genes set out in Table D1 (SWS 0 Classifier) and obtaining a grade indicative of aggressiveness of the breast tumour.

There is provided, according to a 21^{st} aspect of the present invention, use of Statistically Weighted Syndromes (SWS) on gene expression data, preferably microarray gene expression data.

We provide, according to a 22^{nd} aspect of the present invention, use of Statistically Weighted Syndromes (SWS) for gene discovery.

As a 23^{rd} aspect of the present invention, there is provided such use in combination with Prediction Analysis of Microarrays (PAM).

We provide, according to a 24^{th} aspect of the present invention, use of Statistically Weighted Syndromes (SWS) in combination with Prediction Analysis of Microarrays (PAM) to identify gene sets diagnostic of cancer status, preferably breast cancer status, or proliferative status.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855, Lars-Inge Larsson “Immunocytochemistry: Theory and Practice”, CRC Press Inc., Baca Raton, Fla., 1988, ISBN 0-8493-6078-1, John D. Pound (ed.); “Immunochemical Protocols, vol. 80”, in the series: “Methods in Molecular Biology”, Humana Press, Totowa, N.J., 1998, ISBN 0-89603-493-3, Handbook of Drug Screening, edited by Ramakrishna Seethala, Prabhavathi B. Fernandes (2001, New York, N.Y., Marcel Dekker, ISBN 0-8247-0562-9); Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3; and The Merck Manual of Diagnosis and Therapy (17th Edition, Beers, M. H., and Berkow, R, Eds, ISBN: 0911910107, John Wiley & Sons). Each of these general texts is herein incorporated by reference.

### BRIEF DESCRIPTION OF THE FIGURES

FIG. 1. Schema of discovery and validation of the genetic G2a and G2b breast cancer groups. SWS: Statistically Weighted Syndromes method; PAM: Prediction Analysis for Microarray method; CER: Class Error Rate Function; p.s. probe set: G1: Grade 1; G3: Grade 2; G3: Grade 3; G2a: Grade 2a; G2b: Grade 2b; GO: gene ontology.

FIGS. 2A-2F. Probability (Pr) scores from the SWS classifier. Pr scores (0-1) generated by the class prediction algorithm are shown on the y-axes. Number of tumours per classification exercise is shown on the x-axis. Grade 1 tumours and Grade 3 tumours are indicated in FIGS. 2A, 2C, and 2E.

FIGS. 3A-3F. Survival differences between G2a and G2b genetic grade subtypes. Kaplan-Meier survival curves for G2a and G2b subtypes are shown superimposed on survival curves of histologic grades 1, 2, and 3 (see key). Uppsala cohort survival curves are shown for all patients (FIG. 3A), patients who did not receive systemic therapy (FIG. 3B), patients treated with systemic therapy (FIG. 3C), and patients with ER+ disease who received anti-estrogen therapy only (FIG. 3D). Stockholm cohort survival curves are shown for patients treated with systemic therapy (FIG. 3E) and those with ER+ cancer treated with anit-estrogen therapy only (FIG. 3F). The p-value (likelihood ratio test) reflects the significance of the hazard ratio between the G2a and G2b curves.

FIG. 4. Expression profiles of the top 264 grade (G1-G3) associated gene probesets. Gene probesets (rows) and tumours (columns) were hierarchically clustered by average linkage (Pearson correlation), then tumours were grouped according to grade while maintaining original cluster order within groups. Red reflects above mean expression, green denotes below mean expression, and black indicates mean expression. The degree of color saturation reflects the magnitude of expression relative to the mean.

FIGS. 5A-5L. Statistical analysis of clinicopathological markers. Measurements (or percentages of binary measurements) of clinicopathological variables assessed at the time of surgery were compared between different tumour subgroups: G1 vs. G2a, G2a vs. G2b, and G2b vs. G3. P-values are noted below subgroup designations. Average scores (or percentages) within each subgroup are shown as vertical bars with standard deviations.

FIGS. 6A-6D. Stratification of patient risk by classic NPI and ggNPI. (FIG. 6A) Kaplan-Meier survival curves are shown for the classic NPI categories: Good Prognostic Group (GPG); Moderate Prognostic Group (MPG); Poor Prognostic Group (PPG). (FIG. 6B) Kaplan-Meier survival curves are shown for risk groups determined by the classic NPI (black curves) and the NPI calculated with genetic grade assignments (ggNPI; gray curves). (FIG. 6C) Kaplan-Meier survival curves are shown for patients reclassified by ggNPI (gray curves indicate that reclassified patients have survival curves similar to the good, moderate and poor prognostic groups of the classic NPI (black curves)). (FIG. 6D) The disease-specific survival curves of node negative, untreated patients classified into the Excellent Prognostic Group (EPG) by classic NPI (black curve) or ggNPI (gray curve) are compared.

FIGS. 7A and 7B. Classification of Uppsala and Stockholm G3 tumours, showing SWS probability score (FIG. 7A) and SWS probability score scaled to a threshold of >0.8 for G1-like tumours (FIG. 7B).

FIGS. 8A(1)-8C depict 6 TAGs genes as early diagnostic biomarkers in breast cancer. FIGS. 8A(1) and 8A(2) show gene expression values before and after cross normalization for matched pair samples in GSE10780 dataset. The relative mRNA values of 6 TAGs genes are higher in tumour samples in comparison to adjacent normal patient samples. FIGS. 8B(1) and 8B(2) show gene expression values before and after cross normalization for matched pair samples in TCGA datasets. TAGs genes show relatively higher mRNA values in tumour samples compared to adjacent normal tissue of breast cancer patient samples. FIG. 8C represents positive correlation of E2F1 with TAGs genes in breast cancer.

FIG. 9 shows effectiveness of knock down of E2F1 at mRNA levels, relatively compared to control siRNA treated cells. Also notice significant down regulation of mRNA levels of TAGs genes in E2F1 siRNA treated cells relatively compared to control siRNA treated cells.

FIGS. 10A(1)-10A(7) and FIGS. 10B(1)-10B(7) represent relative mean intensity values of all TAGs genes in G1, G2 and G3 patients along with their respective standard errors in Uppsala and US cohort. FIGS. 10C(1)-10C(7) represent relative mean fold change values of all TAGs genes for G1, G2 and G3 breast cancer patient samples. FIGS. 10C(1)-10C(7) strongly support the view that TAGs genes can strongly discriminate the grade signature at RNA level in various independent breast cancer cohorts. FIG. 10D represents protein levels relatively compared between low grade MCF10A breast cell line (as a model of G1-like BC) and high grade invasive MDA-MB-436 breast cell line (as a model of G3-like BC). FIG. 10E shows that the protein expression of CENPW, AURKA, MELK, PRR11, BRRN1 and E2F1 are relatively low in MCF10A with respect to high grade MDA-MB-436 as analysed by densitometry using ImageJ software.

FIGS. 11A(1)-11A(6) show that each of the 6g-TAGs genes efficiently delineates the grade 2 patients into HG1 like or HG3 like groups in BII-US cohort (GSE61304 dataset) with p<0.01. This phenomenon was also shown using qRT-PCR. FIGS. 11B(1)-11B(6) represent the 6g-TAGs genes and their ability to stratify grade 2 patients into HG1 like and HG3 like sub-classes, that are statistically significant with p value<0.01. FIG. 11C is a diagram showing all 6g-TAGs genes efficiently delineating the grade 2 patients into HG1 like or HG3 like groups in BII-US cohort (GSE61304 dataset) with p<0.01 and high accuracy. This plot could be used for personalization of the aggressiveness of cancers in oncological patient prognostic system.

FIG. 12A represents strong interacting network components of 6g-TAGs genes as hub genes. FIGS. 12B(1)-12B(3) represent comprehensive correlation matrix of 6g-TAGs genes and its interacting network hubs. The negatively correlated genes are indicated in green colour and positively correlated genes are indicated in red font. FIG. 12C depicts qPCR validations of TAGs and its positively correlated network components.

FIGS. 13A(a)-13A(p) depict co-localization experiments of 6g-TAGs genes conducted on breast cancer cell line (MDA-MB-436). The top panel shows co-localization studies of PRR11 and BRRN1 proteins. The blue channel represents DNA (FIG. 13A(a), FIG. 13A(d)), green channel is GFP-PRR11 (FIG. 13A(b)), red channel is BRRN1 protein (FIG. 13A(c)). Notice very nice co-localization of PRR11 and BRRN1 protein in overlap (FIG. 13A(d)). The second panel shows co-localization studies of PRR11 and MELK. Nucleus was stained with DAPI, blue channel (FIG. 13A(e), FIG. 13A(h)) and GFP-PRR11 in green channel (FIG. 13A(f)) and BRRN1 in red channel (FIG. 13A(g)). One can notice clear co-localization of PRR11 and BRRN1 in overlap (FIG. 13A(h)). The third panel represents co-localization studies of BRRN1 and MELK, representing nucleus stained with DAPI in blue channel (FIG. 13A(i), FIG. 13A(l)), BRRN1 in red channel (FIG. 13A(j)) and MELK protein in purple channel (FIG. 13A(k)). The overlap shows strong co-localization of MELK and BRRN1 proteins. The FIG. 13A(h), 13A(l) represents overlap of PRR11, BRRN1 and MELK proteins. The bottom panel shows poor co-localization of BRRN1 and CENPW protein with nucleus stained with DAPI in blue channel (FIG. 13A(m), FIG. 13A(p)), green channel GFP-PRR11 (FIG. 13A(n)) and CENPW in red channel (FIG. 13A(o)). The overlap (FIG. 13A(p)) shows no significant co-localization of PRR11 and CENPW.

FIGS. 13B(a)-13B(d) represent Immunoprecipitation studies using CNBR coupled anti-PRR11 antibody (FIG. 13B(a), FIG. 13B(c), FIG. 13B(d)) and anti-BRRN1 antibody in panel b. The lane 1 represents empty beads to check if any non-specific interactions of proteins to CNBR beads. Lane 2 represents total cell lysates of MDA-MB-436 as positive controls. Lane 3 represents protein complex of BRRN1 (FIG. 13B(a)), MELK (FIG. 13B(c)) and AURKA-A (no interaction) against PRR11. Further notice MELK interaction (FIG. 13B(d)) against BRRN1 protein immunocomplex.

FIGS. 14A-14C represent 6g-TAGs genes RT-PCR experiments conducted on MDA-MB breast cancer cell lines after sorting cells at various cell cycle phases (G1, S, G2/M). FIG. 14A represents high expression of AURKA-A, CENPW, E2F1 and PRR11 in G2/M phase. Other genes did not show significant change at various cell cycle phases. FIG. 14B represents siRNA silencing of 6g-TAGs genes and further assess the cell arrest at various phases of cell cycle. The AURKA-A and CENPW silencing accumulates cells at Mitotic phase relative to control siRNA. E2F1 silencing experiments showed accumulation of cells at S-phase. MELK and BRRN1 silencing showed significant accumulation at G1 phase and PRR11 siRNA silencing experiments showed accumulation of cells at sub-G phase. FIG. 14C shows potential decrease in proliferation upon silencing of 6-g TAGs genes respectively relative to control siRNA in MDA-MB-436 breast cancer cell lines.

FIGS. 15A(1)-15F represent potential prognostic significance of 6-g TAGs genes in Uppsala and BII-US cohort microarray breast cancer datasets. All the 6-g TAGs genes show significant prognostic ability in discriminating breast cancer patients into low and high risk patient samples with significant p-value (FIGS. 15A(1)-15A(7), FIGS. 15B(1)-15B(7)). Further qPCR validation (FIGS. 15C(1)-15C(7)) of 6g TAGs genes on BII-US cohort dataset strongly depicts potential prognostic significance of 6 TAGs genes (p value<0.01). FIG. 15D represents prognostic potential ability of the TAGs genes as a group in stratifying low risk and high risk breast cancer patients. FIG. 15E represents similar studies in BII-US cohort and qPCR validations conducted on BII-US cohort are represented in FIG. 15F.

FIGS. 16A(1)-16D(5) are diagrams showing the expression levels of the 6g-TAG genes in G1, G1-like, G3-like and G3 for Uppsala (FIG. 16A(1)-16A(6)), Stockholm (FIG. 16B(1)-16B(6)), Singapore (FIG. 16C(1)-16C(6)), and Illumina (FIG. 16D(1)-16D(5)) data sets is depicted. Statistical characteristics of these figures strongly demonstrate that G1 and G-like tumours could represent the low-grade BCs and G3-like and G3 tumours could represent high-grade BCs.

FIG. 17 is a diagram showing siRNA analysis of PRR11 functions suggesting apoptotic profile.

FIG. 18 is a diagram showing published experimental datum suggesting that 6g-TAG genes are the periodic cell cycle-related genes

FIGS. 19A-19H show survival prediction analysis for van't Veer-Van De Vijver Nature 2002. Dataset analyzed by SurvExpress software. Patient partition was performed into 2 groups. FIG. 19A: data censored by disease recurrence, FIG. 19B: means and variations of the gene expressions in high and low risk groups. The plot shows expression data for the next genes: PRR11 (NM_018304), NCAPH (D38553), AURKA (NM_003600), CENPW (Contig55997_RC), MELK (NM_014791). FIG. 19C: lymph nodes negative patients, FIG. 19D: lymph nodes positive patients, FIG. 19E: ER negative tumors, FIG. 19F: ER positive tumors, FIG. 19G: patients with no metastases, FIG. 19H: patients with metastases.

FIGS. 20A-20J show survival prediction analysis for Enerly Yakhini Breast GSE19536. Dataset analyzed by SurvExpress software. Patient partition was performed into 2 groups. FIG. 20A: Data censored by disease survival, FIG. 20B: means and variations of the gene expressions in high and low risk groups. The plot shows expression data for the next genes: PRR11 (A_23_P207301) NCAPH (A_23_P415443), AURKA (A_23_P131866), CENPW (A_24_P462899), and MELK (A_23_P94422). FIG. 20C: basal subtype, FIG. 20D: ERBB2 subtype, FIG. 20E: Luminal A subtype, FIG. 20F: Luminal B subtype, FIG. 20G: ER negative tumors, FIG. 20H: ER positive tumors, FIG. 20I: p53 mutation tumors, FIG. 20J: p53 wild type tumors

FIGS. 21A-21D show survival prediction analysis for Dataset: Kao Huang Breast GSE20685. Dataset analyzed by SurvExpress software. Patient partition was performed into 2 groups. FIG. 21A: Data censored by disease survival, FIG. 21B: means and variations of the gene expressions in high and low risk groups. The plot shows expression data for the next genes: PRR11 (228273_at), NCAPH (212949_at), AURKA (204092_s_at), AURKA (208079_s_at), CENPW (226936_at), MELK (204825_at). FIG. 21C: patients with no metastases, FIG. 21D: patients with metastases

FIGS. 22A-22F show survival prediction analysis for Dataset: Wang Foekens Breast GSE2034. Dataset analyzed by SurvExpress software. Patient partition was performed into 2 groups. FIG. 22A: Data censored by relapse free survival, FIG. 22B: means and variations of the gene expressions in high and low risk groups. The plot shows expression data for the next genes: PRR11 (219392_x_at), NCAPH (212949_at), AURKA (204092_s_at), AURKA (208079_s_at), MELK (204825_at). FIG. 22C: lymph nodes negative and ER positive tumors, FIG. 22D: lymph nodes negative patients and ER positive tumors, FIG. 22E: Lymph node negative patients, FIG. 22F: ER negative tumors.

FIGS. 23A and 23B show survival prediction analysis for Dataset: Bos Massaque Breast GSE12276. Dataset analyzed by SurvExpress software. Patient partition was performed into 2 groups. FIG. 23A: Data censored by relapse brain metastases, FIG. 23B: means and variations of the gene expressions in high and low risk groups. The plot shows expression data for the next genes: PRR11 (219392_x_at), NCAPH (212949_at), AURKA (204092_s_at), AURKA (208079_s_at), CENPW (226936_at), MELK (204825_at).

FIGS. 24A and 24B show survival prediction analysis for Shaughnessy Multiple Myeloma GSE2658. Dataset analyzed by SurvExpress software. Patient partition was performed into 2 groups. FIG. 24A: Data censored by disease survival, FIG. 24B: means and variations of the gene expressions in high and low risk groups. The plot shows expression data for the next genes: PRR11 (219392_x_at), NCAPH (212949_at), AURKA (204092_s_at), AURKA (208079_s_at), CENPW (226936_at), and MELK (204825_at).

FIGS. 25A-25E show survival prediction analysis for Kidney renal clear cell carcinoma TCGA. Dataset analyzed by SurvExpress software. Patient partition was performed into 2 groups. FIG. 25A: Data censored by disease survival, FIG. 25B: means and variations of the gene expressions in high and low risk groups. The plot shows expression data for the next genes: PRR11 (228273_at), NCAPH (212949_at), AURKA (204092_s_at), AURKA (208079_s_at), CENPW (226936_at), and MELK (204825_at). FIG. 25C: Grade 2, FIG. 25D: Grade 3, FIG. 25E: Grade 4.

FIGS. 26A-26E show survival prediction analysis for Chibon F, Sarcoma GSE21050. Dataset analyzed by SurvExpress software. Patient partition was performed into 2 groups. FIG. 26A: Data censored by metastasis time, FIG. 26B: means and variations of the gene expressions in high and low risk groups. The plot shows expression data for the next genes: PRR11 (228273_at), NCAPH (212949_at), AURKA (204092_s_at), AURKA (208079 s_at), CENPW (226936_at), and MELK (204825_at). FIG. 26C: Leiomyosarcoma, FIG. 26D: dedifferentiated sarcoma, FIG. 26E: undifferentiated sarcoma.

### DETAILED DESCRIPTION

As used in this specification and the appended claims, the singular forms “a,” “an,” and “the” include plural referents, unless the context clearly dictates otherwise. The terms “a” (or “an”), as well as the terms “one or more,” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges are inclusive of the numbers defining the range. The headings provided herein are not limitations of the various aspects or embodiments of the invention, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are included.

### Breast Tumour Grading

Unless the context indicates otherwise, the following acronyms as used in this document have the indicated meanings: “BC”: Breast cancer; “TAG”: Tumour Aggressive Grading; “6g-TAGs”: 6 gene—Tumour Aggressive Grading signature; “G1”: histologic grade 1; “G2”: histologic grade 2; “G1-like”: histologic grade 1-like; “G3-like”: histologic grade 3-like; G3 histologic grade 3; “GLG”: genetic low grade; “GHG”: genetic high grade; “GG1”: Genetic grade 1; “GG3”: Genetic grade 3; “qRT-PCR”: quantitative reverse transcriptase-polymerase chain reaction; “BII-US”: microarray data generated in Bioinformatics Institute of Singapore.

We have identified a number of genes whose expression is indicative of breast tumour aggressiveness. Accordingly, we provide for methods of grading breast tumours, and therefore assigning a measure of their aggressiveness, by detecting the level of expression of one or more of these genes. The genes are provided in a number of gene sets, or classifiers.

We provide for the detection of and/or the determination of the expression level of at least one, a plurality, or all of the genes of a 6 gene set which we term “6g-TAGs”. The 6 genes of the 6g-TAGs gene set comprise BRRN1, AURKA, MELK, PRR11, CENPW and E2F1 and are set out in Table D0 below.

The GenBank Accession Numbers of each of the genes are as follow: BRRN1 (NM_015341), AURKA (NM_003600), MELK (NM_014791), PRR11 (NM_018304), CENPW (NM_001012507) and E2F1 (NM_005225).

In a general aspect, we provide for the detection of any one or more of a small set of 264 gene probesets, which we term the “SWS Classifier 0”. This classifier represents 232 genes. In some embodiments, the expression of all of the 264 gene probesets are detected. For example, the expression of all the 232 genes represented by such probesets may be detected.

The genes comprised in this classifier are set out in Table D1 in the section “SWS Classifier 0” below, in Table S1 in Example 20, as well as in Appendix A1. This and the other tables D2, D3, D4 and D5 (see below) contain the GenBank ID and the Gene Symbol of the gene, as well as the “Affi ID”, or the “Affymetrix ID” number of a probe. Affymetrix probe set IDs and their corresponding oligonucleotide sequences, as well as the GenBank mRNA sequences they are designed from, can be accessed on the world wide web at the ADAPT website, hosted by the Paterson Institute for Cancer Research. Table D0 also contains this information.

In such an embodiment, therefore, our method comprises determining the expression level of at least one of the genes of the 264 gene probesets (for example, at least one of the 232 genes) in the classifier which we term the “SWS Classifier 0”. More than one, for example, a plurality of the genes of such a set may also be detected. The 264 gene probesets of the SWS Classifier 0 gene set are set out in Table D1 below.

In some embodiments, the expression level of more than one gene is detected. For example, the expression level of 5 or more genes may be detected. The expression level of a plurality of genes may therefore be determined. In some embodiments, the expression level of all 264 gene probesets (for example, the expression level of all 232 genes) may be detected, though it will be clear that this does not need to be so, and a smaller subset may be detected.

We therefore provide for the detection of one or more, a plurality or all, of subsets comprising 17 genes and several subsets of 5-17 genes from the 264 gene probesets.

Alternatively, or in addition, our method may comprise determining the expression level of at least one, a plurality, or all of the genes of a 5 gene set which we term the “SWS Classifier 1”. The 5 genes of the SWS Classifier 1 gene set are set out in Table D2 below.

In other embodiments, our method may comprise determining the expression level of at least one, a plurality, or all of the genes of an 17 gene set which we term the “SWS Classifier 2”. The 17 genes of the SWS Classifier 2 gene set are set out in Table D3 below.

In other embodiments, our method may comprise determining the expression level of at least one, a plurality, or all of the genes of a 7 gene set which we term the “SWS Classifier 3”. The 7 genes of the SWS Classifier 3 gene set are set out in Table D4 below.

In other embodiments, our method may comprise determining the expression level of at least one, a plurality, or all of the genes of a 7 gene set which we term the “SWS Classifier 4”. The 7 genes of the SWS Classifier 4 gene set are set out in Table D5 below.

In specific embodiments, the methods comprise detection of the expression level of all of the genes in the gene set of interest. For example, all 6 genes in the “6g-TAGs” are detected, all 5 genes in the “SWS Classifier 1” are detected, all 17 genes in the “SWS Classifier 2” are detected, all 7 genes in the “SWS Classifier 3” are detected and all 7 genes of the SWS Classifier 4 gene set are detected in these embodiments.

Where the 6g-TAGs, SWS Classifier 1, the SWS Classifier 2, the SWS Classifier 3 or the SWS Classifier 4 are used, each of Tables D0, D2, D3, D4 and D5 provide indications of the grades to be assigned to the tumour depending on the level of expression of the relevant gene which is detected (in Columns 7 and 8 respectively).

Thus, the tables also contain columns showing the grades associated with high and low levels of expression of a particular gene, in Columns 7 and 8 of Table D1 for example. Thus, for example, the gene Barren homolog (Drosophila) is annotated to the effect that the “Grade with Higher Expression” is 3, while the “Grade with Lower Expression” is 1. Accordingly, our method provides that the tumour has a grade of 3 if a high level of expression of Barren homolog (Drosophila) is detected in or from the tumour. If a low level of this gene is detected in or from the tumour, then a grade of 1 may be assigned to that tumour.

Detection of gene expression, for example for tumour grading, may suitably be done by any means as known in the art, and as described in further detail below.

The methods described here for gene expression analysis and tumour grading may be automated, or partially or completely controlled by a controller such as a microcomputer. Thus, any of the methods described here may comprise computer implemented methods of assigning a grade to a breast tumour. For example, such a method may comprise processing expression data for one or more genes set out in Table D1 (SWS 0 Classifier) and obtaining a grade indicative of aggressiveness of the breast tumour.

The methods described here are suitably capable of classifying a breast tumour to an accuracy of at least 85%, at least 90% accuracy, or at least 95% accuracy, with reference to the grade obtained by conventional means, such as for example grading of the breast tumour by histological grading. For example, the methods may be capable of classifying tumours with grades corresponding to histological Grade 1 and histological Grade 3 tumours with an accuracy of 70% or above, 80% or above, or 90% or above.

### Detection of Higher and Lower Expression

In a refinement of our methods, we provide for a “cut-off” level of expression, by which the expression of a gene in or from a tumour may be judged in order to establish whether the expression is at a “high” level, or at a “low” level. The cut-off level is set out in Column 9 of Tables D0, D1, D2, D3, D4 and D5.

Accordingly, in some embodiments, our methods include assigning a grade based on whether the level of expression falls below or exceeds the cut-off. In some embodiments, the cut-off values are determined as the natural log transform normalised signal intensity measurement for Affymetrix arrays. In such embodiments, the cut-off values may be determined as a global mean normalisation with a scaling factor of 500.

For example, referring back to Table 1, the cut-off level of expression for the gene Barren homolog (Drosophila) is 5.9167 units (see above and formula (1), Microarray Method). Where a given tumour contains a level of expression of this gene that exceeds this level, then it is determined to be a “high” level of expression. A grade of 3 may then be assigned to that tumour. On the other hand, if the expression of the Barren homologue falls below this cut-off level, then the expression is judged to be a “low” level of expression. A grade of 1 may be assigned to the tumour in this event.

Thus, we provide for a method which comprises detecting a high level of expression of a gene in SWS Classifier 0 and assigning the grade set out in Column 7 of Table D1 to the breast tumour. The method may comprise, or optionally further comprise detecting a low level of expression of the gene and assigning the grade set out in Column 8 of Table D1 to the breast tumour. A high level of expression may be detected if the expression level of the gene is above the expression level set out in Column 9 of Table D1, and a low level of expression is detected if the expression level of the gene is below that level.

We further provide for a method which comprises detecting a high level of expression of a gene in 6g-TAGs and assigning the grade set out in Column 7 of Table D0 to the breast tumour. The method may comprise, or optionally further comprise detecting a low level of expression of the gene and assigning the grade set out in Column 8 of Table D0 to the breast tumour. A high level of expression may be detected if the expression level of the gene is above the expression level set out in Column 9 of Table D0, and a low level of expression is detected if the expression level of the gene is below that level.

### Detection of High Expression of 6G-Tags Genes

Our methods may comprise detecting a high expression level of any one or more of the 6g-TAGs genes.

Our methods may comprise detecting a high level of expression of BRRN1 (GenBank Accession No. NM_015341), a high level of expression of AURKA (GenBank Accession No. NM_003600), a high level of expression of MELK (GenBank Accession No. NM_014791), a high level of expression of PRR11 (GenBank Accession No. NM_018304), a high level of expression of CENPW (GenBank Accession No. NM_001012507) and/or a high level of expression of E2F1 (GenBank Accession No. NM_005225).

Where a high level of expression of a particular gene or genes is detected, this may be used to establish that a tumour is a high-aggressiveness tumour, e.g., a Grade 3 tumour, or to establish that a tumour is a metastatic tumour, or that cell is a highly proliferative cell, etc, as described in detail in this document.

A high level of expression of any single gene, a pair of the above genes, or a set of three, a set of four, a set of five, or all six of the 6g-TAGs genes may be detected for the purposes of this document.

Our methods may comprise detection of a high level of expression of aurora kinase A. Aurora kinase A (AURKA) has an Entrez_ID of 6790 and a Refseq ID of NM_003600. As the term is used in this document, a “high level of expression” of AURKA is an expression level that is above 6.65262, above 6.30082, above 6.77578. In certain embodiments, a “high level of expression” is an expression level of AURKA that is above 6.576406667. Conversely, a “low level of expression” is an expression level of this gene that is below that level.

The expression of AURKA may be detected for example by use of Affymetrix probe set id 208079_s_at.

Our methods may comprise detection of a high level of expression of centromere protein W. Centromere protein W (CENPW) has an Entrez_ID of 387103 and a Refseq ID of NM_001286524. As the term is used in this document, a “high level of expression” of CENPW is an expression level that is above 7.56154, above 7.40448, above 7.46601. In certain embodiments, a “high level of expression” is an expression level of CENPW that is above 7.477343333. Conversely, a “low level of expression” is an expression level of this gene that is below that level.

The expression of CENPW may be detected for example by use of Affymetrix probe set id 226936_at.

Our methods may comprise detection of a high level of expression of maternal embryonic leucine zipper kinase. Maternal embryonic leucine zipper kinase (MELK) has an Entrez_ID of 9833 and a Refseq ID of NM_014791. As the term is used in this document, a “high level of expression” of MELK is an expression level that is above 7.1069, above 6.63834, above 6.9252. In certain embodiments, a “high level of expression” is an expression level of MELK that is above 6.890146667. Conversely, a “low level of expression” is an expression level of this gene that is below that level.

The expression of MELK may be detected for example by use of Affymetrix probe set id 204825_at.

Our methods may comprise detection of a high level of expression of non-SMC condensin I complex, subunit H. non-SMC condensin I complex, subunit H (NCAPH) has an Entrez_ID of 23397 and a Refseq ID of NM_015341. As the term is used in this document, a “high level of expression” of NCAPH is an expression level that is above 5.91723, above 5.33539, above 5.65104. In certain embodiments, a “high level of expression” is an expression level of NCAPH that is above 5.634553333. Conversely, a “low level of expression” is an expression level of this gene that is below that level.

The expression of NCAPH may be detected for example by use of Affymetrix probe set id 12949_at.

Our methods may comprise detection of a high level of expression of proline rich 11. Proline rich 11 (PRR11/FLJ11029) has an Entrez_ID of 55771 and a Refseq ID of NM_018304. As the term is used in this document, a “high level of expression” of PRR11/FLJ11029 is an expression level that is above 7.70616, above 7.16871, above 7.12064. In certain embodiments, a “high level of expression” is an expression level of PRR11/FLJ11029 that is above 7.331836667. Conversely, a “low level of expression” is an expression level of this gene that is below that level.

The expression of PRR11/FLJ11029 may be detected for example by use of Affymetrix probe set id 228273_at.

Our methods may comprise detection of a high level of expression of E2F transcription factor 1. E2F transcription factor 1 (E2F1) has an Entrez_ID of 1869 and a Refseq ID of NM_005225. As the term is used in this document, a “high level of expression” of E2F1 is an expression level that is above 6.47071, above 5.9933, above 6.48464. In certain embodiments, a “high level of expression” is an expression level of E2F1 that is above 6.316216667. Conversely, a “low level of expression” is an expression level of this gene that is below that level.

The expression of E2F1 may be detected for example by use of Affymetrix probe set id 2028 s_at.

### Detection of Gene Expression

There are various methods by which expression levels of a gene may be detected, and these are known in the art. Examples include RT-PCR, RNAse protection, Northern blotting, Western blotting etc. The gene expression level may be determined at the transcript level, or at the protein level, or both. The detection may be manual, or it may be automated. It is envisaged that any one or a combination of these methods may be employed in the methods and compositions described here.

The detection of expression of a plurality of genes is suitably detected in the form of an expression profile of the plurality of genes, by conventional means known in the art. In some embodiments, the detection is by means of microarray hybridisation.

For example, a sample of a tumour may be taken from a patient and processed for detection of gene expression levels. Gene expression levels may be detected in the form of nucleic acid or protein levels or both, for example. Analysis of nucleic acid expression levels may be suitably performed by amplification techniques, such as polymerase chain reaction (PCR), rolling circle amplification, etc. Detection of expression levels is suitably performed by detecting RNA levels. This can be performed by means known in the art, for example, real time polymerase chain reaction (RT-PCR) or RNAse protection, etc. For this purpose, we provide for sets of one or more primers or primer pairs which are capable of amplifying any one or more of the genes in the classifiers disclosed herein. Specifically, we provide for a set of primer pairs capable of amplifying all of the genes in the SWS Classifier 0, 6g-TAGs, SWS Classifier 1, SWS Classifier 2, SWS Classifier 3 or SWS Classifier 4 sets.

Suitably, RNA expression levels may be detected by hybridisation to a microchip or array, for example, a microchip or array comprising the genes or probesets corresponding to the specific classifier of interest, as described in the Examples. In some embodiments, the gene expression data or profile is derived from microarray hybridisation to for example an Affymetrix microarray.

Detection of protein levels may be performed by for example, immunoassays including ELISA or sandwich immunoassays using antibodies against the protein or proteins of interest (for example as described in U.S. Pat. No. 6,664,114. The detection may be performed by use of a “dip stick” which comprises impregnated antibodies against polypeptides of interest, such as described in US2004014094.

We provide therefore for sets of one or more antibodies which are capable of binding specifically to any one or more of the proteins encoded by the genes in the classifiers disclosed herein. Specifically, we provide for a set of antibodies capable of amplifying all of the genes in the SWS Classifier 0, 6g-TAGs, SWS Classifier 1, SWS Classifier 2, SWS Classifier 3 or SWS Classifier 4 sets.

The grade may be assigned by any suitable method. For example, it may be assigned applying a class prediction algorithm comprising a nearest shrunken centroid method (Tibshirani, et al., 2002, Proc Natl Acad Sci USA. 99(10): 6567-6572) to the expression data of the plurality of genes. The class prediction algorithm may suitably comprise Statistically Weighted Syndromes (SWS) or Prediction Analysis of Microarrays (PAM).

In some embodiments, the grade of the tumour may be assigned by applying a class prediction algorithm comprising one or more of the steps set out here. First, a set of predictor parameters (i.e., probesets) may be obtained based on predictors which discriminate the histologic tumours G1 and G3. Next, the potentially predictive parameters (i.e. signal intensity values of micro-array) may be recoded to obtain cut-off values for robust discrete-valued variables. The recoding may be done in such a way as to maximize an informativity measure of discrimination ability of the parameter and minimize its instability to the discrimination object (i.e. patients) belonging to distinct classes (i.e. G1 and G3). Then, statistically robust discrete-valued variables and combinations thereof may be selected for further construction of class prediction algorithm. A sum of the statistically weighted discrete-valued variables and combinations thereof may be obtained based on the Weighted Voting Procedure procedure described in SWS method section. Finally, a predictive outcome (classification) score of breast cancer subtypes based on the sum for sub-typing (re-classification) histologic G2 tumours may be obtained.

### Application to Grade 2 Tumours

In suitable embodiments, the method is applied to grade breast tumours which are traditionally graded as Grade 2 by conventional means, such as by histological grading as known in the art. Our method is capable of distinguishing the aggressiveness of tumours within the group of tumours in Grade 2 (which were hitherto thought to be homogenous) into Grade 1 like tumours (i.e., more aggressive) and Grade 3 like tumours (i.e., less aggressive). This is described in detail in the Examples.

Accordingly, we provide for a method of classifying a histological Grade 2 tumour into a low aggressiveness tumour or a high aggressiveness tumour. In other words, we provide a method for reassigning a more precise grading to a tumour which has been graded histologically as a Grade 2 tumour.

Such a method comprises assigning a grade to the histological Grade 2 tumour according to any of the methods described above. For example, the expression of any one or more genes, for example, all the genes, in any of the SWS Classifiers described here may be detected and a grade of 1 or 3 assigned using Columns 7, 8 or 9 individually or in combination, as described above.

Such a tumour which has been reassigned will suitably have one or more characteristics or features of the reassigned grade. The characteristics or features may include one or more histological or morphological features, susceptibility to treatment, rate of growth or proliferation, degree of differentiation, aggressiveness, etc. As an example, the characteristic or feature may comprise aggressiveness.

For example, a histological Grade 2 breast tumour which has been assigned a low aggressiveness grade by the gene expression detection methods described here may suitably have at least one feature of a histological Grade 1 breast tumour. Similarly, a breast tumour assigned a high aggressiveness grade may have at least one feature of a histological Grade 3 breast tumour.

Such a feature may comprise degree of differentiation (e.g., well-differentiated, moderately differentiated or poorly-differentiated). The feature may comprise rate of growth (e.g., slow-growing, fast-growing). The feature may comprise rate of proliferation (e.g., slow-proliferation, highly-proliferative). The feature may comprise likelihood of tumour recurrence post-surgery. The feature may comprise survival rate. The feature may comprise likelihood of tumour recurrence post-surgery and survival rate. The feature may comprise a disease free survival rate. The feature may comprise susceptibility to treatment.

Accordingly, application of the grading methods described here enables the classification of the histological Grade 2 tumour into a Grade 1 tumour or a Grade 3 tumour, so as to allow the clinician to treat the tumour accordingly in view of its aggressiveness, prognosis, etc.

Such regrading using our methods is suitably capable of classifying histological Grade 2 tumours into Grade 1 like and/or Grade 3 like tumours with an accuracy of 70% or above, 80% or above, or 90% or above.

The histological grading may be performed by any means known in the art. For example, the breast tissue or tumour may be graded by the Nottingham Grading System (NGS) or the Elston-Ellis Modified Scarf, Bloom, Richardson Grading System, both methods being well known in the art.

The information obtained from the regrading may be used to predict any of the parameters which may be useful to the clinician. The parameter may include, for example, likelihood of tumour metastasis, prognosis of the patient, survival rate, possibility of recovery and recurrence, etc, depending on the grade of the tumour which has been reassigned to the histological Grade 2 tumour. We therefore describe a method of determining whether a breast tumour is a metastatic breast tumour, the method comprising assigning a grade to the breast tumour as described using gene expression data.

We describe a method of predicting a survival rate for an individual with a histological Grade 2 breast tumour, the method comprising assigning a grade to the breast tumour using gene expression data as described. A low aggressiveness grade may suitably indicate a high probability of survival and a high aggressiveness grade may suitably indicate a low probability of survival. We also provide for a method of prognosis of an individual with a breast tumour, the method comprising assigning a grade to the breast tumour by a method as described, and a method of diagnosis of aggressive breast cancer in an individual, the method comprising assigning a grade indicative of high aggressiveness to a breast tumour of the individual by a method as described.

The methods of gene expression analysis may be employed for determining the proliferative state of a cell. For example, such a method may comprise detecting the expression of a gene selected from the genes set out in Table D1 (SWS Classifier 0) and/or a gene selected from the genes set out in Table D0 (6g-TAGs). Where a high level of expression of a gene which is annotated “3” in Column 7 is detected, this may indicate a highly proliferative cell. Similarly, where a high level of expression of a gene which is annotated “1” in Column 7 is detected, a non-proliferating cell or a slow-growing cell may be indicated. If a low level of expression of a gene which is annotated “3” in Column 8 is detected, this may indicate a highly proliferative cell and where a low level of expression of a gene which is annotated “1” in Column 8 is detected, this indicates a non-proliferating cell or a slow-growing cell.

The classifiers are described herein as combinations of probesets, and the skilled person will be aware that more than one probeset can correspond to one gene. Accordingly, the SWS Classifier 0 contains 264 probesets which represent 232 genes. It will be clear therefore that the invention encompasses detection of expression level of one or more genes, and/or one or more probesets within the relevant classifiers, or any combination of this.

Furthermore, it will also be clear that the detection of expression level of one or more genes, and/or one or more probesets within for example a 6g-TAGs geneset is also encompassed.

### Diagnosis and Treatment

Suitably, the information obtained by the regarding may also be used by the clinician to recommend a suitable treatment, in line with the grade of the tumour which has been reassigned.

Thus, a tumour which has been reassigned to Grade 1 may require less aggressive treatment than a tumour which has been reassigned to Grade 3, for example. We therefore describe a method of choosing a therapy for an individual with breast cancer, the method comprising assigning a grade to the breast tumour by a method as described herein, and choosing an appropriate therapy based on the aggressiveness of the breast tumour. In general, the method may be employed for the treatment of an individual with breast cancer, by assigning a grade to the breast tumour and administering an appropriate therapy to the individual based on the aggressiveness of the breast tumour.

In general, we disclose a method of treatment of an individual suffering from breast cancer, the method comprising modulating the expression of a gene set out in Table D0 (6g-TAGs), Table D1 (SWS Classifier 0), Table D2 (SWS 1 Classifier), Table D3 (SWS Classifier 2), Table D4 (SWS Classifier 3) and/or Table D5 (SWS Classifier 4).

Treatment of High-Aggressiveness Tumours

Tumours classified as high-aggressive, such as Grade 3 tumours, may be treated by therapeutic agents that work directly by inhibiting dividing (proliferating) cells.

Such therapeutic agents include chemotherapeutic agents. The chemotherapeutic agent may comprise an antiproliferative chemotherapeutic agent. Examples of chemotherapeutic agents include taxanes such as docetaxel and paclitaxel.

The chemotherapeutic agent may comprise a vinca alkaloid or a condensin inhibitors. The chemotherapeutic agent may comprise vinblastine, vincristine, vindesine, vinorelbine, desoxyvincaminol, vincaminol, vinburnine, vincamajine, vineridine, vinburnine or vinpocetine.

A further example of a chemotherapeutic agent suitable for treating high-aggressive cells is a taxane. Taxanes include paclitaxel (taxol), docetaxel (taxotere) and cabazitaxel.

Inhibitors of AURKA or MELK may also be used as agents for treating high-aggressive cells. An example of an AURKA inhibitor is alisertib. An example of a MELK inhibitor is OTS167.

Further examples of chemotherapeutic agents suitable for treating high-aggressive cells include anthracyclines such as doxorubicin, idarubicin and epirubicin.

These are described in further detail in Joerger M, Thürlimann B. Chemotherapy regimens in early breast cancer: major controversies and future outlook. Expert Rev Anticancer Ther. 2013 February; 13(2):165-78. doi: 10.1586/era.12.172.

Other suitable chemotherapeutic agents may include agents that specifically target cell cycle machinery such as a CDK 4/6 inhibitor. A suitable agent may comprise palbociclib.

Agents suitable for targeting cell cycle machinery are described in detail in Mayer EL. Targeting breast cancer with CDK inhibitors. Curr Oncol Rep. 2015 May; 17(5):443. doi: 10.1007/s11912-015-0443-3.

Treatment of Low-Aggressiveness Tumours

Tumours classified as low-aggressive, such as Grade 3 tumours, are expected to be largely resistant to therapies suitable for treating high-aggressiveness tumours.

Such low-aggressiveness tumours are more suitably treated with agents that do not directly target cell division. Such agents may instead target other growth-related requirements of tumours, such as the mTOR pathway that mediates mRNA translation.

Examples of such therapies suitable for treating low-aggressiveness tumours include everolimus and temsirolimus, described in detail in Vicier C, Dieci M V, Arnedos M, Delaloge S, Viens P, Andre F. Clinical development of mTOR inhibitors in breast cancer. Breast Cancer Res. 2014 Feb. 17; 16(1):203. doi: 10.1186/bcr3618.

Further examples of therapies suitable for treating low-aggressiveness tumours include agents which mediate the growth of blood vessels that provide blood supply to tumours.

An example of such an agent is bevacizumab, described in Keating G M. Bevacizumab: a review of its use in advanced cancer. Drugs. 2014 October; 74(16):1891-925. doi: 10.1007/s40265-014-0302-9.

Other examples of therapeutics suitable for treatment of low-aggressive tumours include agents capable of mediating hormone-related growth signaling pathways such as the estrogen signaling pathways in estrogen receptor-positive breast cancers. Such drugs may comprise tamoxifen, anastrozole, letrozole, exemestane and goserelin. These are described in detail in Schiavon G, Smith I E. Status of adjuvant endocrine therapy for breast cancer. Breast Cancer Res. 2014; 16(2):206.

It will be evident that any of the diagnosis and treatment methods may suitably be combined with other methods of assessing the aggressiveness of the tumour, the patient's health and susceptibility to treatment, etc. For example, the diagnosis or choice of therapy may be determined by further assessing the size of the tumour, or the lymph node stage or both, optionally together or in combination with other risk factors

Specifically, the choice of therapy may be determined by assessing the Nottingham Prognostic Index (NPI). The NPI is described in detail in Haybittle, et al., 1982. In combination with the grading methods described here, the method is suitable for assigning a breast tumour patient into a prognostic group. Such a combined method comprises deriving a score which is the sum of the following: (a) (0.2× tumour size in cm); (b) tumour grade in which the tumour grade is assigned by a method according to any of the gene expression detection methods described herein; and (c) lymph node stage; in which the tumour size and the lymph node stage are determined according to the Nottingham Prognostic Index, in which a patient with a score of 2.4 or less is categorised to a EPG (excellent prognostic group), a patient with a score of less than 3.4 is categorised to a GPG (good prognostic group), a patient with a score of between 3.4 and 5.4 is categorised to a MPG (moderate prognostic group), a patient with a score of greater than 5.4 is categorised to a PPG (poor prognostic group).

Alternatively, or in addition, a method of assigning a breast tumour patient into a prognostic group may comprise applying the Nottingham Prognostic Index to a breast tumour, but modified such that the histologic grade score of the breast tumour is replaced by a grade obtained by a gene expression detection method as described in this document.

Other factors which may of course be assessed for determining the choice of therapy may include receptor status, such as oestrogen receptor (ER) or progesterone receptor (PR) status, as known in the art. For example, the choice of therapy may be determined by further assessing the oestrogen receptor (ER) status of the breast tumour.

### Gene Combinations

We further provide for combinations of genes according to the various classifiers disclosed in this document. Such combinations may comprise mixtures of genes or corresponding probes, such as in a form which is suitable for detection of expression. For example, the combination may be provided in the form of DNA in solution.

In other embodiments, a microarray or chip is provided which comprises any combination of genes or probes, in the form of cDNA, genomic DNA, or RNA, within the classifiers. In some embodiments, the microarray or chip comprises all the genes or probes in 6g-TAGs, SWS Classifier 0, SWS Classifier 1, SWS Classifier 2, SWS Classifier 3 or SWS Classifier 4. The genes may be synthesised or obtained by means known in the art, and attached on the microarray or chip by conventional means, as known in the art. Such microarrays or chips are useful in monitoring gene expression of any one or more of the genes comprised therein, and may be used for tumour grading or detection as described here.

We further describe a probe set consisting of a probe or probes having Affymetrix ID numbers as set out in Column 6 of Table D0, Table D1, Table D2, Table D3, Table D4 or Table D5. Specifically, we describe an array, such as a microarray, comprising the probesets set out in Table D0 (6g-TAGs). We also describe an array such as a microarray comprising the probesets set out in Table D1 (SWS Classifier 0). We also describe an array such as a microarray comprising the genes or probesets set out in Table D2 (SWS 1 Classifier), an array such as a microarray comprising the genes or probesets set out in Table D3 (SWS Classifier 2), an array such as a microarray comprising the genes or probesets set out in Table D4 (SWS3 Classifier), and an array such as a microarray comprising the genes or probesets set out in Table D5 (SWS Classifier 4).

The probes or probe sets are suitably synthesised or made by means known in the art, for example by oligonucleotide synthesis, and may be attached to a microarray for easier carriage and storage. They may be used in a method of assigning a grade to a breast tumour as described herein.

We describe the use of Statistically Weighted Syndromes (SWS) on gene expression data which may comprise microarray gene expression data. We describe the use of SWS for gene discovery. We further describe such use in combination with Prediction Analysis of Microarrays (PAM). We describe the use of SWS to identify gene sets diagnostic of cancer status, such as breast cancer status or proliferative status.

### Screening

The methods and compositions described here may be used for identifying molecules capable of treating or preventing breast cancer, which may be used as drugs for cancer treatment. Such a method comprises: (a) grading a breast tumour as described using gene expression data; (b) exposing the breast tumour to a candidate molecule; and (c) detecting a change in tumour grade. The change in tumour grade is suitably determined by grading a breast tumour as described using gene expression data before and after exposure of the breast tumour to a candidate molecule. We provide molecule identified by such a method, for example for use in breast cancer treatment.

Particular screening applications relate to the testing of pharmaceutical compounds in drug research. The reader is referred generally to the standard textbook “In vitro Methods in Pharmaceutical Research”, Academic Press, 1997, and U.S. Pat. No. 5,030,015). Assessment of the activity of candidate pharmaceutical compounds generally involves combining the breast cancer cells with the candidate compound, determining any change in the tumour grade, as determined by the gene expression detection methods described herein of the cells that is attributable to the compound (compared with untreated cells or cells treated with an inert compound), and then correlating the effect of the compound with the observed change.

The screening may be done, for example, either because the compound is designed to have a pharmacological effect on certain cell types such as tumour cells, or because a compound designed to have effects elsewhere may have unintended side effects. Two or more drugs can be tested in combination (by combining with the cells either simultaneously or sequentially), to detect possible drug-drug interaction effects. In some applications, compounds are screened initially for potential toxicity (Castell et al., pp. 375-410 in “In vitro Methods in Pharmaceutical Research,” Academic Press, 1997). Cytotoxicity can be determined in the first instance by the effect on cell viability, survival, morphology, and expression or release of certain markers, receptors or enzymes. Effects of a drug on chromosomal DNA can be determined by measuring DNA synthesis or repair. [³H]thymidine or BrdU incorporation, especially at unscheduled times in the cell cycle, or above the level required for cell replication, is consistent with a drug effect. The reader is referred to A. Vickers (PP 375-410 in “In vitro Methods in Pharmaceutical Research,” Academic Press, 1997) for further elaboration.

Candidate molecules subjected to the assay and which are found to be of interest may be isolated and further studied. Methods of isolation of molecules of interest will depend on the type of molecule employed, whether it is in the form of a library, how many candidate molecules are being tested at any one time, whether a batch procedure is being followed, etc.

The candidate molecules may be provided in the form of a library. In an embodiment, more than one candidate molecule is screened simultaneously. A library of candidate molecules may be generated, for example, a small molecule library, a polypeptide library, a nucleic acid library, a library of compounds (such as a combinatorial library), a library of antisense molecules such as antisense DNA or antisense RNA, an antibody library etc, by means known in the art. Such libraries are suitable for high-throughput screening. Tumour cells may be exposed to individual members of the library, and the effect on tumour grade, if any, cell determined. Array technology may be employed for this purpose. The cells may be spatially separated, for example, in wells of a microtitre plate.

In an embodiment, a small molecule library is employed. By a “small molecule”, we refer to a molecule whose molecular weight may be less than about 50 kDa. In particular embodiments, a small molecule has a molecular weight may be less than about 30 kDa, such as less than about 15 kDa, or less than 10 kDa or so. Libraries of such small molecules, here referred to as “small molecule libraries” may contain polypeptides, small peptides, for example, peptides of 20 amino acids or fewer, for example, 15, 10 or 5 amino acids, simple compounds, etc.

Alternatively or in addition, a combinatorial library, as described in further detail below, may be screened for candidate modulators of tumour function.

### Combinatorial Libraries

Libraries, in particular, libraries of candidate molecules, may suitably be in the form of combinatorial libraries (also known as combinatorial chemical libraries).

A “combinatorial library”, as the term is used in this document, is a collection of multiple species of chemical compounds that consist of randomly selected subunits. Combinatorial libraries may be screened for molecules which are capable of changing the choice by a stem cell between the pathways of self-renewal and differentiation.

Various combinatorial libraries of chemical compounds are currently available, including libraries active against proteolytic and non-proteolytic enzymes, libraries of agonists and antagonists of G-protein coupled receptors (GPCRs), libraries active against non-GPCR targets (e.g., integrins, ion channels, domain interactions, nuclear receptors, and transcription factors) and libraries of whole-cell oncology and anti-infective targets, among others. A comprehensive review of combinatorial libraries, in particular their construction and uses is provided in Dolle and Nelson (1999), Journal of Combinatorial Chemistry, Vol 1 No 4, 235-282. Reference is also made to Combinatorial peptide library protocols (edited by Shmuel Cabilly, Totowa, N.J.: Humana Press, c1998. Methods in Molecular Biology; v. 87). Specific combinatorial libraries and methods for their construction are disclosed in U.S. Pat. No. 6,168,914 (Campbell, et al), as well as in Baldwin et al. (1995), “Synthesis of a Small Molecule Library Encoded with Molecular Tags,” J. Am. Chem. Soc. 117:5588-5589, and in the references mentioned in those documents.

Further references describing chemical combinatorial libraries, their production and use include The Chemical Generation of Molecular Diversity. Michael R. Pavia, Sphinx Pharmaceuticals, A Division of Eli Lilly (Published July, 1995); Combinatorial Chemistry: A Strategy for the Future—MDL Information Systems discusses the role its Project Library plays in managing diversity libraries (Published July, 1995); Solid Support Combinatorial Chemistry in Lead Discovery and SAR Optimization, Adnan M. M. Mjalli and Barry E. Toyonaga, Ontogen Corporation (Published July, 1995); Non-Peptidic Bradykinin Receptor Antagonists From a Structurally Directed Non-Peptide Library. Sarvajit Chakravarty, Babu J. Mavunkel, Robin Andy, Donald J. Kyle*, Scios Nova Inc. (Published July, 1995); Combinatorial Chemistry Library Design using Pharmacophore Diversity Keith Davies and Clive Briant, Chemical Design Ltd. (Published July, 1995); A Database System for Combinatorial Synthesis Experiments—Craig James and David Weininger, Daylight Chemical Information Systems, Inc. (Published July, 1995); An Information Management Architecture for Combinatorial Chemistry, Keith Davies and Catherine White, Chemical Design Ltd. (Published July, 1995); Novel Software Tools for Addressing Chemical Diversity, R. S. Pearlman, Laboratory for Molecular Graphics and Theoretical Modeling, College of Pharmacy, University of Texas (Published June/July, 1996); Opportunities for Computational Chemists Afforded by the New Strategies in Drug Discovery: An Opinion, Yvonne Connolly Martin, Computer Assisted Molecular Design Project, Abbott Laboratories (Published June/July, 1996); Combinatorial Chemistry and Molecular Diversity Course at the University of Louisville: A Description, Arno F. Spatola, Department of Chemistry, University of Louisville (Published June/July, 1996); Chemically Generated Screening Libraries: Present and Future. Michael R. Pavia, Sphinx Pharmaceuticals, A Division of Eli Lilly (Published June/July, 1996); Chemical Strategies For Introducing Carbohydrate Molecular Diversity Into The Drug Discovery Process. Michael J. Sofia, Transcell Technologies Inc. (Published June/July, 1996); Data Management for Combinatorial Chemistry. Maryjo Zaborowski, Chiron Corporation and Sheila H. DeWitt, Parke-Davis Pharmaceutical Research, Division of Warner-Lambert Company (Published November, 1995); and The Impact of High Throughput Organic Synthesis on R&D in Bio-Based Industries, John P. Devlin (Published March, 1996).

Techniques in combinatorial chemistry are gaining wide acceptance among modern methods for the generation of new pharmaceutical leads (Gallop, M. A. et al., 1994, J. Med. Chem. 37:1233-1251; Gordon, E. M. et al., 1994, J. Med. Chem. 37:1385-1401). One combinatorial approach in use is based on a strategy involving the synthesis of libraries containing a different structure on each particle of the solid phase support, interaction of the library with a soluble receptor, identification of the ‘bead’ which interacts with the macromolecular target, and determination of the structure carried by the identified ‘bead’ (Lam, K. S. et al., 1991, Nature 354:82-84). An alternative to this approach is the sequential release of defined aliquots of the compounds from the solid support, with subsequent determination of activity in solution, identification of the particle from which the active compound was released, and elucidation of its structure by direct sequencing (Salmon, S. E. et al., 1993, Proc. Natl. Acad. Sci. USA 90:11708-11712), or by reading its code (Kerr, J. M. et al., 1993, J. Am. Chem. Soc. 115:2529-2531; Nikolaiev, V. et al., 1993, Pept. Res. 6:161-170; Ohlmeyer, M. H. J. et al., 1993, Proc. Natl. Acad. Sci. USA 90:10922-10926).

Soluble random combinatorial libraries may be synthesized using a simple principle for the generation of equimolar mixtures of peptides which was first described by Furka (Furka, A. et al., 1988, Xth International Symposium on Medicinal Chemistry, Budapest 1988; Furka, A. et al., 1988, 14th International Congress of Biochemistry, Prague 1988; Furka, A. et al., 1991, Int. J. Peptide Protein Res. 37:487-493). The construction of soluble libraries for iterative screening has also been described (Houghten, R. A. et al. 1991, Nature 354:84-86). K. S. Lam disclosed the novel and unexpectedly powerful technique of using insoluble random combinatorial libraries. Lam synthesized random combinatorial libraries on solid phase supports, so that each support had a test compound of uniform molecular structure, and screened the libraries without prior removal of the test compounds from the support by solid phase binding protocols (Lam, K. S. et al., 1991, Nature 354:82-84).

Thus, a library of candidate molecules may be a synthetic combinatorial library (e.g., a combinatorial chemical library), a cellular extract, a bodily fluid (e.g., urine, blood, tears, sweat, or saliva), or other mixture of synthetic or natural products (e.g., a library of small molecules or a fermentation mixture).

A library of molecules may include, for example, amino acids, oligopeptides, polypeptides, proteins, or fragments of peptides or proteins; nucleic acids (e.g., antisense; DNA; RNA; or peptide nucleic acids, PNA); aptamers; or carbohydrates or polysaccharides. Each member of the library can be singular or can be a part of a mixture (e.g., a compressed library). The library may contain purified compounds or can be “dirty” (i.e., containing a significant quantity of impurities).

Commercially available libraries (e.g., from Affymetrix, ArQule, Neose Technologies, Sarco, Ciddco, Oxford Asymmetry, Maybridge, Aldrich, Panlabs, Pharmacopoeia, Sigma, or Tripose) may also be used with the methods described here.

In addition to libraries as described above, special libraries called diversity files can be used to assess the specificity, reliability, or reproducibility of the new methods. Diversity files contain a large number of compounds (e.g., 1000 or more small molecules) representative of many classes of compounds that could potentially result in nonspecific detection in an assay. Diversity files are commercially available or can also be assembled from individual compounds commercially available from the vendors listed above.

### Analysis Method—RNA Purification

The breast tumour is surgically resected, processed, and snap frozen. A frozen portion of the tumour is processed for total RNA extraction using the Qiagen RNeasy kit (Qiagen, Valencia, Calif.). Briefly, frozen tumours are cut into minute pieces, and pieces totalling ˜50-100 milligrams (mg) are homogenized for 40 seconds in RNeasy Lysis Buffer (RLT). Proteinase K is added, and the samples are incubated for 10 minutes at 55 degrees C., followed by centrifugation and the addition of ethanol. After transferring the supernatant into RNeasy columns, DNase is added. Collected RNA is then assessed for quality using an Agilent 2100 bioanalyzer (Agilent Technologies, Rockville, Md.) or by agarose gel. The RNA is stored at minus −70 degrees C.

### Microarray Analysis

Labeled cRNA target is generated for microarray hybridization essentially according to the Affymetrix protocol (Affymetrix, Santa Clara, Calif.). Briefly, approximately 5 micrograms (μg) of total RNA are reversed transcribed into first-strand cDNA using a T7-linked oligo-dT primer, followed by second strand synthesis. A T7 RNA polymerase is then used to linearly amplify antisense RNA. This “cRNA” is biotinylated and chemically fragmented at 95° C. Ten μg of the fragmented, biotinylated cRNA is hybridized at 45° C. for 16 hours to an Affymetrix high-density oligonucleotide GenChip array. The array is then washed and stained with streptavidin-phycoerythrin (10 μg/ml). Signal amplification is achieved using a biotinylated anti-streptavidin antibody. The scanned images are inspected for the presence of artifacts. In case of defects, the hybridization procedure is repeated. Expression values and detection calls are computed from raw data following the procedures outlined for the Affymetrix MAS 5.0 analysis software. Global mean normalization of the gene expression by hybridization signals across all arrays is used to control for differences in chip hybridization signal intensity values. To do that for a given array j (j=1, 2, . . . M), we calculated normalization coefficients kⱼ (j=1, 2, . . . , n), by the following formula:

$${k}_{j} = n * ln ( 500 ) / {\sum }_{i = 1}^{n} ln ( {a}_{ij} ) , \qquad ( 1 )$$

where n is the number of observed probe sets, aᵢⱼ is the signal intensity value of the i-th Affymetrix probesets representing a gene expression. Then the natural logarithm of the signal intensity value of the given array j was multiplied by this normalization coefficient. A normalisation coefficient of 500 is used in determining the cut-offs shown in the Tables in this document.

### SWS Analysis

The microarray-derived normalized numerical expression values corresponding to the genetic grade signature genes are used as input for the SWS algorithm.

### Other Methods

The RNA purification and microarray analysis methodologies above reflect only our “preferred methods”, and that other variants exist that could be used in conjunction with our Process for Predicting Patient Outcome. . . . For example, the starting material could be formalin-fixed paraffin-embedded tumour material instead of fresh frozen material, or the RNA might be extracted using a Cesium Chloride Gradient method, or the RNA could be analyzed by NimbleGen Microarrays that include DNA probes corresponding to our genes of interest. And it should also be noted that a microarray may not be necessary at all to determine the expression levels of our signature genes, but rather their expression could be quantitatively measured by PCR-based techniques such as real time-PCR.

### Classifiers, Gene Sets and Probe Sets

TABLE D0
6G-TAGs

| | | | | | | Grade w/ | Grade w/ | Cut-off |
|---|---|---|---|---|---|---|---|---|
| | Entrez | Gene | Gene | | Blank | Higher | Lower | value by |
| No | ID | Name | Symbol | Refseq ID | Col. 6 | Expr. | Expr. | SWS method |
| 1 | 6790 | Aurora | AURKA | NM_003600 | — | 3 | 1 | 6.576406667 |
| | | kinase A | | | | | | |
| 2 | 387103 | Centromere | CENPW | NM_001286524 | — | 3 | 1 | 7.477343333 |
| | | protein W | | | | | | |
| 3 | 9833 | Maternal | MELK | NM_014791 | — | 3 | 1 | 6.890146667 |
| | | embryonic | | | | | | |
| | | leucine | | | | | | |
| | | zipper | | | | | | |
| | | kinase | | | | | | |
| 4 | 23397 | Non-SMC | NCAPH | NM_015341 | — | 3 | 1 | 5.634553333 |
| | | condensin I | | | | | | |
| | | complex, | | | | | | |
| | | subunit H | | | | | | |
| 5 | 55771 | Proline rich | PRR11/ | NM_018304 | — | 3 | 1 | 7.331836667 |
| | | 11 | FLJ11029 | | | | | |
| 6 | 1869 | E2F | E2F1 | NM_005225 | — | 3 | 1 | 6.316216667 |
| | | transcription | | | | | | |
| | | factor 1 | | | | | | |

Table D0. 6g-TAGs Classifier. For any particular gene, where the value of the cell in column 7 “Grade with Higher Expression” is 3, the value of the cell in column 8 “Grade with Lower Expression” is 1, and where the value of cell “Grade with Higher Expression” is 1, the value of column “Grade with Lower Expression” is 3. Colum 9 shows the cut off (Optimal Variance Cut-off) expressed as the natural log transform normalised signal intensity measurement for Affymetrix arrays (global mean normalisation with a scaling factor of 500).

TABLE D1
SWS CLASSIFIER 0

| | UGID | | | | | Grade w/ | Grade w/ | | | |
|---|---|---|---|---|---|---|---|---|---|---|
| | (build | | Gene | Genbank | | Higher | Lower | Cut- | | Instability |
| Order | #177) | UnigeneName | Symbol | Acc | Affi ID | Expr. | Expr. | Off | Chi-2 | indices |
| 1 | Hs.528654 | Hypothetical | PRR11/ | BG165011 | B.228273_at | 3 | 1 | 7.7063 | 95.973 | 0.011 |
| | | protein | FLJ11029 | | | | | | | |
| | | FLJ11029 | | | | | | | | |
| 2 | acc_NM_003158.1 | Serine/threonine | AURKA/ST | NM_003158 | A.208079_s_at | 3 | 1 | 6.6526 | 95.599 | 0.002 |
| | | kinase 6 | K6 | | | | | | | |
| 3 | Hs.308045 | Barren homolog | BRRN1 | D38553 | A.212949_at | 3 | 1 | 5.9167 | 92.640 | 0.006 |
| | | (Drosophila) | | | | | | | | |
| 4 | Hs.35962 | CDNA clone | CENPW | BG492359 | B.226936_at | 3 | 1 | 7.5619 | 92.601 | 0.003 |
| | | IMAGE: 4452583, | | | | | | | | |
| | | partial cds | | | | | | | | |
| 5 | Hs.184339 | Maternal | MELK | NM_014791 | A.204825_at | 3 | 1 | 7.1073 | 90.110 | 0.002 |
| | | embryonic | | | | | | | | |
| | | leucine zipper | | | | | | | | |
| | | kinase | | | | | | | | |
| 6 | Hs.250822 | Serine/threonine | AURKA/ST | NM_003600 | A.204092_s_at | 3 | 1 | 6.7266 | 88.639 | 0.003 |
| | | kinase 6 | K6 | | | | | | | |
| 7 | Hs.9329 | TPX2, | TPX2 | AF098158 | A.210052_s_at | 3 | 1 | 7.4051 | 86.239 | 0.001 |
| | | microtubule- | | | | | | | | |
| | | associated | | | | | | | | |
| | | protein homolog | | | | | | | | |
| | | (X. laevis) | | | | | | | | |
| 8 | Hs.1594 | Centromere | CENPA | NM_001809 | A.204962_s_at | 3 | 1 | 6.344 | 85.316 | 0.037 |
| | | protein A, 17 kDa | | | | | | | | |
| 9 | Hs.198363 | MCM10 | MCM10 | AB042719 | B.222962_s_at | 3 | 1 | 6.1328 | 85.176 | 0.001 |
| | | minichromosome | | | | | | | | |
| | | maintenance | | | | | | | | |
| | | deficient 10 (S. | | | | | | | | |
| | | cerevisiae) | | | | | | | | |
| 10 | Hs.48855 | Cell division | CDCA8 | BC001651 | A.221520_s_at | 3 | 1 | 5.2189 | 85.152 | 0.018 |
| | | cycle associated | | | | | | | | |
| | | 8 | | | | | | | | |
| 11 | Hs.169840 | TTK protein | TTK | NM_003318 | A.204822_at | 3 | 1 | 6.2397 | 82.242 | 0.017 |
| | | kinase | | | | | | | | |
| 12 | Hs.69360 | Kinesin family | KIF2C | U63743 | A.209408_at | 3 | 1 | 7.3717 | 82.105 | 0.006 |
| | | member 2C | | | | | | | | |
| 13 | Hs.55028 | CDNA clone | | BF111626 | B.228559_at | 3 | 1 | 7.2212 | 82.105 | 0.001 |
| | | IMAGE: 6043059, | | | | | | | | |
| | | partial cds | | | | | | | | |
| 14 | Hs.511941 | Forkhead box | FOXM1 | NM_021953 | A.202580_x_at | 3 | 1 | 6.5827 | 81.868 | 0.001 |
| | | M1 | | | | | | | | |
| 15 | Hs.3104 | Kinesin family | KIF14 | AW183154 | B.236641_at | 3 | 1 | 6.4175 | 81.868 | 0.023 |
| | | member 14 | | | | | | | | |
| 16 | Hs.179718 | V-myb | MYBL2 | NM_002466 | A.201710_at | 3 | 1 | 6.0661 | 79.208 | 0.017 |
| | | myeloblastosis | | | | | | | | |
| | | viral oncogene | | | | | | | | |
| | | homolog (avian)- | | | | | | | | |
| | | like 2 | | | | | | | | |
| 17 | Hs.93002 | Ubiquitin- | UBE2C | NM_007019 | A.202954_at | 3 | 1 | 7.8431 | 79.208 | 0.064 |
| | | conjugating | | | | | | | | |
| | | enzyme E2C | | | | | | | | |
| 18 | Hs.344037 | Protein regulator | | NM_003981 | A.218009_s_at | 3 | 1 | 7.3376 | 79.208 | 0.003 |
| | | PRC1 | | | | | | | | |
| | | of cytokinesis 1 | | | | | | | | |
| 19 | Hs.436187 | Thyroid hormone | TRIP13 | NM_004237 | A.204033_at | 3 | 1 | 7.1768 | 78.981 | 0.091 |
| | | receptor | | | | | | | | |
| | | interactor 13 | | | | | | | | |
| 20 | Hs.408658 | Cyclin E2 | CCNE2 | NM_004702 | A.205034_at | 3 | 1 | 6.2055 | 78.603 | 0.019 |
| 21 | Hs.30114 | Cell division | CDCA3 | BC002551 | B.223307_at | 3 | 1 | 7.8418 | 78.603 | 0.084 |
| | | cycle associated | | | | | | | | |
| | | 3 | | | | | | | | |
| 22 | Hs.84113 | Cyclin-dependent | CDKN3 | AF213033 | A.209714_s_at | 3 | 1 | 6.8414 | 78.554 | 0.005 |
| | | kinase inhibitor 3 | | | | | | | | |
| | | (CDK2- | | | | | | | | |
| | | associated dual | | | | | | | | |
| | | specificity | | | | | | | | |
| | | phosphatase) | | | | | | | | |
| 23 | Hs.279766 | Kinesin family | KIF4A | NM_012310 | A.218355_at | 3 | 1 | 6.6174 | 78.212 | 0.013 |
| | | member 4A | | | | | | | | |
| 24 | Hs.104859 | Hypothetical | DKFZp762E | NM_018410 | A.218726_at | 3 | 1 | 6.3781 | 75.507 | 0.036 |
| | | protein | 1312 | | | | | | | |
| | | DKFZp762E131 | | | | | | | | |
| | | 2 | | | | | | | | |
| 25 | Hs.444118 | MCM6 | MCM6 | NM_005915 | A.201930_at | 3 | 1 | 7.9353 | 75.386 | 0.014 |
| | | minichromosome | | | | | | | | |
| | | maintenance | | | | | | | | |
| | | deficient 6 (MISS | | | | | | | | |
| | | homolog, S. | | | | | | | | |
| | | pombe) (S. | | | | | | | | |
| | | cerevisiae) | | | | | | | | |
| 26 | acc_NM_018123.1 | | | NM_018123 | A.219918_s_at | 3 | 1 | 6.5958 | 75.386 | 0.002 |
| 27 | Hs.287472 | BUB1 budding | BUB1 | AF043294 | A.209642_at | 3 | 1 | 6.0118 | 74.136 | 0.058 |
| | | uninhibited by | | | | | | | | |
| | | benzimidazoles 1 | | | | | | | | |
| | | homolog (yeast) | | | | | | | | |
| 28 | Hs.36708 | BUB1 budding | BUB1B | NM_001211 | A.203755_at | 3 | 1 | 6.68 | 73.453 | 0.007 |
| | | uninhibited by | | | | | | | | |
| | | benzimidazoles 1 | | | | | | | | |
| | | homolog beta | | | | | | | | |
| | | (yeast) | | | | | | | | |
| 29 | Hs.77783 | Membrane- | PKMYT1 | NM_004203 | A.204267_x_at | 3 | 1 | 6.9229 | 73.441 | 0.002 |
| | | associated | | | | | | | | |
| | | tyrosine- and | | | | | | | | |
| | | threonine- | | | | | | | | |
| | | specific cdc2- | | | | | | | | |
| | | inhibitory kinase | | | | | | | | |
| 30 | Hs.446554 | RAD51 homolog | RAD51 | NM_002875 | A.205024_s_at | 3 | 1 | 6.3524 | 73.441 | 0.016 |
| | | (RecA homolog, | | | | | | | | |
| | | E. coli) (S. | | | | | | | | |
| | | cerevisiae) | | | | | | | | |
| 31 | Hs.82906 | CDC20 cell | CDC20 | NM_001255 | A.202870_s_at | 3 | 1 | 7.1291 | 72.984 | 0.108 |
| | | division cycle 20 | | | | | | | | |
| | | homolog (S. | | | | | | | | |
| | | cerevisiae) | | | | | | | | |
| 32 | Hs.252712 | Karyopherin | KPNA2 | NM_002266 | A.201088_at | 3 | 1 | 8.4964 | 72.560 | 0.025 |
| | | alpha 2 (RAG | | | | | | | | |
| | | cohort 1, | | | | | | | | |
| | | importin alpha 1) | | | | | | | | |
| 33 | Hs.3104 | | KIF14 | NM_014875 | A.206364_at | 3 | 1 | 6.1518 | 72.560 | 0.067 |
| 34 | Hs.103305 | Chromobox | | BE514414 | B.226473_at | 3 | 1 | 7.5588 | 72.560 | 0.014 |
| | | homolog 2 (Pc | | | | | | | | |
| | | class homolog, | | | | | | | | |
| | | Drosophila) | | | | | | | | |
| 35 | Hs.152759 | Activator of S | ASK | NM_006716 | A.204244_s_at | 3 | 1 | 5.9825 | 72.294 | 0.018 |
| | | phase kinase | | | | | | | | |
| 36 | acc_AL138828 | | | AL138828 | B.228069_at | 3 | 1 | 7.0119 | 72.294 | 0.084 |
| 37 | Hs.226390 | Ribonucleotide | RRM2 | NM_001034 | A.201890_at | 3 | 1 | 7.1014 | 70.961 | 0.002 |
| | | reductase M2 | | | | | | | | |
| | | polypeptide | | | | | | | | |
| 38 | Hs.445890 | HSPC163 protein | HSPC163 | NM_014184 | A.218728_s_at | 3 | 1 | 7.6481 | 70.764 | 0.003 |
| 39 | Hs.194698 | Cyclin B2 | CCNB2 | NM_004701 | A.202705_at | 3 | 1 | 7.0096 | 70.698 | 0.001 |
| 40 | Hs.234545 | Cell division | CDCA1 | AF326731 | B.223381_at | 3 | 1 | 6.4921 | 70.698 | 0.008 |
| | | cycle associated 1 | | | | | | | | |
| 41 | Hs.16244 | Sperm associated | SPAG5 | NM_006461 | A.203145_at | 3 | 1 | 6.4627 | 70.095 | 0.001 |
| | | antigen 5 | | | | | | | | |
| 42 | Hs.62180 | Anillin, actin | ANLN | AK023208 | B.222608_s_at | 3 | 1 | 6.9556 | 69.641 | 0.013 |
| | | binding protein | | | | | | | | |
| | | (scraps homolog, | | | | | | | | |
| | | Drosophila) | | | | | | | | |
| 43 | Hs.14559 | Chromosome 10 | C10orf3 | NM_018131 | A.218542_at | 3 | 1 | 6.4965 | 69.335 | 0.049 |
| | | open reading | | | | | | | | |
| | | frame 3 | | | | | | | | |
| 44 | Hs.122908 | DNA replication | CDT1 | AW075105 | B.228868_x_at | 3 | 1 | 7.0543 | 69.335 | 0.001 |
| | | factor | | | | | | | | |
| 45 | Hs.8878 | Kinesin family | KIF11 | NM_004523 | A.204444_at | 3 | 1 | 6.4655 | 69.318 | 0.005 |
| | | member 11 | | | | | | | | |
| 46 | Hs.83758 | CDC28 protein | CKS2 | NM_001827 | A.204170_s_at | 3 | 1 | 7.8353 | 69.178 | 0.027 |
| | | kinase regulatory | | | | | | | | |
| | | subunit 2 | | | | | | | | |
| 47 | Hs.112160 | Chromosome 15 | PIF1 | AF108138 | B.228252_at | 3 | 1 | 6.6518 | 69.178 | 0.039 |
| | | open reading | | | | | | | | |
| | | frame 20 | | | | | | | | |
| 48 | Hs.79078 | MAD2 mitotic | MAD2L1 | NM_002358 | A.203362_s_at | 3 | 1 | 6.4606 | 68.044 | 0.038 |
| | | arrest deficient- | | | | | | | | |
| | | like 1 (yeast) | | | | | | | | |
| 49 | Hs.226390 | Ribonucleotide | RRM2 | BC001886 | A.209773_s_at | 3 | 1 | 7.2979 | 67.380 | 0.135 |
| | | reductase M2 | | | | | | | | |
| | | polypeptide | | | | | | | | |
| 50 | Hs.462306 | Ubiquitin- | UBE2S | NM_014501 | A.202779_s_at | 3 | 1 | 6.9165 | 67.359 | 0.013 |
| | | conjugating | | | | | | | | |
| | | enzyme E2S | | | | | | | | |
| 51 | Hs.70704 | Chromosome 20 | C20orf129 | BC001068 | B.225687_at | 3 | 1 | 7.2322 | 67.359 | 0.039 |
| | | open reading | | | | | | | | |
| | | frame 129 | | | | | | | | |
| 52 | Hs.294088 | GAJ protein | GAJ | AY028916 | B.223700_at | 3 | 1 | 5.8432 | 67.299 | 0.005 |
| 53 | Hs.381225 | Kinetochore | Spc24 | AI469788 | B.235572_at | 3 | 1 | 6.7839 | 67.299 | 0.002 |
| | | protein Spc24 | | | | | | | | |
| 54 | Hs.334562 | Cell division | CDC2 | AL524035 | A.203213_at | 3 | 1 | 7.0152 | 66.861 | 0.024 |
| | | cycle 2, G1 to S | | | | | | | | |
| | | and G2 to M | | | | | | | | |
| 55 | Hs.109706 | Hematological | HN1 | NM_016185 | A.217755_at | 3 | 1 | 7.9118 | 66.771 | 0.008 |
| | | and neurological | | | | | | | | |
| | | expressed 1 | | | | | | | | |
| 56 | Hs.23900 | Rac GTPase | RACGAP1 | AU153848 | A.222077_s_at | 3 | 1 | 7.1207 | 66.484 | 0.042 |
| | | activating protein | | | | | | | | |
| | | 1 | | | | | | | | |
| 57 | Hs.77695 | Discs, large | DLG7 | NM_014750 | A.203764_at | 3 | 1 | 6.3122 | 66.411 | 0.001 |
| | | homolog 7 | | | | | | | | |
| | | (Drosophila) | | | | | | | | |
| 58 | Hs.46423 | Histone 1, H4c | HIST1H4F | NM_003542 | A.205967_at | 3 | 1 | 8.3796 | 66.411 | 0.005 |
| 59 | Hs.20830 | Kinesin family | KIFC1 | BC000712 | A.209680_s_at | 3 | 1 | 6.9746 | 66.411 | 0.042 |
| | | member C1 | | | | | | | | |
| 60 | Hs.339665 | Similar to Gastric | | AL135396 | B.225834_at | 3 | 1 | 7.2467 | 66.411 | 0.020 |
| | | cancer up- | | | | | | | | |
| | | regulated-2 | | | | | | | | |
| 61 | Hs.94292 | FLJ23311 | FLJ23311 | NM_024680 | A.219990_at | 3 | 1 | 5.0277 | 66.340 | 0.007 |
| | | protein | | | | | | | | |
| 62 | Hs.73625 | Kinesin family | KIF20A | NM_005733 | A.218755_at | 3 | 1 | 7.2115 | 66.267 | 0.001 |
| | | member 20A | | | | | | | | |
| 63 | Hs.315167 | Defective in | MGC5528 | NM_024094 | A.219000_s_at | 3 | 1 | 6.2835 | 66.267 | 0.002 |
| | | sister chromatid | | | | | | | | |
| | | cohesion | | | | | | | | |
| | | homolog 1 (S. | | | | | | | | |
| | | cerevisiae) | | | | | | | | |
| 64 | Hs.85137 | Cyclin A2 | CCNA2 | NM_001237 | A.203418_at | 3 | 1 | 6.194 | 66.208 | 0.001 |
| 65 | Hs.528669 | Chromosome | HCAP-G | NM_022346 | A.218662_s_at | 3 | 1 | 6.0594 | 66.208 | 0.013 |
| | | condensation | | | | | | | | |
| | | protein G | | | | | | | | |
| 66 | Hs.75573 | Centromere | CENPE | NM_001813 | A.205046_at | 3 | 1 | 5.1972 | 65.474 | 0.002 |
| | | protein E, | | | | | | | | |
| | | 312 kDa | | | | | | | | |
| 67 | acc_BE966146 | RAD51 | | BE966146 | A.204146_at | 3 | 1 | 6.3049 | 65.318 | 0.007 |
| | | associated | | | | | | | | |
| | | protein 1 | | | | | | | | |
| 68 | Hs.334562 | Cell division | CDC2 | D88357 | A.210559_s_at | 3 | 1 | 7.0395 | 64.754 | 0.001 |
| | | cycle 2, G1 to S | | | | | | | | |
| | | and G2 to M | | | | | | | | |
| 69 | Hs.108106 | Ubiquitin-like, | UHRF1 | AK025578 | B.225655_at | 3 | 1 | 7.7335 | 64.754 | 0.024 |
| | | containing PHD | | | | | | | | |
| | | and RING finger | | | | | | | | |
| | | domains, 1 | | | | | | | | |
| 70 | Hs.1578 | Baculoviral TAP | BIRC5 | NM_001168 | A.202095_s_at | 3 | 1 | 6.8907 | 64.566 | 0.090 |
| | | repeat-containing | | | | | | | | |
| | | 5 (survivin) | | | | | | | | |
| 71 | acc_NM_021067.1 | | | NM_021067 | A.206102_at | 3 | 1 | 6.714 | 64.566 | 0.013 |
| 72 | Hs.244723 | Cyclin E1 | CCNE1 | AI671049 | A.213523_at | 3 | 1 | 6.082 | 64.566 | 0.001 |
| 73 | Hs.198363 | MCM10 | MCM10 | NM_018518 | A.220651_s_at | 3 | 1 | 5.6784 | 64.175 | 0.081 |
| | | minichromosome | | | | | | | | |
| | | maintenance | | | | | | | | |
| | | deficient 10 (S. | | | | | | | | |
| | | cerevisiae) | | | | | | | | |
| 74 | Hs.155223 | Stanniocalcin 2 | STC2 | AI435828 | A.203438_at | 1 | 3 | 7.5388 | 63.993 | 0.011 |
| 75 | Hs.25647 | V-fos FBJ | FOS | BC004490 | A.209189_at | 1 | 3 | 8.9921 | 63.898 | 0.162 |
| | | murine | | | | | | | | |
| | | osteosarcoma | | | | | | | | |
| | | viral oncogene | | | | | | | | |
| | | homolog | | | | | | | | |
| 76 | Hs.184601 | Solute carrier | SLC7A5 | AB018009 | A.201195_s_at | 3 | 1 | 7.4931 | 63.584 | 0.011 |
| | | family 7 (cationic | | | | | | | | |
| | | amino acid | | | | | | | | |
| | | transporter, y+ | | | | | | | | |
| | | system), member | | | | | | | | |
| | | 5 | | | | | | | | |
| 77 | Hs.528669 | Chromosome | HCAP-G | NM_022346 | A.218663_at | 3 | 1 | 5.7831 | 63.584 | 0.007 |
| | | condensation | | | | | | | | |
| | | protein G | | | | | | | | |
| 78 | Hs.30114 | Cell division | CDCA3 | NM_031299 | A.221436_s_at | 3 | 1 | 6.1898 | 63.584 | 0.002 |
| | | cycle associated | | | | | | | | |
| | | 3 | | | | | | | | |
| 79 | Hs.296398 | Lysosomal | LAPTM4B | T15777 | A.214039_s_at | 3 | 1 | 9.3209 | 63.330 | 0.001 |
| | | associated | | | | | | | | |
| | | protein | | | | | | | | |
| | | transmembrane 4 | | | | | | | | |
| | | beta | | | | | | | | |
| 80 | Hs.442658 | Aurora kinase B | AURKB | AB011446 | A.209464_at | 3 | 1 | 5.9611 | 63.256 | 0.005 |
| 81 | Hs.6879 | DC13 protein | DC13 | NM_020188 | A.218447_at | 3 | | 7.436 | 63.256 | 0.028 |
| 82 | Hs.78913 | Chemokine (C- | CX3CR1 | U20350 | A.205898_at | 1 | 3 | 6.7764 | 63.223 | 0.014 |
| | | X3-C motif) | | | | | | | | |
| | | receptor 1 | | | | | | | | |
| 83 | Hs.406684 | Sodium channel, | SCN7A | AI828648 | B.228504_at | 1 | 3 | 5.8248 | 63.223 | 0.004 |
| | | voltage-gated, | | | | | | | | |
| | | type VII, alpha | | | | | | | | |
| 84 | Hs.80976 | Antigen | MKI67 | BF001806 | A.212022_s_at | 3 | 1 | 6.7255 | 62.415 | 0.125 |
| | | identified by | | | | | | | | |
| | | monoclonal | | | | | | | | |
| | | antibody Ki-67 | | | | | | | | |
| 85 | Hs.406639 | Hypothetical | LOC146909 | AA292789 | A.222039_at | 3 | 1 | 6.4591 | 62.214 | 0.018 |
| | | protein | | | | | | | | |
| | | LOC146909 | | | | | | | | |
| 86 | Hs.334562 | Cell division | CDC2 | NM_001786 | A.203214_x_at | 3 | 1 | 6.588 | 61.528 | 0.002 |
| | | cycle 2, G1 to S | | | | | | | | |
| | | and G2 to M | | | | | | | | |
| 87 | Hs.23960 | Cyclin B1 | CCNB1 | BE407516 | A.214710_s_at | 3 | 1 | 7.1555 | 60.835 | 0.014 |
| 88 | Hs.445098 | DEP domain | SDP35 | AK000490 | B.222958_s_at | 3 | 1 | 6.8747 | 60.835 | 0.003 |
| | | containing 1 | | | | | | | | |
| 89 | Hs.58241 | Serine/threonine | HSA250839 | NM_018401 | A.219686_at | 1 | 3 | 4.5663 | 60.376 | 0.005 |
| | | kinase 32B | | | | | | | | |
| 90 | Hs.5199 | HSPC150 protein | HSPC150 | AB032931 | B.223229_at | 3 | 1 | 7.3947 | 60.376 | 0.010 |
| | | similar to | | | | | | | | |
| | | ubiquitin- | | | | | | | | |
| | | conjugating | | | | | | | | |
| | | enzyme | | | | | | | | |
| 91 | acc_T58044 | | | T58044 | B.227232_at | 1 | 3 | 8.5021 | 60.376 | 0.003 |
| 92 | Hs.421337 | DEP domain | XTP1 | AK001166 | B.226980_at | 3 | 1 | 5.4977 | 60.356 | 0.034 |
| | | containing 1B | | | | | | | | |
| 93 | Hs.238205 | Chromosome 6 | C6orf115 | AF116682 | B.223361_at | 3 | 1 | 8.7555 | 60.138 | 0.003 |
| | | open reading | | | | | | | | |
| | | frame 115 | | | | | | | | |
| 94 | Hs.27860 | Prostaglandin E | | AW242315 | A.213933_at | 1 | 3 | 7.3561 | 59.754 | 0.257 |
| | | receptor 3 | | | | | | | | |
| | | (subtype EP3) | | | | | | | | |
| 95 | Hs.292511 | Neuro- | NOVA1 | NM_002515 | A.205794_s_at | 1 | 3 | 6.7682 | 59.512 | 0.011 |
| | | oncological | | | | | | | | |
| | | ventral antigen 1 | | | | | | | | |
| 96 | Hs.276466 | Hypothetical | FLJ21062 | NM_024788 | A.219455_at | 1 | 3 | 5.5257 | 59.307 | 0.003 |
| | | protein | | | | | | | | |
| | | FLJ21062 | | | | | | | | |
| 97 | Hs.270845 | Kinesin family | KIF23 | NM_004856 | A.204709_s_at | 3 | 1 | 5.1731 | 59.307 | 0.154 |
| | | member 23 | | | | | | | | |
| 98 | Hs.293257 | Epithelial cell | ECT2 | NM_018098 | A.219787_s_at | 3 | 1 | 6.8052 | 59.307 | 0.000 |
| | | transforming | | | | | | | | |
| | | sequence 2 | | | | | | | | |
| | | oncogene | | | | | | | | |
| 99 | Hs.156346 | Topoisomerase | TOP2A | NM_001067 | A.201292_at | 3 | 1 | 7.2468 | 59.071 | 0.011 |
| | | (DNA) II alpha | | | | | | | | |
| | | 170 kDa | | | | | | | | |
| 100 | Hs.31297 | Cytochrome b | CYBRD1 | AL136693 | B.222453_at | 1 | 3 | 9.3991 | 59.071 | 0.001 |
| | | reductase 1 | | | | | | | | |
| 101 | Hs.414407 | Kinetochore | KNTC2 | NM_006101 | A.204162_at | 3 | 1 | 6.017 | 58.653 | 0.076 |
| | | associated 2 | | | | | | | | |
| 102 | Hs.445098 | DEP domain | SDP35 | AI810054 | B.235545_at | 3 | 1 | 6.2495 | 58.653 | 0.133 |
| | | containing 1 | | | | | | | | |
| 103 | Hs.301052 | Kinesin family | DKFZP434G | NM_031217 | A.221258_s_at | 3 | 1 | 5.3649 | 58.160 | 0.158 |
| | | member 18A | 2226 | | | | | | | |
| 104 | Hs.431762 | Tetratricopeptide | LOC118491 | AW024437 | B.229170_s_at | 1 | 3 | 6.2298 | 58.160 | 0.065 |
| | | repeat domain 18 | | | | | | | | |
| 105 | Hs.24529 | CHK1 | CHEK1 | NM_001274 | A.205394_at | 3 | 1 | 5.6217 | 58.087 | 0.017 |
| | | checkpoint | | | | | | | | |
| | | homolog (S. | | | | | | | | |
| | | pombe) | | | | | | | | |
| 106 | Hs.87507 | BRCA1 | BRIP1 | BF056791 | B.235609_at | 3 | 1 | 7.1489 | 58.087 | 0.011 |
| | | interacting | | | | | | | | |
| | | protein C- | | | | | | | | |
| | | terminal helicase | | | | | | | | |
| | | 1 | | | | | | | | |
| 107 | Hs.348920 | FSH primary | FSHPRH1 | BF793446 | A.214804_at | 3 | 1 | 5.0105 | 57.817 | 0.057 |
| | | response (LRPR1 | | | | | | | | |
| | | homolog, rat) 1 | | | | | | | | |
| 108 | Hs.127797 | CDNA | | AI807356 | B.227350_at | 3 | 1 | 6.8658 | 57.782 | 0.014 |
| | | FLJ11381 fis, | | | | | | | | |
| | | clone | | | | | | | | |
| | | HEMBA1000501 | | | | | | | | |
| 109 | Hs.92458 | G protein- | GPR19 | NM_006143 | A.207183_at | 3 | 1 | 5.2568 | 57.642 | 0.002 |
| | | coupled receptor | | | | | | | | |
| | | 19 | | | | | | | | |
| 110 | Hs.552 | Steroid-5-alpha- | SRD5A1 | BC006373 | A.211056_s_at | 3 | 1 | 6.7605 | 57.642 | 0.001 |
| | | reductase, alpha | | | | | | | | |
| | | polypeptide 1 (3- | | | | | | | | |
| | | oxo-5 alpha- | | | | | | | | |
| | | steroid delta 4- | | | | | | | | |
| | | dehydrogenase | | | | | | | | |
| | | alpha 1) | | | | | | | | |
| 111 | Hs.435733 | Cell division | CDCA7 | AY029179 | B.224428_s_at | 3 | 1 | 7.6746 | 57.642 | 0.021 |
| | | cycle associated | | | | | | | | |
| | | 7 | | | | | | | | |
| 112 | Hs.101174 | Microtubule- | MAPT | NM_016835 | A.203929_s_at | 1 | 3 | 7.7914 | 57.600 | 0.003 |
| | | associated | | | | | | | | |
| | | protein tau | | | | | | | | |
| 113 | Hs.436376 | Synaptotagmin | SYNCRIP | NM_006372 | A.217834_s_at | 3 | 1 | 6.8123 | 57.600 | 0.001 |
| | | binding, | | | | | | | | |
| | | cytoplasmic | | | | | | | | |
| | | RNA interacting | | | | | | | | |
| | | protein | | | | | | | | |
| 114 | Hs.122552 | G-2 and S-phase | GTSE1 | NM_016426 | A.204315_s_at | 3 | 1 | 6.4166 | 57.542 | 0.036 |
| | | expressed 1 | | | | | | | | |
| 115 | Hs.153704 | NIMA (never in | NEK2 | NM_002497 | A.204641_at | 3 | 1 | 7.0017 | 57.542 | 0.036 |
| | | mitosis gene a)- | | | | | | | | |
| | | related kinase 2 | | | | | | | | |
| 116 | Hs.208912 | Chromosome 22 | C22orf18 | NM_024053 | A.218741_at | 3 | 1 | 6.3488 | 56.776 | 0.006 |
| | | open reading | | | | | | | | |
| | | frame 18 | | | | | | | | |
| 117 | Hs.81892 | KIAA0101 | KIAA0101 | NM_014736 | A.202503_s_at | 3 | 1 | 8.2054 | 56.644 | 0.029 |
| 118 | Hs.279905 | Nucleolar and | NUSAP1 | NM_016359 | A.218039_at | 3 | 1 | 7.542 | 56.644 | 0.006 |
| | | spindle | | | | | | | | |
| | | associated | | | | | | | | |
| | | protein 1 | | | | | | | | |
| 119 | Hs.170915 | Hypothetical | FLJ10948 | NM_018281 | A.218552_at | 1 | 3 | 7.9778 | 56.041 | 0.010 |
| | | protein | | | | | | | | |
| | | FLJ10948 | | | | | | | | |
| 120 | Hs.144151 | Transcribed | | AI668620 | B.237339_at | 1 | 3 | 9.6693 | 56.041 | 0.029 |
| | | locus | | | | | | | | |
| 121 | Hs.433180 | DNA replication | Pfs2 | BC003186 | A.221521_s_at | 3 | 1 | 6.3201 | 56.036 | 0.059 |
| | | complex GINS | | | | | | | | |
| | | protein PSF2 | | | | | | | | |
| 122 | Hs.47504 | Exonuclease 1 | EXO1 | NM_003686 | A.204603_at | 3 | 1 | 5.927 | 55.961 | 0.001 |
| 123 | Hs.293257 | Epithelial cell | ECT2 | BG170335 | B.234992_x_at | 3 | 1 | 5.1653 | 55.559 | 0.002 |
| | | transforming | | | | | | | | |
| | | sequence 2 | | | | | | | | |
| | | oncogene | | | | | | | | |
| 124 | Hs.385913 | Acidic (leucine- | ANP32E | NM_030920 | A.208103_s_at | 3 | 1 | 6.2989 | 55.557 | 0.001 |
| | | rich) nuclear | | | | | | | | |
| | | phosphoprotein | | | | | | | | |
| | | 32 family, | | | | | | | | |
| | | member E | | | | | | | | |
| 125 | Hs.44380 | Transcribed locus, weakly | | AA938184 | B.236312_at | 3 | 1 | | 55.557 | 0.007 |
| | | similar to NP_060312.1 | | | | | | | | |
| | | hypothetical protein FLJ20489 | | | | | | | | |
| | | [Homo sapiens] | | | | | | | | |
| 126 | Hs.19322 | Chromosome 9 | LOC89958 | AW250904 | B.225777_at | 3 | 1 | 7.8877 | 55.205 | 0.003 |
| | | open reading | | | | | | | | |
| | | frame 140 | | | | | | | | |
| 127 | Hs.188173 | Lymphoid | | AA572675 | B.232286_at | 1 | 3 | 7.169 | 55.205 | 0.008 |
| | | nuclear protein | | | | | | | | |
| | | related to AF4 | | | | | | | | |
| 128 | Hs.28264 | Chromosome 10 | FLJ90798 | AL049949 | A.212419_at | 1 | 3 | 7.6504 | 55.175 | 0.017 |
| | | open reading | | | | | | | | |
| | | frame 56 | | | | | | | | |
| 129 | Hs.387057 | Hypothetical | FLJ13710 | AK024132 | B.232944_at | 1 | 3 | 6.1947 | 55.175 | 0.034 |
| | | protein | | | | | | | | |
| | | FLJ13710 | | | | | | | | |
| 130 | acc_AL031658 | | | AL031658 | B.232357_at | 1 | 3 | 5.9761 | 54.950 | 0.033 |
| 131 | Hs.286049 | Phosphoserine | PSAT1 | BC004863 | B.223062_s_at | 3 | 1 | 6.1035 | 54.930 | 0.003 |
| | | aminotransferase | | | | | | | | |
| | | 1 | | | | | | | | |
| 132 | Hs.19173 | Nucleoporin | | AI806781 | B.235786_at | 1 | 3 | 7.2856 | 54.930 | 0.037 |
| | | 88 kDa | | | | | | | | |
| 133 | Hs.155223 | Stanniocalcin 2 | STC2 | BC000658 | A.203439_s_at | 1 | 3 | 7.6806 | 54.822 | 0.040 |
| 134 | acc_NM_030896.1 | | | NM_030896 | A.221275_s_at | 1 | 3 | 3.9611 | 54.822 | 0.002 |
| 135 | Hs.101174 | Microtubule- | MAPT | AA199717 | B.225379_at | 1 | 3 | 7.8574 | 54.814 | 0.021 |
| | | associated | | | | | | | | |
| | | protein tau | | | | | | | | |
| 136 | Hs.446680 | Retinoic acid | RAI2 | NM_021785 | A.219440_at | 1 | 3 | 6.6594 | 54.307 | 0.057 |
| | | induced 2 | | | | | | | | |
| 137 | Hs.431762 | Tetratricopeptide | LOC118491 | AW024437 | B.229169_at | 1 | 3 | 5.8266 | 53.649 | 0.002 |
| | | repeat domain | | | | | | | | |
| | | 18 | | | | | | | | |
| 138 | acc_NM_005196.1 | | | NM_005196 | A.207828_s_at | 3 | 1 | 7.237 | 53.119 | 0.007 |
| 139 | acc_T90295 | Arsenic | | T90295 | B.226661_at | 3 | 1 | 6.6825 | 52.825 | 0.002 |
| | | transactivated | | | | | | | | |
| | | protein 1 | | | | | | | | |
| 140 | Hs.42650 | ZW10 interactor | ZWINT | NM_007057 | A.204026_s_at | 3 | 1 | 7.5055 | 52.716 | 0.034 |
| 141 | Hs.6641 | | KIF5C | NM_004522 | A.203130_s_at | 1 | 3 | 7.3214 | 52.703 | 0.013 |
| 142 | Hs.23960 | Cyclin B1 | CCNB1 | N90191 | B.228729_at | 3 | 1 | 6.8018 | 52.606 | 0.031 |
| 143 | Hs.72550 | Hyaluronan- | HMMR | NM_012485 | A.207165_at | 3 | 1 | 6.5885 | 52.400 | 0.066 |
| | | mediated | | | | | | | | |
| | | motility | | | | | | | | |
| | | receptor | | | | | | | | |
| | | (RHAMM) | | | | | | | | |
| 144 | Hs.73239 | Hypothetical | FLJ10901 | NM_018265 | A.219010_at | 3 | 1 | 6.9429 | 52.323 | 0.020 |
| | | protein | | | | | | | | |
| | | FLJ10901 | | | | | | | | |
| 145 | Hs.163533 | V-erb-a erythroblastic leukemia | | AK024204 | B.233498_at | 1 | 3 | | 52.208 | 0.002 |
| | | viral oncogene homolog 4 | | | | | | | | |
| | | (avian) | | | | | | | | |
| 146 | Hs.109706 | Hematological | HN1 | AF060925 | B.222396_at | 3 | 1 | 8.4225 | 52.166 | 0.000 |
| | | and | | | | | | | | |
| | | neurological | | | | | | | | |
| | | expressed 1 | | | | | | | | |
| 147 | Hs.165258 | Nuclear | | AA523939 | B.235739_at | 1 | 3 | 7.1874 | 52.022 | 0.000 |
| | | receptor | | | | | | | | |
| | | subfamily 4, | | | | | | | | |
| | | group A, | | | | | | | | |
| | | member 2 | | | | | | | | |
| 148 | Hs.20575 | Growth arrest- | LOC283431 | H37811 | B.235709_at | 3 | 1 | 6.7278 | 51.899 | 0.010 |
| | | specific 2 like 3 | | | | | | | | |
| 149 | Hs.75678 | FBJ murine | FOSB | NM_006732 | A.202768_at | 1 | 3 | 6.1922 | 51.899 | 0.059 |
| | | osteosarcoma | | | | | | | | |
| | | viral oncogene | | | | | | | | |
| | | homolog B | | | | | | | | |
| 150 | Hs.437351 | Cold inducible | CIRBP | AL565767 | B.225191_at | 1 | 3 | 8.033 | 51.899 | 0.002 |
| | | RNA binding | | | | | | | | |
| | | protein | | | | | | | | |
| 151 | Hs.57101 | MCM2 | MCM2 | NM_004526 | A.202107_s_at | 3 | 1 | 7.861 | 51.655 | 0.273 |
| | | minichromosome | | | | | | | | |
| | | maintenance | | | | | | | | |
| | | deficient 2, | | | | | | | | |
| | | mitotin (S. | | | | | | | | |
| | | cerevisiae) | | | | | | | | |
| 152 | Hs.326736 | Ankyrin repeat | NY-BR-1 | AF269087 | B.223864_at | 1 | 3 | 9.4144 | 51.336 | 0.042 |
| | | domain 30A | | | | | | | | |
| 153 | Hs.298646 | ATPase family, | PRO2000 | AI925583 | B.222740_at | 3 | 1 | 6.8416 | 50.763 | 0.130 |
| | | AAA domain | | | | | | | | |
| | | containing 2 | | | | | | | | |
| 154 | Hs.119192 | H2A histone | H2AFZ | NM_002106 | A.200853_at | 3 | 1 | 8.5896 | 50.108 | 0.008 |
| | | family, member | | | | | | | | |
| | | Z | | | | | | | | |
| 155 | Hs.119960 | PHD finger | PHF19 | BE544837 | B.227211_at | 3 | | 6.3487 | 50.108 | 0.084 |
| | | protein 19 | | | | | | | | |
| 156 | Hs.78619 | Gamma- | GGH | NM_003878 | A.203560_at | 3 | 1 | 6.7708 | 49.945 | 0.006 |
| | | glutamyl | | | | | | | | |
| | | hydrolase | | | | | | | | |
| | | (conjugase, | | | | | | | | |
| | | folylpolygamma | | | | | | | | |
| | | glutamyl | | | | | | | | |
| | | hydrolase) | | | | | | | | |
| 157 | Hs.283532 | Uncharacterized | BM039 | NM_018455 | A.219555_s_at | 3 | 1 | 4.1739 | 49.945 | 0.134 |
| | | bone marrow | | | | | | | | |
| | | protein BM039 | | | | | | | | |
| 158 | Hs.221941 | Cytochrome b | | AI669804 | B.232459_at | 1 | 3 | 7.1171 | 49.945 | 0.015 |
| | | reductase 1 | | | | | | | | |
| 159 | Hs.104019 | Transforming, | TACC3 | NM_006342 | A.218308_at | 3 | 1 | 6.1303 | 49.820 | 0.023 |
| | | acidic coiled- | | | | | | | | |
| | | coil containing | | | | | | | | |
| | | protein 3 | | | | | | | | |
| 160 | acc_AK002203.1 | | | AK002203 | B.226992_at | 1 | 3 | 7.9091 | 49.696 | 0.037 |
| 161 | Hs.28625 | Transcribed | | AI693516 | B.228750_at | 1 | 3 | 7.1249 | 49.554 | 0.055 |
| | | locus | | | | | | | | |
| 162 | Hs.206868 | B-cell | | AU146384 | B.232210_at | 1 | 3 | 8.0948 | 49.554 | 0.002 |
| | | CLL/lymphoma | | | | | | | | |
| | | 2 | | | | | | | | |
| 163 | Hs.75528 | Dynein, | HUMAUAN | AW299538 | B.227081_at | 1 | 3 | 7.0851 | 49.549 | 0.003 |
| | | axonemal, light | TIG | | | | | | | |
| | | intermediate | | | | | | | | |
| | | polypeptide 1 | | | | | | | | |
| 164 | acc_AW271106 | | | AW271106 | B.229490_s_at | 3 | 1 | 6.2222 | 49.544 | 0.017 |
| 165 | Hs.298646 | ATPase family, | PRO2000 | AI139629 | B.235266_at | 3 | 1 | 6.1913 | 49.544 | 0.009 |
| | | AAA domain | | | | | | | | |
| | | containing 2 | | | | | | | | |
| 166 | Hs.303090 | Protein | PPP1R3C | N26005 | A.204284_at | 1 | 3 | 7.0275 | 49.520 | 0.011 |
| | | phosphatase 1, | | | | | | | | |
| | | regulatory | | | | | | | | |
| | | (inhibitor) | | | | | | | | |
| | | subunit 3C | | | | | | | | |
| 167 | Hs.83169 | Matrix | MMP1 | NM_002421 | A.204475_at | 3 | 1 | 7.1705 | 49.410 | 0.028 |
| | | metalloproteinase | | | | | | | | |
| | | 1 (interstitial | | | | | | | | |
| | | collagenase) | | | | | | | | |
| 168 | Hs.441708 | Leucine-rich | MGC45866 | AI638593 | B.230021_at | 3 | 1 | 6.424 | 49.410 | 0.005 |
| | | repeat kinase 1 | | | | | | | | |
| 169 | acc_AV733950 | | | AV733950 | A.201693_s_at | 1 | 3 | 7.9061 | 48.773 | 0.005 |
| 170 | Hs.171695 | Dual specificity | DUSP1 | NM_004417 | A.201041_s_at | 1 | | 9.7481 | 48.672 | 0.003 |
| | | phosphatase 1 | | | | | | | | |
| 171 | Hs.87491 | Thymidylate | TYMS | NM_001071 | A.202589_at | 3 | 1 | 7.8242 | 48.672 | 0.041 |
| | | synthetase | | | | | | | | |
| 172 | Hs.434886 | Cell division | CDCA5 | BE614410 | B.224753_at | 3 | 1 | 4.9821 | 48.488 | 0.106 |
| | | cycle associated | | | | | | | | |
| | | 5 | | | | | | | | |
| 173 | Hs.24395 | Chemokine (C- | CXCL14 | NM_004887 | A.218002_s_at | 1 | 3 | 8.2513 | 48.231 | 0.003 |
| | | X-C motif) | | | | | | | | |
| | | ligand 14 | | | | | | | | |
| 174 | Hs.104741 | T-LAK cell- | TOPK | NM_018492 | A.219148_at | 3 | 1 | 6.4626 | 48.155 | 0.001 |
| | | originated | | | | | | | | |
| | | protein kinase | | | | | | | | |
| 175 | Hs.272027 | F-box protein 5 | FBXO5 | AK026197 | B.234863_x_at | 3 | 1 | 6.935 | 48.155 | 0.037 |
| 176 | Hs.101174 | Microtubule- | MAPT | J03778 | A.206401_s_at | 1 | 3 | 6.4557 | 48.155 | 0.021 |
| | | associated | | | | | | | | |
| | | protein tau | | | | | | | | |
| 177 | Hs.7888 | V-erb-a erythroblastic leukemia | | AW772192 | A.214053_at | 1 | 3 | | 48.155 | 0.029 |
| | | viral oncogene homolog 4 | | | | | | | | |
| | | (avian) | | | | | | | | |
| 178 | Hs.372254 | Lymphoid | | AI033582 | B.244696_at | 1 | 3 | 7.4158 | 48.155 | 0.002 |
| | | nuclear protein | | | | | | | | |
| | | related to AF4 | | | | | | | | |
| 179 | Hs.435861 | Signal peptide, | SCUBE2 | AI424243 | A.219197_s_at | 1 | 3 | 8.3819 | 47.983 | 0.037 |
| | | CUB domain, | | | | | | | | |
| | | EGF-like 2 | | | | | | | | |
| 180 | Hs.385998 | WD repeat and | WDHD1 | AK001538 | A.216228_s_at | 3 | 1 | 4.541 | 47.687 | 0.001 |
| | | HMG-box DNA | | | | | | | | |
| | | binding protein 1 | | | | | | | | |
| 181 | Hs.306322 | Neuron navigator | NAV3 | NM_014903 | A.204823_at | 1 | 3 | 5.8235 | 47.678 | 0.004 |
| | | 3 | | | | | | | | |
| 182 | Hs.21380 | CDNA | | AV709727 | B.225996_at | 1 | 3 | 7.5715 | 47.581 | 0.038 |
| | | FLJ36725 fis, | | | | | | | | |
| | | clone | | | | | | | | |
| | | UTERU2012230 | | | | | | | | |
| 183 | Hs.89497 | Lamin B1 | LMNB1 | NM_005573 | A.203276_at | 3 | 1 | 7.11 | 47.281 | 0.004 |
| 184 | acc_NM_017669.1 | | | NM_017669 | A.219650_at | 3 | 1 | 5.0422 | 47.281 | 0.004 |
| 185 | Hs.12532 | Chromosome 1 | C1orf21 | NM_030806 | A.221272_s_at | 1 | 3 | 5.6228 | 47.104 | 0.066 |
| | | open reading | | | | | | | | |
| | | frame 21 | | | | | | | | |
| 186 | Hs.399966 | Calcium channel, | CACNA1D | BE550599 | A.210108_at | 1 | 3 | 6.2612 | 46.990 | 0.063 |
| | | voltage- | | | | | | | | |
| | | dependent, L | | | | | | | | |
| | | type, alpha 1D | | | | | | | | |
| | | subunit | | | | | | | | |
| 187 | Hs.159264 | Clone 23948 | | U79293 | A.215304_at | 1 | 3 | 6.9317 | 46.990 | 0.066 |
| | | mRNA sequence | | | | | | | | |
| 188 | Hs.212787 | KIAA0303 | KIAA0303 | AW971134 | A.222348_at | 1 | 3 | 4.964 | 46.984 | 0.002 |
| | | protein | | | | | | | | |
| 189 | Hs.325650 | EH-domain | EHD2 | AI417917 | A.221870_at | 1 | 3 | 6.4774 | 46.013 | 0.002 |
| | | containing 2 | | | | | | | | |
| 190 | Hs.388347 | Hypothetical | | AW242720 | B.227550_at | 1 | 3 | 7.657 | 45.314 | 0.001 |
| | | protein | | | | | | | | |
| | | LOC143381 | | | | | | | | |
| 191 | Hs.283853 | MRNA full | | AL360204 | B.232855_at | 1 | 3 | 4.6288 | 45.314 | 0.006 |
| | | length insert | | | | | | | | |
| | | cDNA clone | | | | | | | | |
| | | EUROIMAGE | | | | | | | | |
| | | 980547 | | | | | | | | |
| 192 | Hs.57301 | High mobility | HMGA1 | NM_002131 | A.206074_s_at | 3 | 1 | 7.6723 | 44.940 | 0.001 |
| | | group_at-hook 1 | | | | | | | | |
| 193 | Hs.529285 | Solute carrier | | AA588092 | B.239723_at | 1 | 3 | 6.9222 | 44.838 | 0.052 |
| | | family 40 (iron- | | | | | | | | |
| | | regulated | | | | | | | | |
| | | transporter), | | | | | | | | |
| | | member 1 | | | | | | | | |
| 194 | Hs.252938 | Low density | LRP2 | R73030 | B.230863_at | 1 | | 7.4648 | 44.706 | 0.003 |
| | | lipoprotein- | | | | | | | | |
| | | related protein 2 | | | | | | | | |
| 195 | Hs.552 | Steroid-5-alpha- | SRD5A1 | NM_001047 | A.204675_at | 3 | 1 | 7.1002 | 44.684 | 0.000 |
| | | reductase, alpha | | | | | | | | |
| | | polypeptide 1 (3- | | | | | | | | |
| | | oxo-5 alpha- | | | | | | | | |
| | | steroid delta 4- | | | | | | | | |
| | | dehydrogenase | | | | | | | | |
| | | alpha 1) | | | | | | | | |
| 196 | Hs.156346 | Topoisomerase | TOP2A | NM_001067 | A.201291_s_at | 3 | 1 | 7.3566 | 44.552 | 0.110 |
| | | (DNA) II alpha | | | | | | | | |
| | | 170 kDa | | | | | | | | |
| 197 | Hs.413924 | Chemokine (C- | CXCL10 | NM_001565 | A.204533_at | 3 | 1 | 7.9131 | 44.552 | 0.070 |
| | | X-C motif) | | | | | | | | |
| | | ligand 10 | | | | | | | | |
| 198 | Hs.287466 | CDNA | | AK021990 | B.232699_at | 1 | 3 | 5.8675 | 44.552 | 0.002 |
| | | FLJ11928 fis, | | | | | | | | |
| | | clone | | | | | | | | |
| | | HEMBB1000420 | | | | | | | | |
| 199 | acc_X07868 | | | X07868 | A.202409_at | 1 | 3 | 7.9917 | 44.537 | 0.002 |
| 200 | Hs.101174 | Microtubule- | MAPT | NM_016835 | A.203928_x_at | 1 | 3 | 6.9103 | 44.537 | 0.005 |
| | | associated | | | | | | | | |
| | | protein tau | | | | | | | | |
| 201 | Hs.334828 | Hypothetical | FLJ10719 | BG478677 | A.213008_at | 3 | 1 | 6.4461 | 44.494 | 0.009 |
| | | protein | | | | | | | | |
| | | FLJ10719 | | | | | | | | |
| 202 | Hs.326035 | Early growth | EGR1 | NM_001964 | A.201694_s_at | 1 | 3 | 8.6202 | 44.199 | 0.025 |
| | | response 1 | | | | | | | | |
| 203 | Hs.122552 | G-2 and S-phase | GTSE1 | BF973178 | A.215942_s_at | 3 | 1 | 5.4688 | 44.199 | 0.041 |
| | | expressed 1 | | | | | | | | |
| 204 | Hs.24395 | Chemokine (C- | CXCL14 | AF144103 | B.222484_s_at | 1 | 3 | 9.3366 | 44.199 | 0.006 |
| | | X-C motif) | | | | | | | | |
| | | ligand 14 | | | | | | | | |
| 205 | Hs.102406 | Melanophilin | | AI810764 | B.229150_at | 1 | 3 | 8.078 | 44.199 | 0.031 |
| 206 | Hs.164018 | Leucine zipper | FKSG14 | BC005400 | B.222848_at | 3 | 1 | 6.6517 | 43.845 | 0.001 |
| | | protein FKSG14 | | | | | | | | |
| 207 | Hs.19114 | High-mobility | HMGB3 | NM_005342 | A.203744_at | 3 | 1 | 7.5502 | 43.661 | 0.007 |
| | | group box 3 | | | | | | | | |
| 208 | Hs.103982 | Chemokine (C- | CXCL11 | AF002985 | A.211122_s_at | 3 | 1 | 6.1001 | 43.014 | 0.003 |
| | | X-C motif) | | | | | | | | |
| | | ligand 11 | | | | | | | | |
| 209 | Hs.356349 | Transcribed | ZNF145 | AI492388 | B.228854_at | 1 | 3 | 6.8198 | 43.014 | 0.001 |
| | | locus | | | | | | | | |
| 210 | Hs.1657 | Estrogen receptor | ESR1 | NM_000125 | A.205225_at | 1 | 3 | 7.4943 | 42.966 | 0.188 |
| | | 1 | | | | | | | | |
| 211 | Hs.144479 | Transcribed | | BF433570 | B.237301_at | 1 | 3 | 6.3171 | 42.831 | 0.003 |
| | | locus | | | | | | | | |
| 212 | acc_BF508074 | | | BF508074 | B.240465_at | 1 | 3 | 6.0041 | 42.720 | 0.002 |
| 213 | Hs.326391 | Phytanoyl-CoA | PHYHD1 | AL545998 | B.226846_at | 1 | 3 | 7.2214 | 42.425 | 0.100 |
| | | dioxygenase | | | | | | | | |
| | | domain | | | | | | | | |
| | | containing 1 | | | | | | | | |
| 214 | Hs.338851 | FLJ41238 | FLJ41238 | AW629527 | B.229764_at | 1 | 3 | 6.5319 | 42.334 | 0.033 |
| | | protein | | | | | | | | |
| 215 | Hs.65239 | Sodium channel, | SCN4B | AW026241 | B.236359_at | 1 | 3 | 5.5526 | 42.084 | 0.106 |
| | | voltage-gated, | | | | | | | | |
| | | type IV, beta | | | | | | | | |
| 216 | Hs.88417 | Sushi domain | SUSD3 | AW966474 | B.227182_at | 1 | 3 | 8.195 | 41.808 | 0.015 |
| | | containing 3 | | | | | | | | |
| 217 | Hs.16530 | Chemokine (C-C | CCL18 | Y13710 | A.32128_at | 3 | 1 | 6.2442 | 41.317 | 0.004 |
| | | motif) ligand 18 | | | | | | | | |
| | | (pulmonary and | | | | | | | | |
| | | activation- | | | | | | | | |
| | | regulated) | | | | | | | | |
| 218 | Hs.384944 | Superoxide | SOD2 | X15132 | A.216841_s_at | 3 | 1 | 6.0027 | 41.317 | 0.115 |
| | | dismutase 2, | | | | | | | | |
| | | mitochondrial | | | | | | | | |
| 219 | Hs.406050 | Dynein, | DNALI1 | NM_003462 | A.205186_at | 1 | 3 | 4.2997 | 40.911 | 0.009 |
| | | axonemal, light | | | | | | | | |
| | | intermediate | | | | | | | | |
| | | polypeptide 1 | | | | | | | | |
| 220 | Hs.458430 | N- | NAT1 | NM_000662 | A.214440_at | 1 | 3 | 7.7423 | 40.775 | 0.001 |
| | | acetyltransferase | | | | | | | | |
| | | 1 (arylamine N- | | | | | | | | |
| | | acetyltransferase) | | | | | | | | |
| 221 | Hs.437023 | Nucleoporin | IL4I1 | AI859620 | B.230966_at | 3 | 1 | 6.4289 | 40.567 | 0.041 |
| | | 62 kDa | | | | | | | | |
| 222 | Hs.279905 | Nucleolar and | NUSAP1 | NM_018454 | A.219978_s_at | 3 | 1 | 6.3357 | 40.119 | 0.011 |
| | | spindle | | | | | | | | |
| | | associated | | | | | | | | |
| | | protein 1 | | | | | | | | |
| 223 | Hs.505337 | Claudin 5 | CLDN5 | NM_003277 | A.204482_at | 1 | 3 | 6.1516 | 40.053 | 0.001 |
| | | (transmembrane | | | | | | | | |
| | | protein deleted in | | | | | | | | |
| | | velocardiofacial | | | | | | | | |
| | | syndrome) | | | | | | | | |
| 224 | Hs.44227 | Heparanase | HPSE | NM_006665 | A.219403_s_at | 3 | 1 | 5.2989 | 40.005 | 0.253 |
| 225 | Hs.512555 | Collagen, type | COL14A1 | BF449063 | A.212865_s_at | 1 | 3 | 7.2876 | 39.981 | 0.001 |
| | | XIV, alpha 1 | | | | | | | | |
| | | (undulin) | | | | | | | | |
| 226 | Hs.511950 | Sirtuin (silent | SIRT3 | AF083108 | A.221562_s_at | 1 | 3 | 5.9645 | 39.981 | 0.019 |
| | | mating type | | | | | | | | |
| | | information | | | | | | | | |
| | | regulation 2 | | | | | | | | |
| | | homolog) 3 (S. | | | | | | | | |
| | | cerevisiae) | | | | | | | | |
| 227 | Hs.371357 | RNA binding | | AW338699 | B.241789_at | 1 | 3 | 6.3656 | 39.981 | 0.009 |
| | | motif, single | | | | | | | | |
| | | stranded | | | | | | | | |
| | | interacting | | | | | | | | |
| | | protein | | | | | | | | |
| 228 | Hs.81131 | Guanidinoacetate | GAMT | NM_000156 | A.205354_at | 1 | 3 | 5.9474 | 39.852 | 0.005 |
| | | N- | | | | | | | | |
| | | methyltransferase | | | | | | | | |
| 229 | Hs.158992 | FLJ45983 | | AI631850 | B.240192_at | 1 | 3 | 5.2898 | 39.852 | 0.344 |
| | | protein | | | | | | | | |
| 230 | Hs.104624 | Aquaporin 9 | AQP9 | NM_020980 | A.205568_at | 3 | 1 | 4.9519 | 39.848 | 0.010 |
| 231 | Hs.437867 | Homo sapiens, | | AW970881 | A.222314_x_at | 1 | 3 | 5.2505 | 39.816 | 0.042 |
| | | clone | | | | | | | | |
| | | IMAGE: 5759947, | | | | | | | | |
| | | mRNA | | | | | | | | |
| 232 | Hs.296049 | Microfibrillar- | MFAP4 | R72286 | A.212713_at | 1 | 3 | 6.5149 | 39.749 | 0.001 |
| | | associated | | | | | | | | |
| | | protein 4 | | | | | | | | |
| 233 | Hs.109439 | Osteoglycin | OGN | NM_014057 | A.218730_s_at | 1 | 3 | 4.9325 | 39.749 | 0.015 |
| | | (osteoinductive | | | | | | | | |
| | | factor, mimecan) | | | | | | | | |
| 234 | Hs.29190 | Hypothetical | MGC24047 | AI732488 | B.229381_at | 1 | 3 | 7.2281 | 39.749 | 0.069 |
| | | protein | | | | | | | | |
| | | MGC24047 | | | | | | | | |
| 235 | Hs.252418 | Elastin | ELN | AA479278 | A.212670_at | 1 | 3 | 6.8951 | 39.489 | 0.149 |
| | | (supravalvular | | | | | | | | |
| | | aortic stenosis, | | | | | | | | |
| | | Williams-Beuren | | | | | | | | |
| | | syndrome) | | | | | | | | |
| 236 | Hs.252938 | Low density | LRP2 | NM_004525 | A.205710_at | 1 | 3 | 5.9845 | 39.154 | 0.003 |
| | | lipoprotein- | | | | | | | | |
| | | related protein 2 | | | | | | | | |
| 237 | Hs.32405 | MRNA; cDNA | | AL137566 | B.228554_at | 1 | 3 | 7.1124 | 38.597 | 0.015 |
| | | DKFZp586G032 | | | | | | | | |
| | | 1 (from clone | | | | | | | | |
| | | DKFZp586G032 | | | | | | | | |
| | | 1) | | | | | | | | |
| 238 | Hs.288720 | Leucine rich | LRRC17 | NM_005824 | A.205381_at | 1 | 3 | 7.217 | 38.493 | 0.279 |
| | | repeat containing | | | | | | | | |
| | | 17 | | | | | | | | |
| 239 | Hs.203963 | Helicase, | HELLS | NM_018063 | A.220085_at | 3 | 1 | 5.2886 | 38.493 | 0.001 |
| | | lymphoid- | | | | | | | | |
| | | specific | | | | | | | | |
| 240 | Hs.361171 | Placenta-specific | PLAC9 | AW964972 | B.227419_x_at | 1 | 3 | 6.689 | 38.195 | 0.000 |
| | | 9 | | | | | | | | |
| 241 | Hs.396595 | Flavin containing | FMO5 | AK022172 | A.215300_s_at | 1 | 3 | 4.1433 | 37.488 | 0.002 |
| | | monooxygenase | | | | | | | | |
| | | 5 | | | | | | | | |
| 242 | Hs.105434 | Interferon | ISG20 | NM_002201 | A.204698_at | 3 | 1 | 6.2999 | 37.448 | 0.003 |
| | | stimulated gene | | | | | | | | |
| | | 20 kDa | | | | | | | | |
| 243 | Hs.460184 | MCM4 | MCM4 | X74794 | A.212141_at | 3 | 1 | 6.7292 | 36.577 | 0.176 |
| | | minichromosome | | | | | | | | |
| | | maintenance | | | | | | | | |
| | | deficient 4 (S. | | | | | | | | |
| | | cerevisiae) | | | | | | | | |
| 244 | Hs.169266 | Neuropeptide Y | NPY1R | NM_000909 | A.205440_s_at | 1 | 3 | 5.8305 | 36.029 | 0.011 |
| | | receptor Y1 | | | | | | | | |
| 245 | acc_R38110 | | | R38110 | B.240112_at | 1 | 3 | 5.1631 | 35.441 | 0.021 |
| 246 | Hs.63931 | Dachshund | DACH | A1650353 | B.228915_at | 1 | 3 | 7.6716 | 35.346 | 0.319 |
| | | homolog 1 | | | | | | | | |
| | | (Drosophila) | | | | | | | | |
| 247 | Hs.102541 | Netrin 4 | NTN4 | AF278532 | B.223315_at | 1 | 3 | 8.2693 | 35.233 | 0.132 |
| 248 | Hs.418367 | Neuromedin U | NMU | NM_006681 | A.206023_at | 3 | 1 | 5.1017 | 34.589 | 0.035 |
| 249 | Hs.232127 | MRNA; cDNA | | AL512727 | A.215014_at | 1 | 3 | 4.8334 | 34.570 | 0.035 |
| | | DKFZp547P042 | | | | | | | | |
| | | (from clone | | | | | | | | |
| | | DKFZp547P042) | | | | | | | | |
| 250 | Hs.212088 | Epoxide | EPHX2 | AF233336 | A.209368_at | 1 | 3 | 6.4031 | 34.531 | 0.154 |
| | | hydrolase 2, | | | | | | | | |
| | | cytoplasmic | | | | | | | | |
| 251 | Hs.439760 | Cytochrome | CYP4X1 | AA557324 | B.227702_at | 1 | 3 | 8.5972 | 34.531 | 0.015 |
| | | P450, family 4, | | | | | | | | |
| | | subfamily X, | | | | | | | | |
| | | polypeptide 1 | | | | | | | | |
| 252 | acc_BF513468 | | | BF513468 | B.241505_at | 1 | 3 | 7.1517 | 34.140 | 0.001 |
| 253 | Hs.413078 | Nudix | NUDT1 | NM_002452 | A.204766_s_at | 3 | 1 | 5.6705 | 33.955 | 0.069 |
| | | (nucleoside | | | | | | | | |
| | | diphosphate | | | | | | | | |
| | | linked moiety | | | | | | | | |
| | | X)-type motif 1 | | | | | | | | |
| 254 | acc_AI492376 | | | AI492376 | B.231195_at | 3 | 1 | 5.1967 | 33.602 | 0.029 |
| 255 | acc_AW512787 | | | AW512787 | B.238481_at | 1 | 3 | 8.5117 | 33.572 | 0.005 |
| 256 | Hs.74369 | Integrin, alpha 7 | ITGA7 | AK022548 | A.216331_at | 1 | 3 | 5.1535 | 33.290 | 0.003 |
| 257 | Hs.63931 | Dachshund | DACH | NM_004392 | A.205472_s_at | 1 | 3 | 3.9246 | 33.177 | 0.002 |
| | | homolog 1 | | | | | | | | |
| | | (Drosophila) | | | | | | | | |
| 258 | Hs.225952 | Protein tyrosine | PTPRT | NM_007050 | A.205948_at | 1 | 3 | 6.7634 | 32.152 | 0.190 |
| | | phosphatase, | | | | | | | | |
| | | receptor type, T | | | | | | | | |
| 259 | acc_BF793701 | Musculoskeletal, | | BF793701 | B.226856_at | 1 | 3 | 5.5626 | 31.816 | 0.002 |
| | | embryonic | | | | | | | | |
| | | nuclear protein 1 | | | | | | | | |
| 260 | Hs.283417 | Transcribed | | AI826437 | B.229975_at | 1 | 3 | 6.381 | 31.307 | 0.009 |
| | | locus | | | | | | | | |
| 261 | Hs.21948 | Zinc finger | | H15261 | B.243929_at | 1 | 3 | 4.7165 | 30.259 | 0.144 |
| | | protein 533 | | | | | | | | |
| 262 | Hs.31297 | Cytochrome b | CYBRD1 | NM_024843 | A.217889_s_at | 1 | 3 | 5.6427 | 27.628 | 0.056 |
| | | reductase 1 | | | | | | | | |
| 263 | Hs.180142 | Calmodulin-like | CALML5 | NM_017422 | A.220414_at | 3 | 1 | 5.994 | 27.417 | 0.009 |
| | | 5 | | | | | | | | |
| 264 | Hs.176588 | Cytochrome | CYP4Z1 | AV700083 | B.237395_at | 1 | 3 | 8.7505 | 24.383 | 0.400 |
| | | P450, family 4, | | | | | | | | |
| | | subfamily Z, | | | | | | | | |
| | | polypeptide 1 | | | | | | | | |

Table D1: SWS Classifier 0: 264 Probesets. For any particular gene, where the value of the cell in column 7 “Grade with Higher Expression” is 3, the value of the cell in column 8 “Grade with Lower Expression” is 1, and where the value of cell “Grade with Higher Expression” is 1, the value of column “Grade with Lower Expression” is 3. Colum 9 shows the cut off (Optimal Variance Cut-off) expressed as the natural log transform normalised signal intensity measurement for Affymetrix arrays (global mean normalisation with a scaling factor of 500).

TABLE D2
SWS CLASSIFIER 1

| | | | | | | Grade w/ | Grade w/ | |
|---|---|---|---|---|---|---|---|---|
| | UGID | | Gene | Genbank | | Higher | Lower | |
| No | (build #183) | Unigene Name | Symbol | Acc | Affi ID | Expression | Expression | Cut-off |
| 1 | Hs.528654 | Proline rich 11(PRR11); | PRR11/ | BG165011 | B.228273_at | 3 | 1 | 7.706303 |
| | | Hypothetical protein FLJ11029 | FLJ11029 | | | | | |
| 2 | acc_NM_003158.1 | Serine/threonine kinase 6. | AURKA/ | NM_003158 | A.208079_s_at | 3 | 1 | 6.652593 |
| | | transcript 1 | STK6 | | | | | |
| 3 | Hs.35962 | Centromere protein W, | CENPW | BG492359 | B.226936_at | 3 | 1 | 7.561905 |
| | | transcript variant 4; CDNA clone | | | | | | |
| | | IMAGE: 4452583, partial cds | | | | | | |
| 4 | Hs.308045 | Barren homolog (Drosophila) | BRRN1 | D38553 | A.212949_at | 3 | 1 | 5.916703 |
| 5 | Hs.184339 | Maternal embryonic leucine | MELK | NM_014791 | A.204825_at | 3 | 1 | 7.107259 |
| | | zipper kinase | | | | | | |
| 6 | Hs.250822 | Serine/threonine kinase 6, | AURKA/ | NM_003600 | A.204092_s_at | 3 | 1 | 6.726571 |
| | | transcript 2 | STK6 | | | | | |

Table D2. SWS Classifier 1: 6 Probe Sets (5 Genes). For any particular gene, where the value of the cell in column 7 “Grade with Higher Expression” is 3, the value of the cell in column 8 “Grade with Lower Expression” is 1, and where the value of cell “Grade with Higher Expression” is 1, the value of column “Grade with Lower Expression” is 3. Colum 9 shows the cut off (Optimal Variance Cut-off) expressed as the natural log transform normalised signal intensity measurement for Affymetrix arrays (global mean normalisation with a scaling factor of 500).

TABLE D3
SWS CLASSIFIER 2

| | | | | | | Grade w/ | Grade w/ | |
|---|---|---|---|---|---|---|---|---|
| | UGID | | Gene | | | Higher | Lower | |
| No | (build #183) | Unigene Name | Symbol | GenbankAcc | Affi ID | Expression | Expression | Cut-off |
| 1 | Hs.184339 | Maternal embryonic leucine | MELK | NM_014791 | A.204825_at | 3 | 1 | 5.437105 |
| | | zipper kinase | | | | | | |
| 2 | Hs.308045 | Barren homolog (Drosophila) | BRRN1 | D38553 | A.212949_at | 3 | 1 | 5.504552 |
| 3 | Hs.9329 | TPX2, microtubule-associated | TPX2 | AF098158 | A.210052_s_at | 3 | 1 | 5.872187 |
| | | protein homolog (Xenopus | | | | | | |
| | | laevis) | | | | | | |
| 4 | Hs.486401 | CDNA clone IMAGE: 4452583, | | BG492359 | B.226936_at | 3 | 1 | 7.569926 |
| | | partial cds | | | | | | |
| 5 | Hs.75573 | Centromere protein E, 312 kDa | CENPE | NM_001813 | A.205046_at | 3 | 1 | 6.943423 |
| 6 | Hs.528654 | Hypothetical protein FLJ11029 | FLJ11029 | BG165011 | B.228273_at | 3 | 1 | 7.711138 |
| 7 | acc_NM_003158 | | | NM_003158 | A.208079_s_at | 3 | 1 | 6.571034 |
| 8 | Hs.524571 | Cell division cycle associated 8 | CDCA8 | BC001651 | A.221520_s_at | 3 | 1 | 6.894196 |
| 9 | Hs.239 | Forkhead box M1 | FOXM1 | NM_021953 | A.202580_x_at | 3 | 1 | 5.211513 |
| 10 | Hs.179718 | V-myb myeloblastosis viral | MYBL2 | NM_002466 | A.201710_at | 3 | 1 | 6.269081 |
| | | oncogene homolog (avian)-like 2 | | | | | | |
| 11 | Hs.169840 | TTK protein kinase | TTK | NM_003318 | A.204822_at | 3 | 1 | 8.230804 |
| 12 | Hs.75678 | FBJ murine osteosarcoma viral | FOSB | NM_006732 | A.202768_at | 1 | 3 | 8.761579 |
| | | oncogene homolog B | | | | | | |
| 13 | Hs.25647 | V-fos FBJ murine osteosarcoma | FOS | BC004490 | A.209189_at | 1 | 3 | 7.085984 |
| | | viral oncogene homolog | | | | | | |
| 14 | Hs.524216 | Cell division cycle associated 3 | CDCA3 | NM_031299 | A.221436_s_at | 3 | 1 | 6.29283 |
| 15 | Hs.381225 | Kinetochore protein Spc24 | Spc24 | AI469788 | B.235572_at | 3 | 1 | 6.340503 |
| 16 | Hs.62180 | Anillin, actin binding protein | ANLN | AK023208 | B.222608_s_at | 3 | 1 | 6.84578 |
| | | (scraps homolog, Drosophila) | | | | | | |
| 17 | Hs.434886 | Cell division cycle associated 5 | CDCA5 | BE614410 | B.224753_at | 3 | 1 | 5.290668 |
| 18 | Hs.523468 | Signal peptide, CUB domain, | SCUBE2 | AI424243 | A.219197_s_at | 3 | 1 | 5.792164 |
| | | EGF-like 2 | | | | | | |

Table D3. SWS Classifier 2: 18 Probe Sets (17 Genes). For any particular gene, where the value of the cell in column 7 “Grade with Higher Expression” is 3, the value of the cell in column 8 “Grade with Lower Expression” is 1, and where the value of cell “Grade with Higher Expression” is 1, the value of column “Grade with Lower Expression” is 3. Column 9 shows the cut off (Optimal Variance Cut-off) expressed as the natural log transform normalised signal intensity measurement for Affymetrix arrays (global mean normalisation with a scaling factor of 500).

TABLE D4
SWS CLASSIFIER 3

| | | | | | | Grade w/ | Grade w/ | |
|---|---|---|---|---|---|---|---|---|
| | UGID | | Gene | Genbank | | Higher | Lower | |
| Order | (build #183) | Unigene Name | Symbol | Acc | Affi ID | Expression | Expression | Cut-off |
| 1 | Hs.9329 | TPX2, microtubule-associated protein | TPX2 | AF098158 | A.210052_s_at | 3 | 1 | 8.7748 |
| | | homolog (Xenopus laevis) | | | | | | |
| 2 | Hs.344037 | Protein regulator of cytokinesis 1 | PRC1 | NM_003981 | A.218009_s_at | 3 | 1 | 8.2222 |
| 3 | Hs.292511 | Neuro-oncological ventral antigen 1 | NOVA1 | NM_002515 | A.205794_s_at | 1 | 3 | 6.7387 |
| 4 | Hs.155223 | Stanniocalcin 2 | STC2 | AI435828 | A.203438_at | 1 | 3 | 8.0766 |
| 5 | Hs.437351 | Cold inducible RNA binding protein | CIRBP | AL565767 | B.225191_at | 1 | 3 | 8.2308 |
| 6 | Hs.24395 | Chemokine (C-X-C motif) ligand 14 | CXCL14 | NM_004887 | A.218002_s_at | 1 | 3 | 7.086 |
| 7 | Hs.435861 | Signal peptide, CUB domain, EGF-like 2 | SCUBE2 | AI424243 | A.219197_s_at | 1 | 3 | 7.2545 |

Table D4. SWS Classifier 3: 7 Probe Sets (7 Genes). For any particular gene, where the value of the cell in column 7 “Grade with Higher Expression” is 3, the value of the cell in column 8 “Grade with Lower Expression” is 1, and where the value of cell “Grade with Higher Expression” is 1, the value of column “Grade with Lower Expression” is 3. Column 9 shows the cut off (Optimal Variance Cut-off) expressed as the natural log transform normalised signal intensity measurement for Affymetrix arrays (global mean normalisation
with a scaling factor of 500).

TABLE D5
SWS CLASSIFIER 4

| | | | | | | Grade w/ | Grade w/ | |
|---|---|---|---|---|---|---|---|---|
| | UGID | | Gene | | | Higher | Lower | |
| Order | (build #183) | Unigene Name | Symbol | GenbankAcc | Affi ID | Expression | Expression | Cut-off |
| 1 | Hs.48855 | cell division cycle associated 8 | CDCA8 | BC001651 | A.221520_s_at | 3 | 1 | 5.5046 |
| 2 | Hs.75573 | centromere protein E, 312 kDa | CENPE | NM_001813 | A.205046_at | 3 | 1 | 5.2115 |
| 3 | Hs.552 | steroid-5-alpha-reductase, alpha | SRD5A1 | BC006373 | A.211056_s_at | 3 | 1 | 6.9192 |
| | | polypeptide 1 (3-oxo-5 alpha-steroid | | | | | | |
| | | delta 4-dehydrogenase alpha 1) | | | | | | |
| 4 | Hs.101174 | microtubule-associated protein tau | MAPT | NM_016835 | A.203929_s_at | 1 | 3 | 4.8246 |
| 5 | Hs.164018 | leucine zipper protein FKSG14 | FKSG14 | BC005400 | B.222848_at | 3 | 1 | 6.1846 |
| 6 | acc_R38110 | N.A. | | R38110 | B.240112_at | 1 | 3 | 6.2557 |
| 7 | Hs.325650 | EH-domain containing 2 | EHD2 | AI417917 | A.221870_at | 1 | 3 | 7.6677 |

Table D5. SWS Classifier 4: 7 Probe Sets (7 Genes). For any particular gene, where the value of the cell in column 7 “Grade with Higher Expression” is 3, the value of the cell in column 8 “Grade with Lower Expression” is 1, and where the value of cell “Grade with Higher Expression” is 1, the value of column “Grade with Lower Expression” is 3. Column 9 shows the cut off (Optimal Variance Cut-off) expressed as the natural log transform normalised signal intensity measurement for Affymetrix arrays (global mean normalisation with a scaling factor of 500).

### EXAMPLES

### Example 1

### Materials and Methods: Patients and Tumour Specimens

Clinical characteristics of patient and tumour samples of the Uppsala, Stockholm and Singapore cohorts are summarized in Table E1.

TABLE E1
Distribution of patients and tumour characteristics.

| | Name of cohorts | | | | | | | | |
|---|---|---|---|---|---|---|---|---|---|
| | Uppsala n = 254 | | | Stockholm n = 147 | | | Singapore n = 98 | | |
| | G1 | G2 | G3 | G1 | G2 | G3 | G1 | G2 | G3 |
| Patients, by grade | n = 68 | n = 126 | n = 55 | n = 28 | n = 58 | n = 61 | n = 11 | n = 40 | n = 47 |
| Age, median yrs | 62 | 63 | 62 | 55 | 58 | 52 | 59 | 52 | 50 |
| <55 years, % | 26 | 25 | 44 | 50 | 41 | 56 | 37 | 60 | 68 |
| Tumour size, cm | 1.8 | 2.2 | 2.9 | 1.9 | 2.5 | 2.0 | 3.4 | 2.8 | 3.1 |
| Nodes, positive, % | 15 | 35 | 55 | 33 | 50 | 32 | 36 | 40 | 51 |
| ER negative tumours, % | 3 | 9 | 38 | 0 | 7 | 33 | 0 | 28 | 53 |
| Follow up, median yrs | 11 | 9 | 6 | 8 | 7 | 7 | — | — | — |
| All recurrences, % | 26 | 39 | 50 | 7 | 24 | 36 | — | — | — |
| Endocrine therapy, % | 18 | 37 | 36 | 75 | 62 | 49 | — | — | — |
| Chemotherapy, % | 4 | 6 | 22 | 4 | 5 | 13 | — | — | — |
| Combine therapy, % | 2 | 3 | 0 | 11 | 16 | 10 | — | — | — |
| No systemic therapy, % | 77 | 54 | 45 | 11 | 17 | 28 | — | — | — |

All cohorts are of unselected populations, and in each case, the original tumour material was collected at the time of surgery and freshly frozen on dry ice or in liquid nitrogen and stored under liquid nitrogen or at −70° C.

### Example 2

### Methods: Details of Uppsala, Singapore and Stockholm Cohorts

### Uppsala Cohort

The Uppsala cohort originally comprised of 315 women representing 65% of all breast cancers resected in Uppsala County, Sweden from Jan. 1, 1987 to Dec. 31, 1989. Information pertaining to patient therapies, clinical follow up, and sample processing are described elsewhere (41).

Histological Grading

For histological grading, new tumour sections are prepared from the original paraffin blocks, stained with eosin, and graded in a blinded fashion by H. N. according to the Nottingham grading system (6, Haybittle et al., 1982) as follows:

Tubule Formation: 3=poor, if <10% of the tumour showed definite tubule formation, 2=moderate, if ≧10% but ≦75%, and 1=well, if >75%.

Mitotic Index: 1=low, if <10 mitoses, 2=medium, if 10-18 mitoses, and 3=high, if >18 mitoses (per 10 high-power fields). The field diameter was 0.57 mm.

Nuclear Grade: 1=low, if there was little variation in the size and shape of the nuclei, 2=medium for moderate variation, and 3=high for marked variation and large size.

Scores are then summed, and tumour samples with scores ranging from 3-5 are classified as Grade I; 6-7 as Grade II; and 8-9 as Grade III.

Protein Assays

Protein levels of Estrogen Receptor (ER) and Progesterone Receptor (PgR) are assessed by immunoassay (monoclonal 6F11 anti-ER and monoclonal NCL-PGR, respectively, Novocastra Laboratories Ltd, Newcastle upon Tyne, UK) and deemed positive if >0.1 fmol/ug DNA. VEGF was measured in tumour cytosol by a quantitative immunoassay kit (Quantikine-human VEGF; R&D Systems, Minneapolis, Minn., USA) as described (42). Protein levels of Ki-67 are analyzed using anti-Ki67 antibody (MIB-1) by the grid-graticula method with cut-offs: low=2, medium>2 and <6, high=6. Cyclin E was measured using the antibody HE12 (Santa Cruz Inc., USA) with cutoffs: low=0-4%, medium=5-49%, and high=50-100% stained tumour cells (43).

S-phase fraction was determined by flow cytometry and defined as high if >7% in diploid tumours, or >12% in aneuploid tumours. TP53 mutational status was determined by cDNA sequencing as previously described (41). The Uppsala tumour samples are approved for microarray profiling by the ethical committee at the Karolinska Institute, Stockholm, Sweden.

Stockholm Cohort

The Stockholm samples are derived from breast cancer patients that were operated on at the Karolinska Hospital from Jan. 1, 1994 through Dec. 31, 1996 and identified in the Stockholm-Gotland breast cancer registry.

Information on patient age, tumour size, number of metastatic axillary lymph nodes, hormonal receptor status, distant metastases, site and date of relapse, initial therapy, and date and cause of death are obtained from patient records filed with the Stockholm-Gotland registry.

Tumour sections are classified using the Nottingham grading system (Haybittle et al., 1982). The Stockholm tumour samples are approved for microarray profiling by the ethical committee at the Karolinska Hospital, Stockholm, Sweden.

Singapore Cohort

The Singapore samples are derived from patients that were operated on at the National University Hospital (Singapore) from Feb. 1, 2000 through Jan. 31, 2002.

Information on patient age, tumour size, number of metastatic lymph nodes and hormonal receptor status are obtained from hospital records.

Tumour sections are graded in a blinded fashion according to the Nottingham grading system (Haybittle et al., 1982) as applied to the Uppsala and Stockholm cohorts, with the following exception: Mitotic Index: 1=low, if <8 mitoses, 2=medium, if 9-16 mitoses, and 3=high, if >16 mitoses (per 10 high-power fields). The field diameter is 0.55 mm. The Singapore tumour samples are approved for microarray profiling by the Singapore National University Hospital ethics board.

After exclusions based on tissue availability, RNA integrity, clinical annotation and microarray quality control, expression profiles of 249, 147, and 98 tumours from the Uppsala, Stockholm and Singapore cohorts, respectively, were deemed suitable for further analysis.

### Example 3

### Materials and Methods: Microarray Expression Profiling and Processing

All tumour samples are profiled on the Affymetrix U133A and B genechips. Microarray analysis of the Uppsala and Singapore samples was carried out at the Genome Institute of Singapore (44). The Stockholm samples are analyzed by microarray at Bristol-Myers Squibb, Princeton, N.J., USA. RNA processing and microarray hybridizations are carried out essentially as described (44).

Microarray data processing: all microarray data are processed as previously described (44).

### Example 4

### Materials and Methods: Statistical Analysis of Gene Ontology (GO) Terms

GO analysis is facilitated by PANTHER software, available on the Applied Biosystems' website (46). Selected gene lists are statistically compared (Mann-Whitney) with a reference list (ie, NCBI Build 35) comprised of all genes represented on the microarray to identify significantly over- and under-represented GO terms.

### Example 5

### Materials and Methods: Survival Analysis

The Kaplan Meier estimate is used to compute survival curves, and the p-value of the likelihood-ratio test is used to assess the statistical significance of the resultant hazard ratios. For standardization, events occurring beyond 10 years are censored. All cases of contralateral disease are censored. Disease-free survival (DFS) is defined as the time interval from surgery until the first recurrence or last follow-up.

Multivariate analysis by Cox proportional hazard regression, including a stepwise model selection procedure based on the Akaike information criterion, and all survival statistics are performed in the R survival package. Remaining predictors in the Cox models are assessed by Likelihood-ratio test p-values.

### Example 6

### Methods: Scoring by the Nottingham Prognostic Index (NPI)

NPI scores (Haybittle et al., 1982) are calculated according to the following formula: NPI score=(0.2× tumour size (cm))+grade (1, 2 or 3)+LN stage (1, 2 or 3)

Tumour size is defined as the longest diameter of the resected tumour. LN stage is 1, if lymph node negative, 2, if 3 or fewer nodes involved, and 3, if >3 nodes involved (47). As the number of cancerous lymph nodes are not available for the Uppsala cohort, a stage score of 2 is assigned if 1 or more nodes are involved, and a score of 3 is assigned if nodal involvement showed evidence of periglandular growth. For ggNPI calculations, grade scores (1, 2 or 3) are replaced by genetic grade predictions (1 or 3).

NPI scores <3.4=GPG (good prognostic group); scores of 3.4 to 5.4=MPG (moderate prognostic group); scores >5.4 PPG (poor prognostic group). Scores of 2.4 or less=EPG (excellent prognostic group).

### Example 7

### Methods: Descriptive Statistics

For inter-group comparisons using the clinicopathological measurements, non-parametric Mann-Whitney U-test statistics are used for continuous variables and one-sided Fisher's exact test used for categorical variables. This work is facilitated by the Statistica-6 and StatXact-6 software packages.

### Example 8

### Materials and Methods: Details of Genetic Reclassification Algorithm of Grade 2 Tumours Based on SWS Approach

In simplified terms, the algorithm of genetic re-classification of Grade 2 tumour, based on SWS approach can be described as follows.

A training set consisting of samples of known classes (eg, histologic Grade I (G1) and histologic Grade III (G3) tumours is used to select the variables e, gene expression measurements; probesets or predictors), that allow the most accurate discrimination (or prediction) of the samples in the training set. Once the SWS algorithm is trained on the optimal set of variables, it is then applied to an independent exam set (ie, a new set of samples not used in training) to validate it's prediction accuracy. More details are given below.

Briefly, for constructing the class prediction function, the SWS method uses the training set \tilde{S}₀ (comprised of G1 and G3 tumour samples) to evaluate statistically the weight of the graduated “informative” variables (predictors), and all possible pairs of these predictors. The predictors are automatically selected by SWS from n (n=44,500) probe sets (which represents the gene expression measurements) on U133A and U1133B Affymetrix Genechips.

The description of each patient includes n (potential) prognostic variables X₁, . . . , Xₙ (signals from probe sets of the U133A and U1133B chips) and information about class to which a patient belongs. In particular, the predictors might be able to discriminate G1 and G3 tumours with minimum “a posteriori probability”. Reliability of the SWS class prediction function is based on the standard “leave-one-out procedure” and on an additional exam of the class prediction ability on one or more independent sample populations (ie, patient cohorts). In this application of SWS, the G2 tumour samples of the Uppsala cohort and two other cohorts (NUH and Stockholm cohorts; see Methods) have been used as exam datasets to test the SWS class prediction function.

Let us consider the available n-dimension domain of the variables (the probesets) X₁, . . . , Xₙ as prognostic variable space. The SWS algorithm is based on calculating the a posteriori probabilities of the tumours belonging to one of two classes using a weighted voting scheme involving the sets of so called “syndromes”. A syndrome is the sub-region of prognostic variable space. For a syndrome to be useful in the algorithm, within the syndrome, one class of samples (for instance, G3 tumours) must be significantly highly represented than another class (for instance, G1s), and in other sub-region(s) the inverse relationship should be observed. In the present version of the SWS method, one-dimensional and two-dimensional sub-regions (syndromes) are used.

Let b′ᵢ and b″ᵢ, denote the boundaries of the sub-region for the variable Xᵢ (the i-th probe set); bᵢ′≧Xᵢ>bᵢ″. One-dimensional syndrome for the variable Xᵢ is defined as the set of points in variable space for which inequalities bᵢ′≧Xᵢ>bᵢ″ are satisfied. Two-dimensional syndrome for variables X_{i′} and X_{i″} is defined as a set of points in variable space for which inequalities b_{i′}′≧X_{i′}>b_{i′}″, and b_{i″}′≧X_{i″}>b_{i″}″ are satisfied. The syndromes are constructed at the initial stage of training using the optimal partitioning (OP) algorithm described below.

SWS Training Algorithm

SWS training algorithm is based on three major steps:

1) optimal recoding (partitioning) of the given covariates (signal intensity values) to obtain discrete-valued variables with low and high gradation;

2) selection of the most informative and robust of these discrete-valued variables and their paired combinations (termed syndromes) that together best characterize the classes of interest;

3) tallying the statistically weighted votes of these syndromes to allow us to compute the value of the outcome prediction function.

Optimal Partitioning (OP)

The OP method is used for constructing the optimal syndromes for each class (G1 and (13) using the training set \tilde{S}₀. The OP is based on the optimal partitioning of some potential prognostic variable Xᵢ range that allows the best separation of the samples belonging to different classes. To evaluate the separating ability of partition R (see below) in the training set \tilde{S}₀ the chi-2 functional is used (Kuznetsov et al, 1998). The optimal partitions are searched inside observed variable domain that contain partitions with cut-off values not greater than a fixed threshold (defined below). The partition with the maximal value of the chi-2 functional is considered optimal for the given variable.

Stability of Partitioning

Another important characteristic that allows evaluation the prognostic ability of partitioning model for specific variables is the index of boundary instability. Let Rₒ, Rₗ, . . . , Rₘ be optimal partitions of variable Xᵢ ranges that is calculated by training set \tilde{S}₀, \tilde{S}₁, . . . , \tilde{S}ₘ, where \tilde{S}ₖ is the training set without description of the k^{th} sample. Let Kⱼ denote the different classes (j=1, 2). Let b₁^{k}, . . . , bᵣ₋₁^{k} be boundary points of optimal partition Rₖ found by training set \tilde{S}ₖ; Dᵢ is the variance of variable Xᵢ. The boundary instability index κ(\tilde{S}₀, Kⱼ, r) for partitioning with r elements is calculated as the ratio (Kuznetsov et al, 1996):

$$κ ( {\tilde{S}}_{0} , {K}_{j} , r ) = \frac{1}{{D}_{i} ( r - 1 )} [ \begin{matrix}{\sum }_{k = 1}^{m} & {\sum }_{l = 1}^{r - 1} & {( {b}_{l}^{k} - {b}_{l}^{0} )}^{2}\end{matrix} ] .$$

Selecting of Optimal Variables Set

The OP can be used at the initial stage of training for reducing the dimension of the prognostic variables set. Selection of the optimal set of prognostic variables depends on a sufficiently high partition value determined by the Chi-2 function. The additional criterion of selection of prognostic variables is the instability index κ(\tilde{S}₀, Kⱼ, r). The variable is used if value κ(\tilde{S}₀, Kⱼ, r) is less than threshold κ₀, defined a priori by the user. When the partition of the given variable is instable (κ(\tilde{S}₀, Kⱼ, r)<κ₀), the variable is removed from the final optimal set of prognostic variables. Finally, the optimal set of prognostic variables is defined if both selection criteria are fulfilled.

The Weighted Voting Procedure

Let \tilde{Q}ⱼ⁰ denote the set of constructed syndromes for class Kⱼ. Let x* denote the point of parametric space. The SWS estimates a posteriori probability Pⱼ^{sv}(x*) of the class Kⱼ at the point x* that belongs to the intersection of syndromes q₁, . . . , qᵣ from \tilde{Q}ⱼ⁰ as follows:

$${P}_{j}^{sv} ( {x}^{*} ) = \frac{{\sum }_{i = 1}^{r} {w}_{i}^{j} {v}_{i}^{j}}{{\sum }_{i = 1}^{r} {w}_{i}^{j}} , \qquad ( 1 )$$

where vᵢ^{j} is the fraction of class Kⱼ among objects with prognostic variables vectors belonging to syndrome qᵢ, wᵢ is the so-called “weight” of syndrome qᵢ. The weight wᵢ is calculated by the formula.

$${w}_{i} = \frac{{m}_{i}}{{m}_{i} + 1} \frac{1}{{{d}^{⋒}}_{i}} ,$$

where

$${{d}^{⋒}}_{i} = ( 1 - {v}_{i}^{i} ) {v}_{i}^{i} + \frac{1}{{m}_{i}} ( 1 - {v}_{0}^{j} ) {v}_{0}^{j}$$

(Kuznetsov, 1996.) The estimate of fraction vᵢ^{j} variance has the second term

$$\frac{1}{{m}_{i}} ( 1 - {v}_{0}^{j} ) {v}_{0}^{j} ,$$

which is used to avoid a value \hat{d}ᵢ equal to zero in cases when the given syndrome is associated only with objects of one class from the training set.

The results of testing applied and simulated tasks have demonstrated that formula (1) gives too low of estimates of conditional probabilities for classes that are of smaller fraction in the training set. So the additional correction of estimates in (1) has been implemented. The final estimates of conditional probability at point x* are calculated as Pⱼ^{sws}(x*)=Pⱼ^{sv}(x*)χ(\tilde{S}₀,Kⱼ),

where

$$χ ( {S}_{0} , {K}_{j} ) = \frac{1}{{\sum }_{k = 1}^{m} {P}_{j}^{sv} ( {x}_{i} )}$$

$$and$$

$${x}_{k}$$

is the vector of prognostic variables for the k-th samples from the training set.

### Example 9

### Derivation of a Classifier Comprising 264 Probe Sets (SWS Classifier 0)

### Schema of the SWS-Based Discovery Method of Novel Classes of Tumours

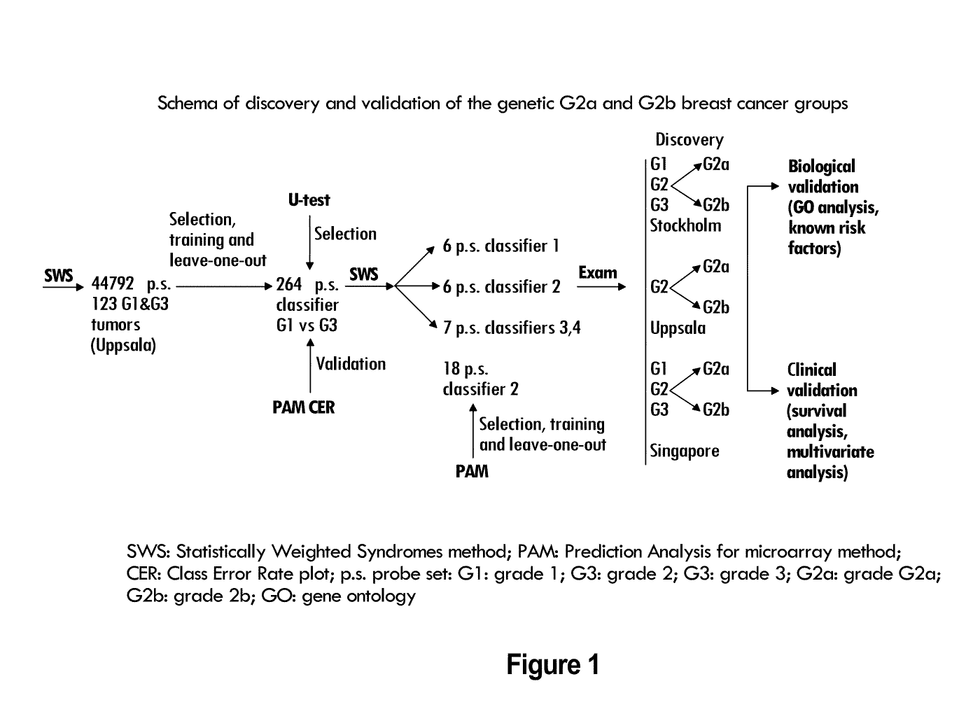

Our methodology is based on the schema presented in FIG. 1.

Beginning with the Uppsala dataset comprised of 68 G1 and 55 G3 tumours, we used SWS optimal partitioning (OP) at the initial stage of training to reduce the dimension of the prognostic variables set. SWS rank orders the set of probes according to specific algorithmic criteria for assessing differential expression between classes.

Based on this two-criteria (chi-2 and instability index) selection algorithm, we used SWS chi-2 values bigger than 24.38 (at p-value less then 0.00001); in combination with low boundary instability index criteria (κ₀<0.1 for 90% of the selected informative variables and κ₀<0.4 for 10% of the other informative variables). Visual presentation on scatchard plot (log κ₀, chi-2) distribution of probesets, these two cut-off values discriminated the relatively small and compact group of probesets. We observed that this group of probesets provide a local minima on the Class Error Rate (CER) function and provide an optimal selection of 264 probesets classifier of G1 and G3. Using these 264 probe sets, the both SWS and PAM methods provide a small misclassification error (4.5% for G1, and 5.5% for G3, respectively) when the leave-one-out cross-validation procedure is used. We also used the U-test with critical value p=0.05 (with Bonferroni correction) and all 264 probesets follow this cut-off value.

Based on our selection criteria, we selected a classifier comprising 264 probe sets, which we term the “SWS Classifier 0”. See Table D1 in section “SWS Classifier 0” of the Description as well as Appendix 1.

Details are shown in Appendix 1A, Appendix 2, Appendix 3 and Appendix 4.

### Example 10

### A Posteriori Probability for SWS Classifier 0 (264 Probe Sets) G1 and G3 Estimated by SWS Classifier 2

A posterior probability for G1 and G3 was also estimated by SWS Classifier 2 for each tumour sample by the classical leave-one-out cross-validation procedure.

We estimated the class error rate based on the misclassification error rate plot (Tibshirani et al, 2002) and found that for the 264 selected probe sets, CER consists of 5% for G1, and 6% for G3, respectively. Similar discrimination was obtained with SWS methods (see above).

Based on consistency between SWS and U-tests and PAM CER validation of the selection procedure, we further considered the classification results using the 264 variables. In two-group comparisons, high CER were observed in the G1-G2 and G2-G3 predictions (data not shown), while G1-G3 classification accuracy was high (<5% errors). Complementary to SWS classification method, the PAM method confirms that G2 tumours are not molecularly distinct from either low or high grade tumours, possibly owing to substantial molecular heterogeneity within the G2 class.

### Example 11

### Derivation of Classifiers of 6 Genes (SWS Classifier 1)

To extract the smallest possible classifier from the 264 variables, we varied the initial parameters of the SWS algorithm to minimize the number of predictors in training set providing the maximum correlation coefficient between posteriori probabilities and true class indicators (specifically, 1 was the indicator of G1 tumours, and 3 was the indicator of G3 tumours in the G1-G3 comparison). The predictive power of the predictor set was estimated using standard leave-one-out procedure and counting the numbers of errors of class predictions.

We derived a classifier comprising 6 gene probe sets (5 genes) which we term the “SWS Classifier 1”. 4.4% for class G1; and 5.5% for class G3 CERs were obtained with the SWS Classifier 1. See FIG. 1 and Table D2 in section “SWS Classifier 1” of the Description.

Appendix 5A, Appendix 5B and Appendix 5C show detailed information about selected gene probe sets, optimal partition boundaries, true classes, posterior probabilities and clinical significance of the SWS Classifier 1 predictor (estimated by patient survival analysis).

### Example 12

### Derivation of Classifiers of 18 Genes (SWS Classifier 2)

By SWS, for the G1-G3 comparisons, maximal prediction accuracies are obtained with 18 probe sets (17 genes). We refer to this 18 probe set as the “SWS Classifier 2”. See Table D3 in section “SWS Classifier 2” of the Description. This classifier includes all five genes represented by SWS Classifier 1.

Appendix 6A, Appendix 6B and Appendix 6C show detailed information about selected gene probe sets, optimal partition boundaries, true classes, posterior probabilities and clinical significance of the SWS Classifier 2 (estimated by patient survival analysis).

With the 18 probe sets, both the SWS Classifier 2 and PAM correctly classify ˜96% (65/68) of the G1s and ˜95% (52/55) of the G3s (by leave one-out method).

The smaller number of probes sets required by SWS Classifier 1 (6 probe sets) compared to PAM (18 probe sets, data not presented) may reflect the ability of SWS to use more diverse interaction and/or co-expression patterns during variable selection.

The posterior probability (Pr) is an estimate of the likelihood that a sample from the exam group of tumours belongs to one class (termed “G1-like”) or the other (ie, “G3-like”). Both 18 probesets SWS and PAM classifiers scored the vast majority of G1 and G3 tumours with high probabilities of class membership.

### Example 13

### The SWS Classifier 0 (264 Gene Probe Set) Contains Many Small Subsets which can Provide Equally High Discrimination Ability of the Genetic G2a and G2b Tumours

Due to the highly informative and stable nature of each gene (represented by Affymetrix probe-sets) of the 264 predictor set we hypothesized that there are many small alternative gene sub-sets that could be used to classify tumours with high accuracy (and therefore classify patients according to outcome with high prognostic significance). For example, high Pr scores for the class assignments of G1 and G3 by SWS classifier 1 (6 probesets, as shown in Table D2 in section “SWS classifier 1” of the Description and Appendix 5A) and SWS class assignments of G1-like and G3-like classes within G2 class were observed.

Notably, 95% of the tumours of the Uppsala cohort showed >75% probability of belonging to either the G1-like or G3-like class, indicating a highly discriminant statistical basis for the class prediction function of the SWS classifier 1 for the G2 class.

### Example 14

### SWS Classifier 3 and SWS Classifier 4

To find other classifiers, we excluded the best 6 probe sets (SWS classifier 1) from the 264 probe sets, and randomly selected two non-overlapping subsets (each of 40 probe sets) from the remaining 258 probe sets and applied the SWS algorithm to each subset.

In this way, we selected two additional classifiers: SWS classifier 3 (6-probe sets; Table D4 in section “SWS Classifier 3” of the Description and Appendix 7A) and SWS classifier 4 (7-probe sets; Table D5 in section “SWS Classifier 4” of the Description and Appendix 8A).

Tables D4 and D5 are organized as Table D3. For Uppsala, Stockholm and Singapore cohorts, each of three SWS classifiers provide similar high accuracy of classification in G1-G3 comparisons (Tables D3-D5). SWS also provided high and reproducible levels of separation of G2a and G2b sub-groups for different cohorts and highly significant differences in G2a-G2b comparison based on survival analysis (Tables D3-D5).

These tables show the values of parameters of SWS algorithm for selected classifies, predicted individual probabilities of belonging to the given class, and gene annotation, clinical significance etc.

Thus, we could consider the 264 probe sets as a general genetic classifier of the G2a (G1-like) and G2b (G3-like) tumour types.

### Example 15

### Dichotomy of G2 Tumours by 264 Probe Sets Gene Grade Classifier

We next applied our grade classifiers directly to the 126 G2 tumours of the Uppsala cohort to ask if these genetic determinants of low and high grade might resolve moderately differentiated G2 tumours into separable classes. Using SWS for the 264 predictor set, we observed that the G2 tumours could be separated into G1-like (n=83) and G3-like (n=43) classes with few tumours exhibiting intermediate Pr scores (Appendix 2).

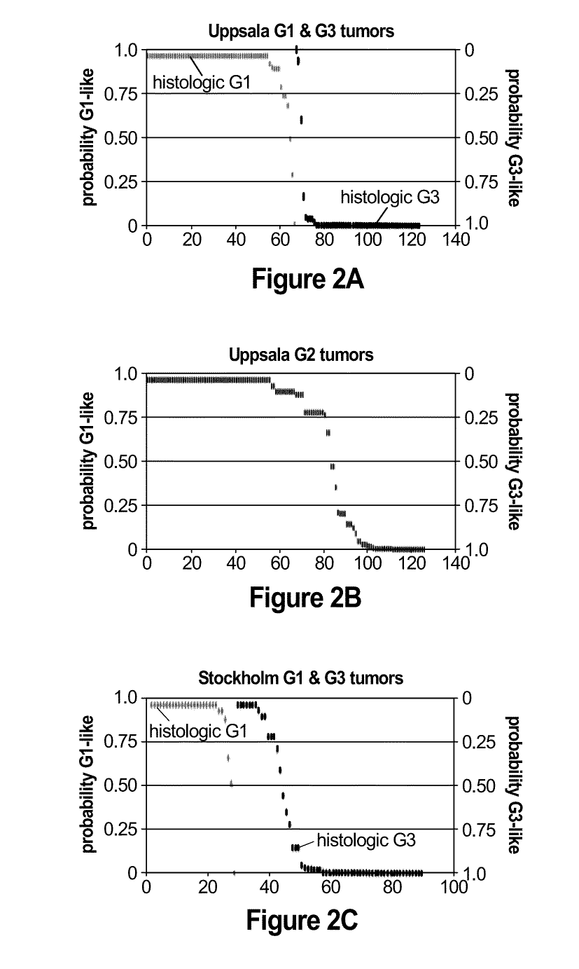

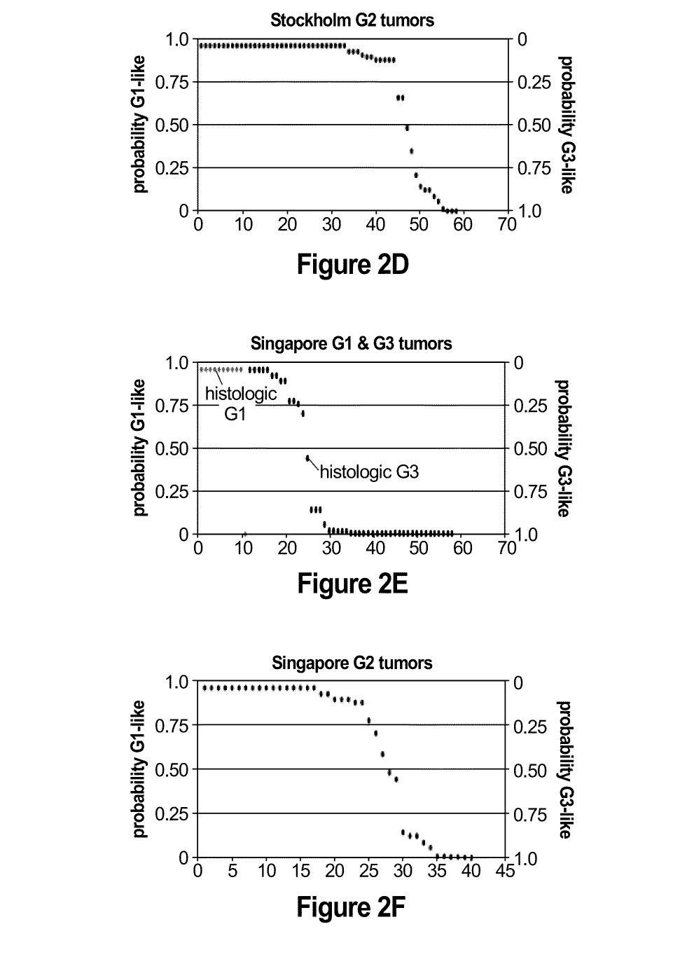

The probabilities of the SWS class assignments are shown in FIG. 2B (FIG. 2, Panel B) and more detailed information in Appendix 2.

We found 96% of the G2 tumours were assigned by the SWS classifier (and 94% by the PAM classifier, data not shown) to either the G1-like or G3-like classes with >75% probability, indicating that almost all G2 tumours can be molecularly well separated into distinct low- and high-grade-like classes (henceforth referred to as “G2a” and “G2b” genetic grades) (Appendix 2).

We validated the separation ability of G1a and G2b based on individual predictors and showed that all of them are statistically significant by U-test and t-test (Appendix 3).

Clinical validation (survival analysis) of G2a and G2b tumour subtypes based on the predictor set (or genetic classifier), showed a highly significant difference between survival curves of the G1a and G2b patients (Appendix 4).

### Example 16

### Genetic Grade is Prognostic of Tumour Recurrence

To determine if the genetic grade classification correlates with patient outcome, we compared the disease-free survival (DFS) of patients with histologic G2 tumours classified as G2a or G2b by the SWS algorithm. (Due to space limitations and high concordance between the SWS and PAM classifiers, only data for the SWS classifier are presented hereafter.)

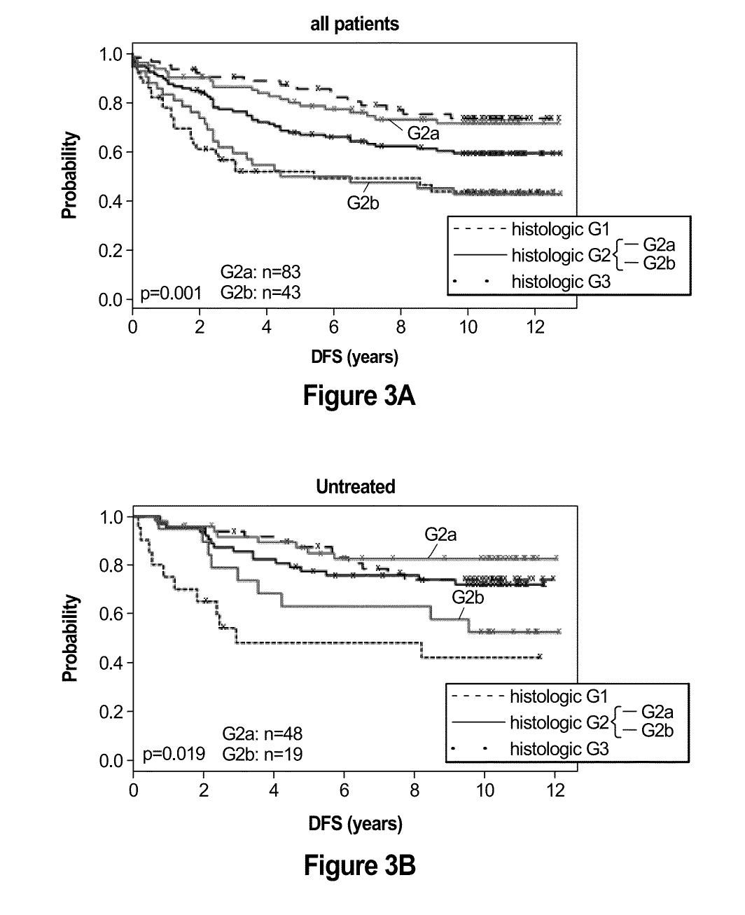

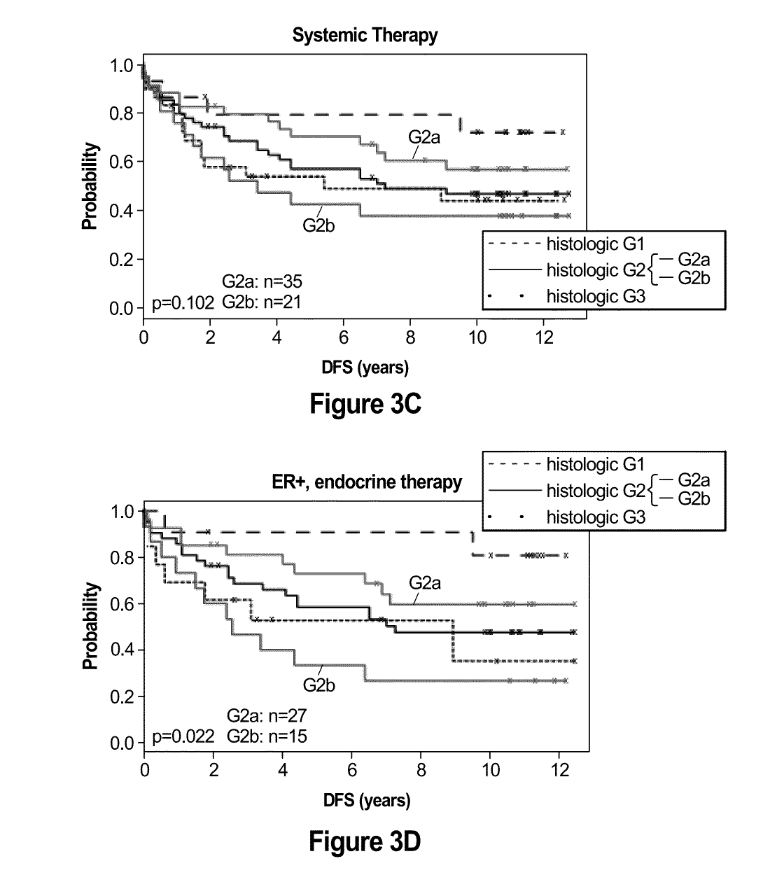

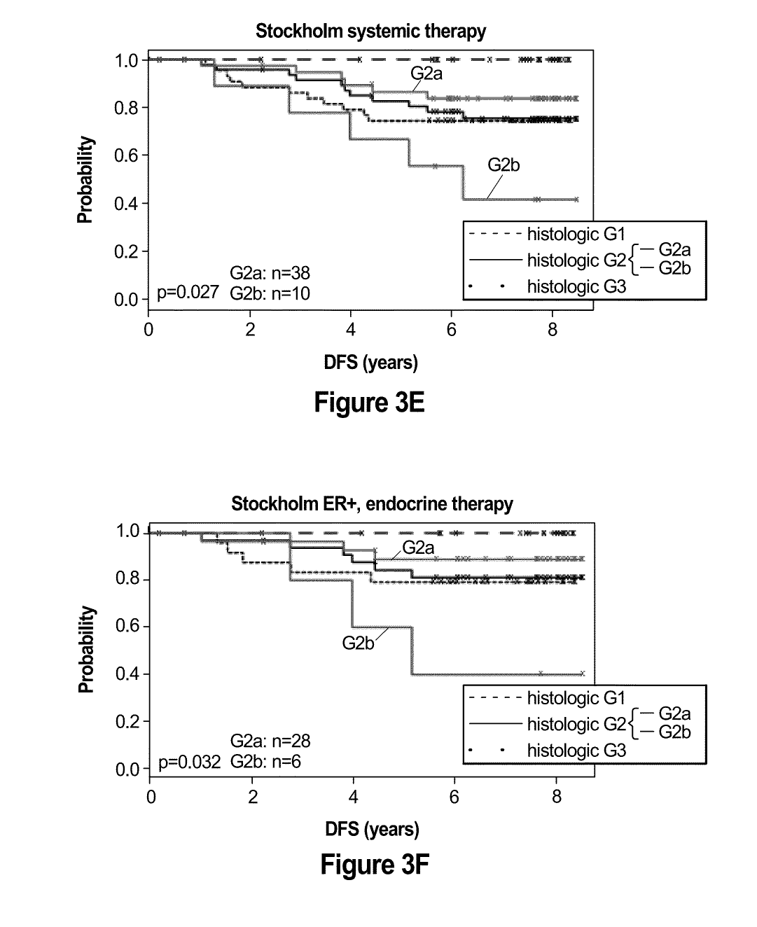

The Kaplan-Meier survival curves for these patients are shown in FIGS. 3A-3F (green and red curves) superimposed on the survival curves of histologic G1, G2 and G3 patients (black curves) for comparison. Patients with G2a tumours showed a significantly better disease-free survival than those with G2b disease, regardless of therapeutic background (p=0.001; FIG. 3A).

This finding is consistent in specific therapeutic contexts including untreated patients (FIG. 3B), systemic therapy (FIG. 3C), and hormone therapy only (FIG. 3D) with survival differences significant at p=0.019, p=0.10 and p=0.022, respectively. These findings demonstrate a robust prognostic power of the genetic grade classifier in moderately differentiated tumours independent of therapeutic effects.

### Example 17

### External Validation of the Genetic Grade Signature on the Stockholm and Singapore Cohorts

For external validation, we directly applied the SWS classifier to two large independent cohorts of primary breast cancer cases that are also graded according to the NGS guidelines and profiled on the Affymetrix platform (albeit at different times and in different laboratories). The results of the grade classifications are shown in FIGS. 2A-2F.

In both the Stockholm and Singapore cohorts, the G1 tumours are correctly classified with high accuracies similar to that observed in the training set: 96% (27/28) for Stockholm and 91% (10/11) for Singapore (FIG. 2C and FIG. 2E). However, both cohorts showed less accuracy in classifying the G3 tumours: 75% (46/61) for Stockholm and 72% (34/47) for Singapore. Despite this, the classifier remained capable of dividing the vast majority of the tumour samples into G1-like and G3-like classes with high Pr scores, and this remained true for the G2 tumours of both the Stockholm and Singapore cohorts (FIG. 2D and FIG. 2F).

As clinical histories are available on the Stockholm patients, we tested the prognostic performance of the classifier on this new G2 population of which 79% (46/58) of tumours are classified as G2a and 21% (12/58) are classified as G2b. Though this set is considerably smaller than the Uppsala G2 set, similar survival associations are observed.

As FIG. 3E and FIG. 3F show, patients with the G2a subtype are significantly less likely to relapse than those with tumours of the G2b subtype, indicating that the prognostic performance of the genetic grade classifier is reproducible in a second, independent population of G2 patients.

### Example 18

### The Prognostic Power of Genetic Grade is Independent of Other Risk Factors

To assess the prognostic novelty of the classifier, we used multivariate Cox regression models to compare its performance to that of other conventional prognostic indicators assessed in the Uppsala cohort including lymph node status, tumour size, patient age, and estrogen (ER) and progesterone (PgR) receptor status. See Table E3 below.

TABLE E3

| | | | | | Systemic therapy- | | ER+, Tamoxifen- | |
|---|---|---|---|---|---|---|---|---|
| | All patients | | Untreated patients | | treated patients | | treated patients | |
| | | Hazard | | Hazard | | Hazard | | Hazard |
| | | ratio | | ratio | | ratio | | ratio |
| Variables | p-value | (95% CI) | p-value | (95% CI) | p-value | (95% CI) | p-value | (95% CI) |
| genetic grade | 0.001 | 1.50-5.09 | 0.046 | 1.02-7.77 | 0.038 | 1.49-8.01 | 0.009 | 1.39-9.99 |
| signature | | | | | | | | |
| LN status | 0.031 | 0.27-0.94 | 0.700 | 0.01-23.53 | 0.091 | 0.13-1.16 | 0.096 | 0.11-1.20 |
| Tumour size | 0.054 | 0.99-1.07 | 0.950 | 0.91-1.10 | 0.016 | 1.01-1.11 | 0.250 | 0.97-1.09 |
| Age | 0.500 | 0.97-1.06 | 0.820 | 0.96-1.03 | 0.440 | 0.96-1.02 | 0.450 | 0.94-1.02 |
| ER status | 0.061 | 0.46-1.06 | 0.640 | 0.15-3.18 | 0.110 | 0.01-1.55 | — | — |
| PgR status | 0.300 | 0.57-6.10 | — | — | 0.270 | 0.56-7.76 | 0.990 | 0.10-9.50 |

The genetic grade signature is a strong independent indicator of disease-free survival in a multivariate analysis with conventional risk factors.

As Table E3 shows, the genetic grade signature remained significantly associated with outcome in the different therapeutic contexts independent of the classical predictors, and is superior to both LN status and tumour size in all four treatment subgroups with the exception of systemic therapy where only tumour size is more significant.

This finding is further substantiated by a robust model selection approach (the Akaike Information Criterion) whereby the genetic grade classifier remained more significant than LN status and tumour size in all therapeutic subgroups (data not shown). These results demonstrate a powerful and additive contribution of the genetic grade classifier to patient prognosis.

### Example 19

### G2a and G2b Subtypes are Molecularly and Pathologically Distinct

The prognostic performance of the classifier suggests that G2a and G2b genetic grades may in fact represent distinct pathological entities previously unrecognized. We investigated this possibility by several approaches.

First we examined the histopathological composition of the G2a and G2b tumours and found that the predominant histologic subtypes—ductal, lobular and tubular—are equally distributed within the two classes and therefore not correlated with genetic grade (data not shown). Next, we analyzed the expression levels of the selected 264 probesets (i.e., representing ˜232 genes) as the maximum number of probesets capable of recapitulating a high G1/G3 classification accuracy (see Methods). These genes represent the top most significantly differentially expressed genes between G1 and G3 tumours after correcting for false discovery (see Table D1 above).

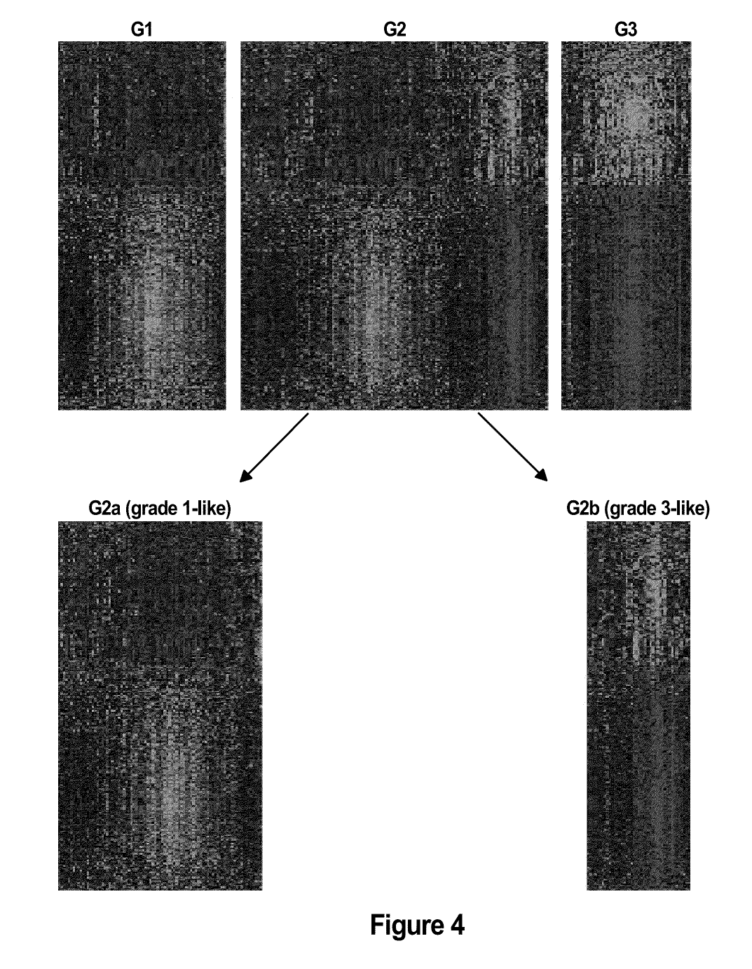

As shown in FIG. 4, hierarchical cluster analysis using this set of genes shows a striking separation of the G2 population into two primary tumour profiles highly resembling the G1 and G3 profiles and that separate well into the G2a and G2b classes. Indeed, all but 11 of these 264 gene probesets are also differentially expressed (at p<0.05, Wilcoxon rank-sum test) between the G2a and G2b tumours.

This finding shows that extensive molecular heterogeneity exists within the G2 tumour population, and this heterogeneity is robustly defined by the major determinants of G1 and G3 cancer. It also demonstrates that a much larger and pervasive transcriptional program underlies the genetic grade predictions of the SWS signature—despite its composition of a mere 5 genes. Furthermore, statistical analysis of the gene ontology (GO) terms associated with the G2a-G2b differentially expressed genes revealed the significant enrichment of numerous biological processes and molecular functions.

Table E4 displays a selected set of significantly enriched GO categories which includes cell cycle, inhibition of apoptosis, cell motility and stress response, suggesting an imbalance of these cellular processes between the G2a- and G2b-type tumour cells.

TABLE E4
Gene ontology analysis of differentially expressed genes.
Selected terms are shown with corresponding
p-values that reflect significance of term enrichment

| | G1 vs G2a | G2a vs G2b | G2b vs G3 |
|---|---|---|---|
| Biological Process | | | |
| Cell cycle | 6.2E−06 | 5.7E−28 | 2.5E−06 |
| Chromatin packaging | 1.3E−02 | 2.5E−02 | |
| and remodeling | | | |
| Mitosis | 2.7E−02 | 6.8E−15 | 1.1E−03 |
| Inhibition of apoptosis | | 4.4E−03 | 4.9E−03 |
| Oncogenesis | 1.6E−02 | 5.5E−04 | 5.5E−03 |
| Cell motility | | 3.6E−02 | 4.4E−02 |
| Stress response | | 5.0E−03 | |
| Molecular Function | | | |
| Kinase activator | 1.1E−03 | 7.2E−06 | |
| Histone | 3.5E−03 | 5.0E−02 | |
| Nucleic acid binding | 1.3E−02 | | |
| Microtubule family | | 7.6E−07 | 4.2E−04 |
| cytoskeletal protein | | | |
| Chemokine | | | 7.5E−03 |
| Non-receptor serine/ | | 7.8E−04 | |
| threonine protein kinase | | | |
| Extracellular matrix | | 1.9E−02 | |
| linker protein | | | |
| Pathway | | | |
| Insulin/IGF pathway- | 4.9E−02 | | |
| MAPKK/MAPK cascade | | | |
| Apoptosis signaling pathway | | | 4.9E−02 |
| Ubiquitin proteasome pathway | | 3.0E−02 | |

Table S2 below shows the complete list of GO categories and their p values.

TABLE S2
Comprehensive table of significant gene ontology terms identified
in the different tumour group comparisons.

| NCBI REFLIST | expected | observed | |
|---|---|---|---|
| (23481) | ratio | ratio | P value |

G2a vs. G2b tumours

| | | | | |
|---|---|---|---|---|
| Biological Process | | | | |
| Cell cycle | 853 | 7.08 | 50 | 5.69E−28 |
| Mitosis | 287 | 2.38 | 22 | 6.78E−15 |
| Cell proliferation and differentiation | 751 | 6.24 | 32 | 4.21E−14 |
| Cell cycle control | 390 | 3.24 | 23 | 3.50E−13 |
| Chromosome segregation | 102 | 0.85 | 10 | 2.00E−08 |
| Cell structure | 624 | 5.18 | 17 | 2.16E−05 |
| Protein targeting and localization | 225 | 1.87 | 10 | 2.27E−05 |
| Cell structure and motility | 1021 | 8.48 | 22 | 4.73E−05 |
| DNA metabolism | 305 | 2.53 | 11 | 5.82E−05 |
| Oncogenesis | 600 | 4.98 | 14 | 5.52E−04 |
| DNA replication | 89 | 0.74 | 5 | 9.62E−04 |
| Protein phosphorylation | 592 | 4.92 | 13 | 1.49E−03 |
| Meiosis | 68 | 0.56 | 4 | 2.65E−03 |
| Inhibition of apoptosis | 127 | 1.05 | 5 | 4.43E−03 |
| Stress response | 187 | 1.55 | 6 | 5.03E−03 |
| Biological process unclassified | 9457 | 78.54 | 61 | 5.89E−03 |
| Protein biosynthesis | 598 | 4.97 | 0 | 6.54E−03 |
| Carbohydrate metabolism | 512 | 4.25 | 0 | 1.36E−02 |
| Cytokinesis | 116 | 0.96 | 4 | 1.65E−02 |
| Protein modification | 1013 | 8.41 | 15 | 2.27E−02 |
| Chromatin packaging and remodeling | 196 | 1.63 | 5 | 2.47E−02 |
| Sensory perception | 642 | 5.33 | 1 | 2.91E−02 |
| Cytokine/chemokine mediated immunity | 83 | 0.69 | 3 | 3.26E−02 |
| Other cell cycle process | 4 | 0.03 | 1 | 3.27E−02 |
| Proteolysis | 813 | 6.75 | 2 | 3.35E−02 |
| Chemosensory perception | 399 | 3.31 | 0 | 3.54E−02 |
| Cell motility | 291 | 2.42 | 6 | 3.57E−02 |
| Apoptosis | 459 | 3.81 | 8 | 3.91E−02 |
| DNA recombination | 38 | 0.32 | 2 | 4.03E−02 |
| Olfaction | 364 | 3.02 | 0 | 4.75E−02 |
| Molecular Function | | | | |
| Microtubule binding motor protein | 74 | 0.61 | 10 | 9.86E−10 |
| Microtubule family cytoskeletal protein | 233 | 1.93 | 12 | 7.63E−07 |
| Kinase activator | 54 | 0.45 | 6 | 7.21E−06 |
| Kinase modulator | 126 | 1.05 | 8 | 1.27E−05 |
| Replication origin binding protein | 19 | 0.16 | 4 | 2.21E−05 |
| Non-receptor serine/threonine protein | 289 | 2.4 | 9 | 7.79E−04 |
| kinase | | | | |
| Protein kinase | 526 | 4.37 | 12 | 1.64E−03 |
| Voltage-gated sodium channel | 14 | 0.12 | 2 | 6.23E−03 |
| Cytoskeletal protein | 824 | 6.84 | 14 | 9.42E−03 |
| Kinase | 692 | 5.75 | 12 | 1.36E−02 |
| Extracellular matrix linker protein | 25 | 0.21 | 2 | 1.87E−02 |
| Ribosomal protein | 431 | 3.58 | 0 | 2.70E−02 |
| KRAB box transcription factor | 640 | 5.31 | 1 | 2.95E−02 |
| DNA strand-pairing protein | 6 | 0.05 | 1 | 4.86E−02 |
| Histone | 99 | 0.82 | 3 | 5.03E−02 |
| Pathway | | | | |
| Cell cycle | 22 | 0.18 | 3 | 8.75E−04 |
| Ubiquitin proteasome pathway | 80 | 0.66 | 3 | 2.97E−02 |
| DNA replication | 43 | 0.36 | 2 | 5.03E−02 |

G1 vs. G2a tumours

| | | | | |
|---|---|---|---|---|
| Biological Process | | | | |
| Cell cycle control | 390 | 0.35 | 6 | 9.19E−07 |
| Cell cycle | 853 | 0.76 | 7 | 6.19E−06 |
| Chromatin packaging and remodeling | 196 | 0.18 | 2 | 1.32E−02 |
| Oncogenesis | 600 | 0.54 | 3 | 1.57E−02 |
| Nucleoside, nucleotide and nucleic acid | 3372 | 3.02 | 7 | 2.31E−02 |
| metabolism | | | | |
| Mitosis | 287 | 0.26 | 2 | 2.69E−02 |
| Calcium ion homeostasis | 32 | 0.03 | 1 | 2.82E−02 |
| Developmental processes | 2150 | 1.92 | 5 | 3.77E−02 |
| mRNA transcription regulation | 1553 | 1.39 | 4 | 4.63E−02 |
| Molecular Function | | | | |
| Kinase activator | 54 | 0.05 | 2 | 1.08E−03 |
| Histone | 99 | 0.09 | 2 | 3.54E−03 |
| Kinase modulator | 126 | 0.11 | 2 | 5.65E−03 |
| Select regulatory molecule | 979 | 0.88 | 4 | 1.02E−02 |
| Nucleic acid binding | 3014 | 2.7 | 7 | 1.29E−02 |
| Nuclear hormone receptor | 48 | 0.04 | 1 | 4.21E−02 |
| Other transcription factor | 387 | 0.35 | 2 | 4.64E−02 |
| Pathway | | | | |
| Axon guidance mediated by semaphorins | 50 | 0.04 | 1 | 4.38E−02 |
| Insulin/IGF pathway-mitogen activated | 56 | 0.05 | 1 | 4.89E−02 |
| protein kinase kinase/MAP kinase cascade | | | | |

G2b vs. G3 tumours

| | | | | |
|---|---|---|---|---|
| Biological Process | | | | |
| Cell cycle | 853 | 2.29 | 12 | 2.50E−06 |
| Cell proliferation and differentiation | 751 | 2.01 | 10 | 3.03E−05 |
| Cell cycle control | 390 | 1.05 | 7 | 8.55E−05 |
| Mitosis | 287 | 0.77 | 5 | 1.06E−03 |
| Chromosome segregation | 102 | 0.27 | 3 | 2.68E−03 |
| Inhibition of apoptosis | 127 | 0.34 | 3 | 4.93E−03 |
| Oncogenesis | 600 | 1.61 | 6 | 5.45E−03 |
| Apoptosis | 459 | 1.23 | 5 | 7.85E−03 |
| Meiosis | 68 | 0.18 | 2 | 1.46E−02 |
| Chromatin packaging and remodeling | 196 | 0.53 | 3 | 1.59E−02 |
| Protein targeting and localization | 225 | 0.6 | 3 | 2.28E−02 |
| Developmental processes | 2150 | 5.77 | 11 | 2.69E−02 |
| Oncogene | 98 | 0.26 | 2 | 2.88E−02 |
| Skeletal development | 108 | 0.29 | 2 | 3.43E−02 |
| Determination of dorsal/ventral axis | 14 | 0.04 | 1 | 3.69E−02 |
| Cytokinesis | 116 | 0.31 | 2 | 3.91E−02 |
| Cell motility | 291 | 0.78 | 3 | 4.36E−02 |
| Embryogenesis | 131 | 0.35 | 2 | 4.86E−02 |
| Molecular Function | | | | |
| Microtubule family cytoskeletal protein | 233 | 0.63 | 5 | 4.19E−04 |
| Chromatin/chromatin-binding protein | 132 | 0.35 | 3 | 5.49E−03 |
| Chemokine | 48 | 0.13 | 2 | 7.51E−03 |
| Non-motor microtubule binding protein | 52 | 0.14 | 2 | 8.76E−03 |
| Microtubule binding motor protein | 74 | 0.2 | 2 | 1.71E−02 |
| Other transcription factor | 387 | 1.04 | 4 | 2.04E−02 |
| Cytoskeletal protein | 824 | 2.21 | 6 | 2.31E−02 |
| Reductase | 108 | 0.29 | 2 | 3.43E−02 |
| Pathway | | | | |
| Apoptosis signaling pathway | 131 | 0.35 | 2 | 4.86E−02 |

To extend our analysis beyond the transcript level, we investigated the differences between G2a and G2b tumours using conventional clinicopathological markers.

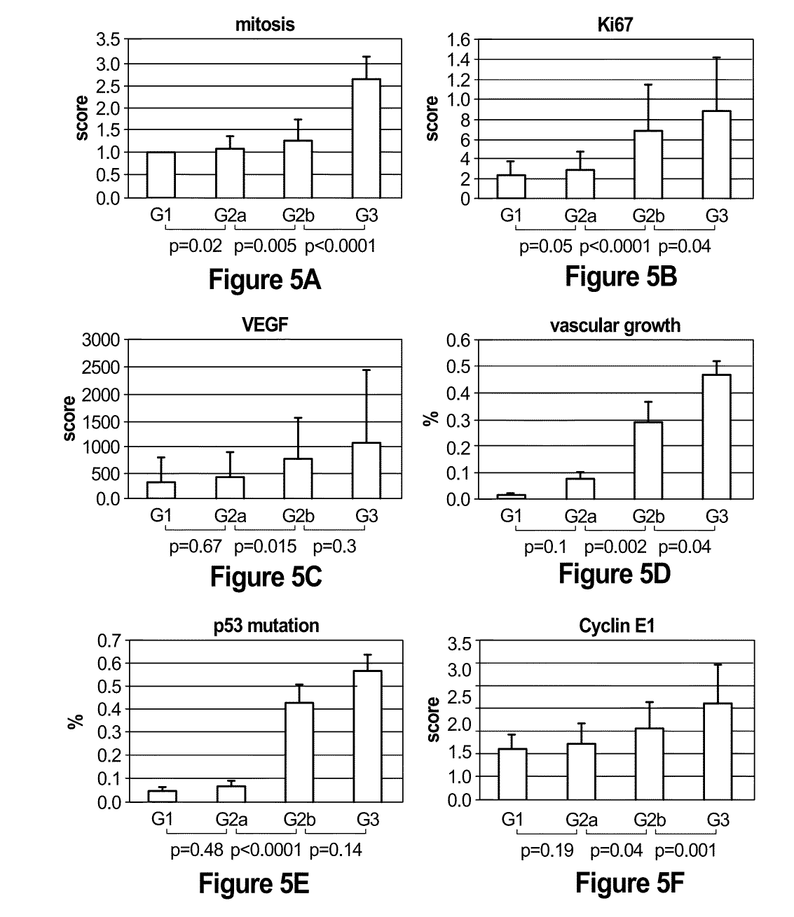

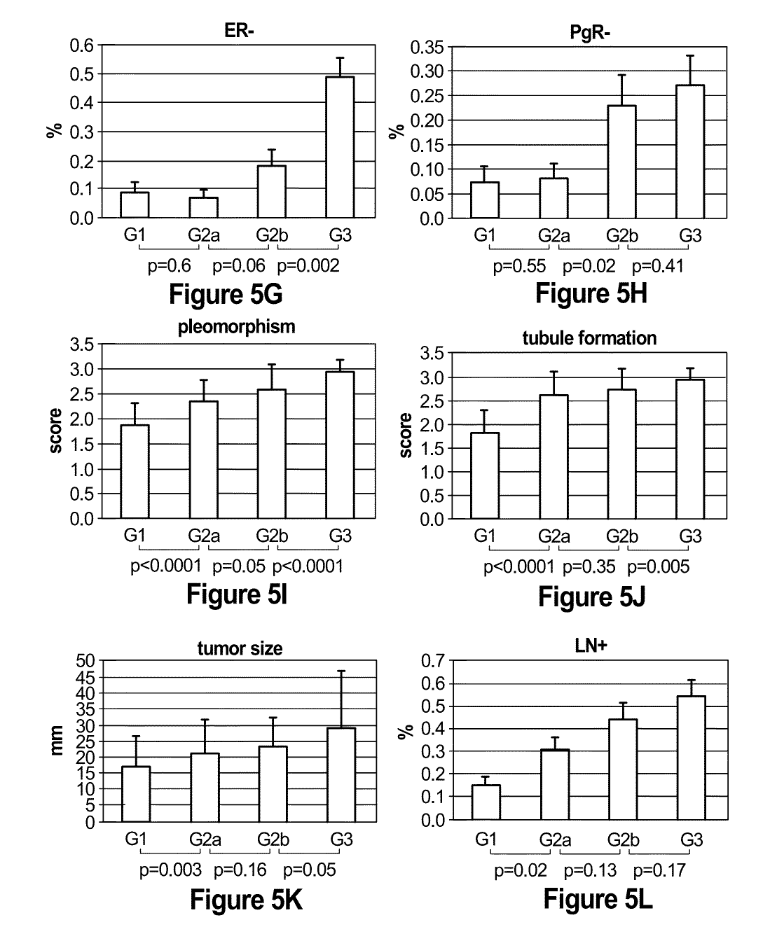

Of the three histologic grading criteria, both mitotic count and nuclear pleomorphism are found to significantly vary between the G2a and G2b tumours (p=0.007 and p=0.05; FIG. 5A and FIG. 5I). Protein levels of the proliferation marker Ki67 are also found to be significantly different between the G2a and G2b tumours (p<0.0001; FIG. 5B).

These findings, together with those of the gene ontology analysis, suggest that the genetic grade classifier may largely mirror cell proliferation and thus reflect the replicative potential of the breast tumour cells. However, proliferation is not the only oncogenic factor found to be associated with genetic grade. In the G2b tumours, protein levels of VEGF (FIG. 5C), a major inducer of angiogenesis, and the degree of vascular growth (FIG. 5D) are both found to be significantly higher compared to the G2a samples (p=0.015 and p=0.002, respectively) suggesting that a difference in angiogenic potential also distinguishes the two genetic grade classes.

Further analysis of bio-markers revealed yet more oncogenic differences. P53 mutations are found in only 6% of the G2a tumours, whereas 44% of the G2b tumours are p53 mutants (p<0.0001; FIG. 5E) consistent with their higher replicative potential, and likely conferring a further survival advantage to these tumours via decreased apoptotic potential. We also observed higher levels of cyclin E1 protein (p=0.04; FIG. 5F) in the G2b tumours which, in addition to contributing to enhanced proliferation (20), may also confer greater genomic instability (21, 22).

Finally, we observed a significant difference in hormonal status between the G2a and G2b tumours, with an increasing fraction of ER negative (7% versus 19%; p=0.06) and PgR negative (8.5% versus 23%; p=0.02) tumours in the G2b class, indicating differences in hormone sensitivity and dependence.

Taken together, these results show that multiple tumourigenic properties measured at the RNA, DNA, protein, and cellular levels can subdivide the G2a and G2b tumour subtypes—a finding that may explain, in part, the different patient survival outcomes observed between these two genetic classes.

### Example 20

### The Grade Signature is More than a Proliferative Marker

The genetic and clinicopathological evidence suggests that the genetic grade signature reflects, among other properties, the proliferative capacity of tumour cells. That proliferation rate is positively correlated with poor outcome in breast cancer (23) could explain the prognostic capacity of the genetic grade signature.

To further investigate this possibility, we analyzed the major proliferation markers, Ki67, S-phase fraction and mitotic index, together with the genetic grade signature, for survival correlations in Cox regression models (Table S3).

TABLE S3
Multivariate analysis of proliferation markers and the
genetic grade signature for disease-free survival
correlations among patients with Grade II tumours.

| | Uppsala G2 patients | |
|---|---|---|
| | | Hazard ratio |
| Variables | p-value | (95% CI) |
| Genetic grade | 0.0075 | 1.28-4.88 |
| signature | | |
| Ki67 | 0.9300 | 0.92-1.08 |
| S-phase fraction | 0.9200 | 0.50-1.86 |
| Mitotic index | 0.6900 | 0.56-2.40 |

Multivariate analysis showed that the genetic grade signature remained a significant independent predictor of recurrence (p=0.0075) in the presence of these proliferation markers, suggesting that the prognostic power of the grade signature derives from more than just and association with cell proliferation.

### Example 21

### G2a and G2b Tumours are not Identical to Histologic G1 and G3 Cancers

In the survival analysis (FIGS. 3A-3F), we observed no significant survival differences between patients with G1 and G2a tumours, nor those with G3 and G2b tumours. This observation, together with the transcriptional analysis in FIG. 4, suggests that the G2a and G2b classes may be clinically and molecularly indistinguishable from histologic G1 and G3 tumours, respectively.

To address this, we further analyzed the expression patterns of the 264 grade-associated probesets described in FIG. 4. We discovered 14 genes and 57 genes significantly differentially expressed (p<0.01, Mann-Whitney U-test) between the G1 and G2a tumours and G3 and G2b tumours, respectively.

Notably, FOS and FOSB, central components of the AP-1 transcription factor complex, are expressed at higher levels in the G1 tumours, while genes involved in cell cycle progression such as CCNE2, MAD2L1, ASK and ECT2 are expressed at higher levels in the G2a tumours. In a similar fashion, the G3 tumours showed higher expression of cell cycle genes such as CDC20, BRRN1 and TTK as well as proliferative genes with oncogenic potential including MYBL2, ECT2 and CCNE1 when compared to the G2b tumours, while the anti-apoptotic gene, BCL2, is expressed at higher levels in the G2b tumours.

GO analysis of these differentially expressed genes indicated larger biological differences. In the G1-G2a comparison, the differentially expressed genes pointed to differences primarily in cell cycle-related processes and oncogenesis, while differences between the G2a and G3 tumours included cell cycle-related processes, inhibition of apoptosis, oncogenesis and cell motility (Table E4, Table S2).

Statistical analysis of conventional clinicopathological markers revealed further distinctions in the G1-G2a and the G2b-G3 tumour comparisons. As shown in FIGS. 5A-5L, G2a tumours showed significant increases in tumour size (FIG. 5K), lymph node positivity (FIG. 5L), cellular mitoses (FIG. 5A), tubule formation (FIG. 5J) and Ki67 levels (FIG. 5B) compared to histologic G1 tumours, and the G3 population showed significant increases in tumour size (FIG. 5K), vascular growth (FIG. 5D), mitoses (FIG. 5A), tubule formation (FIG. 5J), cyclin E1 (FIG. 5F) and ER negative status (FIG. 5G) when compared to the G2b tumours.

Taken together, these data indicate that the G2a and G2b populations, though highly similar to G1 and G3 tumours in terms of survival and transcriptional configuration, remain separable at multiple molecular and clinicopathological levels.

### Example 22

### Prognostic Potential of the Genetic Grade Signature in G3 Tumours

The prognostic performance of the genetic grade signature in the G2 population suggests that the molecular “misclassifications” in the G1-G3 comparisons might correlate with survival differences. Of the 68 Uppsala and 28 Stockholm G1 tumours, too few are classified as G3-like (ie, 4 in total) for a reliable Kaplan-Meier estimate.

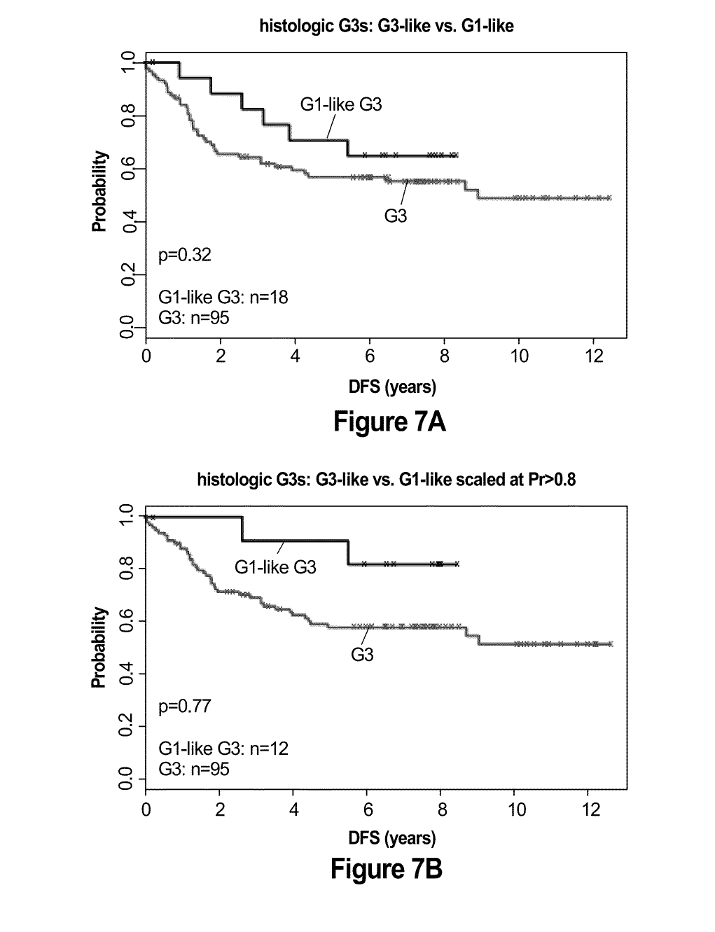

However, among the 55 Uppsala and 61 Stockholm G3 tumours, a total of 18 are classified as G1-like. Kaplan-Meier analysis could not confirm a significant disease-free-survival advantage for these patients, though a trend is observed (FIG. 7A). Interestingly, scaling of the SWS probability (Pr) score to a threshold of Pr>0.8 (for G1-like) resulted in the selection of 12 G1-like G3 tumours associated with only two relapse events (one being a local recurrence only), thus having a survival curve moderately different from that of the remaining G3 population (p=0.077; FIG. 7A).

This finding suggests that the prognostic significance of the classifier may extend also to the poorly differentiated G3 tumours, and that scaling based on the classifier Pr score may allow the fine tuning of prognostic sensitivity and/or specificity, depending on the clinical application.

### Example 23

### Genetic Grade Improves Prognosis by the Nottingham Prognostic Index

The Nottingham Prognostic Index (NPI) is a widely accepted method of stratifying patients into prognostic groups (good (GPG), moderate (MPG) and poor (PPG)) based on lymph node stage, tumour size, and histologic grade (24). It is described in detail in Haybittle et al., 1982. We investigated whether incorporating genetic grade into the NPI could improve patient stratification. A simplified substitution method was explored.

For all tumours of the Uppsala and Stockholm cohorts for which NPI scores and survival information could be obtained (n=382), histologic grade (1, 2 or 3) is replaced by the genetic grade prediction (1 or 3) and new NPI (ie, ggNPI) scores are computed (see Methods). The survival of patients stratified into risk groups is then compared between classic NPI and ggNPI.

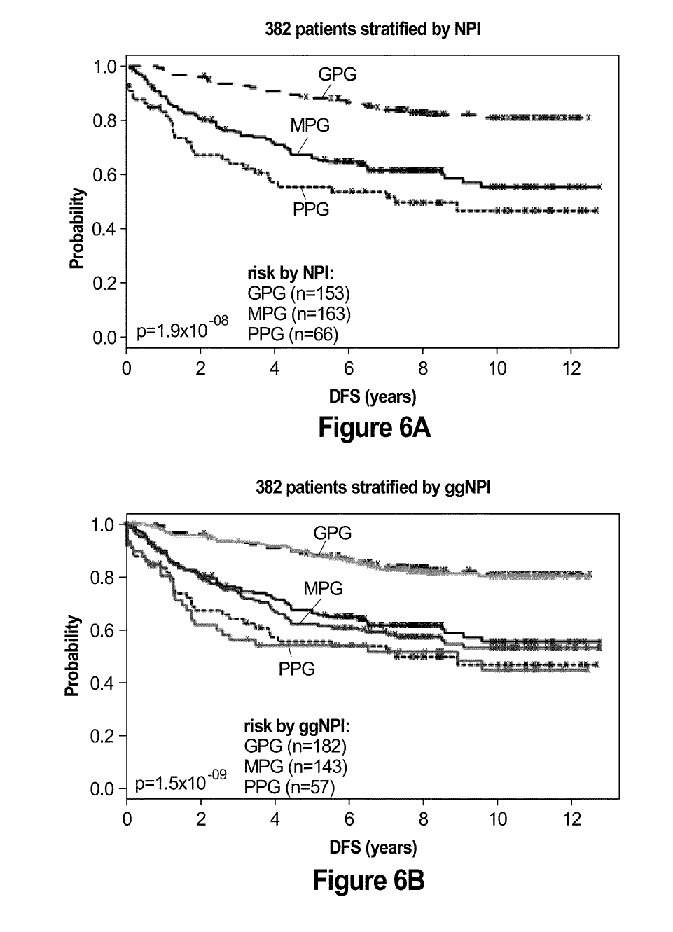

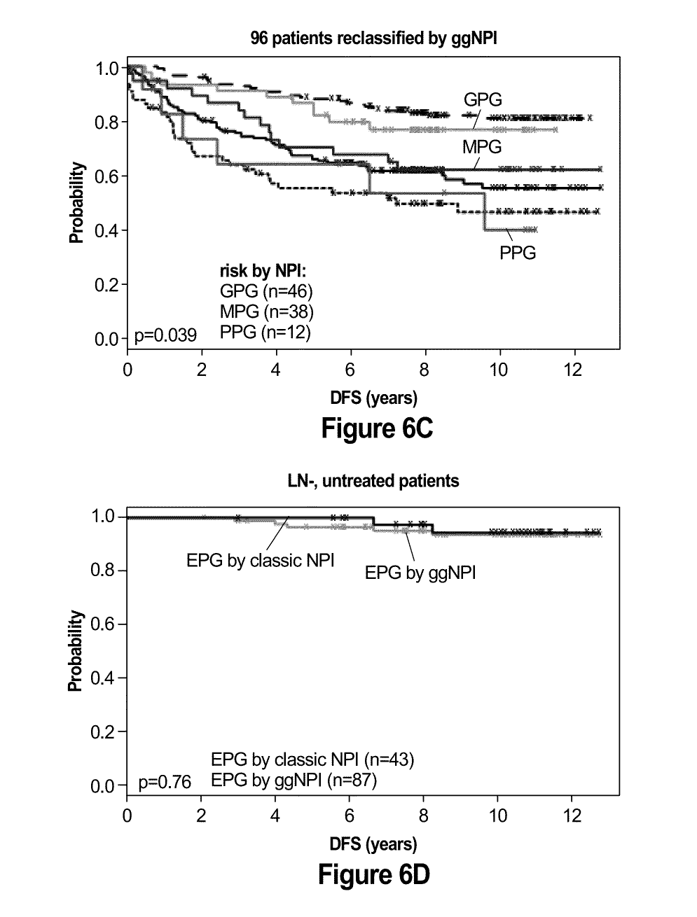

Though the survival curves of the NPI and ggNPI prognostic groups are comparable (FIG. 6A and FIG. 6B), the ggNPI reclassified 96 patients into different prognostic groups (ie, 46 into GPG, 36 into MPG, and 13 into PPG). The survival curves of these reclassified patients are highly similar to the GPG, MPG and PPG of the classic NPI (FIG. 6C) indicating that reclassification by genetic grade improves prognosis of patient risk.

Practical guidelines that use the NPI in therapeutic decision making often recognize an excellent prognostic group (EPG) comprised of patients with NPI scores </=2.4 (25, 26). Untreated patients in this group with lymph node negative disease have a 95% 10-year survival probability—equivalent to that of an age-matched female population without breast cancer (26). Thus, patients in this group are routinely not recommended for post-operative adjuvant therapy (25-27).

We compared the NPI and ggNPI stratifications on a subset of 161 lymph-node-negative patients who received no adjuvant systemic therapy. Forty-three and 87 patients are classified into the EPG by the classic NPI and ggNPI, respectively. Of the 43 patients classified into the EPG by the classic NPI, only one was considered different by the ggNPI; whereas, of those classified as needing adjuvant therapy by the classic NPI (ie, scores >2.4), 45 are reclassified by the ggNPI into the EPG.

When examined for outcome, the survival curves of the 43 and 87 EPG patients by NPI and ggNPI, respectively, are statistically indistinguishable, both showing ˜94% survival at 10 years (FIG. 6D).

Thus, twice as many patients could be accurately classified into the EPG by the ggNPI, suggesting that the use of genetic grade can improve prediction of which patients should be spared systemic adjuvant therapy.

### Example 24

### Discussion

The clinical subtyping of cancer directly impacts disease management. Subtypes indicative of tumour recurrence or drug resistance indicate the need for more aggressive or specific therapeutic strategies, while those that suggest less aggressive disease may specify milder therapeutic options. While clinical subtyping has historically been based primarily on the phenotypic properties of cancer, comprehensive genomic and transcriptomic analyses are beginning to reveal robust genotypic determinants of tumour subtype. In this context, we have studied the transcriptomes of primary invasive breast cancers using expression microarray technology to elucidate the genetic underpinnings of histologic grade, and to use this information to resolve the clinical heterogeneity associated with histologic grade.

Using two different supervised learning algorithms, SWS and PAM, we identified small gene subsets capable of classifying histologic Grade I and Grade III tumours with high accuracy. The smallest gene signature (SWS), comprised of a mere 5 genes (6 probesets), partitioned the large majority of G2 tumours into two highly distinguishable subclasses with G1-like and G3-like properties (G2a and G2b, respectively). Not only are the G2a and G2b tumours molecularly similar to those of histologic G1 and G3, respectively, but the disease-free survival curves of G2a and G2b patients are also highly resemblant of those of G1 and G3 patients. Moreover, these observations are confirmed in a large independent breast cancer cohort. Further analysis revealed that extensive genetic differences between the G2a and G2b classes are accompanied by a host of biological and tumourigenic differences know to separate low and high grade cancer (28) including proliferation rate (mitotic index, Ki67), angiogenic potential (VEGF, vascular growth), p53 mutational status, and estrogen and progesterone dependence, to name a few. Together, these findings demonstrate that the genetic grade signature recognizes and delineates two novel grade-related clinical subtypes among moderately differentiated G2 tumours.

Ma et. al. (2003) were the first to report a histologic grade signature capable of distinguishing low and high grade breast tumours. Using 12K cDNA microarrays to analyse material from 10 G1, 11G2 and 10 G3 micro-dissected tumours, they identified from a list of 1,940 variably expressed, well-measured genes (the top 200 differentially expressed between G1 and G3 tumours (p<0.01 after false discovery correction) (29). Using these genes to cluster their graded tumours, they observed that the majority of G2 tumours possessed a hybrid signature intermediate to that of G1 and G3 with few exceptions (see FIG. 3 in Ma et. al., PNAS, 2003). Notably, this finding is in contrast with our discovery that the majority of G2 tumours do not display hybrid signatures (FIG. 4; profiles of the top 264 gene probesets), but rather possess clear G1-like or G3-like gene features. According to our SWS classifiers, only a small percentage (6%) of the Grade 2 tumours has intermediate grade measurements (i.e. Pr score<0.75 for G1-like and G3-like).

To address this discrepancy, we cross-compared their list of 200 grade-associated genes to our list of 232 and observed a significant overlap of 35 genes (p<1.0×10-7; Monte Carlo simulation) including 2 of our 5 SWS signature genes, MELK and STK6. However, this overlap, despite its significance, represents only a small percentage of either gene list. That the two lists are mostly dissimilar in composition, and that the Ma et. al. study included both invasive (IDC) and noninvasive (DCIS) tumours could explain, to some degree, the variable results observed. Nevertheless, our finding that G2 tumours are predominantly G1-like or G3-like is clinically substantiated by the significant and reproducible survival differences observed between the G2a and G2b classes. It is also possible that differences in sample size (we have much larger number of patients than in Ma etc work), sample preparation, sample size, RNA purification, data normalization could have contributed to the variable results.

To better understand the prognostic value of the genetic grade signature, we compared its performance to other major indicators of outcome in multivariate Cox regression models. In G2 tumours, not only did the classifier remain an independent predictor of disease recurrence, but it is consistently a more powerful predictor than lymph node status and tumour size, underscoring its value as a new prognostic indicator. When incorporated into the Nottingham Prognostic Index (Haybittle et al., 1982), the genetic grade signature improved risk stratification for 25% of patients (compared to the classic NPI) and more than doubled the fraction of lymph node negative patients that should be classified into the excellent prognostic group and thus spared adjuvant treatment.

Breast cancer is thought to progress from a hyperplastic state, to a noninvasive malignant form (carcinoma in situ), to invasive carcinoma and, ultimately, to metastatic disease (30-32). Both the noninvasive and invasive forms can be stratified according to histologic grade. Whether grade is a continuum through which breast cancer progresses, or whether it is merely the endpoint of distinct genetic pathways has been debated (33-38). Studies comparing primary tumours to their subsequent metastases have supported the grade progression model, particularly when multiple metachronous recurrences are analyzed (38, 39). However, comparative genomic studies have identified reproducible chromosomal alterations that distinguish low and high grade disease including a 16q deletion unique to G1 carcinomas (36, 37, 40). These studies argue against the progression model and point to genetic origins of histologic grade. In our study of 494 invasive primary tumours, 94% could be molecularly classified with high probability of being G1-like or G3-like, while only 6% showed intermediate Pr scores (ie, <0.75 for G1-like or G3-like). Notably, we observed these same percentages in the G2 population of 224 tumours. These findings support the genetic pathways model of grade origin, as they suggest that the large majority of breast cancers fundamentally exist in one of two predominant forms marked by the molecular and clinical essence of low or high grade. Whether these forms correlate with the grade-specific genomic alterations previously reported (36, 37, 40) remains to be elucidated.

It should also be noted that although a small percentage (˜6%) of the tumours in our study had intermediate genetic grade measurements (ie, analogous to the hybrid signature observed in Ma et. al. (2003)), too few were discovered to determine the clinical relevance of this intermediate genotype. Furthermore, it is unclear whether these intermediates arise as homogeneous cells that truly borderline low and high grade, or rather represent heterogeneous tumours comprised of distinct low and high grade cell types, such as that observed in tubular mixed carcinoma (38). Alternatively, that we observed the same percentage of intermediacy in tumour classification of all grades and across cohorts, suggests that this class represent a baseline level of uncertainty owing the technical noise.

In conclusion, our results show that the genetic essence of histologic grade can be distilled down to the expression patterns of a mere 5 genes with powerful prognostic implications, particularly in the Grade II setting and in the context of the NPI. The results indicate that G2 invasive breast cancer, at least in genetic terms, does not exist as a significant clinical entity. Indeed, our genetic grade signature dichotomized G2 tumours into two biologically and clinically distinct subtypes that could further be distinguished from G1 and G3 populations. Thus histologic grading, together with measurements of genetic grade, provide a rational basis for the refinement of the G2 subtype into subgrades “2a” and “2b” with immediate clinical ramifications.

Furthermore, our finding that the genetic grade signature could further resolve outcome prediction in G3 tumours, and in a manner dependent on Pr score thresholding, suggests that the genetic grade classifier, viewed as a scalable continuous variable, may have robust prognostic benefit in the diagnosis of all breast tumours. How to optimally weight the genetic grade measurement in combination with other risk factors for greatest prognostic return is a clinical challenge that must next be addressed.

### Example 25

### Introduction: E2F1-Regulates Five Cell Cycle Gene Subset as Early Diagnostic, Low- and High-Aggressive Classifier and as Recurrence Risk Predictor in Breast Cancer

Breast cancer (BC) is one of common malignant disease in women [1-4]. BC comprises heterogeneous tumours with different clinical characteristics, distinct molecular subtypes, and responses to specific treatments.

One of the major challenges of breast cancer therapy is lack of uniform, accurate and reproducible molecular signatures/classifiers that can assist clinicians for treatment decisions across different clinical factors, including histologic grades, clinical stage, tumour mass, ER (+/−)-status or LN(+/−)-status etc. The current existing microarray gene expression or qRT-PCR prognostic/predictive assays in the market [5-8] still have their own limitations in the assisting only specific patient subgroups for treatment recommendation [9-11]. Significant discordance remains between clinical assay-defined subsets and intrinsic subtype. Such situation is occurred for tumours with borderline hormone receptor (HR; ER, PG, HER2) expression are highly biologically heterogeneous, which raises the question of whether these tumours should be considered indeterminate. A significant proportion of clinically defined HER2-negative tumours were defined as molecular HER2-positive subtype; however, whether they are suitable for anti-HER2 therapy needs to be determined [85].

Clinical influence of the most popular in USA the RT-PCR-based 21-gene recurrence score assay (Oncotype DX) in woman with early-stage, estrogen receptor-positive, lymph node-negative breast cancers was recently evaluated in 70,802 Medicare recipients diagnosed with breast cancer between 2005 and 2009 [12]. In 2005-th assay was used for just 1.1% of woman compared to 10.1% in 2009. The test was assumed to be informative regarding the potential benefits of adjuvant chemotherapy. Nevertheless, the authors noted that chemotherapy rates in this sub-set intermediate-risk BC patients, was not significantly changed from 2005 to 2009 year and concluded that factor influencing adoption of the assay and its impact on adjuvant chemotherapy use in clinical practice remain important area of study.

An extensive search is still on-going to assess the patient's treatment modalities. Majority of gene signature-based assay panels are problematic due to lack of robust performance (reproducibility) at the level of multi-cohort datasets and their inability to stratify effectively distinct patient groups and intra-tumour heterogeneity. Further, the computational predicted post-surgery treatment breast cancer risk recurrence lack extensive experimental screening methods [13-17] leading to poor prediction and suboptimal therapeutic capabilities. Moreover, majority of micro-array or qRT-PCR-based prognostic/prediction assays are inconsistent in digging underlying regulatory mechanisms of the genes included and/or associated with the signatures, leading to a scepticism of the oncologists and poorly prognostic performance.

It is known that overexpression of cell cycle/mitotic genes play a major role in BC stem cell initiation, clonal expansion, tumour progression and they determined outcome of the disease and therapeutic intervention. Expression of the proliferative genes correlate with BC histologic grading system(s), scoring tumour aggressiveness based on proliferative rate and a level of dedifferentiation of breast epithelial cells, accompanied with morphological disorder in transformed mammary tissue. In general, tumours are graded as 1, 2, 3, or 4, depending on the amount of abnormality. In histologic grade 1 (G1) tumours, the tumour cells and the organization of the tumour tissue appear close to normal. These tumours tend to grow and spread slowly. In contrast, the cells and tissue of histologic grades 3 and 4 (HG3, HG4) tumours do not look like normal cells and tissue. G3 and G4 tumours tend to grow rapidly and often spread faster than tumours with a lower grade (G1). Histologic grade 2 (G2), consist of about 50% of breast cancer patients and is classified as moderately differentiated (intermediate grade). However, G2 is not homogeneous; for instance, it includes ER-positive and ER-negative BC tumours. Farther more HG2 ER-positive tumours consist of two clinically distinct intrinsic subtypes classified molecularly as Luminal A and Luminal B [84].

The genetic tumour aggressiveness grading signature (TAGs), included 232 genes [18] is a computationally-derived microarray-based molecular analogue of the histologic grading system of BC, consisting of mostly the transcribed genes related to mitosis, chromosome condensation, chromosome segregation, mitosis, and kinetochore machineries [18] which are the cell cycle/proliferation genes,—key hallmark of cancers [19, 20]. Moreover, 232g-TAGs reclassifies the histologic grade II (G2) breast tumours in histologic grade I-like (G1-like) and in histologic grade 3-like (G3-like) molecular sub-classes, stratifying G2 tumours of BC patients onto low- and high-aggressive types with significantly distinct clinical outcomes. Several small representative signatures have been also derived, which independently from ER, PR, tumour size, lymph node status of the patients provided a very similar and robust genetic and clinical features, as the 232g-TAGs [18].

There exist various prognostic gene signature panels in market such as Mammaprint, Theraprint, Targetprint, OncotypeDx, and PAM50 that could assess the risk of disease development of breast cancer patients [21-24].

In contrast to the conventional prognostic signatures (e.g. MammaPrint, Oncotype DX, or MiK67 test), TAGs quantitatively stratifies BC patients with respect to clinical outcome equally well, without pre-selection of the patient based on ER, PR and LN status, tumour size and also assists in re-classifying the histologic grade II BC patients onto low- and high-risk subgroups, which are similar to the histologic grade I and grade III, respectively [18], which are well-known are strongly correlated with p53 status and chromosome alteration pattern in low and high-aggressive breast cancers.

Herein, we study patho-biological and clinical values of six cell-cycle genes (BRRN1 (NM_015341), AURKA (NM_003600), MELK (NM_014791), PRR11 (NM_018304), CENPW (NM_001012507) and E2F1 (NM_005225)), called hear 6g-TAG), representing the 232g-TAGgenes, reported previously (Ivshina et al, 2006)). We test the hypotheses that these 6 genes and their products could be coincident in cancer cell functions and potentially utilized in clinical practice as (i) the early diagnostic multi-gene biomarker having the recurrence free survival and treatment outcome significances; (ii) the accurate and reproducible cell cycle-based clinical classifier of the low- and high-grade aggressive tumours (including primary tumours, local and distant metastases).

We proposed a method of quantification of pathobiological and clinical significance of the six cell-cycle genes. We demonstrated that these six genes and their products (RNA, proteins) could be transcriptionally co-regulated by E2F1 transcription factor in cell cycle, over-performed in the comparison with commercial BC prognostic assays and potentially can be utilized in clinical practice as (i) a reproducible cell cycle-based clinical classifier of the low- and high-molecular grade aggressive tumours and (ii) the early diagnostic multi-gene biomarker and (iii) the predicting function of the recurrence within the patient cohorts of the given histological grade, ER-status, LN-status, molecular tumour subtype and metastatic states of breast cancers.

A prototype of qPCR-based assay is developed and validated. We characterized the functions of the genes and validated the 6g-TAGs in several BC microarray data and over our sets tumour samples via qRT-PCR analysis. We showed that these 6 genes and their products are co-expressed in G1/S, G2/M transition of cell cycle, and form in BR CA cells the interactive network hubs transcriptionally controlled by E2F1. At protein-protein interaction level, we demonstrated that PRR11, BRRN1 and MELK can be co-localized and realize their functions within breast individual cancer cells. Our bioinformatics and statistical analyses suggested that the 6g-TAGs genes act collectively as inter-connecting network hubs, with critical regulatory role in G1/S, G2/M transition. The 6g-TAGs dichotomized of the histologic grade-2 (G2) tumours onto histologic grade 1-like (G1-like) and histologic grade 3-like (G3-like) sub-classes and robustly stratified BC patient' survival pattern according the recurrence risks onto genetically low-grade (GLG=G1+G1-like) and genetically high-grade (GHG=G3-like+G3) tumour classes. In summary, our integrative microarray and qRT-PCR analysis in combination with experimental and clinical data suggests that 6g-TAGs assay is a perspective clinical biomarker with strong early cancer diagnostic, classification, prognostic and therapeutic value.

### Example 26

### Materials and Methods: Patients Samples and Microarrays

Commercial total RNA samples of 58 breast adenocarcinoma patients and 4 normal breast tissue samples were obtained from OriGene. BC patients were classified based on comprehensive clinical information including TNM, stage, histological grade (grade 1 (G1): 5 samples; grade 2 (G2): 16 samples; grade 3 (G3): 37 samples) and survival information.) Microarray gene expression studies were carried out using U133 Plus 2.0 Affymetrix. The microarray dataset was normalized using RMA (Robust Multichip Average) method. The dataset was uploaded recently to NCBI Gene Expression Omnibus (GSE61304).

The quality of total RNA of each patient samples obtained was analysed using Agilent 2100 Bio Analyzer (all samples have RIN value of above 8). The GeneChip 3′ in vitro transcription (IVT) protocol that includes reverse transcription to synthesize first strand cDNA, second-strand cDNA, biotin-modified RNA labelling, RNA purification and fragmentation have been carried out using Affymterix manufacturer's protocol. A total of 500 ng of RNA were used from each RNA sample for the above procedure. Positive control RNA provided by manufacturer's were used for quality control checking. Hybridization, subsequent washing, and staining of the arrays were carried out as outlined in the GeneChip® Expression Technical Manual. All the hybridization and scanning procedures were done at Biopolis Shared Facility (BSF), A-STAR.

Additionally, three microarray gene expression datasets based on Affimetrix U133 A&B platform, called Stockholm, Uppsala and Singapore cohorts were used along with in-house microarray dataset (GSE61304)

To assess diagnostic significance of TAGs genes, GSE10780 data set was downloaded from GEO NCBI. These samples are categorized into three different histological types Normal, IDC-normal like and IDC [24].

### Example 27

### Materials and Methods: Cell Lines

Two breast cancer cell lines were selected, MCF10A (normal like, non-tumourigenic, low grade, and MDA-MB-436 (invasive tumourigenic high grade) to quantify the protein expression levels of 6 TAGs genes. MCF-10A and MDA-MB-436 cells were obtained from the ATCC. MCF-10A cells were cultured in supplements of Insulin, Cholera toxin and epidermal growth factors along with 10% fetal bovine serum (FBS). MCF-10A cells were dissociated using trypsin 5% for 15 minutes at 37° C. and then cells were then spun down at 1000 rpm for 5 minutes. The supernatant was subsequently aspirated and the pellet of cells was supplanted with based media for further downstream processes. For MBA-MB-436 DMEM F-12 medium with essential amino acids along with 10% fetal bovine serum were used.

### Example 28

### Materials and Methods: RT-PCR and qPCR Studies

cDNA was synthesized from 62 total RNA samples using Qiagen cDNA synthesis kit. These cDNA were tested initially with endogenous control b-actin (primers provided by OriGene), to ensure equal amount of cDNA loaded in each plate well. The 58 tumour cDNA samples were used for further downstream qPCR analysis. Primers were designed for CENPW (Forward—CGTCATACGGACCGGATTGT (SEQ ID NO: 1), Reverse—GGAGACTATGGTCGACAGCG (SEQ ID NO: 2)), PRR11 (Forward—CAAAGCTGCTACTGCCATTG (SEQ ID NO: 3), Reverse—CTGGTTGCCATTCAGTCTCA(SEQ ID NO: 4)), MELK (Forward—CAAACTTGCCTGCCATATCCT (SEQ ID NO: 5), Reverse—GGCTGTCTCTAGCACATGGTA (SEQ ID NO: 6)), AURKA(Forward—AGCTAGAGGCATCATGGACCG (SEQ ID NO: 7), Reverse—GCTCAGCTGGAGAAAGCCGGA (SEQ ID NO: 8)), and BRRN1 (Forward—TGCCAAAAAGATGGACATGA (SEQ ID NO: 9), Reverse—CCGCTAAGCATCTTCTCGTC(SEQ ID NO: 10)), E2F1 (forward—GCTGTTCTTCTGCCCCATAC (SEQ ID NO: 11), Reverse—GAAGGCCCATCTCATATCCA(SEQ ID NO: 12)) and run q-PCR experiment and further extracted CT values using ABI 7300. Relative quantification was estimated using ddCT method [25-27] for each gene and further estimated mean average mRNA levels of G1 and G3 patients for the genes. Applied Biosystems 7300 Real Time PCR machine was used with compatible SYBR green master mix.

### Example 29

### Materials and Methods: Western Blotting/Immunoblotting Assays

Breast cancer MDA-MB-436 cells were isolated at G1, S and G2/M cell cycle phases using propidium iodide (PI) dye by FACS analysis (detailed in Flow cytometer method) and further extracted total RNA from each sub-population of cells and carried out cDNA synthesis followed by PCR amplification using above specified primers of TAGs genes and run DNA agarose gel for further RT-PCR analysis.

Pelleted cells were lysed using lysis buffer (commercial Bio-Rad) and estimated proteins (Bio-Rad protein assay) and loaded equal amount of protein and separated by SDS-PAGE [28-30]. After transfer, the membranes were probed with commercial rabbit polyclonal antibodies of Actin, C6orf173, AURKA, MELK and PRR11 (Cell sciences, Sigma Aldrich). Commercial mouse monoclonal antibody available for BRRN1 was obtained from Cell Signalling. Commercial rabbit polyclonal antibody of E2F1 obtained from Thermo Scientific. B-Actin (cell signalling) was used as internal control to relatively compare the expression levels of 6-TAGs genes. Secondary antibodies (anti-rabbit and anti-mouse IgG horseradish peroxidase-conjugated) were purchased from GE Healthcare Bio-Sciences AB. Proteins were visualized using an enhanced chemiluminescence (ECL) reagent kit (GE Healthcare Bio-Sciences AB). Densitometry analysis of Western Blot images was done using ImageJ open source software.

### Example 30

### Materials and Methods: Immunostaining and Imaging

MDA-MB-436 cells (primary and transfected (GFP-PRR11) were cultured at 370 C, described above with appropriate antibiotics. Prior to immunostaining experiments, the cells were grown on coverslips. Immunostaining and digital image capturing was performed as described earlier [31]. Briefly, cells on coverslips were fixed in a 1:1 mixture of cold methanol and acetone (−20° C.). After re-hydration in phosphate buffer saline, cells were stained with antibodies. Hoechst 33258 (Sigma-Aldrich) was added at a concentration of 0.4 μg/ml to the secondary antibody for DNA staining when necessary. 510 laser scanning confocal microscope with ORCA-ER CCD camera (Hamamatsu). Confocal microscopy images of MDA-MB-436 cells were acquired in a point scanning confocal microscope Zeiss LSM 510 Meta (Zeiss, Germany), with a 40×EC Plan-Neofluar oil immersion objective, and diode (405 nm), argon (488 nm), DPSS (561 nm) and helium-neon (633 nm) lasers; cells were excited at 405 nm (Hoechst 33342), 488 nm (FAM) and 561 nm (rhodamine). Differential interference contrast (DIC) images were obtained using the helium-neon laser (633 nm). Digital images were acquired using the LSM 510 Meta software. All instrumental parameters pertaining to fluorescence detection and image analyses were held constant to allow sample comparison.

### Example 31

### Materials and Methods: Immunoprecipitation Studies

For the immunoprecipiation 5 ug of the antibodies (rabbit anti-PRR11 and mouse anti-BRRN1) were coupled to the CN-Br sepharose 4 Fast Flow according manufacturer protocol (GE Healthcare Bio-Sciences AB) and such supports were used to capture the corresponding proteins from the NP40 cell lysates (usually 1×107 MDA-MB-436 cells were used for one probe). After extensive washing with NP40 lysis buffer once and PBS (at least 20 volumes) the protein complexes were eluted by a heat (940 C) and separated on the SDS-PAGE.

### Example 32

### Materials and Methods: Flow Cytometry

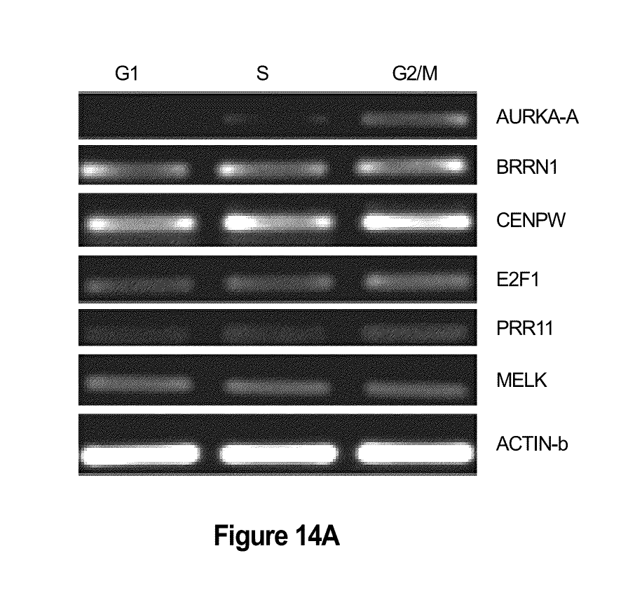

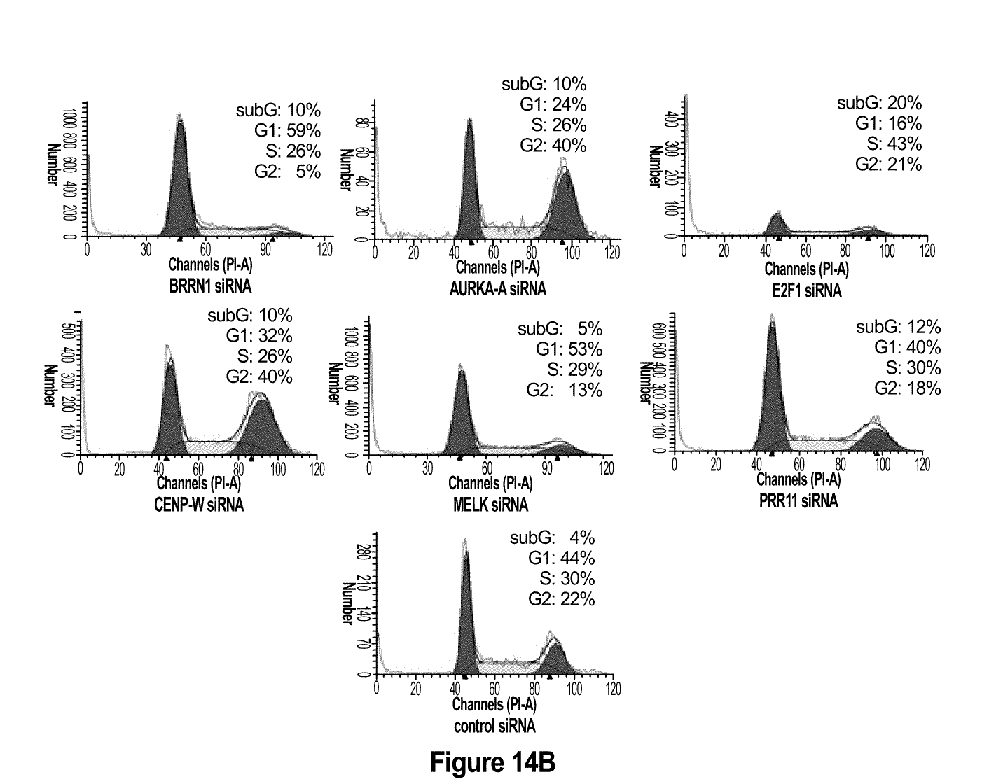

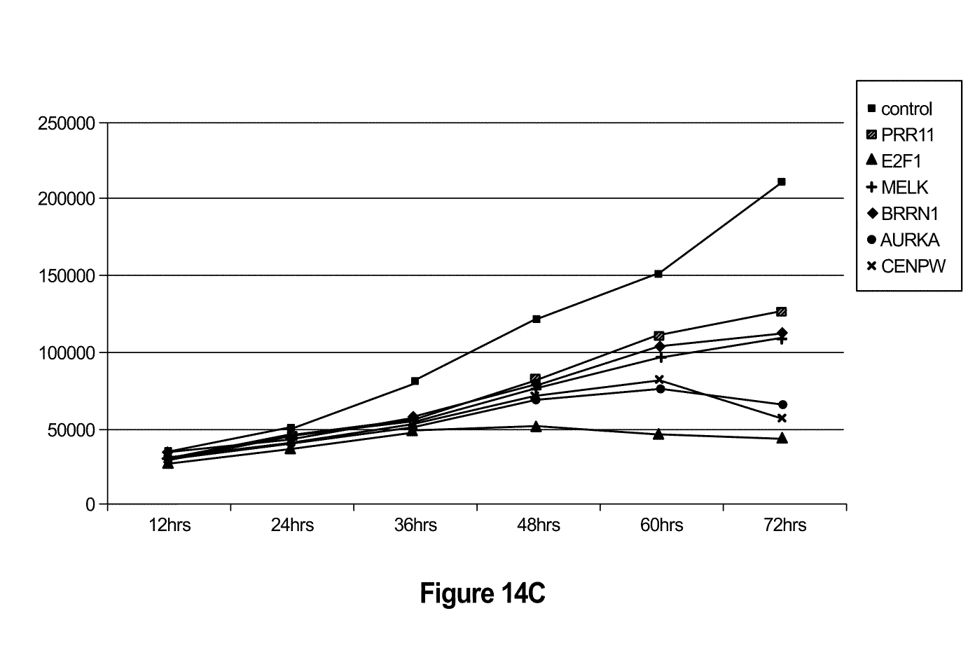

MDA-MB-436 breast cancer cells were harvested and spun down and remove supernatant and resuspend pelleted cells and add 1 ml of fresh medium (described above) and filter cells trough cup with cell stainer filter (BD commercial) to avoid clumps add working solution of Hoechst 15 ul (stock: 1 mg/ml in DMSO) and foil (Aluminum) the tube to avoid light incubate @37 C for 15 min prepare one more tube with 1 ml of fresh medium for cells to be collected for cell cycle analysis (re-suspend cells) using BD FACs Ariallu SORT available at our Bioshared Facility services. The collected cells at various cell cycle phases were subjected for RNA isolation followed by cDNA synthesis and RT-PCR experiments. Verity Software (Modfit LT3.3) was used to assess percentage of cells at various cell cycle phases after siRNA silencing of the 6g-TAGs genes. To measure the proliferation rate of MDA-MB-436 in siRNA treated 6g-TAGs genes, we seeded 5,000 cells in 12-well plates and counted cells at various time points indicated and compared relatively with control siRNA treatment as represented in FIGS. 14A-14C.

### Example 33

### Materials and Methods: Statistics and Bioinformatics—Data-Driven Grouping (DDg) Method

Data driven grouping (DDg) is a computational method for the genome wide identification/selection of the survival significant genes and patient grouping/stratification in to disease development risk groups, reflecting training patient set groping according the disease survival events and last follow up of the patients. This method, based on fitting a semi-parametric Cox proportional hazard regression model, is used to fit patients' survival times/last follow-up and events to gene expression value data. In this study, disease free survival (DFS) data were used. One dimensional data driven grouping (1D DDg) method [32] was used for fast and efficient screening of massive gene expression datasets to identify/select potential individual genes-candidates (predictors) and these gene expression discriminative cut-off values for construction rule of the prognostic/predictive patient stratification [33]. The model estimates the optimal partition (cut-off) of expression level values of a gene by maximizing the separation of the survival (Kaplan-Meier) curves related to the different (high- and low-) risks of the disease behaviour [32]. We also used SurvExpress web resource and the online Kaplan-Meier Plotter for selection of multi-gene classifiers, stratification of the patients into significant survival subgroups, comparison of these groups These two programs were used on validation stage of our prognostic classifiers.

### Example 34

### Materials and Methods: Statistically Weighted Voting Grouping (SWVg) Method

Statistically weighted Syndrome grouping (SWVg) grouping method is based on a dichotomization of survival data and selection of optimal (best) prognostic features and weighted used to obtain consensus grouping decisions from the patient survival grouping information generated by multiple prognostic covariates (e.g., expression values of genes) [32, 34]. SWVg is a multivariate voting classification and feature selection algorithm deriving the prognostic covariate (e.g. expressed gene subset) composed of a prognostic signature that is able to robustly separate the patients of two (or more) groups. It has taken all the grouping information across the list of SWVg-selected the selected prognostic covariate (selected genes). Each survival significant covariate after applying DDg provides patients' grouping and SWVg further synergizes survival information of all such prognostic covariate and separates the patients into robust (overall) survival groups discriminated by SWVg with log-rank statistics p-value smaller then each of the selected prognostic covariate along.

### Example 35

### Materials and Methods: HG2 Sub-Classification of Breast Cancer Patient Samples Using Balanced Statistically Weighted Syndrome (SWS) Classification Method

The sub-classification of G2 was performed using Statistically Weighted Syndrome (SWS) algorithm based on G1 and G3 tumours [Kuznetsov et al, 1996; Kuznetsov 2006]. G1 and G3 tumours were used as training subsets and the G2 tumours were used as class discovery set. The classifier assigned each tumour of G2 as either G1-like or G3-like tumours with the estimated probability. We applied this procedure for classification of testing group consists of 62 tumours. These tumour samples include 4 normal, 5 G1, 16 G2 and 37 G3 tumours. Due to the limited number of G1 tumours we combined the 4 normal tumours with HG1 tumours to obtain 9 tumours as low grades during the training of the classifier. Also there is an imbalance between low grade (LG) and G3 tumours, therefore we split G3 tumours randomly into two non-overlapping subgroups and performed two training-prediction iterations. The obtained training accuracies for both balanced iterations were accuracy was 96.4% and 92.6%, respectively. (Table EE6).

TABLE EE6
Genes significantly correlated with 6 TAGs genes. In combination, the positively and negatively correlated
gene sets could be considered separately or together as a novel combined TAG-defined BC prognostic,
predictive and diagnostic signature(s).

| | | |
|---|---|---|
| Gene | | Affymetrix |
| Symbol | Gene Name | Probe set ID |

A. List of the genes positively correlated with 6 TAGs genes

| | | |
|---|---|---|
| ACTR2 | ARP2 actin-related protein 2 homolog (yeast) | 200728_at |
| ACTR3 | ARP3 actin-related protein 3 homolog (yeast) | 200996_at |
| ACTR3B | ARP3 actin-related protein 3 homolog B (yeast) | 218868_at |
| AKAP8 | A kinase (PRKA) anchor protein 8 | 203847_s_at |
| ANAPC1 | anaphase promoting complex subunit 1 | 218575_at |
| ANAPC10 | anaphase promoting complex subunit 10 | 207845_s_at |
| ANAPC11 | anaphase promoting complex subunit 11 | 226414_s_at |
| ANAPC5 | anaphase promoting complex subunit 5 | 200098_s_at |
| ANAPC7 | anaphase promoting complex subunit 7 | 225554_s_at |
| ARPC1A | actin related protein 2/3 complex, subunit 1A, 41 kDa | 200950_at |
| ARPC1B | actin related protein 2/3 complex, subunit 1B, 41 kDa | 201954_at |
| ARPC2 | actin related protein 2/3 complex, subunit 2, 34 kDa | 213513_x_at |
| ARPC3 | actin related protein 2/3 complex, subunit 3, 21 kDa | 208736_at |
| ARPC5 | actin related protein 2/3 complex, subunit 5, 16 kDa | 211963_s_at |
| AURKB | aurora kinase B | 209464_at |
| BCL2L14 | BCL2-like 14 (apoptosis facilitator) | 234191_at |
| BRCA1 | breast cancer 1, early onset | 204531_s_at |
| CCNB1 | cyclin B1 | 214710_s_at |
| CCNB2 | cyclin B2 | 202705_at |
| CDC2 | cyclin-dependent kinase 1 | 203213_at |
| CDC20 | cell division cycle 20 | 202870_s_at |
| CDC23 | cell division cycle 23 | 223651_x_at |
| CDC25A | cell division cycle 25A | 204695_at |
| CDC25B | cell division cycle 25B | 201853_s_at |
| CDC25C | cell division cycle 25C | 205167_s_at |
| CDC26 | cell division cycle 26 | 225422_at |
| CENPA | centromere protein A | 204962_s_at |
| CENPE | centromere protein E, 312 kDa | 205046_at |
| DLGAP5 | discs, large (Drosophila) homolog-associated protein 5 | 203764_at |
| DYNC1LI1 | dynein, cytoplasmic 1, light intermediate chain 1 | 222479_s_at |
| DYNLRB1 | dynein, light chain, roadblock-type 1 | 217917_s_at |
| DYNLT1 | dynein, light chain, Tctex-type 1 | 201999_s_at |
| E2F1 | E2F transcription factor 1 | 2028_s_at |
| E2F4 | E2F transcription factor 4, p107/p130-binding | 202248_at |
| EIF2AK1 | eukaryotic translation initiation factor 2-alpha kinase 1 | 217736_s_at |
| ETV4 | ets variant 4 | 211603_s_at |
| FAF1 | Fas (TNFRSF6) associated factor 1 | 224217_s_at |
| FBXW7 | F-box and WD repeat domain containing 7, E3 ubiquitin protein ligase | 229419_at |
| GABPA | GA binding protein transcription factor, alpha subunit 60 kDa | 210188_at |
| HIST1H3B | histone cluster 1, H3b | 208576_s_at |
| HIST1H3F | histone cluster 1, H3f | 208506_at |
| HIST1H3G | histone cluster 1, H3g | 208496_x_at |
| HNF4A | hepatocyte nuclear factor 4, alpha | 214851_at |
| INCENP | inner centromere protein antigens 135/155 kDa | 219769_at |
| KIF2A | kinesin heavy chain member 2A | 203087_s_at |
| KIF2C | kinesin family member 2C | 209408_at |
| LATS2 | large tumour suppressor kinase 2 | 230348_at |
| MAP9 | microtubule-associated protein 9 | 235550_at |
| MAX | MYC associated factor X | 210734_x_at |
| NCAPD2 | non-SMC condensin I complex, subunit D2 | 201774_s_at |
| NCAPD3 | non-SMC condensin II complex, subunit D3 | 212789_at |
| NCAPG | non-SMC condensin I complex, subunit G | 218662_s_at |
| NCAPG2 | non-SMC condensin II complex, subunit G2 | 219588_s_at |
| NDC80 | NDC80 kinetochore complex component | 204162_at |
| NR1I2 | nuclear receptor subfamily 1, group I, member 2 | 207203_s_at |
| NUF2 | NUF2, NDC80 kinetochore complex component | 223381_at |
| PPP1CA | protein phosphatase 1, catalytic subunit, alpha isozyme | 200846_s_at |
| PPP1CB | protein phosphatase 1, catalytic subunit, beta isozyme | 201407_s_at |
| PPP1CC | protein phosphatase 1, catalytic subunit, gamma isozyme | 200726_at |
| PPP1R8 | protein phosphatase 1, regulatory subunit 8 | 207830_s_at |
| PPP2CA | protein phosphatase 2, catalytic subunit, alpha isozyme | 208652_at |
| PRKACB | protein kinase, cAMP-dependent, catalytic, beta | 235780_at |
| RALA | v-ral simian leukemia viral oncogene homolog A (ras related) | 214435_x_at |
| SKP2 | S-phase kinase-associated protein 2, E3 ubiquitin protein ligase | 203625_x_at |
| SMAD2 | SMAD family member 2 | 203075_at |
| SMC2 | structural maintenance of chromosomes 2 | 204240_s_at |
| SMC4 | structural maintenance of chromosomes 4 | 201663_s_at |
| SP3 | Sp3 transcription factor | 229217_at |
| TUBA1B | tubulin, alpha 1b | 201090_x_at |
| TUBA1C | tubulin, alpha 1c | 209251_x_at |
| TUBA3D | tubulin, alpha 3d | 216323_x_at |
| TUBA4A | tubulin, alpha 4a | 212242_at |
| TUBB | tubulin, beta class I | 209026_x_at |
| TUBB1 | tubulin, beta 1 class VI | 230690_at |
| TUBB2C | tubulin, beta 4B class IVb | 213726_x_at |
| TUBB3 | tubulin, beta 3 class III | 202154_x_at |
| ZNF622 | zinc finger protein 622 | 225152_at |

B. List of the genes negatively correlated with 6 TAGs genes

| | | |
|---|---|---|
| ANAPC2 | anaphase promoting complex subunit 2 | 218555_at |
| ANAPC4 | anaphase promoting complex subunit 4 | 226917_s_at |
| AR | androgen receptor | 211110_s_at |
| ARHGEF2 | Rho/Rac guanine nucleotide exchange factor (GEF) 2 | 235595_at |
| BTRC | beta-transducin repeat containing E3 ubiquitin protein ligase | 224471_s_at |
| CPEB1 | cytoplasmic polyadenylation element binding protein 1 | 219578_s_at |
| ERG | v-ets avian erythroblastosis virus E26 oncogene homolog | 213541_s_at |
| ESR1 | estrogen receptor 1 | 211234_x_at |
| ESR2 | estrogen receptor 2 (ER beta) | 210780_at |
| ETV1 | ets variant 1 | 217053_x_at |
| EWSR1 | EWS RNA-binding protein 1 | 229966_at |
| FLI1 | Fli-1 proto-oncogene, ETS transcription factor | 210786_s_at |
| NEDD9 | neural precursor cell expressed, developmentally down-regulated 9 | 202149_at |
| PARD3 | par-3 family cell polarity regulator | 221527_s_at |
| SMAD3 | SMAD family member 3 | 205397_x_at |
| SMAD4 | SMAD family member 4 | 235725_at |
| SP1 | Sp1 transcription factor | 224754_at |
| SPIN1 | spindlin 1 | 217813_s_at |
| TEAD1 | TEA domain family member 1 (SV40 transcriptional enhancer factor) | 214600_at |
| TP53 | tumour protein p53 | 211300_s_at |
| TUBA4B | tubulin, alpha 4b (pseudogene) | 207490_at |
| ZBTB17 | zinc finger and BTB domain containing 17 | 203601_s_at |

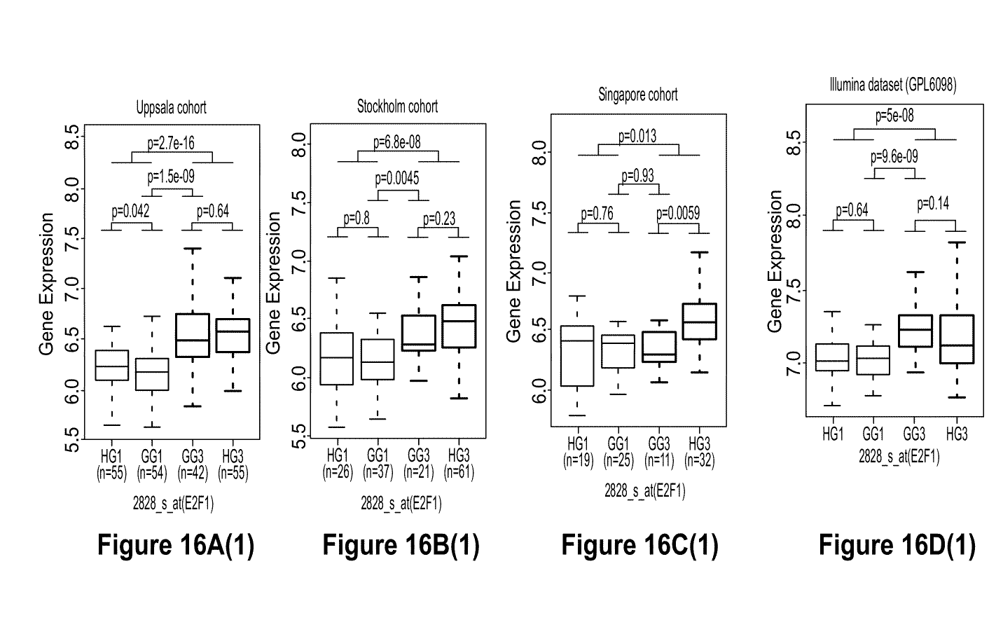

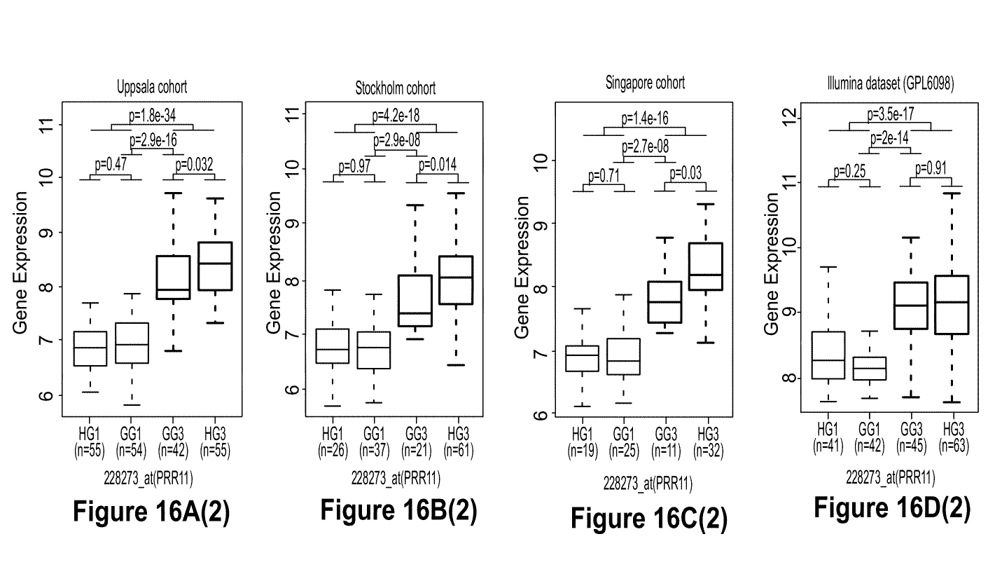

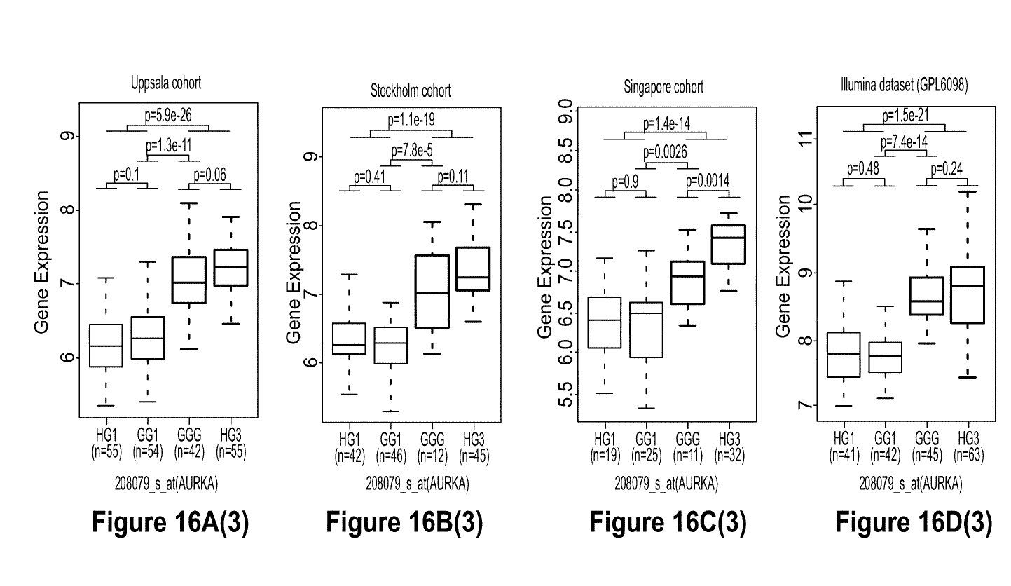

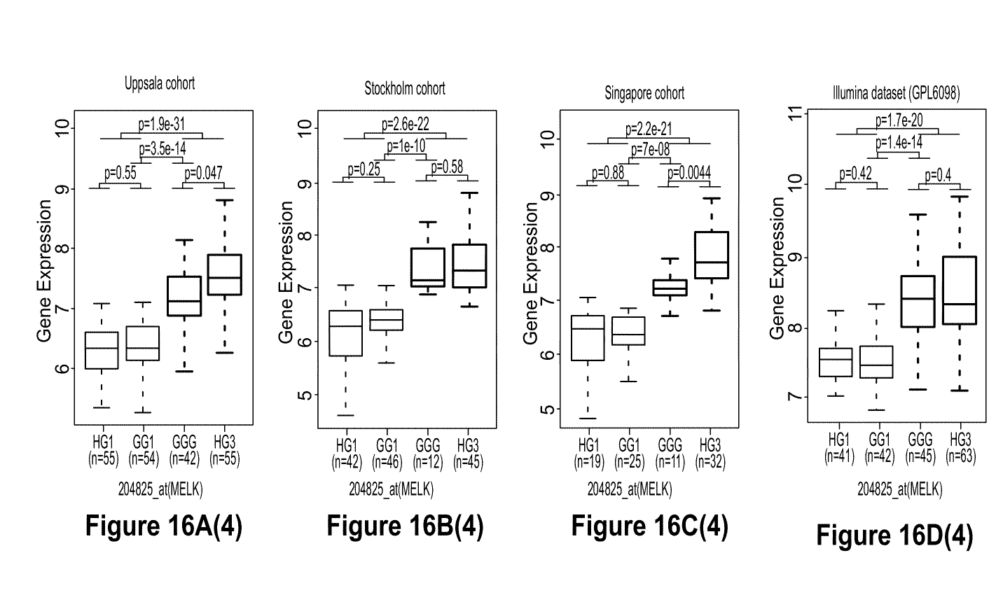

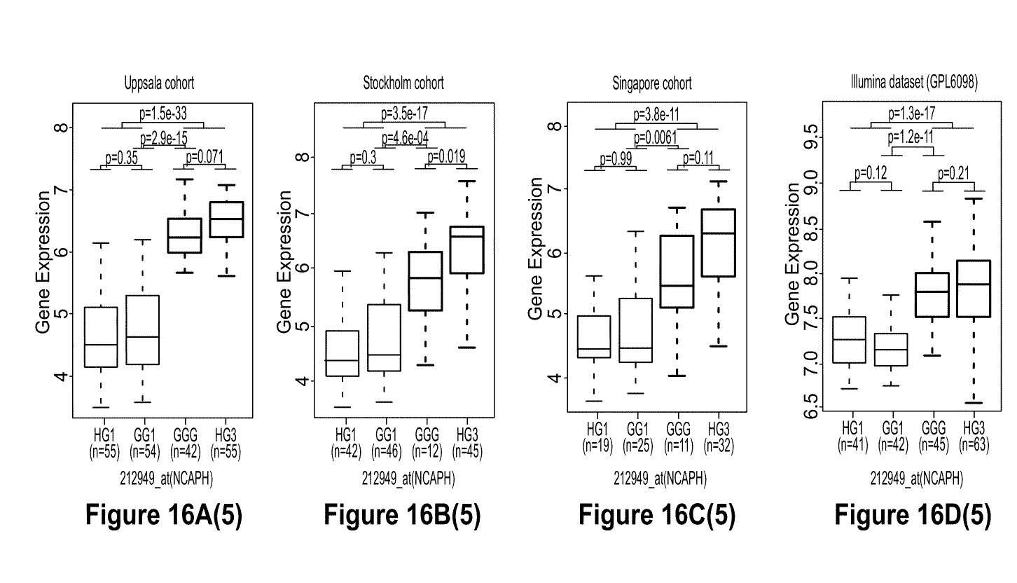

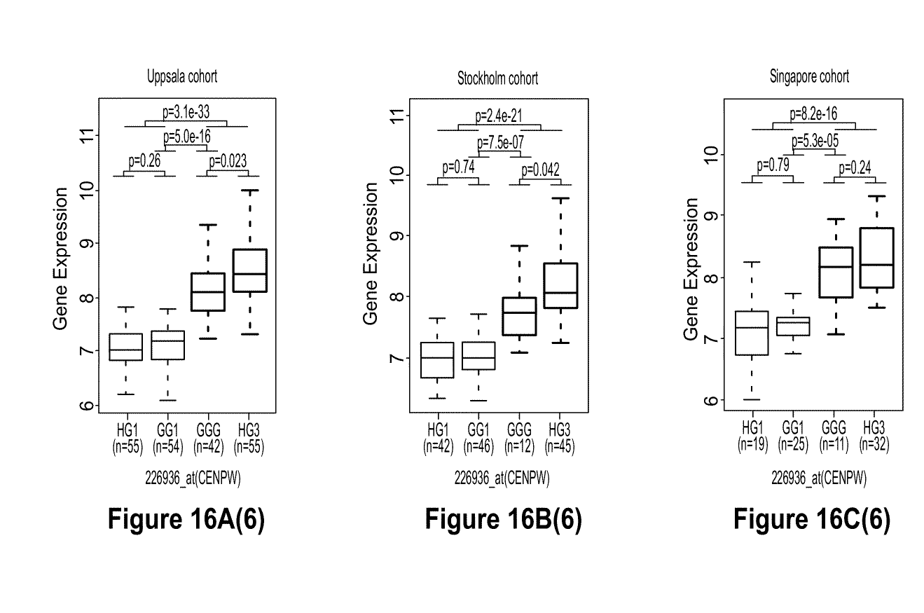

G2 tumour samples were used as class prediction set and sub-classified into HG1-like and G3-like tumours based on the assigning probability of both training-prediction iterations. According to this procedure, six tumours were assigned to G1-like and 10 tumours were assigned to G3-like subclasses. The expression levels of the 6g -TAG genes in G1, G1-like, G3-like and G3 for Uppsala, Stockholm and Illumina data sets is depicted in FIGS. 16A(1)-16D(5). Statistical characteristics of these figures strongly demonstrate that G1 and G-like tumours cold represent the low-grade BCs and G3-like and G3 tumours could represent high-grade BCs.

### Example 36

### Materials and Methods: Tests and Correlation Analysis

For analysis of the gene co-expression patterns and for selection of potential gene network interactors, microarray expression probes with significant Kendall correlation coefficients (|τ|≧0.2 and P(τ, FDR)≦0.01) correlated with a given target gene, were selected. Next, strongly correlating probes were separately analyzed using the “1-D DDg algorithm” [19]. The probes with significant impact on the survival of the patients were selected according to the criterion FDR≦0.05.

### Example 37

### Materials and Methods: Metacore Network Analyses

Network analysis of the 6-TAGs genes was carried out using MetaCore™ software. The genes PRR11, MELK, BRRN1, AURKA, and MELK were used as seed nodes to extent the network using MetaCore, automatic expand to 50 nodes network building option had been used to build the TAGs network. Result in the network consists of nodes (protein or protein complex) among them AURKA, MELK, and E2F1 forms a network hub. The network nodes were extracted for further gene co-expression analysis. David gene ontology studies were conducted in parallel comparison to metacore for better statistical reliability [35, 36].

### Example 38

### Materials and Methods: Cyclebase Web Tool for Periodic Cell Cycle Gene Data Analysis

Cyclebase 3.0 is a web tool with a overview of cell-cycle regulation and phenotypes for a given gene of interest. Its main features include (a) aiming to provide a concise overview of cell-cycle regulation and phenotypes for a gene. (b) For a more detailed view of the transcriptome data, the tool normalizes and aligns the individual time course studies, to allow all expression data for a gene to be plotted on a common time scale (percentage of cell cycle). (c) Further detail on PTMs, degradation signals and organism-specific phenotypes is provided in the form of tables with linkouts to the original sources whenever possible. [37-39].

### Example 39

### Results: TAGs Genes could be Considered as Early Detection Markers of Breast Cancer

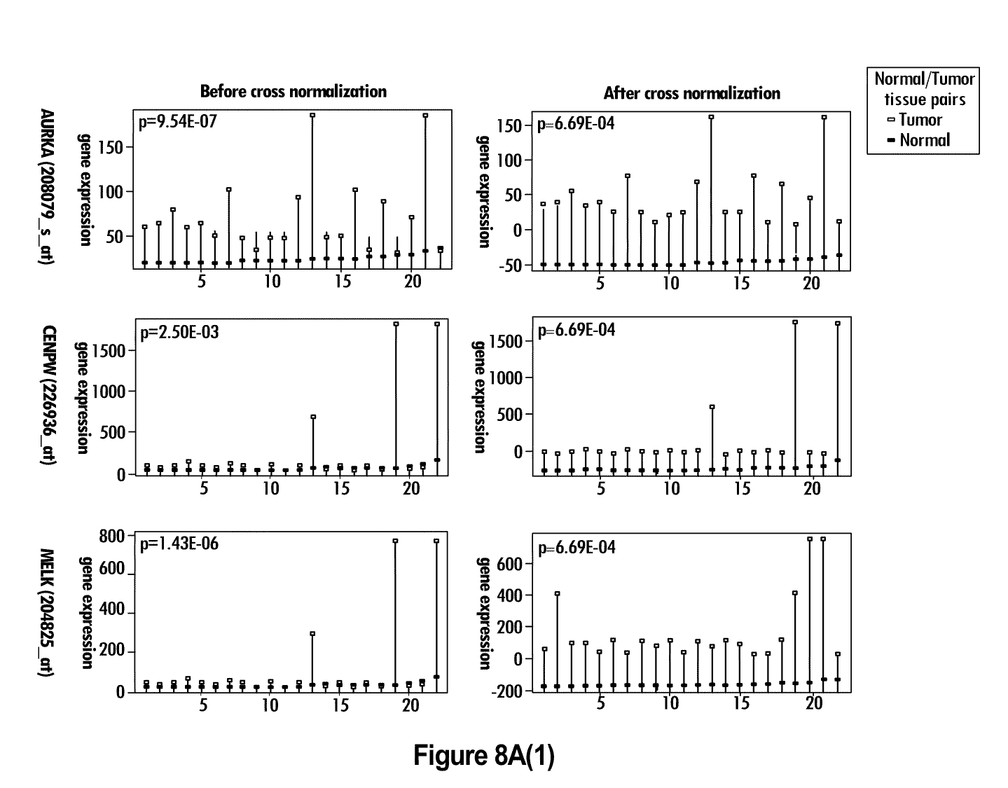

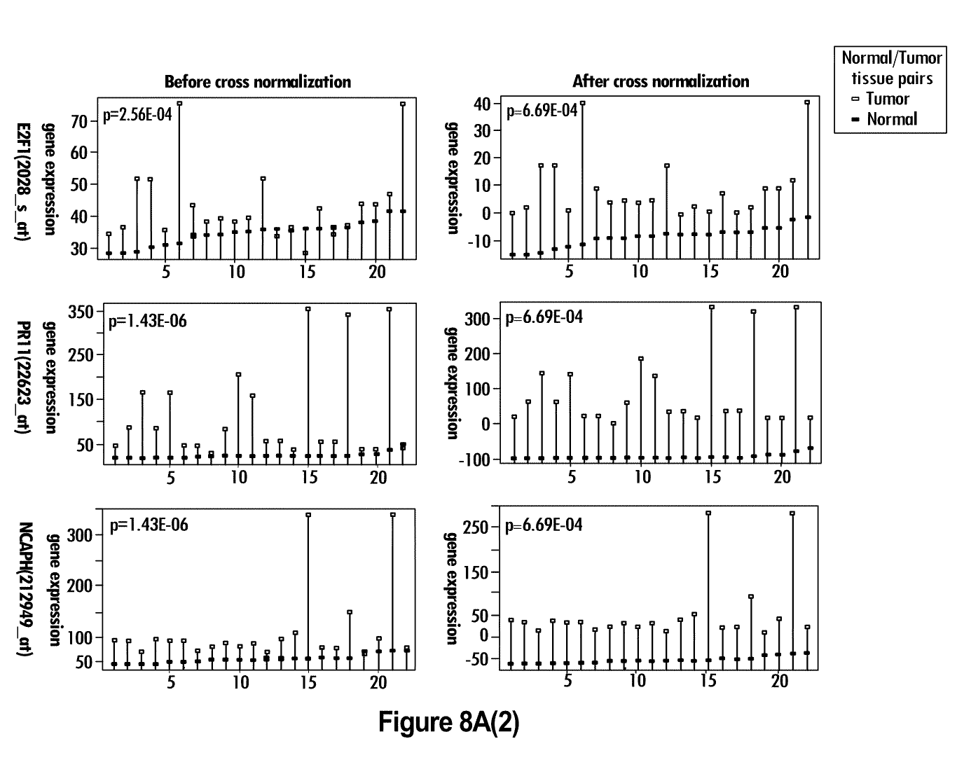

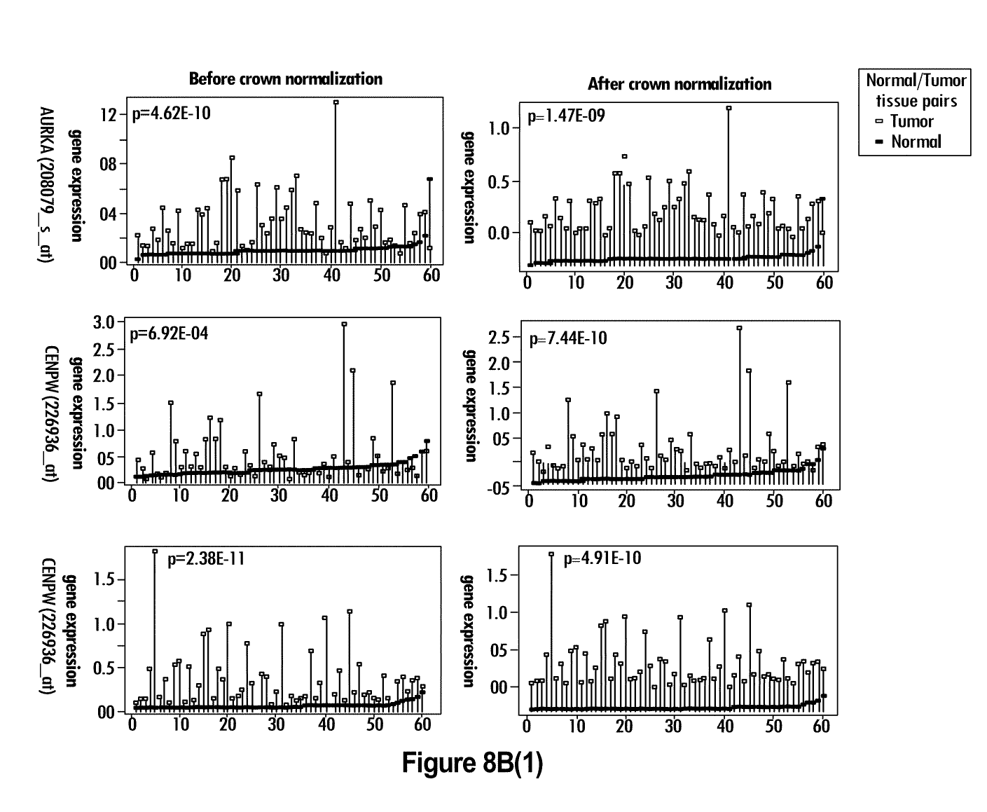

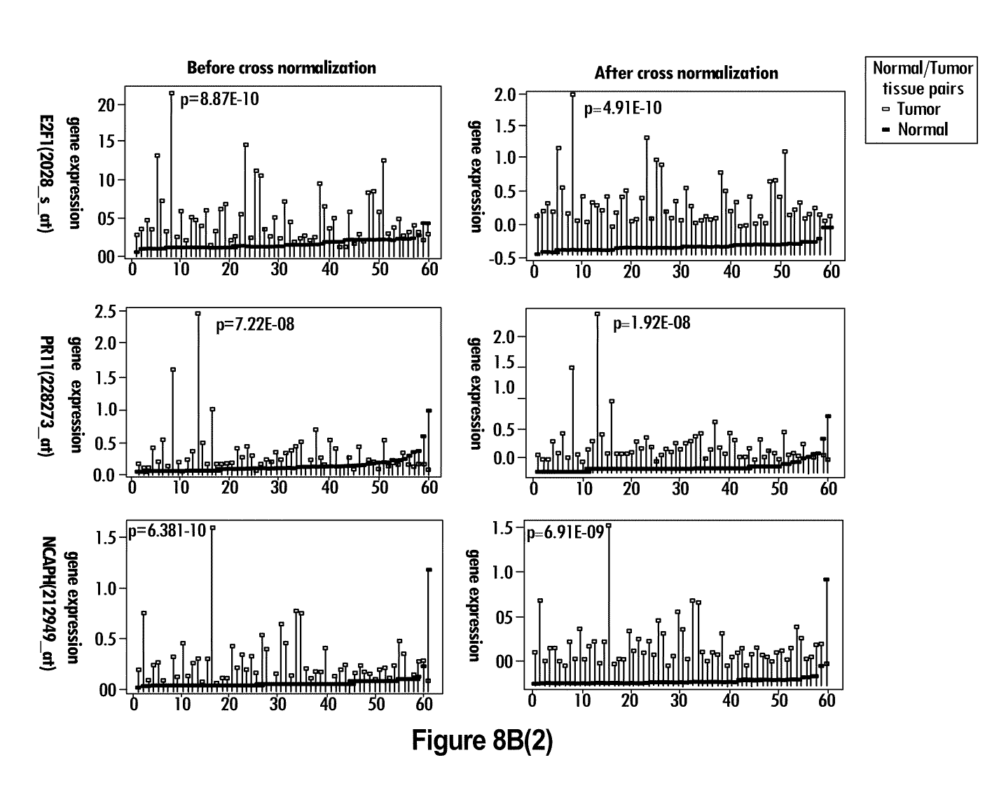

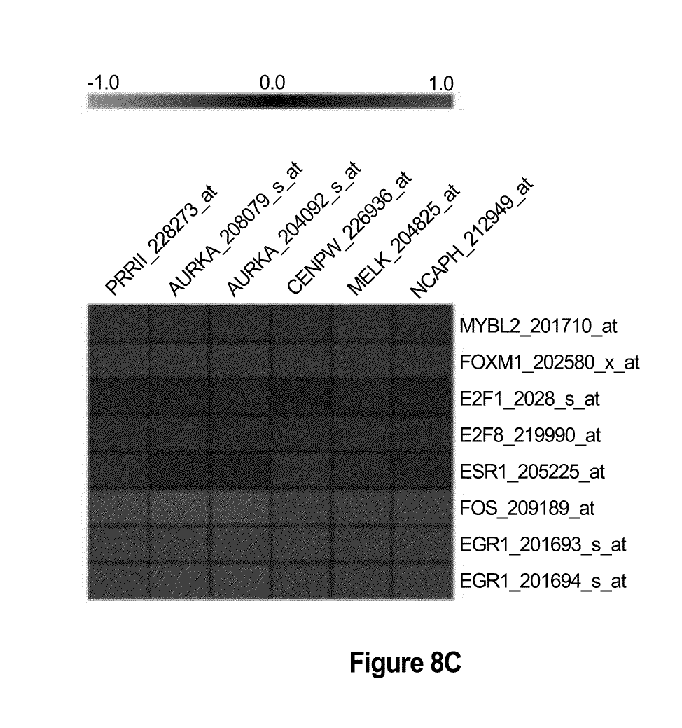

Proliferative or cell cycle/mitotic genes, transcription factors, oncogenes and tumour suppressors are highly-enriched and consist of a major fraction of the 232g-TAGs (represented by 264 U133A&B probsets). This genetic tumour grading classifier provides a classification of the breast cancers of two major tumour classes (G1+G1-like and G3-like+G3) [5], [21] strongly associated with low- and high-risk of BC recurrence, p53 wide-type and p53-mutation status, low- and high-aggressive tumour and patient survival outcomes across many conventional clinical factors including ER-status, LN-status and tumour size. To better understand the regulatory mechanism of the TAGs genes in breast cancers and its ability to use some of these genes as breast cancer clinical biomarkers, we first provided a meta-analysis of various transcription factors that are positively correlated with TAGs genes in various breast cancer datasets (Uppsala, Stockholm and Singapore and GSE61304 dataset (in-house)). Further we found that the representative genes of the 232g-TAGs (BRRN1 (NM_015341), AURKA (NM_003600), MELK (NM_014791), PRR11 (NM_018304), CENPW (NM_001012507) and E2F1 (NM_005225)) have higher expression levels in various stages of breast cancer relative to normal breast tissue (FIG. 8A(1), 8A(2) and FIG. 8B(1), 8B(2)). To test the early diagnostic capability of these TAGs genes, we analysed two breast cancer matched pairs: adjacent normal to tumour from dataset [40] (GSE10780) and TCGA breast cancer dataset, available online at the National Cancer Institute's Cancer Genome Atlas Data Portal.

In this study, we used the extreme discriminative analysis using Modified Wilcoxon Test (MWT) and binomial tests [41]. The method used a cross normalization for matched pair samples. Each gene of 6g-TAG demonstrates a strong discrimination between the tumour and adjacent breast tissue samples (Table EE4).

TABLE EE4
Fold changes in the 6g-TAGs genes in E2F1 siRNA
treated cells. Significant down regulation of mRNA
levels of TAGs genes in E2F1 siRNA treated cells
relatively to control siRNA treated cells.

| Gene | Mean Fold change | Standard | Control |
|---|---|---|---|
| Symbol | E2F1_siRNA_sample | Deviation | siRNA sample |
| E2F1 | 0.067 | 0.003 | 1 |
| AURKA | 0.056 | 0.014 | 1 |
| BRRN1 | 0.081 | 0.032 | 1 |
| CENPW | 0.067 | 0.004 | 1 |
| MELK | 0.168 | 0.012 | 1 |
| PRR11 | 0.129 | 0.027 | 1 |

FIGS. 8A(1) and 8A(2) show the gene expression values in paired samples of GSE10780 dataset. These pairs consist of the expression data for BC and adjacent breast tissue samples before after cross normalization for the matched pair samples. Our application of the cross-normalization method provides an essential improvement in discrimination the BC and adjacent breast tissue samples for almost all matched pair samples. FIGS. 8A(1) and 8A(2) show that each of the six genes shows the higher relative mRNA levels in all tumours versus to normal adjacent breast tissues with high statistical significance (Table EE4). FIGS. 8B(1) and 8B(2) show that application of cross-normalization methods and our statistical models leads to similar results for the paired samples found in TCGA datasets. All genes of TAGs show relatively higher mRNA values in tumour samples compared to adjacent (‘normal’) tissue of breast cancer patient samples. FIGS. 8A(1), 8A(2) and FIGS. 8B(1) and 8B(2) strongly indicate that the studied genes could be used as the early diagnostic markers of breast cancer.

We further investigated the regulatory role of various transcription factors (TF) on TAG genes in breast cancer. E2F1 is a key regulator of transcription activity in breast and many other cancers. We found that E2F1 (which gene is belonging to 232g-TAGs) correlates positively with many other TAGs genes (FIG. 8C), indicating possible (direct or indirect) regulatory role of E2F1 in the expression of the TAG genes in BC cells.

### Example 40

### Results: E2F1 Transcription Factor Regulates the TAGs Genes

We suggested that E2F1 could play regulatory role as a transcription factor (TF) controlling the proliferation, cell cycle/mitosis genes included in our TAG signature. We screened ChIP-seq (Chromatin immunoprecipitation sequencing) tracks in UCSC genome browser and investigated MCF-7 breast cancer cell line dataset (Chromatin Immunoprecipitation using HA tagged E2F1 antibody) and found that all the TAGs genes showed significant ChIP-seq E2F1 binding peaks in their upstream promoter regions. We observed significant E2F1 promoter binding ChIP-seq peaks at upstream promoter regions of the 6g-TAGs genes.

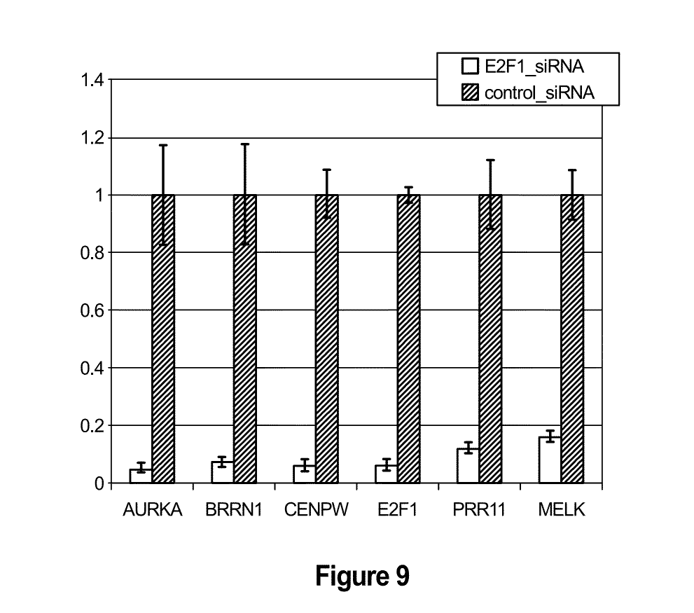

Based on co-expression analysis and promoter binding site studies, we suggest that E2F1 could regulate our o TAGs genes. To check if TAGs genes act as targets of E2F1 transcription factor, we conducted siRNA silencing experiments by knocking down E2F1 transcript in breast cancer cell line (MDA-MB-436) and estimated the mRNA levels of TAGs genes using qPCR studies. FIG. 9 represents E2F1 siRNA silencing experiment relatively compared with control siRNA of MDA-MB-436 breast cancer cell line. FIG. 9 shows effective knock down of E2F1 mRNA levels relatively compared to control siRNA treated cells. FIG. 9 further shows significant down regulation of mRNA levels of the TAGs genes in E2F1 siRNA treated cells relatively to control cells (Table EE5).

TABLE EE5
Estimates of the expression values of the 6 genes detected in G1 and G3 sub-groups.
And results of SWS classification G1 vs G3. A: Uppsala cohort, B: Stockholm cohort, C:
Singapore cohort.

| | | | | | | | |
|---|---|---|---|---|---|---|---|
| | | | | | | | Cut-off |
| | | | | Affymetrix | Grade w/ | Grade w/ | value by |
| | | Gene | | probe sets | Higher | Lower | SWS |
| Entrez_ID | Gene Name | symbol | Refseq ID | ID | Expr. | Expr. | method |

A. Uppsala:

| | | | | | | | |
|---|---|---|---|---|---|---|---|
| 6790 | aurora kinase | AURKA | NM_003600 | 208079_s_ | G3 | G1 | 6.65262 |
| | A | | | at | | | |
| 387103 | centromere | CENPW | NM_ | 226936_at | G3 | G1 | 7.56154 |
| | protein W | | 001286524 | | | | |
| 9833 | maternal | MELK | NM_014791 | 204825_at | G3 | G1 | 7.1069 |
| | embryonic | | | | | | |
| | leucine | | | | | | |
| | zipper kinase | | | | | | |
| 23397 | non-SMC | NCAPH | NM_015341 | 212949_at | G3 | G1 | 5.91723 |
| | condensin I | | | | | | |
| | complex, | | | | | | |
| | subunit H | | | | | | |
| 55771 | proline rich | PRR11/ | NM_018304 | 228273_at | G3 | G1 | 7.70616 |
| | 11 | FLJ11029 | | | | | |
| 1869 | E2F | E2F1 | NM_005225 | 2028_s_at | G3 | G1 | 6.47071 |
| | transcription | | | | | | |
| | factor 1 | | | | | | |

B. Stockholm:

| | | | | | | | |
|---|---|---|---|---|---|---|---|
| 6790 | aurora kinase | AURKA | NM_003600 | 208079_s_ | G3 | G1 | 6.30082 |
| | A | | | at | | | |
| 387103 | centromere | CENPW | NM_ | 226936_at | G3 | G1 | 7.40448 |
| | protein W | | 001286524 | | | | |
| 9833 | maternal | MELK | NM_014791 | 204825_at | G3 | G1 | 6.63834 |
| | embryonic | | | | | | |
| | leucine | | | | | | |
| | zipper kinase | | | | | | |
| 23397 | non-SMC | NCAPH | NM_015341 | 212949_at | G3 | G1 | 5.33539 |
| | condensin I | | | | | | |
| | complex, | | | | | | |
| | subunit H | | | | | | |
| 55771 | proline rich | PRR11/F | NM_018304 | 228273_at | G3 | G1 | 7.16871 |
| | 11 | LJ11029 | | | | | |
| 1869 | E2F | E2F1 | NM_005225 | 2028_s_at | G3 | G1 | 5.9933 |
| | transcription | | | | | | |
| | factor 1 | | | | | | |

C. Singapore

| | | | | | | | |
|---|---|---|---|---|---|---|---|
| 6790 | aurora kinase | AURKA | NM_003600 | 208079_s_ | G3 | G1 | 6.77578 |
| | A | | | at | | | |
| 387103 | centromere | CENPW | NM_ | 226936_at | G3 | G1 | 7.46601 |
| | protein W | | 001286524 | | | | |
| 9833 | maternal | MELK | NM_014791 | 204825_at | G3 | G1 | 6.9252 |
| | embryonic | | | | | | |
| | leucine | | | | | | |
| | zipper kinase | | | | | | |
| 23397 | non-SMC | NCAPH | NM_015341 | 212949_at | G3 | G1 | 5.65104 |
| | condensin I | | | | | | |
| | complex, | | | | | | |
| | subunit H | | | | | | |
| 55771 | proline rich | 228273_at | NM_018304 | PRR11 | G3 | G1 | 7.12064 |
| | 11 | | | | | | |
| 1869 | E2F | 2028_s_at | NM_005225 | E2F1 | G3 | G1 | 6.48464 |
| | transcription | | | | | | |
| | factor 1 | | | | | | |

Based on co-expression studies on various breast cancer datasets and E2F1 promoter binding analysis of the TAGs genes, along with siRNA-E2F1 validation experiments, we strongly suggest that E2F1 transcription factor could regulate the TAGs genes in breast cancer. This led to further extend gene panel by including E2F1 transcription factor and investigate further by experiments the proliferative potential and prognostic significance of TAGs genes. In all our future sections, we included E2F1 (NM_005225) along with the origin 5 TAGs genes (BRRN1 (NM_015341), AURKA (NM_003600), MELK (NM_014791), PRR11 (NM_018304), CENPW (NM_001012507), Table EE1) as the 6g-TAGs.

TABLE EE1
Annotation of 6g-TAGs Genes

| | | | Genbank |
|---|---|---|---|
| Gene Symbol | Affy ID | Gene symbol | accession no. |
| Serine/threonine- | A.204092_s_at | AURKA | NM_003600 |
| protein kinase 6 | | | |
| Serine/threonine- | A.208079_s_at | AURKA | BC027464 |
| protein kinase 6 | | | |
| Barren homologue | A.212949_at | BRRN1 | D38553 |
| (Drosophila) | | | |
| Chromosome 6 open | B.226936_at | C6orf173/ | BG492359 |
| reading frame 173 | | CENPW | |
| E2F transcription factor 1 | A.204947_at | E2F1 | NM_005225 |
| Hypothetical protein | B.228273_at | PRR11 | BG165011 |
| FLJ11029 | | | |
| Maternal embryonic | A.204825_at | MELK | NM_014791 |
| leucine zipper kinase | | | |

### Example 41

### Results: TAGs Genes Demonstrates Robust Grade Signature Potential in Breast Adenocarcinoma

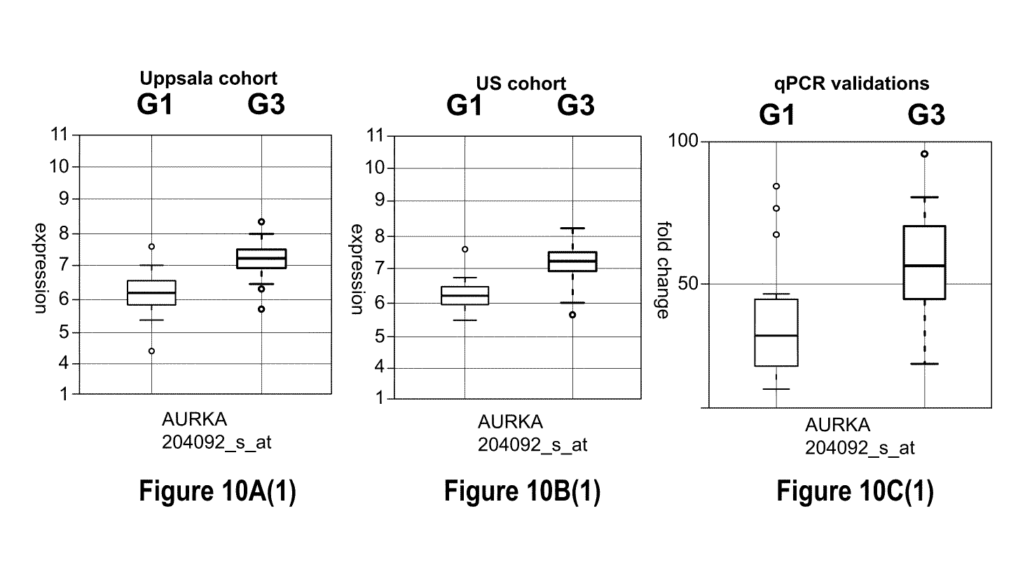

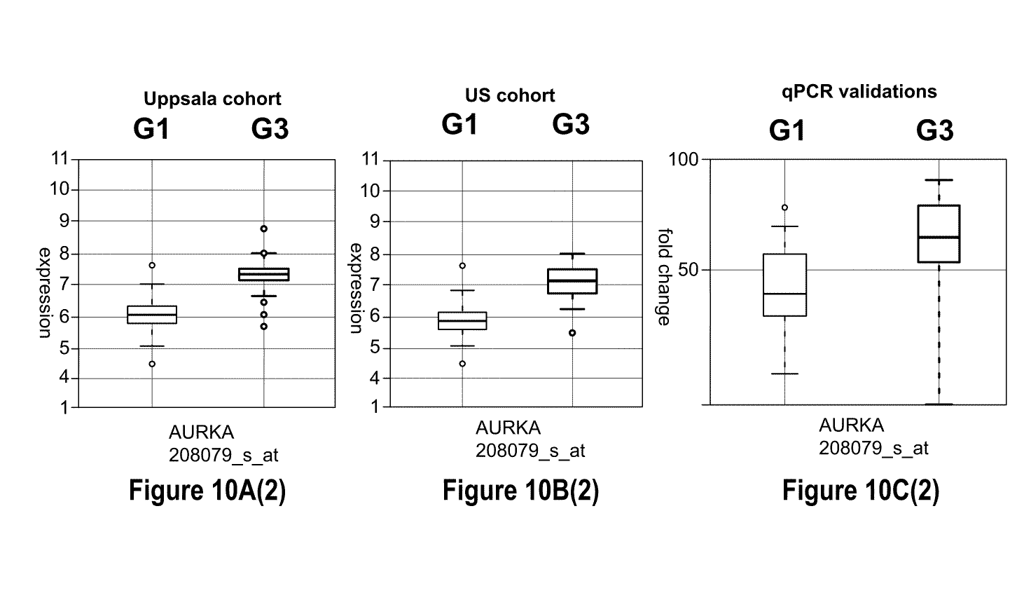

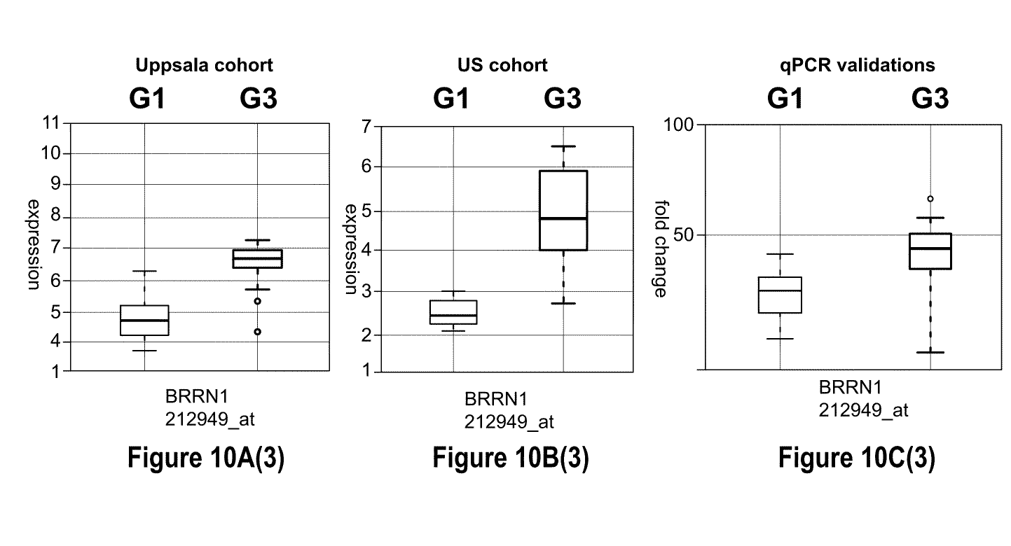

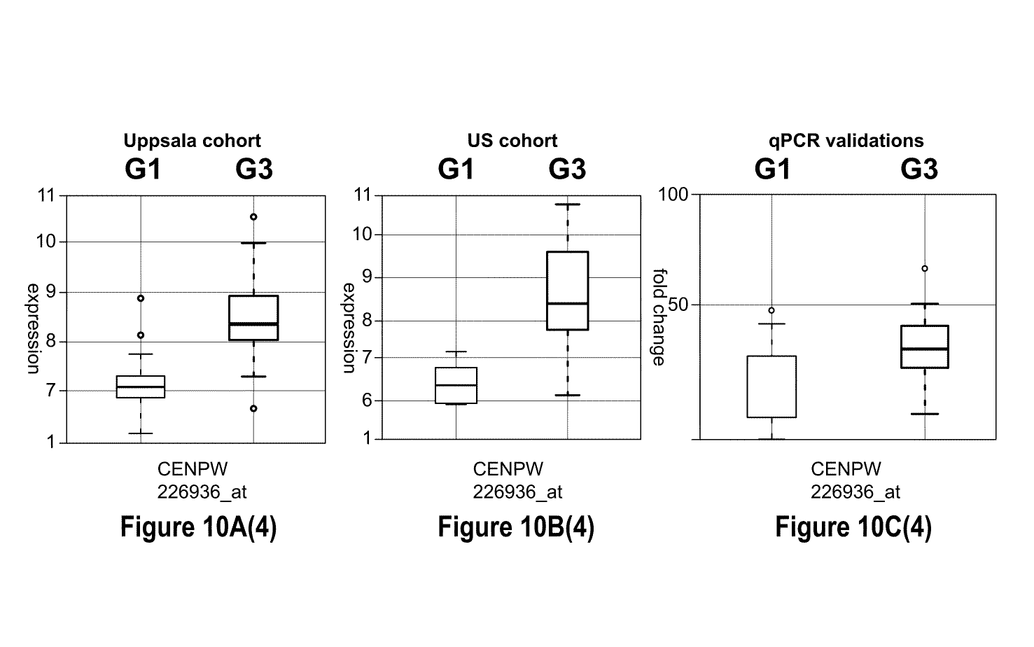

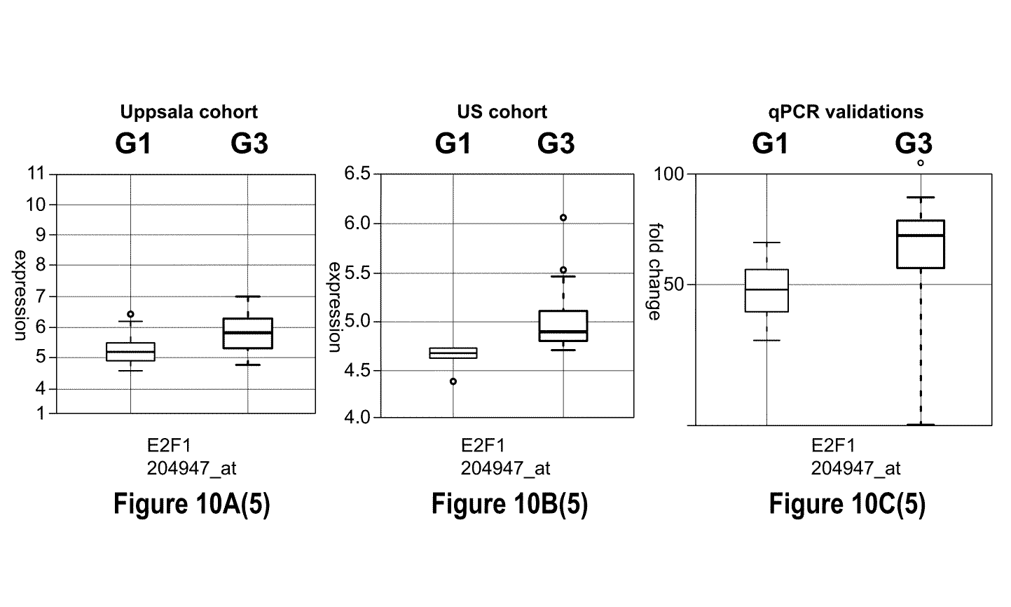

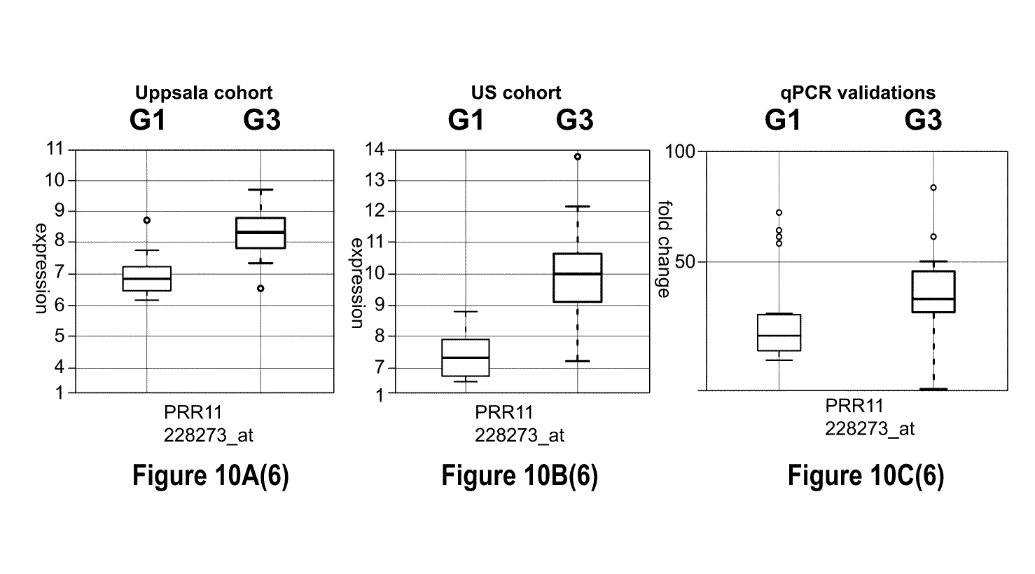

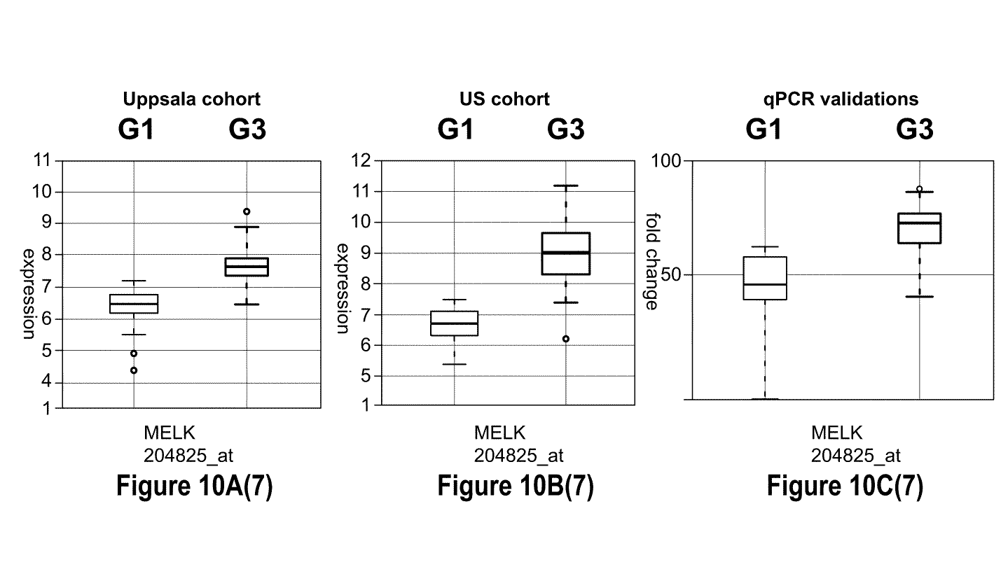

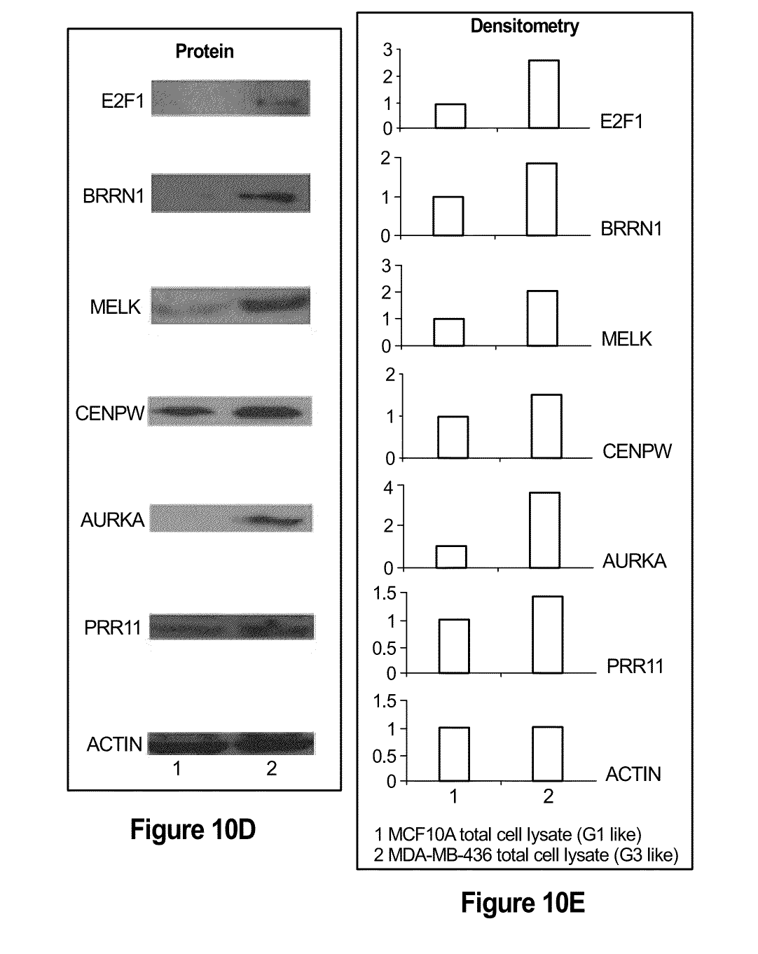

To understand the grade signature potential of 6 TAGs genes, we extracted Affymetrix probsets intensity values in various Uppsala, Stockholm and Singapore cohort public microarray datasets. FIGS. 10A(1)-10A(7) represent relative mean intensity values of G1 and G3 patients along with their respective standard error in Uppsala cohort. The mRNA levels of all six genes (Table ST) (BRRN1 (NM_015341), AURKA (NM_003600), MELK (NM_014791), PRR11 (NM_018304), CENPW (NM_001012507) and E2F1 (NM_005225) have relatively higher levels in G3 patients compared to G1 patient samples. Similar results were observed for all the TAGs genes in Stockholm and Singapore breast cancer microarray datasets (Table EE6). These tables demonstrate high reproducibility of stratification characteristics our methods based on 6g-TAGs genes across different datasets and ethnic groups (Asian and European).

To reconfirm this phenomenon, Affymetrix microarray probe intensity values of the 6g-TAGs genes were extracted from in-house cohort microarray dataset (GSE61304) and estimated mean values for G1 and G3 patient samples respectively. FIGS. 10B(1)-10B(7) represent the relatively mean intensity values of G1 and G3 patients along with their respective standard error. Based on FIGS. 10B(1)-10B(7) it is clearly evident that all TAGs genes shows clear grade discrimination at mRNA expression, which is in concordance with all public breast cancer datasets (Uppsala, Stockholm, Singapore cohorts) studied.

To validate further the observations based on microarray experiments, we conducted real time quantitative PCR (qRT-PCR) using commercial tissue array experiments. FIGS. 10C(1)-10C(7) represent relative mean fold change values of all TAGs genes for grade 1 and G3 BC patient samples. FIGS. 10C(1)-10C(7) strongly support the view that 6g-TAGs genes can consistently discriminate the grade signature at RNA level in various independent breast cancer cohorts.

Then we further checked if these 6g-TAGs genes also show similar expression pattern and discriminate grades at protein level. To test this phenomenon, we selected two well established breast cancer cell lines, MCF10A (immortal, non-tumourigenic, low grade), and MDA-MB-436 (invasive tumourigenic high grade) to quantify the protein expression levels of 6g-TAGs genes. FIG. 10D shows relative protein expression of all 6g-TAGs genes using Western/Immunoblotting experiments. FIG. 10D represents protein levels relatively compared between low grade MCF10A breast cell line (G1 like) and high grade invasive aggressive MDA-MB-436 breast cell line (G3 like). The protein expression of CENPW, AURKA, MELK, PRR11, BRRN1 and E2F1 were relatively low in MCF10A with respect to high grade MDA-MB-436. This observation is in support with the phenomenon observed at mRNA level for 6g-TAGs genes (FIGS. 10A(1)-10A(7), FIGS. 10B(1)-10B(7), and FIGS. 10C(1)-10C(7)) among G1 and G3 patient samples.

### Example 42

### Results: TAGs Genes can Stratify Grade 2 Heterogeneity in Breast Cancer Samples

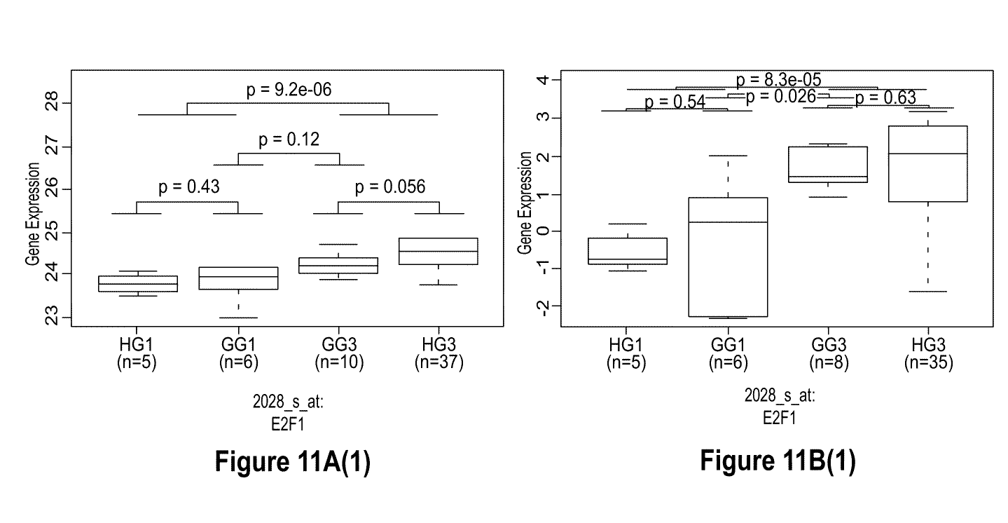

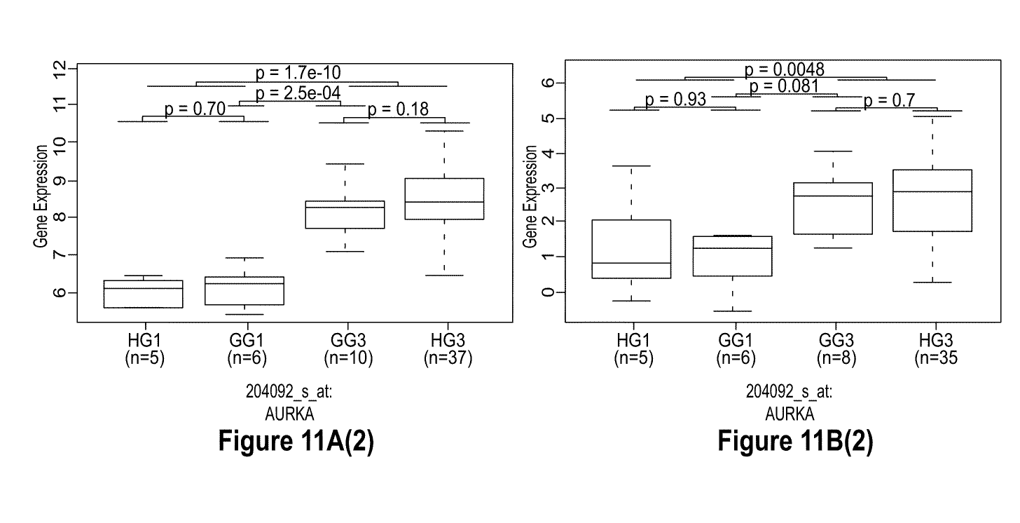

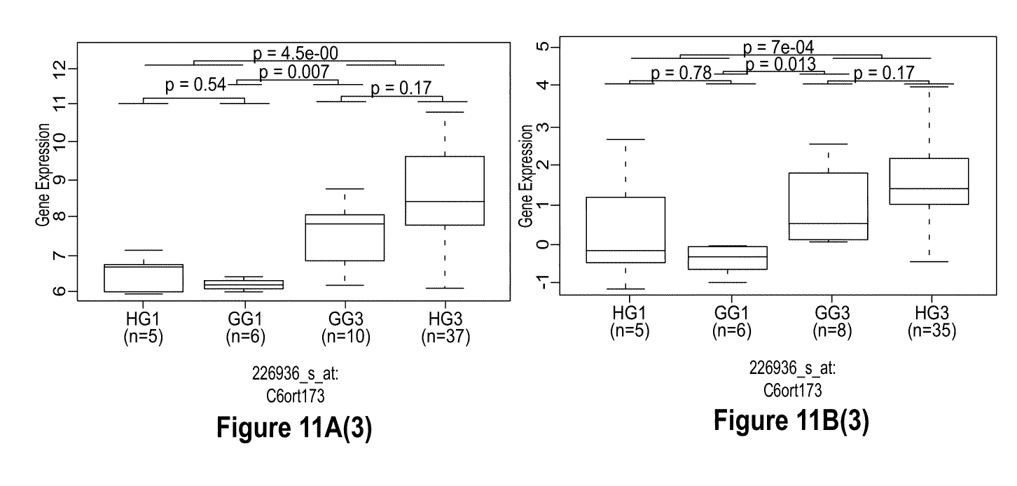

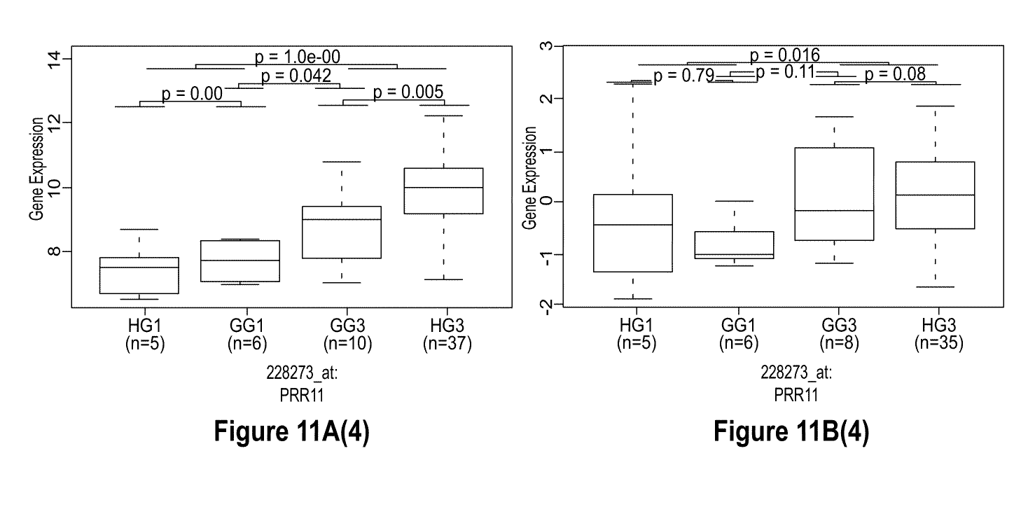

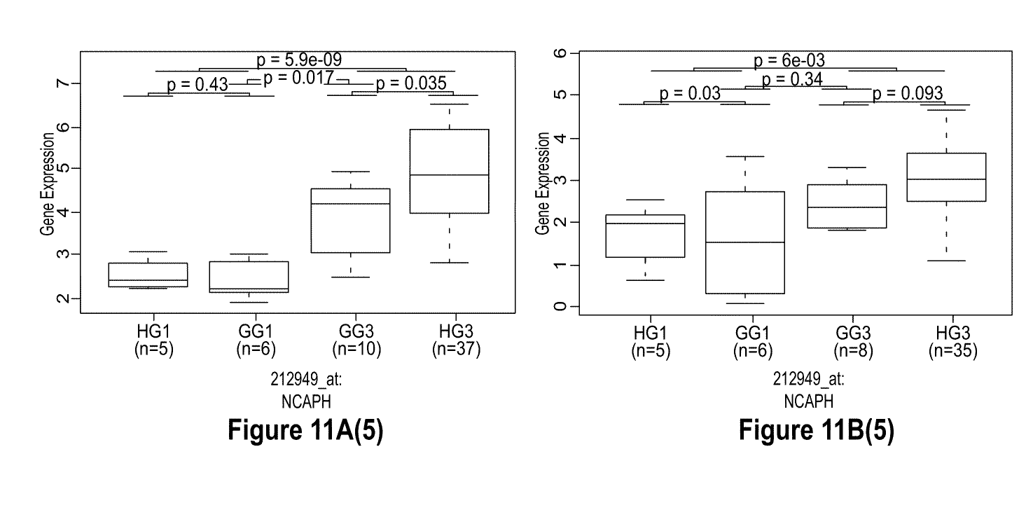

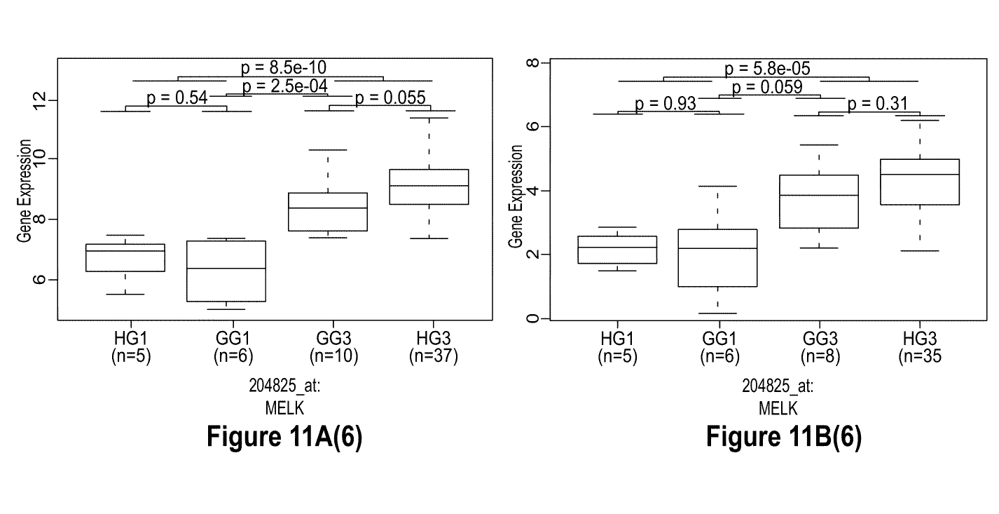

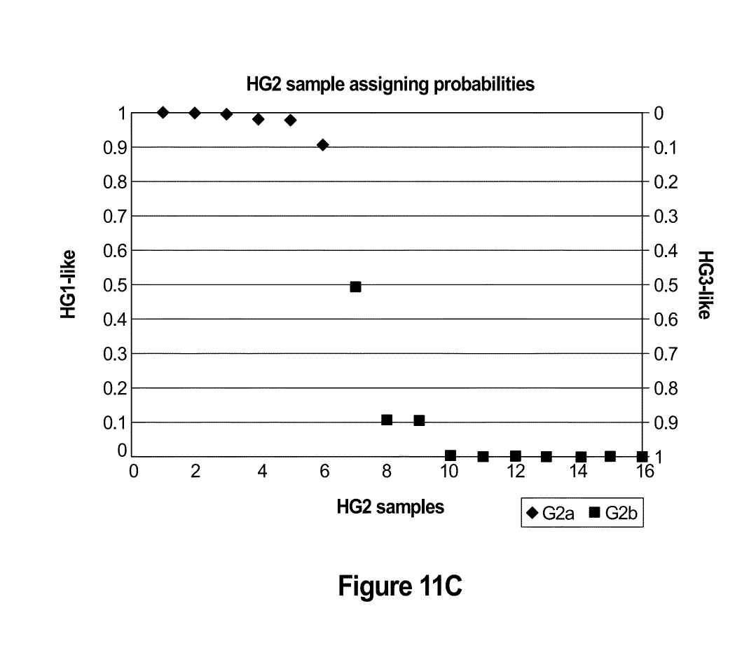

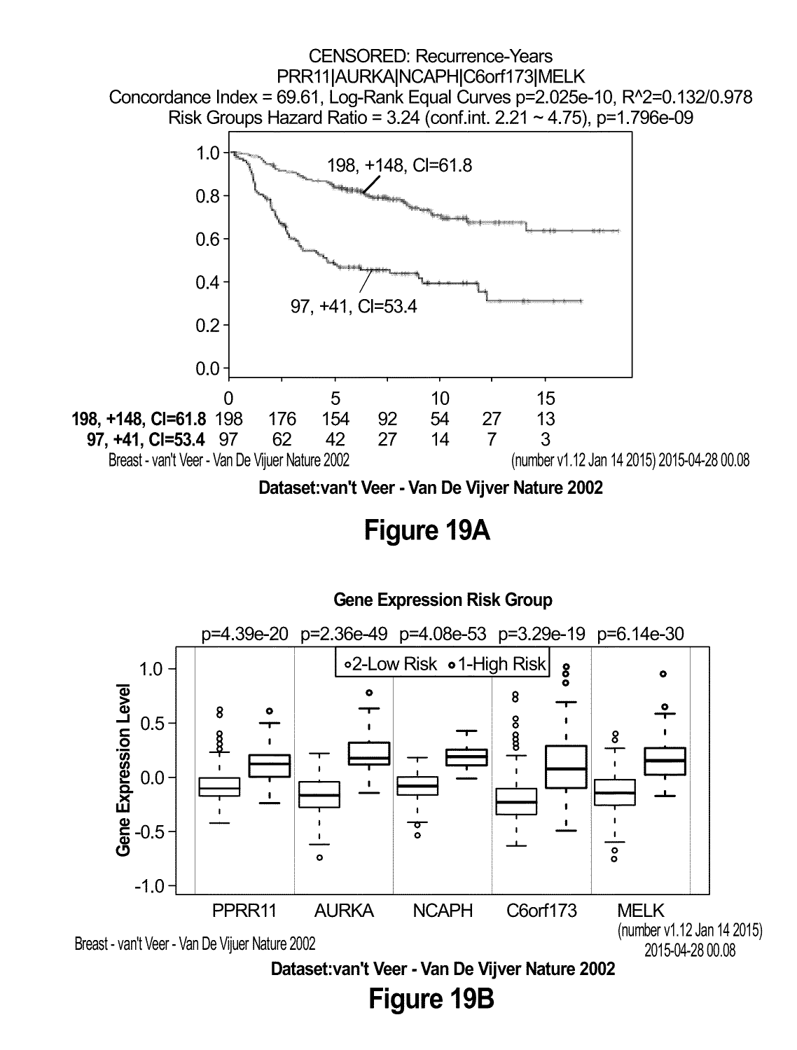

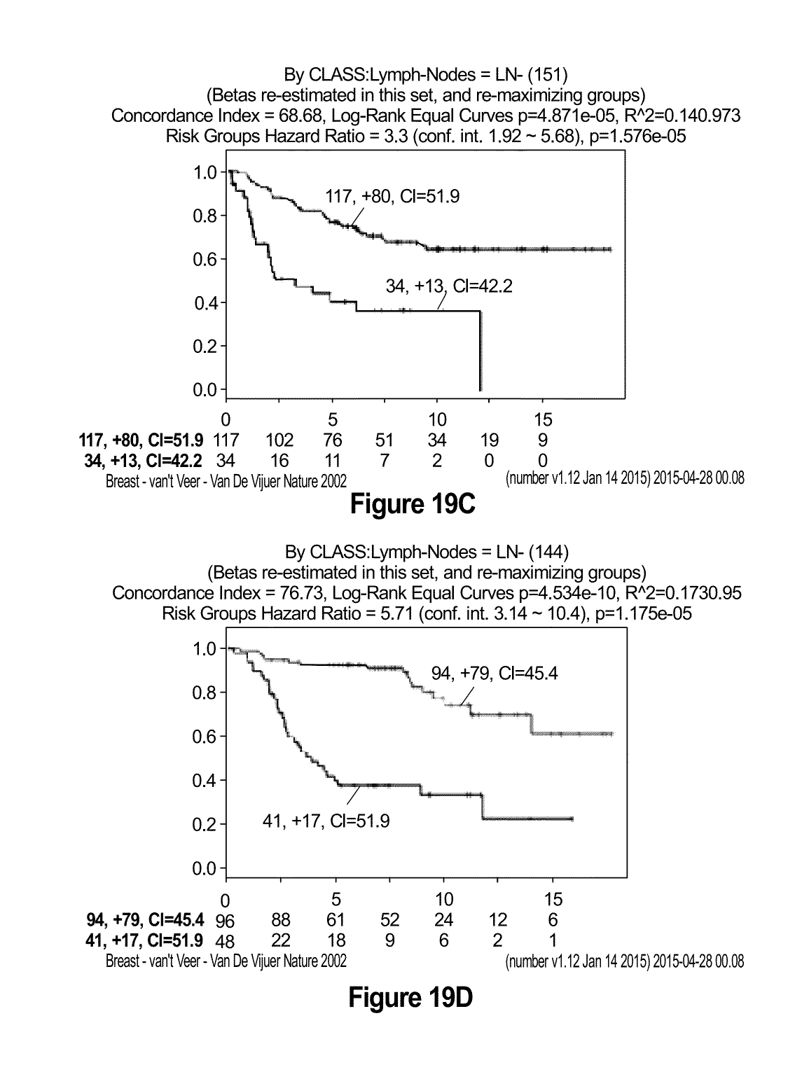

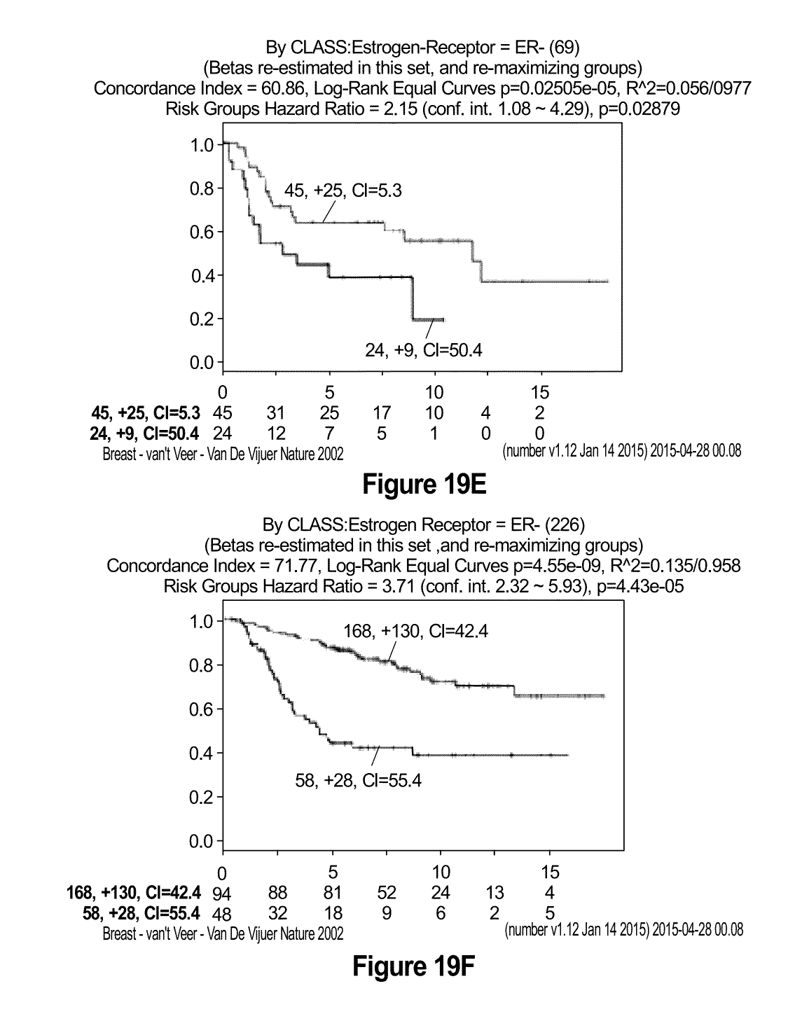

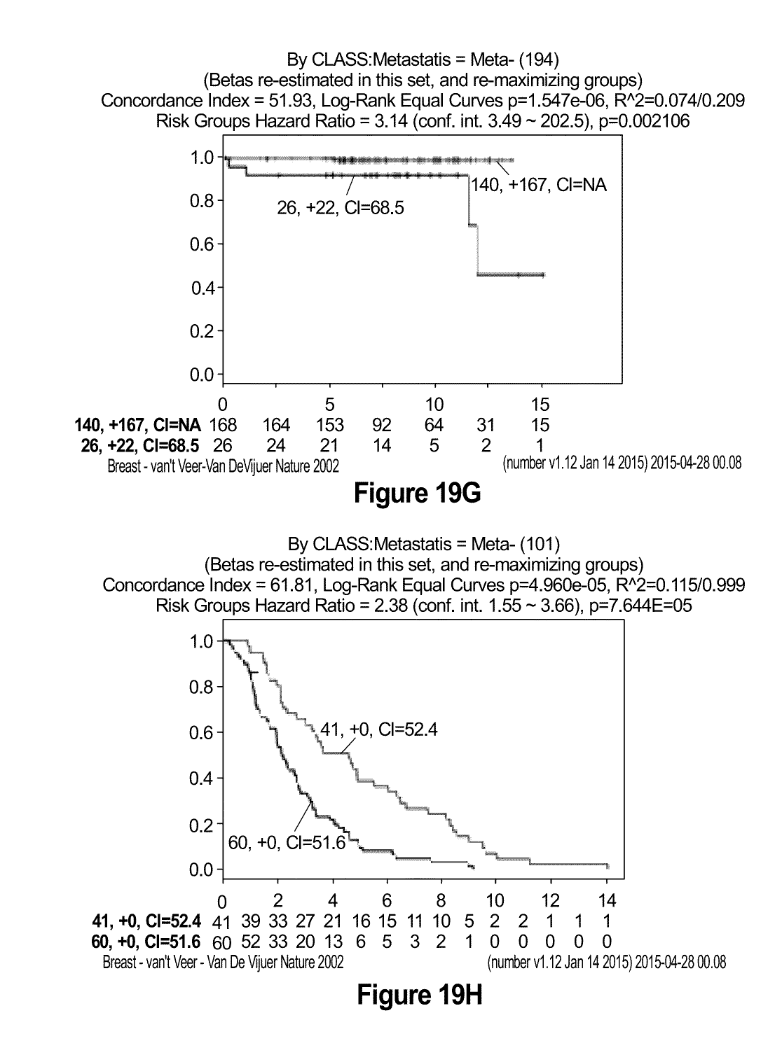

Patients with histological G2 have ‘moderate’ risk BC development on average. A better treatment options can be provided, if underlying heterogeneity of G2 tumours be delineated further into G1 like and G3 like categories [42]. We analysed 4 different breast cancer datasets to test if 6g-TAGs can delineate G2 patients into either HG1 like and/or HG3 like groups. FIGS. 11A(1)-11A(6) show all 6g-TAGs genes efficiently delineating the G2 patients into HG1-like or HG3 like groups in US cohort (GSE61304 dataset) with p<0.01. This suggests that the G2 patients belong to sub-class of either G1 (low risk) or G3 (high risk) category This phenomenon was further validated experimentally using qRT-PCR and FIGS. 11B(1)-11B(6) represent the 6g-TAGs genes and their ability to stratify G2 tumours into G1 like and G3 like sub-classes, that are statistically significant (p<0.01) and high accuracy. SWS probability estimates and its visual presentation on FIG. 11C could be used for a prediction of the aggressiveness of BC in personalized patient prognostic system. Similar observations were found on various cohorts and found strong consistency in sub-classifying G2 histological patients in to G1 like and G3 like as shown in FIGS. 11A(1)-11C and FIGS. 19A-19H.

### Example 43

### Results: 6g-TAGs Genes Co-Express and Act as Interacting Network Hubs

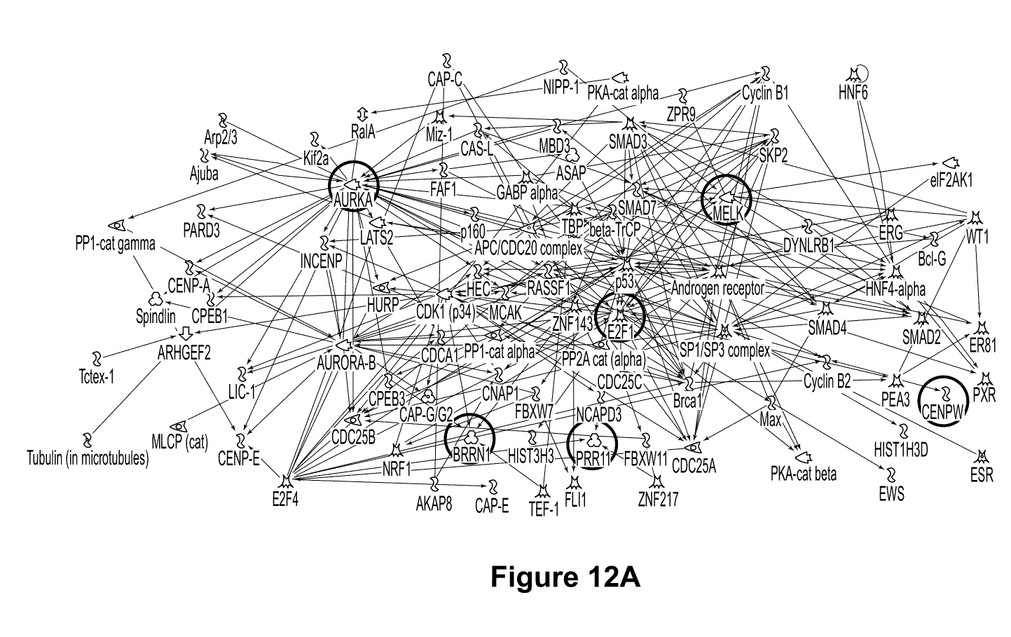

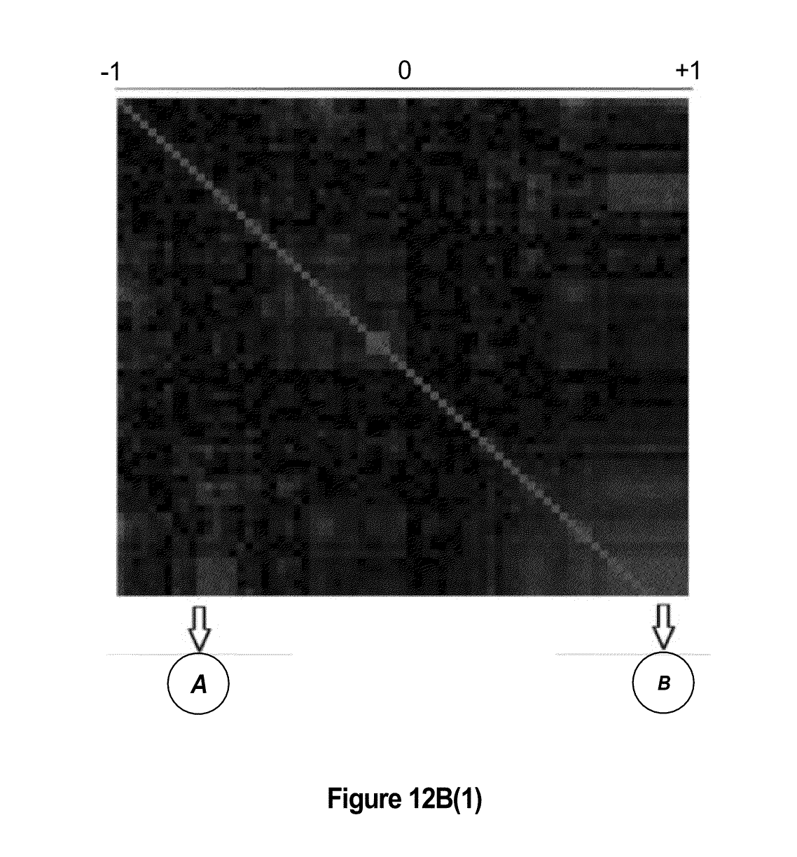

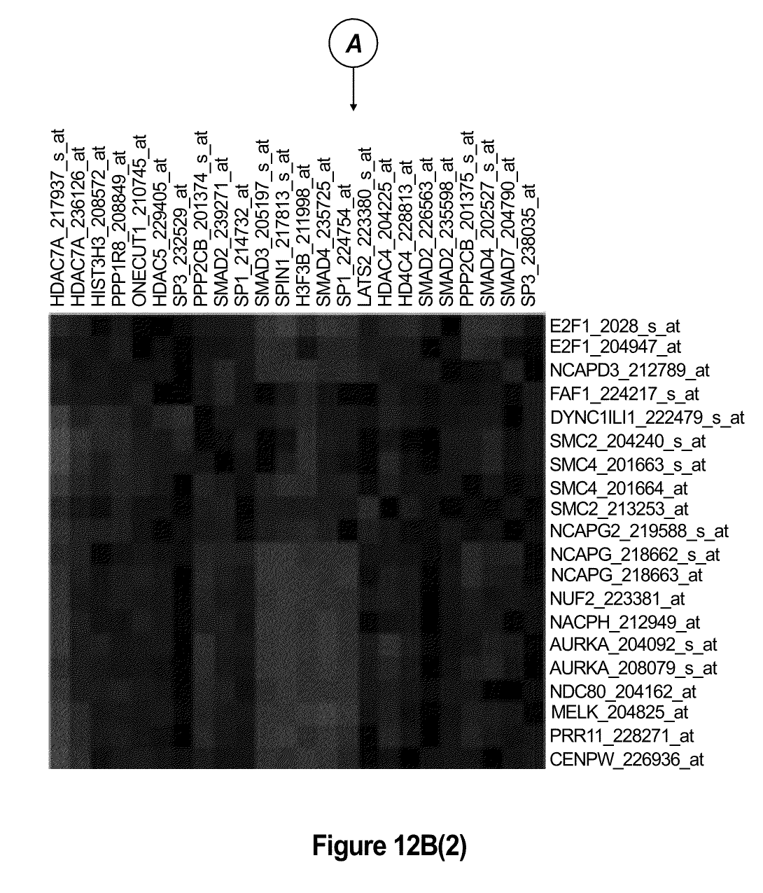

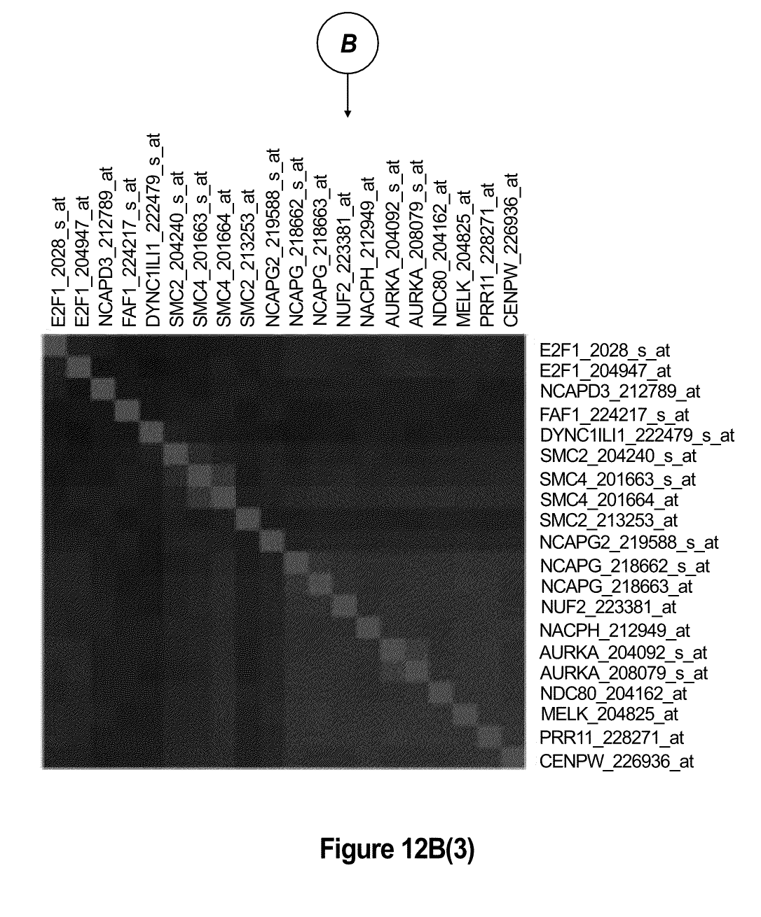

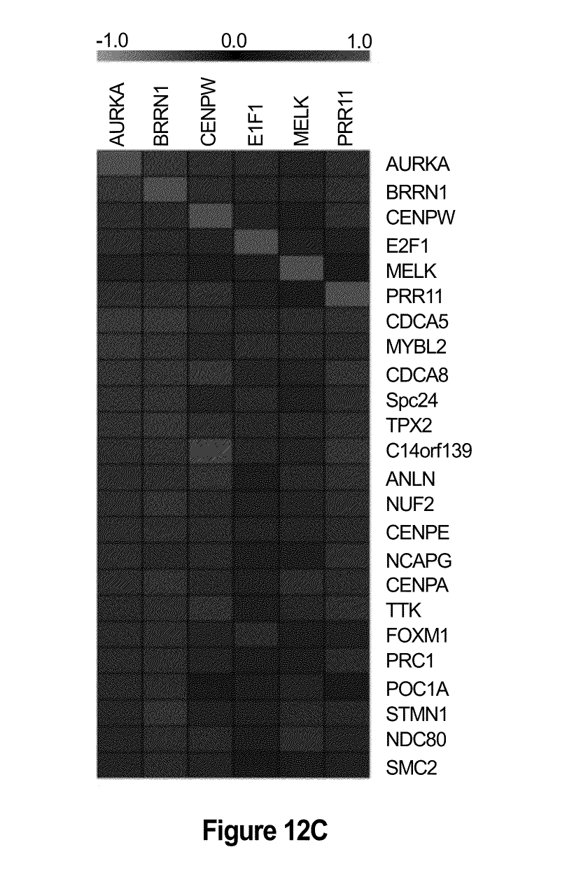

To understand the underlying mechanisms of breast cancer with respect to 6g-TAGs genes, we conducted co-expression studies on various breast cancer microarray datasets (Uppsala, Stockholm, Singapore, US). Based on 6g-TAGs genes, we extended the interacting gene network components using Metacore (GeneGo) software with an arbitrary cut-off of 50 nodes (genes). FIG. 12A represents strong interacting network hubs of 6g-TAGs genes and their respective components. To understand, if these network components co-express with 6g-TAGs genes in breast cancer cohort datasets, Affymetrix probesets intensity values (mRNA expression) were extracted for all the 50 genes including our TAGs genes and independently estimated co-efficient of correlation (Kendall tau) for all breast cancer cohort datasets.

FIGS. 12B(1)-12B(3) represent statistically significant (p<0.01) correlation matrix of Uppsala dataset containing both positive and negative correlated network components with respect to 6g-TAGs genes. FIGS. 12B(1)-12B(3) represent strong positively correlated network components with respect to 6g-TAGs genes. Among the set of positive correlated genes, 6g-TAGs genes are strongly co-expressed with each other, consistent in all BC datasets studied. FIGS. 12B(1)-12B(3) represent strong positive and negative correlated gene network components with respect to 6g-TAGs genes. Table EE7 represents the list of the gene network components that are significantly positively or negatively correlated with respect to 6g-TAGs genes network. These transcribed sequences of these two gene expression profiles (positive and negative correlated with 6g-TAGs) can be considered as a novel BC diagnostic and prognostic sets which could separately or together consist of a BC detection platform for assay development. Some of these genes have been reported as the members of other BC gene signatures. However, in combination these subsets could be considered as the combined BC signature TAG-associated signature with strong potential of diagnostics, prognosis, and prediction of low- and high-aggressive BCs, including G1-like and G3-like (intermediated) tumour subtypes.

TABLE EE7
The prognostic significance of TAGs genes observed in microarray (Uppsala, BII-US) and qPCR (BII-US) experiments.
Grouping based on 1D DDg method.

| | | | | mean | | | | | | | |
|---|---|---|---|---|---|---|---|---|---|---|---|
| | | | | signal | | | | # of | # of | Cut- | |
| | | | | intensity | mean signal | | | patients | patients | off | |
| | | | | for low | intensity for | | | in low- | high- | value | |
| | Affymetrix | | 1D pvalue | risk | high-risk | fold | Wilcoxon | risks | risks | of 1D | Hazard |
| | ID | Gene | (log rank) | subgroup | subgroup | change | p-value | patients | patients | DDg | ratio |
| 1 | 208079_s_at | AURKA | 0.000249 | 5.985575 | 7.30868 | 2.50 | 1.66E−40 | 151 | 98 | 6.62 | 2.18 |
| 2 | 204092_s_at | AURKA | 0.000586 | 6.026485 | 7.082975 | 2.08 | 3.71E−42 | 116 | 133 | 6.49 | 2.16 |
| 3 | 212949_at | BRRN1 | 1.74E−05 | 4.19631 | 5.942195 | 3.35 | 7.43E−39 | 88 | 161 | 4.64 | 3.28 |
| 4 | 226936_at | CENPW | 6.90E−06 | 7.010145 | 8.301832 | 2.45 | 1.02E−41 | 140 | 109 | 7.53 | 2.66 |
| 5 | 204825_at | MELK | 1.31E−05 | 6.284949 | 7.545683 | 2.40 | 2.09E−39 | 158 | 91 | 6.87 | 2.53 |
| 6 | 228273_at | PRR11 | 1.46E−06 | 6.716112 | 8.129601 | 2.66 | 2.71E−42 | 120 | 129 | 7.32 | 3.12 |
| 7 | 204947_at | E2F1 | 6.55E−05 | 5.252048 | 6.614766 | 2.57 | 2.48E−16 | 224 | 25 | 6.31 | 3.03 |
| 8 | 2028_s_at | E2F1 | 0.001845 | 6.166082 | 6.682863 | 1.43 | 7.63E−35 | 178 | 71 | 6.47 | 1.98 |

B. BII-US patients groupping by microarray data:

| | | | | mean | | | | number | number | | |
|---|---|---|---|---|---|---|---|---|---|---|---|
| | | | | signal | | | | of | of | Cut- | |
| | | | 1D | intensity | mean signal | | | patients | patients | off | |
| | | | pvalue | for low | intensity for | | | in low- | high- | value | |
| | Affymetrix | | (log | risk | high-risk | fold | Wilcoxon | risks | risks | of 1D | Hazard |
| | ID | Gene | rank) | subgroup | subgroup | change | p-value | patients | patients | DDg | ratio |
| 1 | 208079_s_at | AURKA | 0.012915 | 6.710945 | 8.818444 | 4.31 | 2.27E−16 | 23 | 35 | 6.98 | 329275676.58 |
| 2 | 204092_s_at | AURKA | 0.013261 | 6.731982 | 8.799534 | 4.19 | 2.27E−16 | 23 | 35 | 6.94 | 329275676.58 |
| 3 | 212949_at | BRRN1 | 0.011663 | 2.854773 | 5.198879 | 5.08 | 1.56E−16 | 24 | 34 | 3.22 | 10.80 |
| 4 | 226936_at | CENPW | 0.003341 | 6.660905 | 8.953091 | 4.90 | 1.56E−16 | 24 | 34 | 7.13 | 485883905.54 |
| 5 | 204825_at | MELK | 0.001047 | 7.829347 | 9.935686 | 4.31 | 1.01E−14 | 41 | 17 | 9.13 | 4.65 |
| 6 | 228273_at | PRR11 | 0.010035 | 7.79849 | 10.17515 | 5.19 | 2.27E−16 | 23 | 35 | 8.36 | 11.08 |
| 7 | 204947_at | E2F1 | 0.003103 | 4.720146 | 5.092555 | 1.29 | 9.03E−17 | 26 | 32 | 4.81 | 18.08 |
| 8 | 2028_s_at | E2F1 | 0.003356 | 2.393908 | 2.660206 | 1.20 | 6.88E−17 | 28 | 30 | 2.43 | 6.31 |

C. BII-US patients grouping by qPCR assay:

| | 1D cutoff | | | | | | | | |
|---|---|---|---|---|---|---|---|---|---|
| | (Fold | | | | | | | | |
| | Changes | | | | | | | | Ratio of mean |
| | with | 1D | number | number | | | mean fold | mean fold | values of high |
| | respect to | pvalue | of | of | | | changes of | changes of | risk with respect |
| gene- | Normal | (log | low- | high- | coxph | | ddCt (low- | ddCt (high- | to low risk |
| name | tissue) | rank) | risks | risks | ratio | design | risk) | risk) | groups |
| AURKA | 3.1230 | 0.0065 | 16 | 39 | 9.73 | 2 | 2.06 | 10.03 | 4.86 |
| BRRN1 | 10.2785 | 0.0086 | 27 | 28 | 3.57 | 2 | 5.22 | 17.35 | 3.32 |
| CENPW | 1.5595 | 0.0041 | 18 | 37 | 10.58 | 2 | 1.10 | 5.61 | 5.10 |
| MELK | 8.1813 | 0.0003 | 30 | 25 | 5.42 | 2 | 3.59 | 14.84 | 4.14 |
| PRR11 | 4.2266 | 0.2103 | 10 | 45 | 2.47 | 2 | 1.96 | 16.20 | 8.25 |
| E2F1 | 1.5690 | 0.0061 | 17 | 38 | 4.84 | 2 | 0.87 | 6.31 | 7.24 |

To understand the biological and functional significance of these co-expressed network components of 6g-TAGs genes in BC cells, we conducted gene ontology functional studies using David, GeneGo software's. Table EE2 enlists various gene ontology (GO) functions of the gene network components obtained based on Metacore software (Methods). These genes and it network components have a strong functional role in cell cycle (p=7.19 E-26), chromosome condensation (p=7.19E-26), regulation at G1/S (p=1.56 E-13), G2/M transition (p=4.43 E-35), regulation at kinetochore complex and chromosome segregation (1.26 E-12). Further represents list of various other gene ontology functions obtained using 6g-TAGs-related genes and its gene interaction network components.

TABLE EE2
Gene Ontology enrichment analysis. Various gene ontology
functions obtained using TAGs genes and its network
components using Metacore softweare.

| Top GeneGo Pathway Maps | p-value |
|---|---|
| Cell cycle_Chromosome condensation in prometaphase | 7.19E−26 |
| Cell cycle_Role of APC in cell cycle regulation | 3.05E−16 |
| Cell cycle_Regulation of G1/S transition (part 1) | 1.56E−13 |
| Cell cycle_Spindle assembly and chromosome separation | 1.26E−12 |
| Reproduction_Progesterone-mediated oocyte maturation | 1.08E−11 |
| Cell cycle_The metaphase checkpoint | 3.9E−09 |
| Cell cycle_Role of SCF complex in cell cycle regulation | 2.07E−08 |
| DNA damage_Brca1 as a transcription regulator | 2.67E−08 |
| Cell cycle_Role of Nek in cell cycle regulation | 4.35E−08 |
| Cell cycle_ESR1 regulation of G1/S transition | 5.47E−08 |
| Top GeneGo Process Networks | p-value |
| Cell cycle_Mitosis | 2.35E−46 |
| Cell cycle_G2-M | 4.43E−35 |
| Cytoskeleton_Spindle microtubules | 3.52E−21 |
| Cell cycle_Core | 2.52E−19 |
| Proteolysis_Proteolysis in cell cycle and apoptosis | 4.9E−15 |
| Cell cycle_G1-S | 1.58E−13 |
| DNA damage_Checkpoint | 5.43E−12 |
| Cytoskeleton_Regulation of cytoskeleton rearrangement | 7.82E−10 |
| Cytoskeleton_Cytoplasmic microtubules | 1.26E−09 |
| Cell cycle_Meiosis | 2.86E−08 |

To reconfirm the above observation we submitted the TAGs gene network components in DAVID Bioinformatics GO software, representing various biological functions attributing to 6g-TAGs genes and its gene interaction network components having strong statistical significance at FDR. Interestingly, both the software showed similar biological functions, re-affirming that TAGs network components have strong functional role in breast cancer via cell cycle and other downstream biological processes.

To validate the above co-expression phenomenon observed in breast cancer cohort datasets, we conducted qRT-PCR experiments using tissue array qPCR experiments. cDNA was synthesized from 58 breast tumour samples of RNA's from GSE61304 dataset and conducted qRT-PCR studies and estimated relative fold change values with respect to normal samples. Co-efficient of correlation was estimated for 6g-TAGs genes using 58 breast cancer patient samples. FIG. 12C shows that all the 6g-TAGs genes that are positively correlated in breast cancer microarray dataset (Uppsala, Singapore, Stockholm, BII-US) were in concordance with qPCR experiments. This strongly supports that all the 6-g TAGs genes are co-expressed in breast cancer patients and might have strong functional role in breast cancer.

### Example 44

### Results: TAGs Genes can Co-Localized and Form Complexes at Protein Level Attributing Critical Role in Breast Cancer

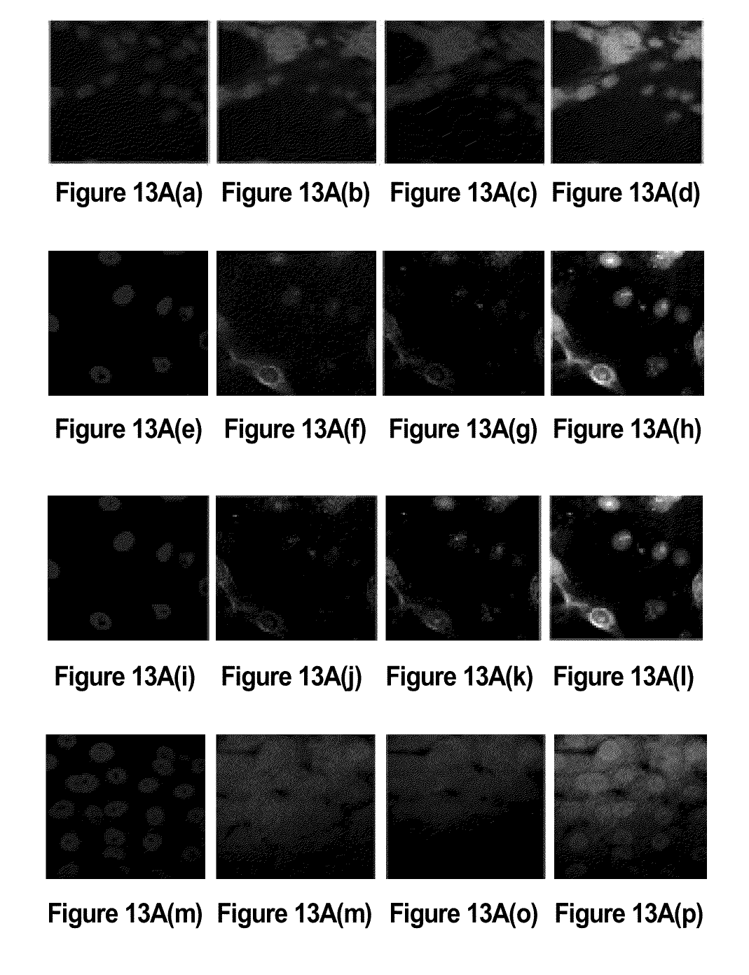

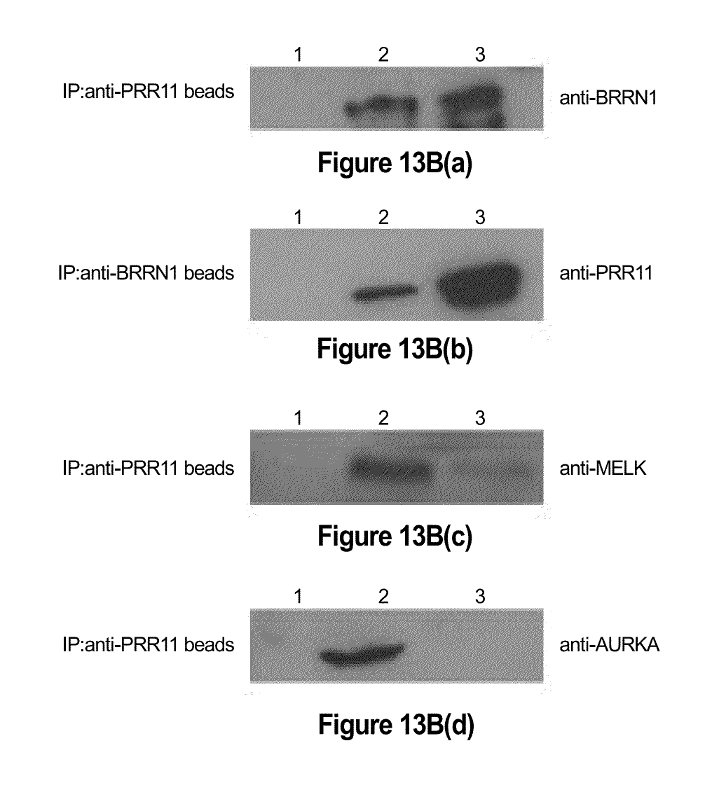

Based on previous publications [43-45] it was shown that co-expressed genes may be co-regulated and might have a possibility to interact with each other and attributing to critical biological functions. To assess further, if the positively correlated 6g-TAGs genes co-occurrence in BC, we conducted co-localization studies on PRR11, BRRN1, MELK and CENPW (part of TAGs genes) using immuno-fluorescent experiments (confocal microscopy). FIG. 13A(a-d) represents co-localization experiments conducted between PRR11 and BRRN1 in MDA-MB-436. FIG. 13A(a) represents DAPI nuclear stain (blue channel), 13A(b,f) green channel for GFP-PRR11, and 13A(c,g) red channel for BRRN1 and 6A-d is overlap showing strong co-localization of PRR11 and BRRN1 protein. Similar kinds of experiments were conducted to test other combination of 6g-TAGs gene. FIG. 13A(e-h) represents co-localization studies between PRR11 and BRRN1. FIG. 13A(h) represents data of co-localization of PRR11 and BRRN1. FIG. 13A(i-l) represents data of co-localization studies between BRRN1 and MELK, wherein, we can see clear co-localization of BRRN1 and MELK. FIG. 13A(m-p) represents data of co-localization studies between PRR11 and CENPW, wherein, there is no co-localization between PRR11 and CENPW proteins. Based on co-localization studies, we could clearly infer that PRR11, BRRN1 and MELK proteins form complexes with each other.

To support above observation, we tested if the above mentioned proteins (PRR11, BRNN1, MELK, and AURKA) form any complexes with each other by performing immunoprecipitations of MDA-MB-436 cell lysates, using anti-PRR11 and anti-BRRN1 antibodies coupled to the surface of CNBr sepharose beads. FIG. 13B(a-d) shows Western blotting with anti-BRRN1 antibody after immunoprecipitation with rabbit anti-PRR11 serum. BRRN1 is expressed in MDA-MB-436, and was detected in immunocomplexes with endogenous PRR11. From the converse experiments MDA-MB-436 lysates were immunoprecipitated using anti-BRRN1 antibody CNBr sepharose beads. FIG. 13B(a-d) shows Western blotting with anti-GFP to detect GFP-PRR11. PRR11 and BRRN1 were found in one protein complex. The negative control (CNBr sepharose beads) showed no PRR11 or BRRN1 in these experiments (FIG. 13B(a,b) lane 1). Further we noticed MELK forming complex with PRR11 which is evident from FIG. 13B(c) lane 3. FIG. 13B(d) shows no interaction between PRR11 and AURKA.

### Example 45

### Results: TAGs Genes Play Critical Role at G2/M and G1/S Cell Cycle Checkpoints in Breast Cancer

Gene ontology functions of TAGs genes and its interacting gene network components showed that these genes have a significant role at various check points of cell cycle (G1/S, G2/M). To understand the functional role of TAGs genes at various cell cycle phases, MDA-MB 436 cells were synchronized and cells were further sorted at G1, S and G2/M phases. RT-PCR gene expression studies were carried out on 6g-TAGs genes at various synchronized cell cycle phases. FIG. 14A shows expression of TAGs genes at various phases of cell cycle. FIG. 14A shows that AURKA-A is highly expressed at G2/M check point which is evident as AURKA plays a crucial role during Mitotic chromosomal segregation. E2F1 is highly expressed in G1/S and G2/M check points. BRRN1, CENPW are relatively higher in G2/M compared to other cell cycle phases. PRR11 which is poorly characterized in breast cancer is highly expressed in G2/M, but very low in G1 and G1/S of breast cancer cell line.

Further siRNA silencing studies were conducted on all TAGs genes to check at which phase of cell cycle these siRNA treated cells were arrested. FIG. 14B shows FACS analysis using Propidium Iodide (PI) studies conducted using independent siRNA silencing experiments of various TAGs genes relatively compared with control siRNA on MDA-MB-436 breast cancer cell line. Silencing of AURKA, CENPW showed cells getting arrested at G2/M transition. E2F1 depletion experiments show that the cells are arrested at S phase of cell cycle. Further, silencing of MELK shows that the cells arresting at G1 phase of cell cycle. However PRR11 silencing experiments show that there are at least 13% of cells accumulating in sub-G fraction assuming cells undergoing apoptosis. Further, experiments were conducted to assess the proliferation potential of 6g-TAGs genes. Cells were treated with siRNA of each individual 6g-TAGs genes and counted cells at various time points until 72 hrs. FIG. 14C shows independent silencing of 6g-TAGs genes depleting cell proliferation ability when relatively compared to control siRNA treated MDA-MB436 cells. This clearly shows that all the TAGs genes have potential proliferation capability.

### Example 46

### Results: 6g-TAGs Genes are Strong Prognostic Biomarkers in Breast Cancer, Validated Both by Computational Predictions and by qPCR

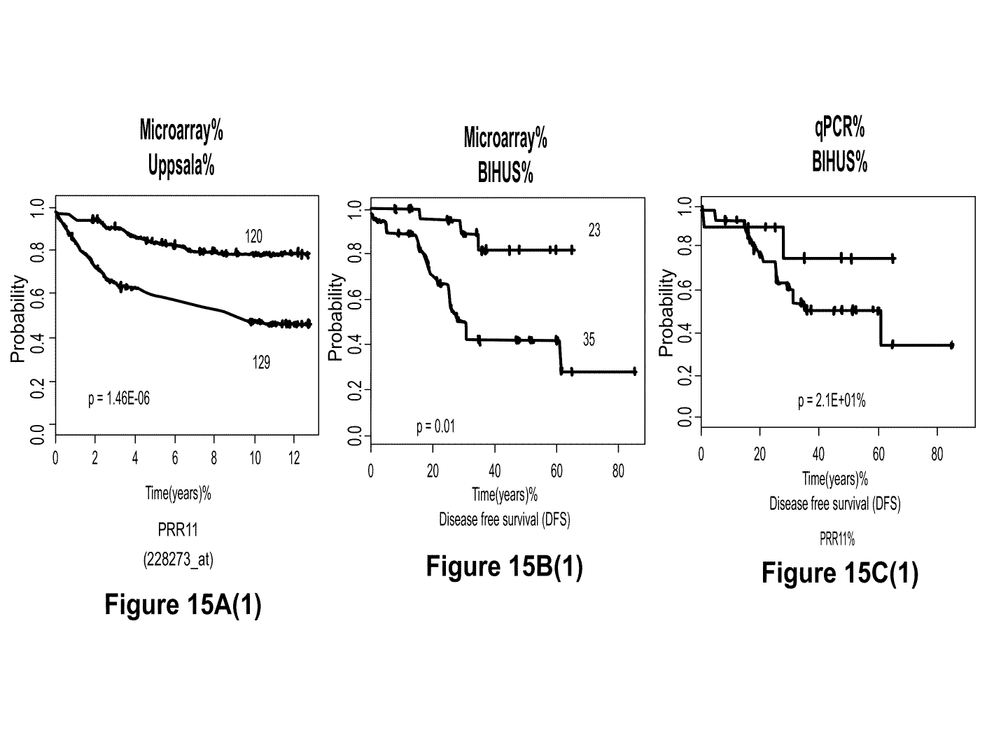

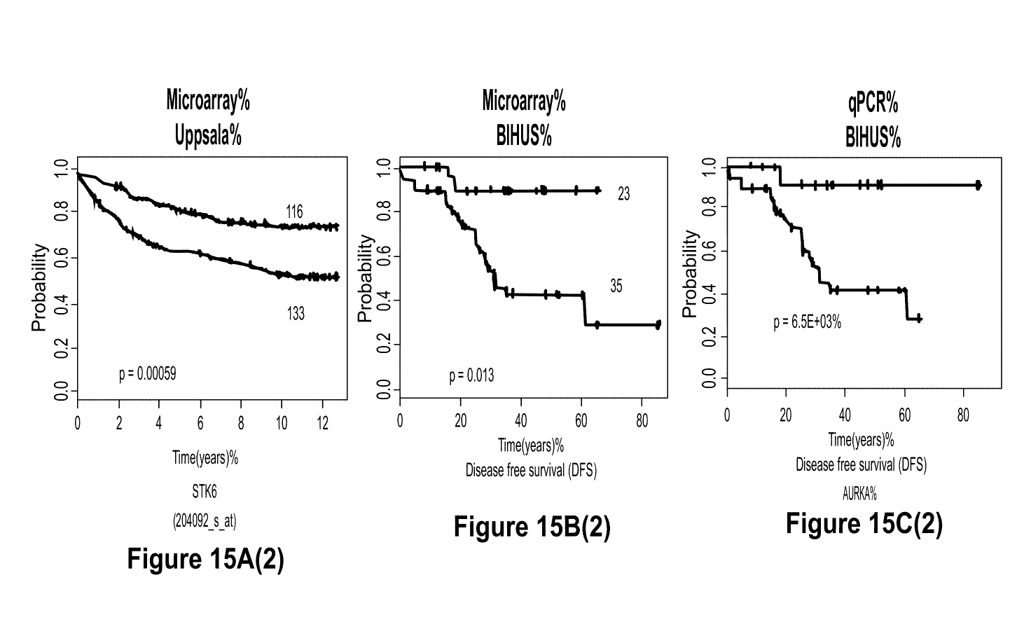

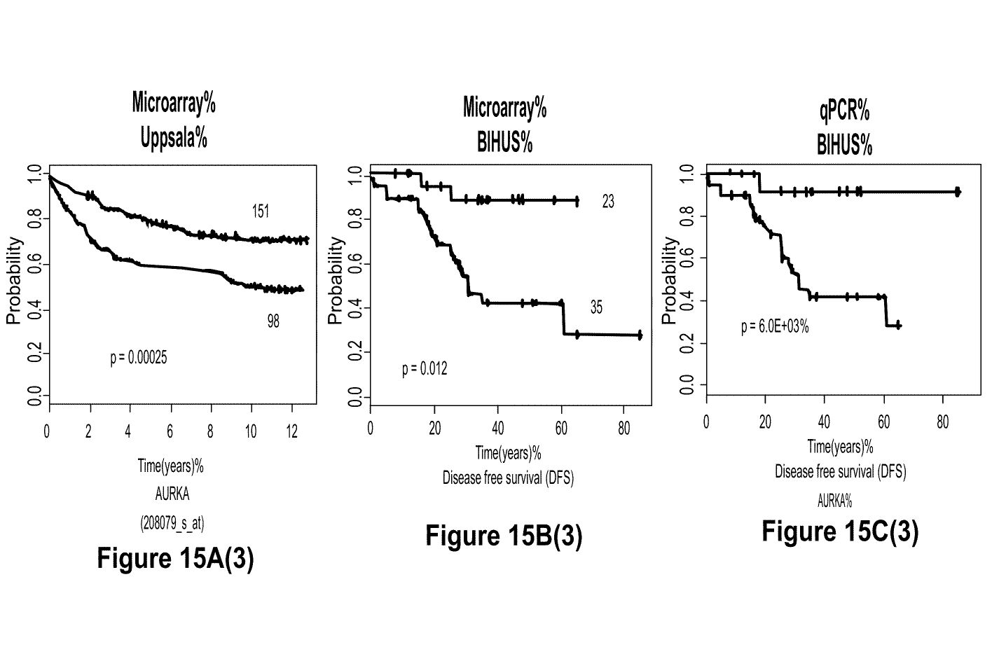

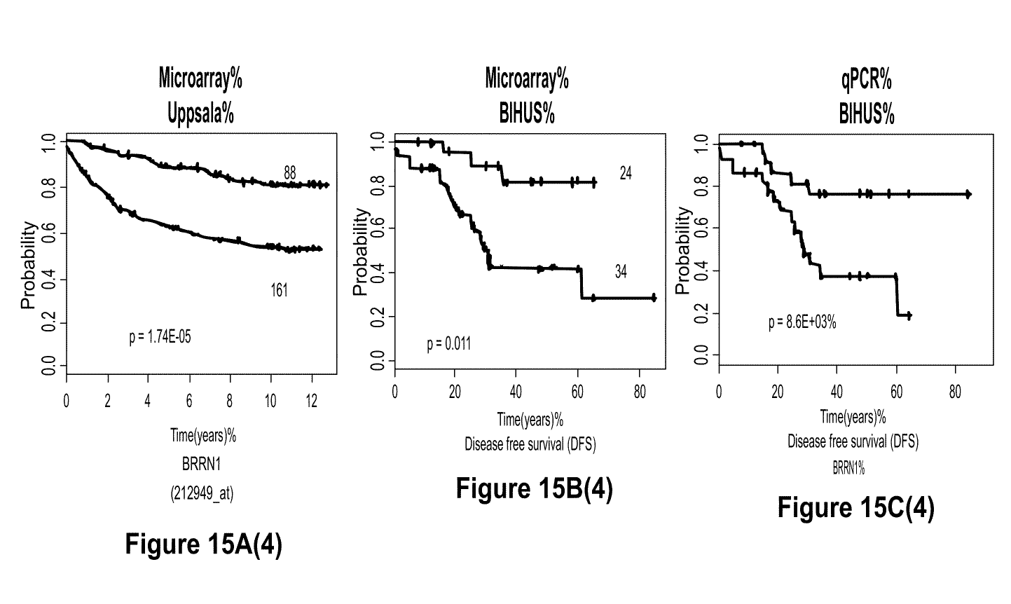

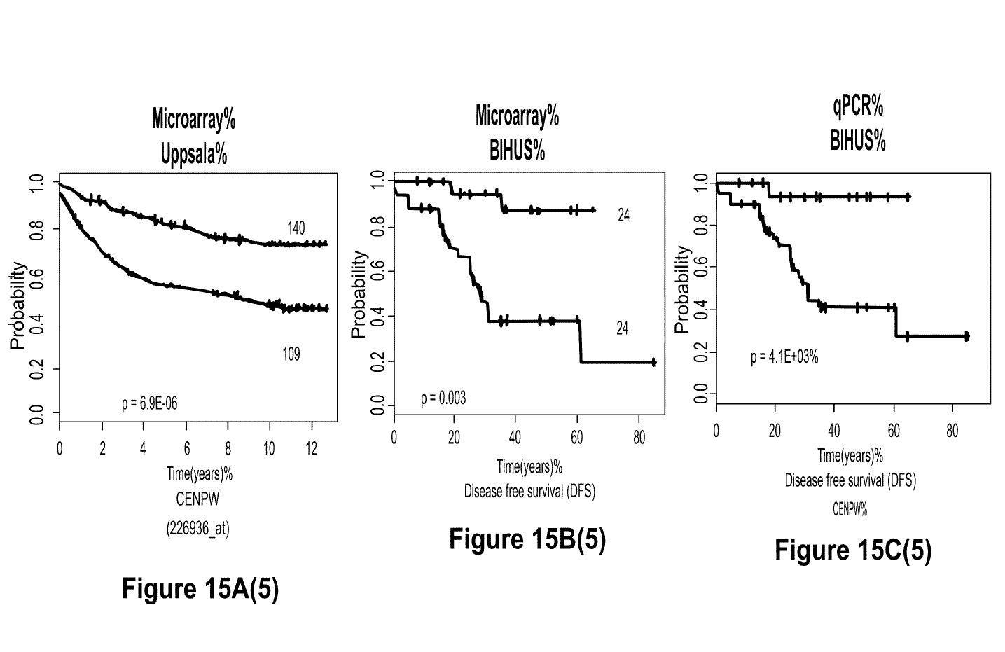

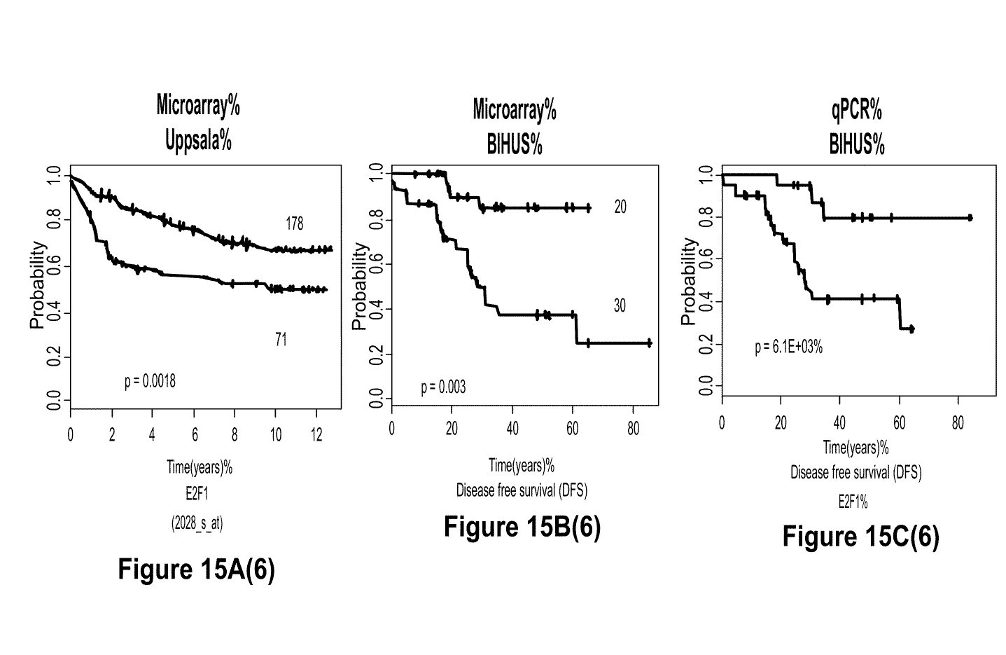

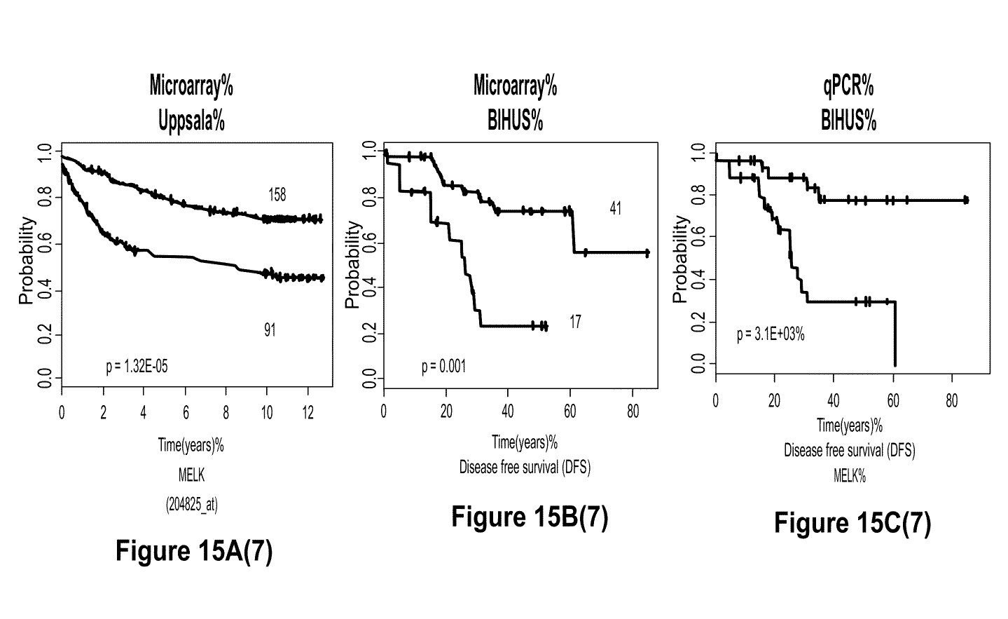

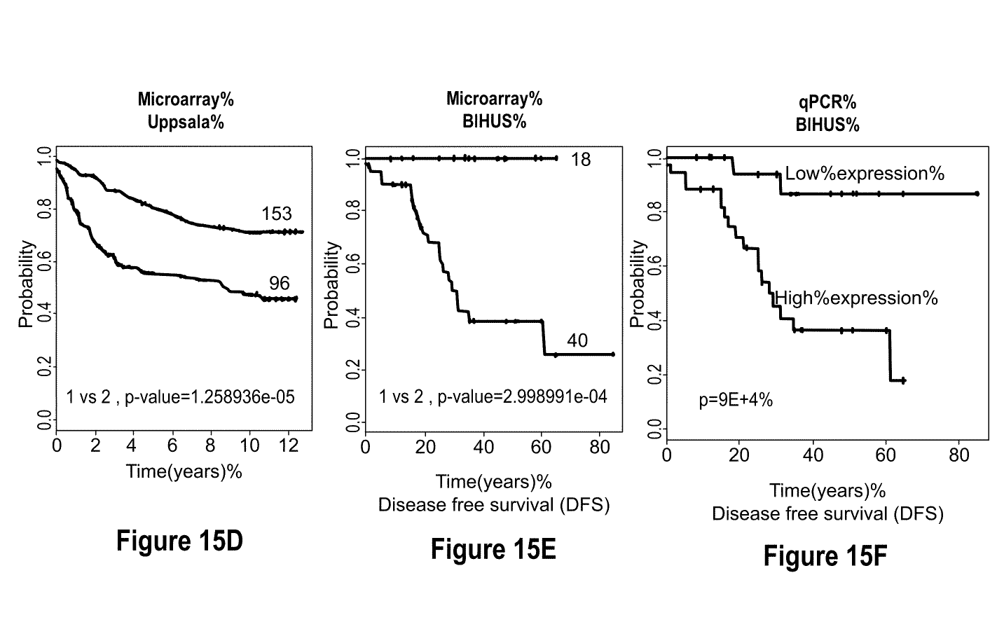

One of the key questions to be addressed is to check if 6g-TAGs genes can show prognostic potential indiscriminate low risk and high risk patients with respect to recurrence free survival. We investigated this phenomenon in various breast cancer microarray cohort datasets (Uppsala, Stockholm, Singapore and BII-US). All the breast cancer microarray datasets have been analysed using disease free survival information defined as, the time interval from surgery until the first recurrence (local, regional, or distant) or last date of follow-up. FIGS. 15A(1)-15A(7) and FIGS. 15B(1)-15B(7) clearly show strong prognostic ability of all 6g-TAGs genes in Uppsala and BII-US breast cancer microarray cohort dataset. This observation is consistent with various other breast cancer microarray cohorts datasets analysed. These 6g-TAGs genes either independently or as a group can act potential prognostic biomarkers with respect to recurrence free survival.

To validate prognostic ability of the 6g-TAGs genes observed in various microarray breast cancer cohort datasets, we conducted qPCR experiments of all 6g-TAGs genes using cDNA of 62 breast cancer patient samples (with DFS clinical information). qPCR assay delta Ct-values were extracted as explained in Methods section and used in our 1D DDg analysis. FIGS. 15C(1)-15C(7) show prognostic ability of the 6-g TAGs genes tested using qPCR validations. FIGS. 15C(1)-15C(7) and (Table EE8) clearly show that the prognostic significance of the 6g-TAGs genes observed in qPCR experiments is in concordance with the microarray breast cancer cohort datasets.

TABLE EE8
Results the Modified Wilcoxon Test MWT p-values for matched pair samples in
TCGA (A) and GSE10780 (B) dataset, A: Agilent platform G4502A. B: Affymetrix U133 A&B
probesets.
A

| | | | # of cancer | | | | |
|---|---|---|---|---|---|---|---|
| | | | samples | # of cancer | | | |
| probesets ID | | | where the | samples | | | |
| Agilent | | | genes are | where the | # of | | |
| platform | Gene | Entrez | down | genes are up | misclass- | Accuracy | MWT p- |
| G4502A | symbol | ID | regulated | regulated | ifications | % | values |
| A_23_P131866 | AURKA | 6790 | 1 | 59 | 1 | 98.33 | 1.47E−09 |
| A_24_P462899 | CENPW | 387103 | 1 | 59 | 1 | 98.33 | 7.44E−10 |
| A_23_P94422 | MELK | 9833 | 0 | 60 | 0 | 100 | 4.91E−10 |
| A_23_P415443 | BRRN1 | 23397 | 1 | 59 | 1 | 98.33 | 6.91E−09 |
| A_23_P207307 | PRR11 | 55771 | 4 | 56 | 4 | 93.33 | 1.92E−08 |
| A_23_P80032 | E2F1 | 1869 | 0 | 60 | 0 | 100 | 4.91E−10 |

B

| | | | # of cancer | | | | |
|---|---|---|---|---|---|---|---|
| | | | samples | # of cancer | | | |
| | | | where the | samples | | | |
| Affymetrix | | | genes are | where the | # of | | |
| U133 A&B | Gene | Entrez | down | genes are up | misclass- | Accuracy | MWT p- |
| probesets IDs | symbol | ID | regulated | regulated | ifications | % | values |
| 208079_s_at | AURKA | 6790 | 0 | 22 | 0 | 100 | 0.000669 |
| 226936_at | CENPW | 387103 | 0 | 22 | 0 | 100 | 0.000669 |
| 204825_at | MELK | 9833 | 0 | 22 | 0 | 100 | 0.000669 |
| 212949_at | BRRN1 | 23397 | 0 | 22 | 0 | 100 | 0.000669 |
| 228273_at | PRR11 | 55771 | 0 | 22 | 0 | 100 | 0.000669 |
| 2028_s_at | E2F1 | 1869 | 0 | 22 | 0 | 100 | 0.000669 |
| 204947_at | E2F1 | 1869 | 2 | 20 | 2 | 90.91 | 0.001417 |
| 204092_s_at | AURKA | 6790 | 0 | 22 | 0 | 100 | 0.000669 |

Further experiments were carried out to check the synergistic prognostic potential of the 6g-TAGs genes indiscriminating low and high risk breast cancer patients. This was tested using our Statistical Weighted Voting classification method (see Methods). We used 6g-TAGs genes of Uppsala cohort microarray data. FIG. 15D shows discrimination of the patients into low- and high-risk the disease development groups. These observations were corroborated with BII-US cohort, when the 6-gTAGs dataset (FIG. 15E; Table EE8) was used for stratification of the patients based on the both microarray and qPCR data sets (FIG. 15F). Collectively, these findings suggest the high levels of the patient's separation ability and reproducibility of the 6g-TAGs genes as the potential diagnostic biomarkers (Table EE7).

### Example 47

### Results: Univariate and Multivariate Analysis of 6g-TAGs in Various Breast Cancer Datasets

We compared the prognostic performance of the 6-g TAGs classification with several other known clinical risk factors in various breast cancer cohorts using univariate and multivariate Cox regression analyses (Table EE3).

TABLE EE3

| | | | | | | | | | |
|---|---|---|---|---|---|---|---|---|---|
| coef | HR | P value | lower .95 | upper .95 | coef | HR | P value | lower .95 | upper .95 |

| | |
|---|---|
| Univariate analysis | Multivariate analysis |

Uppsala

| | | | | | | | | | | | |
|---|---|---|---|---|---|---|---|---|---|---|---|
| AGE | −0.003 | 0.997 | 7.222652E−01 | 0.982 | 1.013 | AGE | 0.012 | 1.012 | 1.574743E−01 | 0.995 | 1.030 |
| ER | −0.153 | 0.858 | 6.229746E−01 | 0.467 | 1.578 | ER | 0.284 | 1.328 | 4.023533E−01 | 0.684 | 2.579 |
| PR | −0.378 | 0.685 | 1.724700E−01 | 0.398 | 1.180 | PR | −0.028 | 0.972 | 9.266691E−01 | 0.533 | 1.773 |
| LN | 0.745 | 2.109 | 4.730000E−04 | 1.388 | 3.204 | LN | 0.437 | 1.548 | 6.495241E−01 | 0.973 | 2.462 |
| SIZE | 0.016 | 1.016 | 2.081677E−03 | 1.005 | 1.026 | SIZE | 0.006 | 1.006 | 3.690716E−01 | 0.993 | 1.018 |
| TAGs | 1.045 | 2.844 | 1.130000E−06 | 1.867 | 4.331 | TAGs | 1.043 | 2.838 | 2.270000E−05 | 1.752 | 4.598 |

Stockholm

| | | | | | | | | | | | |
|---|---|---|---|---|---|---|---|---|---|---|---|
| AGE | −0.006 | 0.994 | 6.670945E−01 | 0.969 | 1.020 | AGE | −0.010 | 0.990 | 4.741863E−01 | 0.962 | 1.018 |
| ER | −0.521 | 0.594 | 1.719180E−02 | 0.281 | 1.254 | ER | 0.155 | 1.168 | 7.456238E−01 | 0.458 | 2.980 |
| PR | −0.726 | 0.484 | 2.725972E−02 | 0.254 | 0.922 | PR | −0.571 | 0.565 | 1.712169E−01 | 0.249 | 1.280 |
| LN | 0.028 | 1.028 | 9.343363E−02 | 0.533 | 1.982 | LN | −0.013 | 0.987 | 9.704824E−01 | 0.483 | 2.014 |
| SIZE | 0.013 | 1.013 | 2.501451E−01 | 0.991 | 1.037 | SIZE | 0.015 | 1.015 | 2.991181E−01 | 0.986 | 1.045 |
| TAGs | 1.135 | 3.112 | 7.572640E−03 | 1.607 | 6.024 | TAGs | 0.910 | 2.484 | 1.321597E−02 | 1.209 | 5.102 |

Singapore

| | | | | | | | | | | | |
|---|---|---|---|---|---|---|---|---|---|---|---|
| AGE | 0.000 | 1.000 | 9.940446E−01 | 0.961 | 1.041 | AGE | 0.005 | 1.005 | 8.412947E−01 | 0.959 | 1.053 |
| ER | −0.927 | 0.396 | 4.611286E−02 | 0.159 | 0.984 | ER | −0.298 | 0.742 | 6.169688E−01 | 0.231 | 2.386 |
| PR | −1.157 | 0.314 | 1.910935E−02 | 0.119 | 0.828 | PR | −0.759 | 0.468 | 2.257860E−01 | 0.137 | 1.598 |
| LN | 0.973 | 2.647 | 4.886981E−02 | 1.005 | 6.973 | LN | 0.929 | 2.531 | 6.619116E−02 | 0.940 | 6.819 |
| SIZE | 0.022 | 1.022 | 1.674054E−01 | 0.991 | 1.054 | SIZE | 0.010 | 1.010 | 5.740352E−01 | 0.975 | 1.046 |
| TAGs | 1.522 | 4.580 | 6.897331E−03 | 1.519 | 13.813 | TAGs | 1.096 | 2.993 | 6.482386E−02 | 0.935 | 9.583 |

US Cohort

| | | | | | | | | | | | |
|---|---|---|---|---|---|---|---|---|---|---|---|
| AGE | −0.001 | 0.990 | 9.290000E−01 | 0.970 | 1.030 | AGE | 0.035 | 1.036 | 8.360000E−02 | 0.021 | 1.730 |
| ER | −1.367 | 0.250 | 8.000000E−03 | 0.089 | 0.690 | ER | −2.210 | 0.109 | 1.942000E−01 | 0.004 | 3.090 |
| PR | −0.654 | 0.520 | 2.330000E−01 | 1.900 | 0.170 | PR | 1.810 | 6.160 | 2.972000E−01 | 0.200 | 187.800 |
| Stage | 0.520 | 1.680 | 6.000000E−02 | 0.970 | 2.900 | Stage | 0.750 | 2.120 | 9.680000E−02 | 0.870 | 5.200 |
| TAGs_5 | 2.660 | 14.300 | 9.500000E−03 | 1.910 | 107.00 | TAGs_5 | 2.430 | 11.400 | 4.000000E−02 | 1.100 | 118.100 |

In the Uppsala cohort, LN (4.73E-04), Size (2.08E-03) and 6g-TAGs genes (1.13E-06) have statistically significant Hazard ratio (>1). In Stockholm, Singapore and BII-US cohort the univariate hazard ratio for 6g-TAGs genes was relatively higher than other clinical risk factors with p value of 7.57E-04, 6.89e-03, and 9.5E-03 respectively. We then included all significant clinical variables in a multivariate Cox regression analysis; the 6g-TAGs classification retained its independent prognostic value with p values of 2.2E-05, 1.32E-02, 6.48E-02 and 4.0E-02 for Uppsala, Stockholm, Singapore and BII-US cohorts respectively. Table EE3 clearly represented details risk hazard ratios of various clinical risk factors selected from various datasets.

### Example 48

### Results: Reproducibility of the 6g-TAG Signature Across Different Cohorts, and Histo-Pathological Forms and within Tumour Subtypes

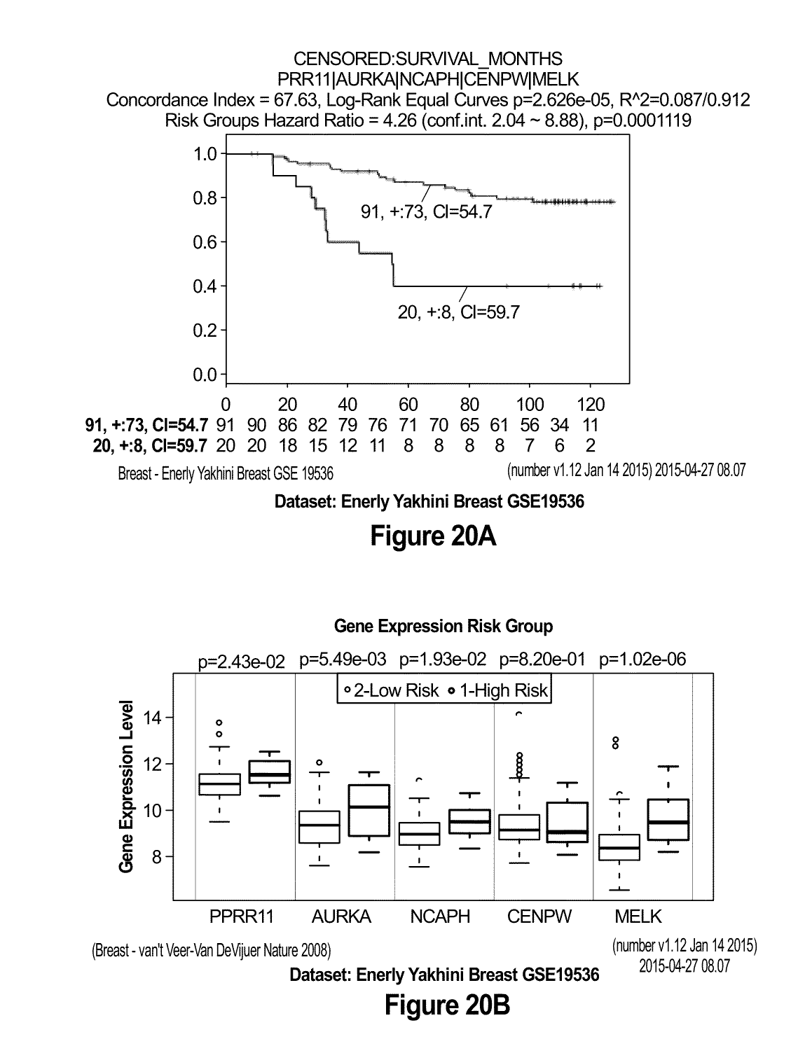

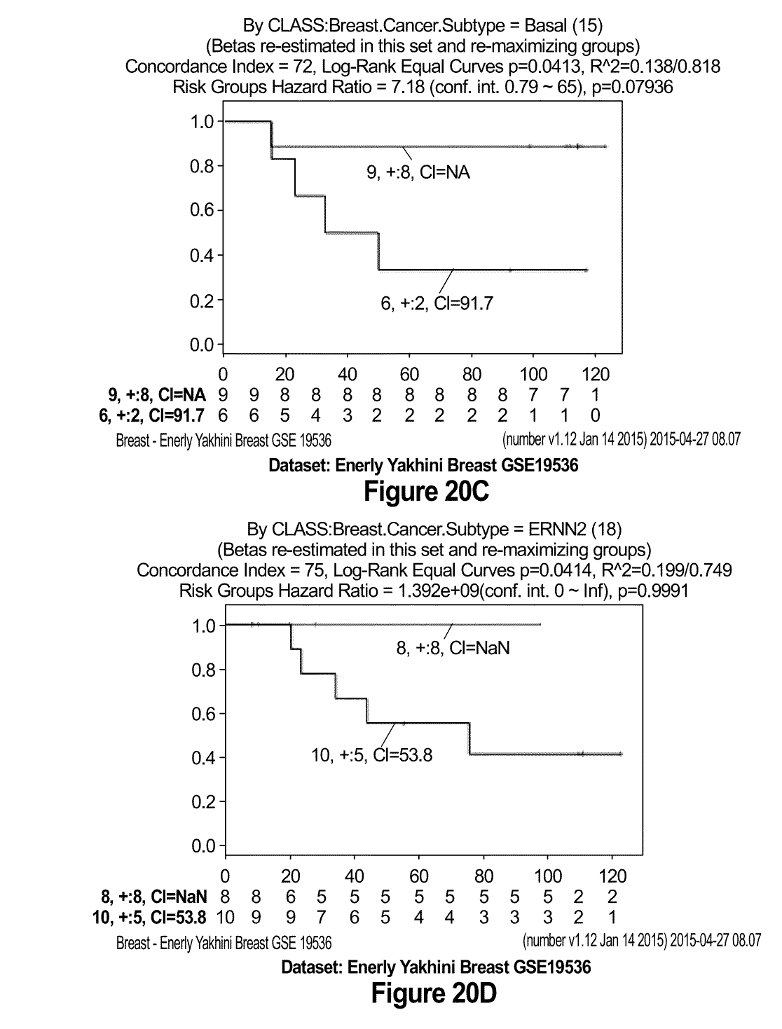

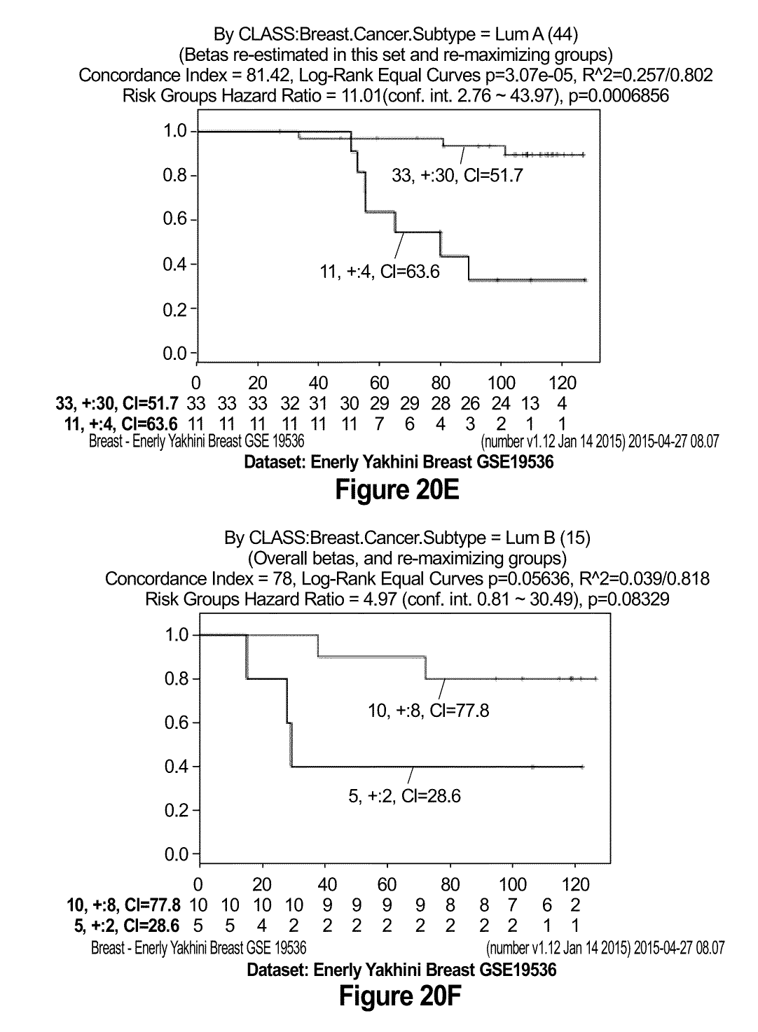

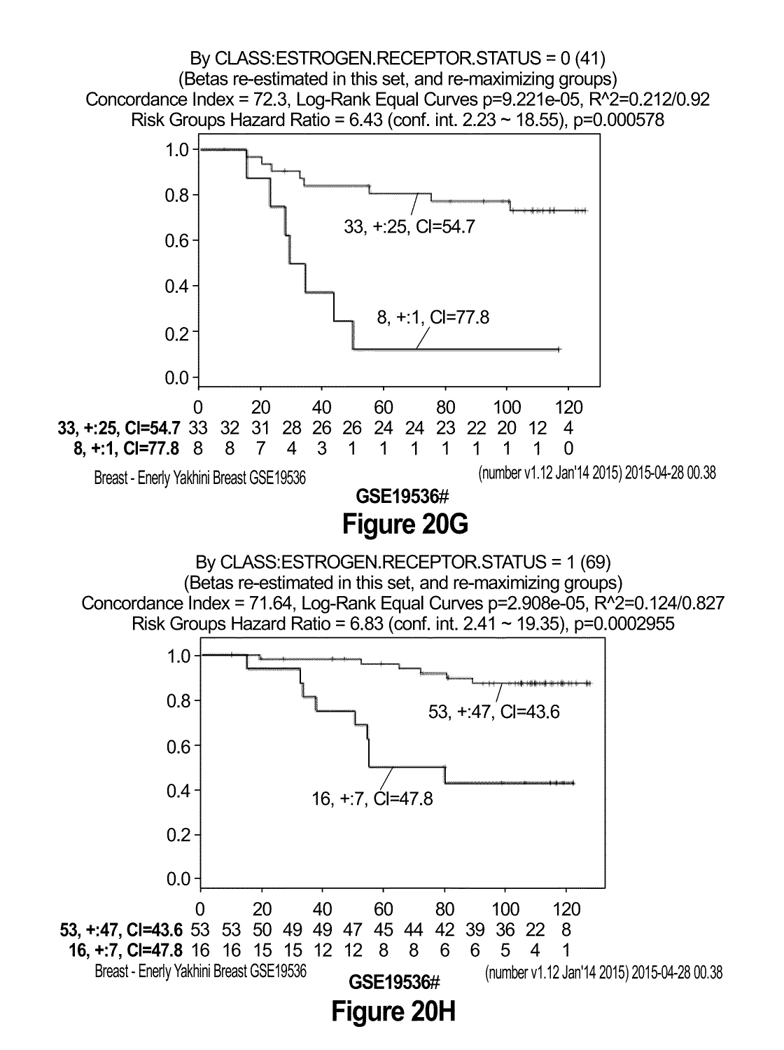

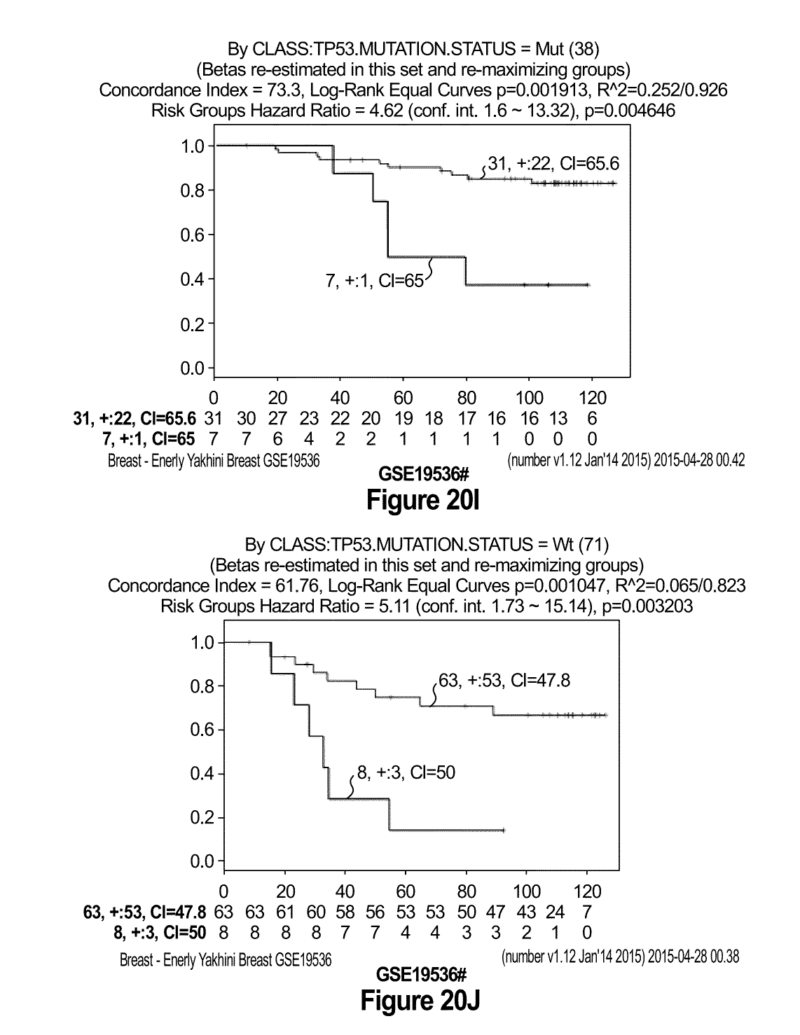

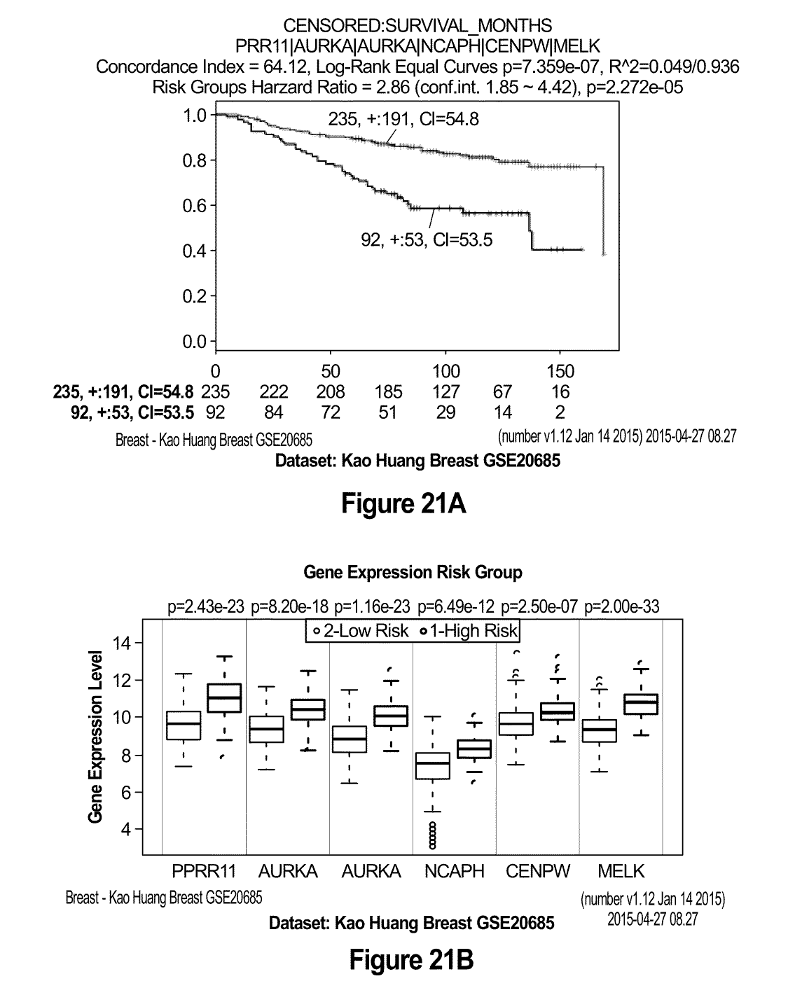

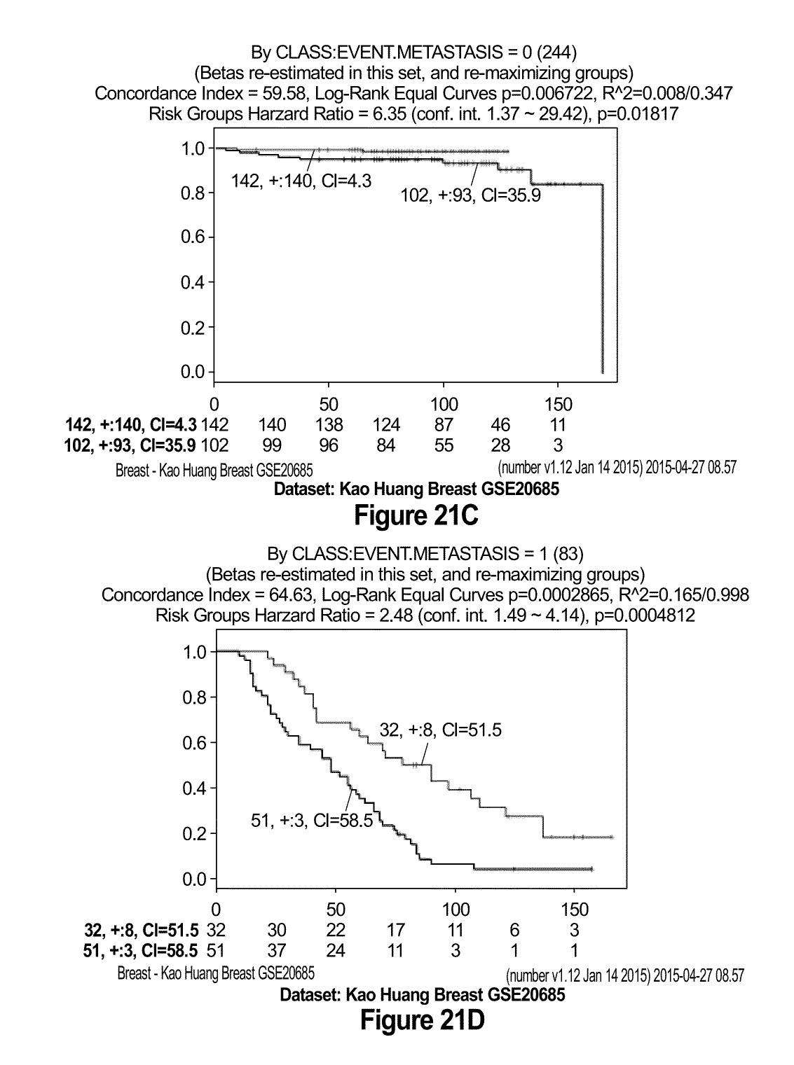

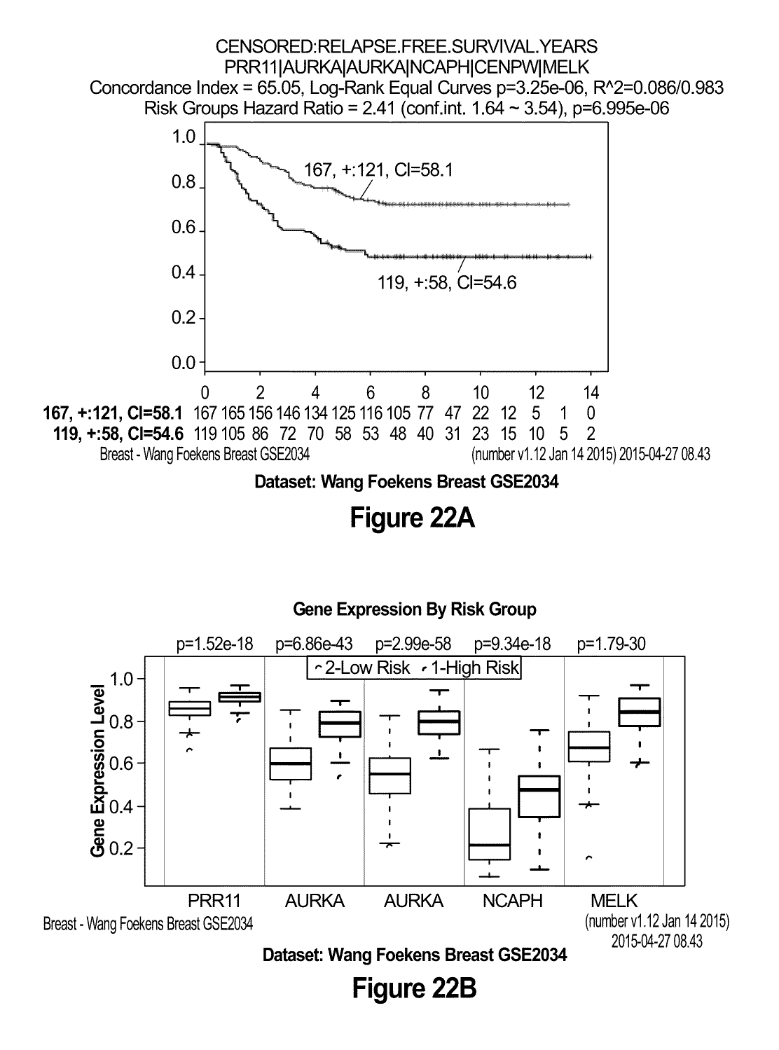

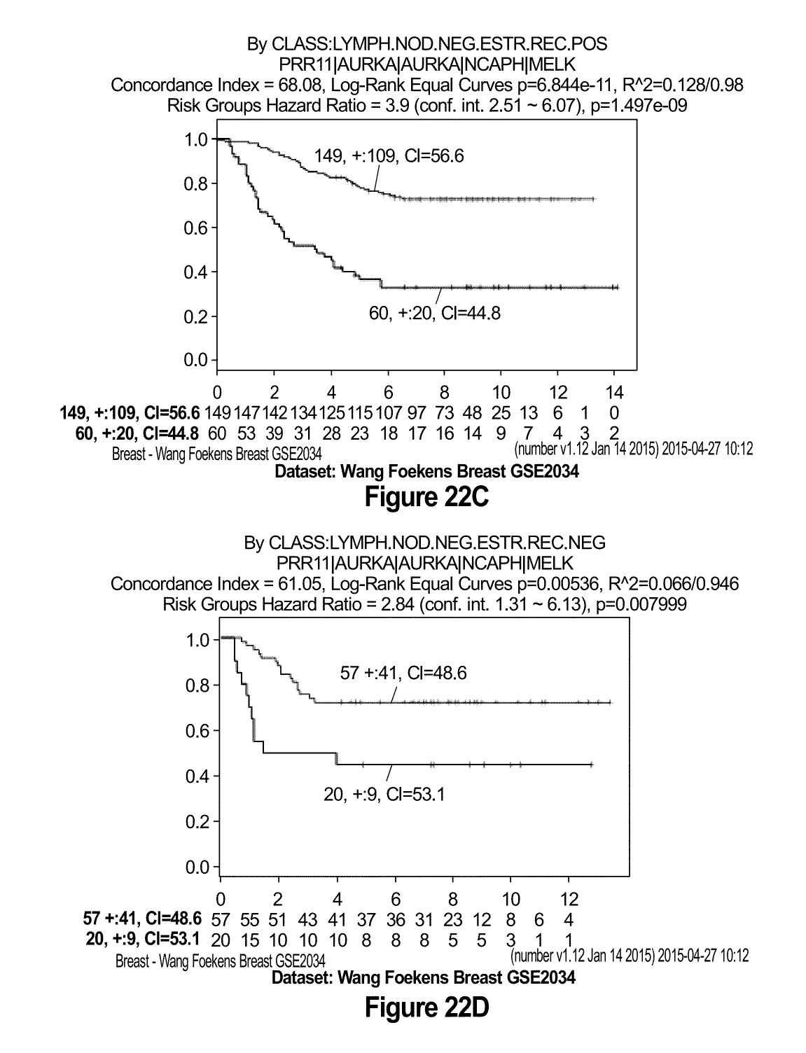

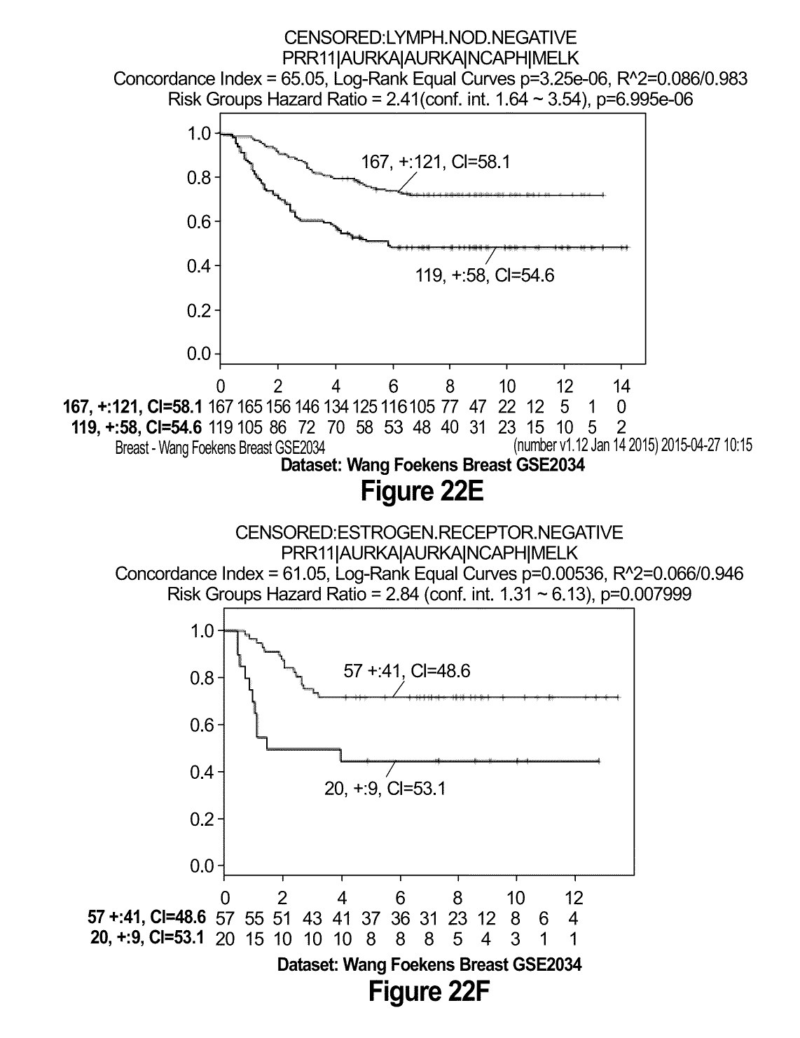

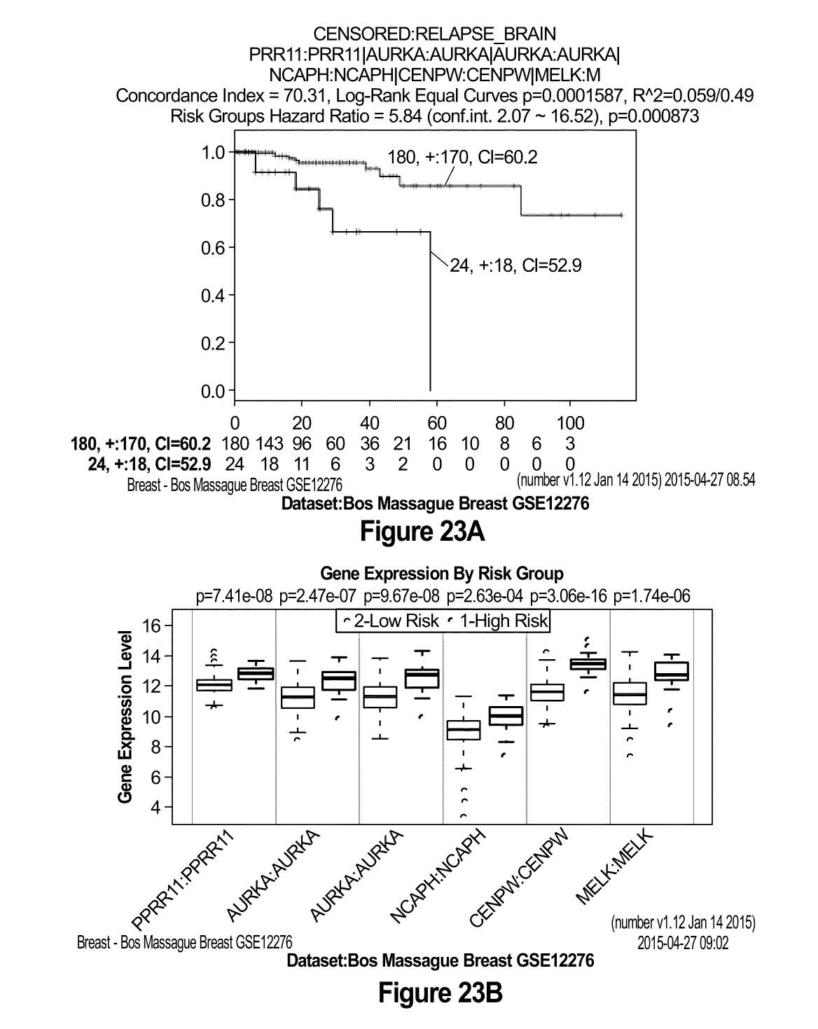

To test the robustness of the 6g-TAG genes prognostic ability, we explored Express Survival Web application containing multiple datasets within breast cancer. We selected various breast cancer datasets (FIG. 19A to FIG. 23B) and compared 6g-TAG genes with other clinical factors. Interestingly, TAG genes demonstrated strong prognostic ability in stratifying low risk and high risk groups, relative to other clinical factors. FIG. 19A to FIG. 23B demonstrate that reproducibility of prognostic significance of 6g-TAG gene prediction across different cohorts out performing other clinical variables with p<0.01. It includes comparing multiple data sets reproducing the low- and high-aggressive patterns of the tumour across different cohorts.

Importantly, the 6g-TAG signature able to stratify the patients within very specific clinical and molecular BC sub-classes (FIG. 19A to FIG. 23B). The method well reflects quantitatively the cancer cell cycle/mitosis rate, transcriptome over-expression and tumour aggressiveness of the different tumour types, subtypes and subclasses. Our TAG signature detection method could be implemented as uniform and objective prognostic factor, because it i) reflects and improves a measure of tumour aggressiveness previously based on clinical classification of tumours on low- and high-grade tumour classes and ii) it predicts outcome of BC patients without patient's preselection for assay conduction; our method could be apply for any cohorts regardless nuclear receptor status; tumour mass, tumour stages and subtypes.

### Example 49

### Results: 6g-TAG Signature Provides Disease Prediction Outcomes in Cohorts with Other Tumour Types

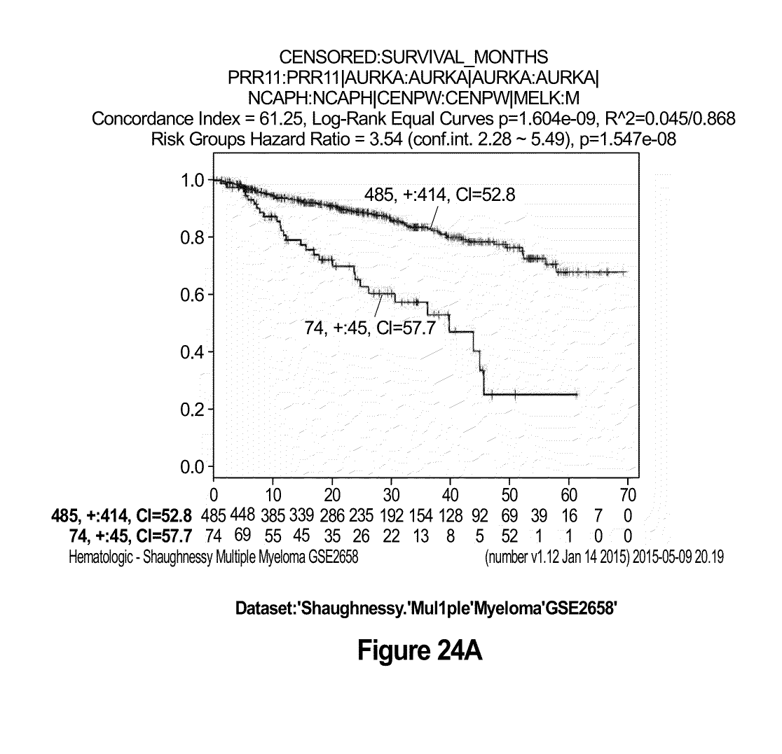

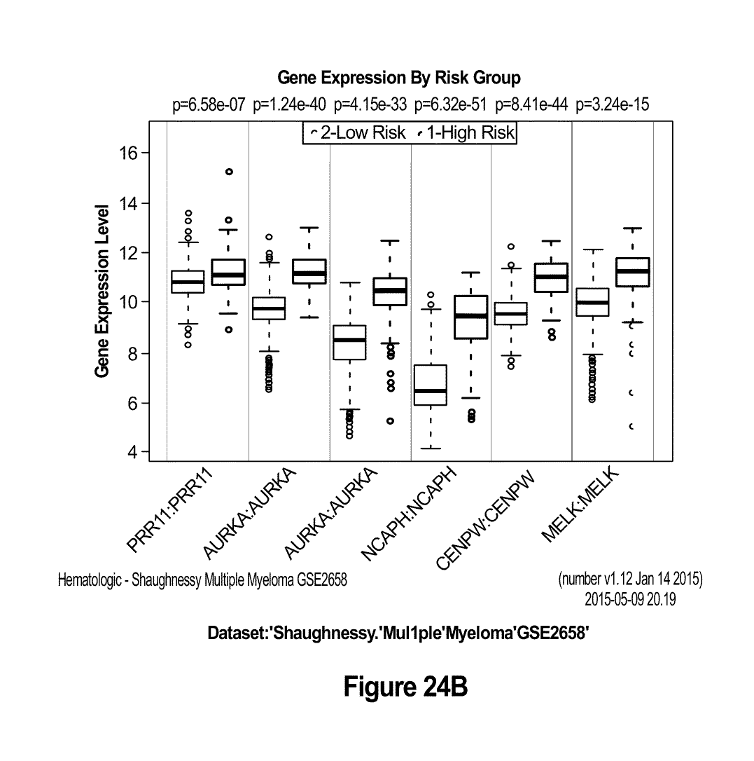

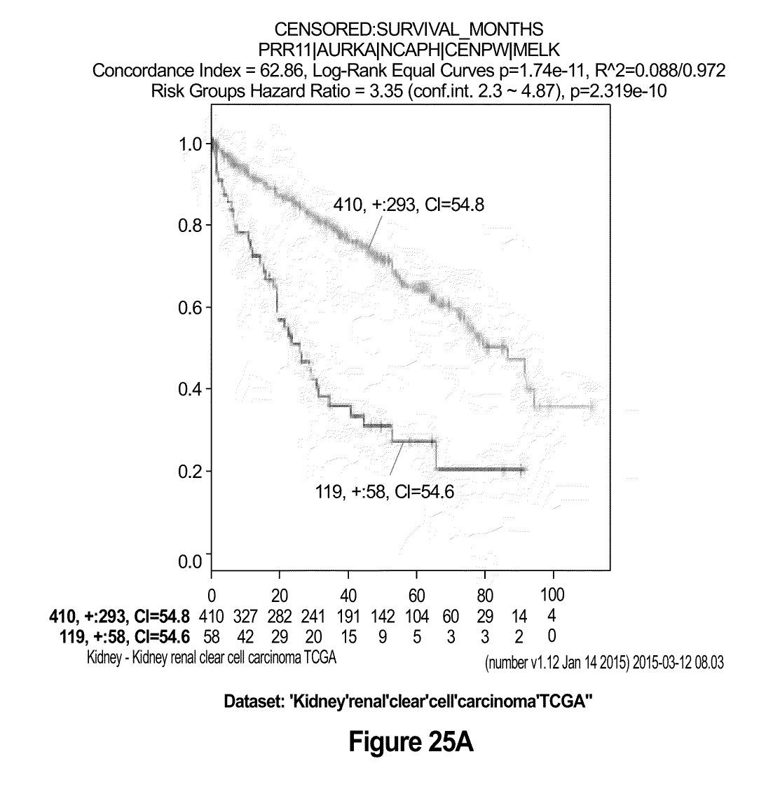

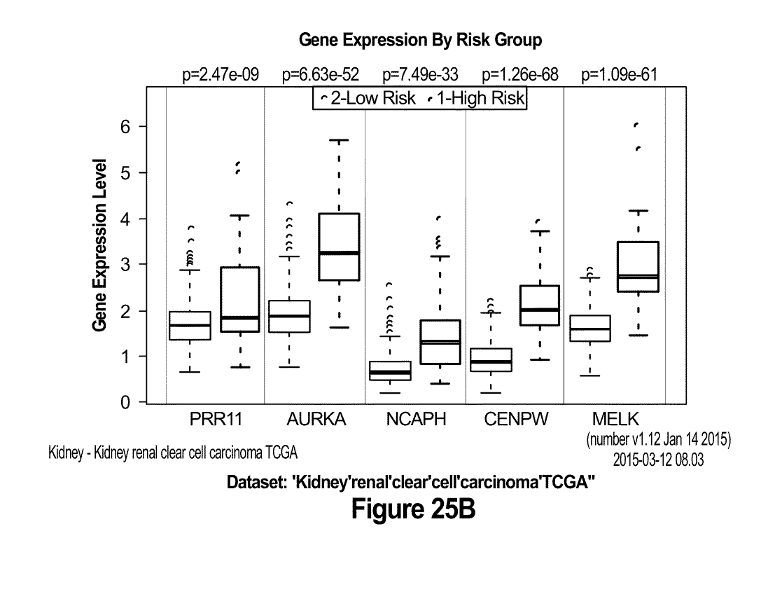

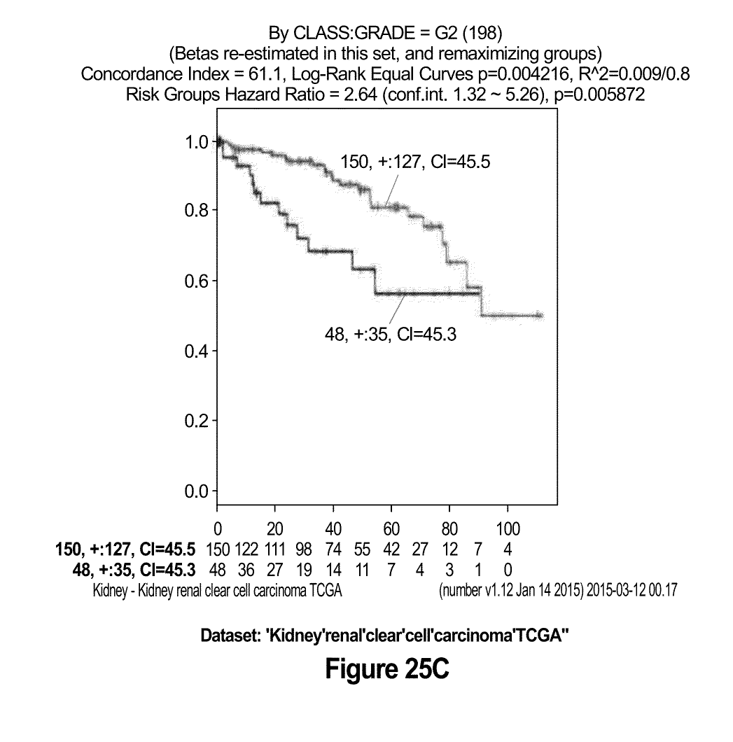

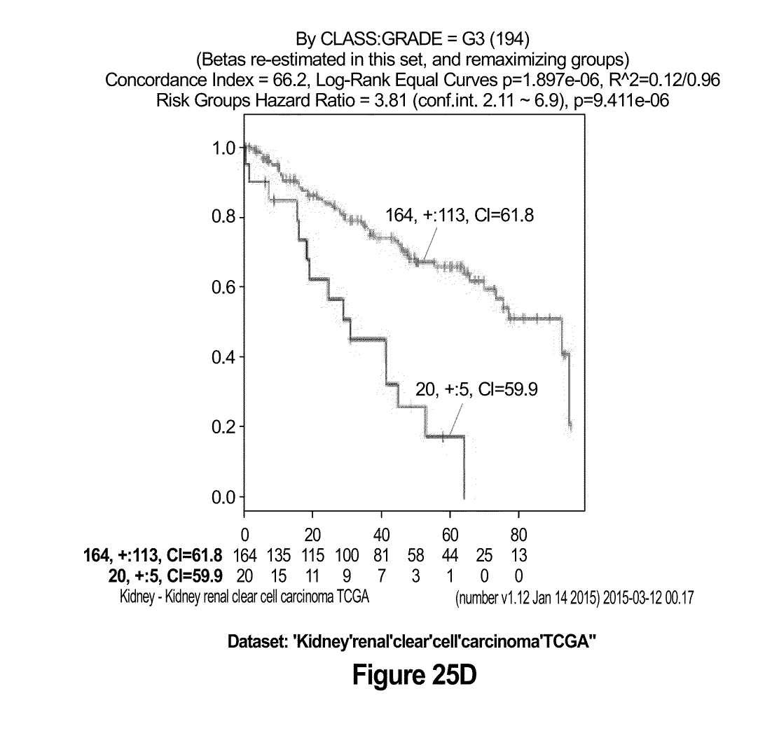

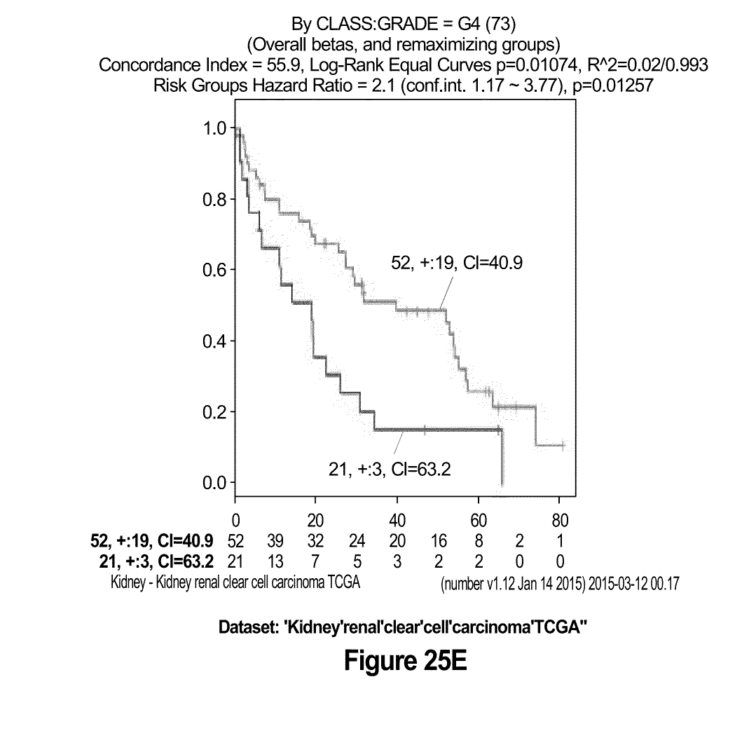

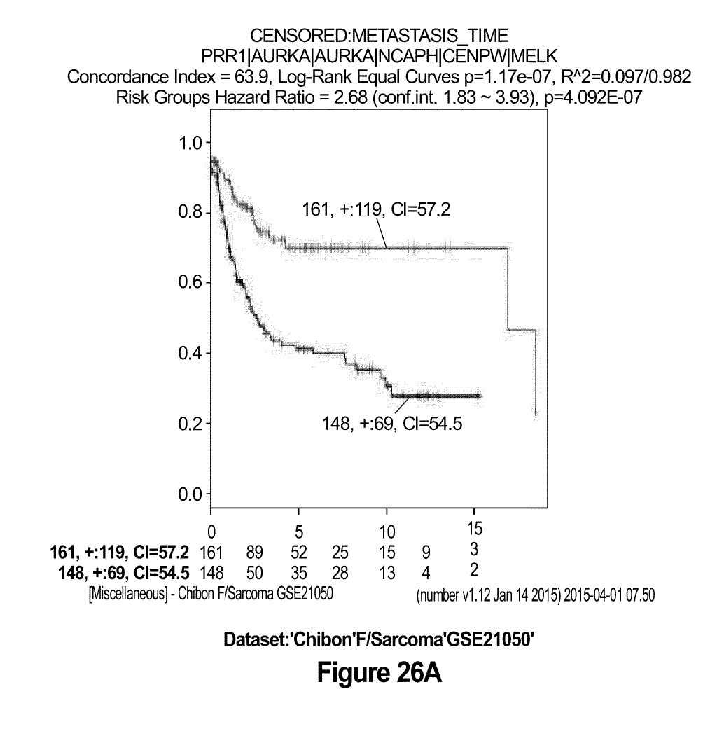

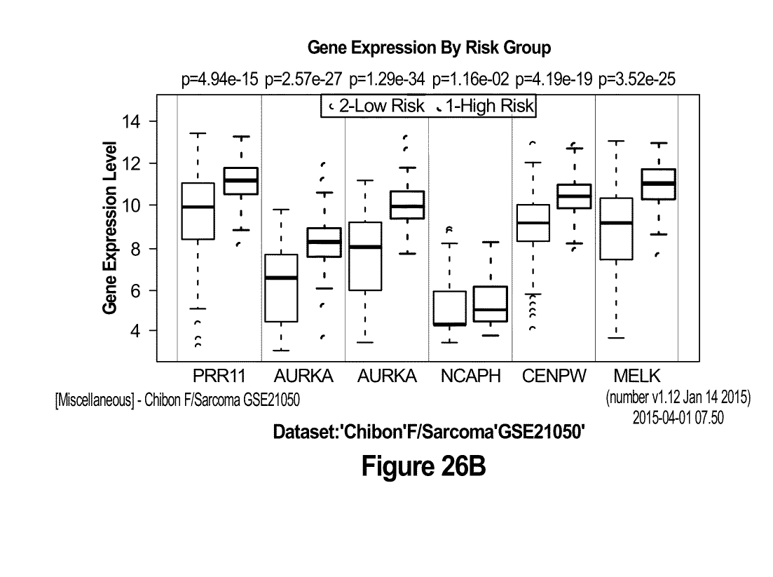

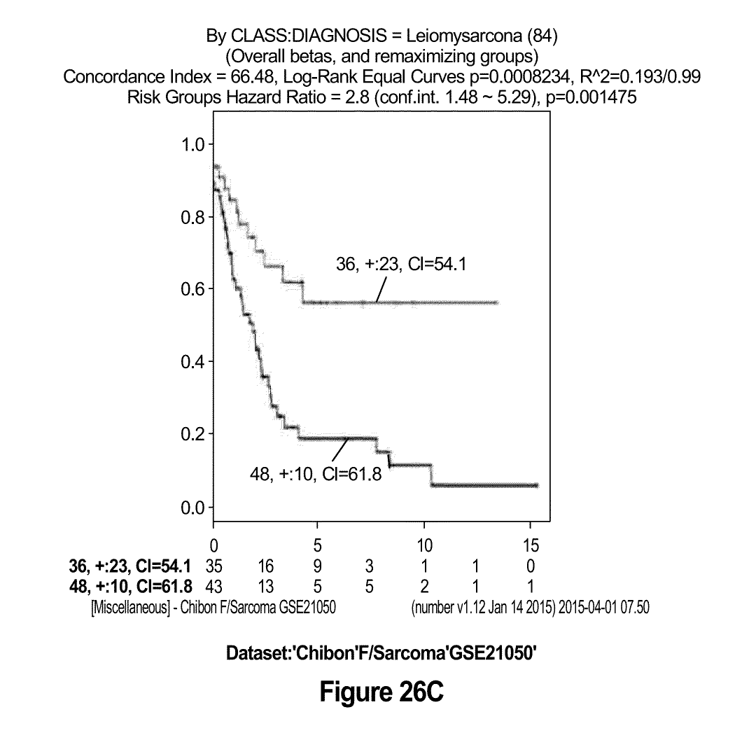

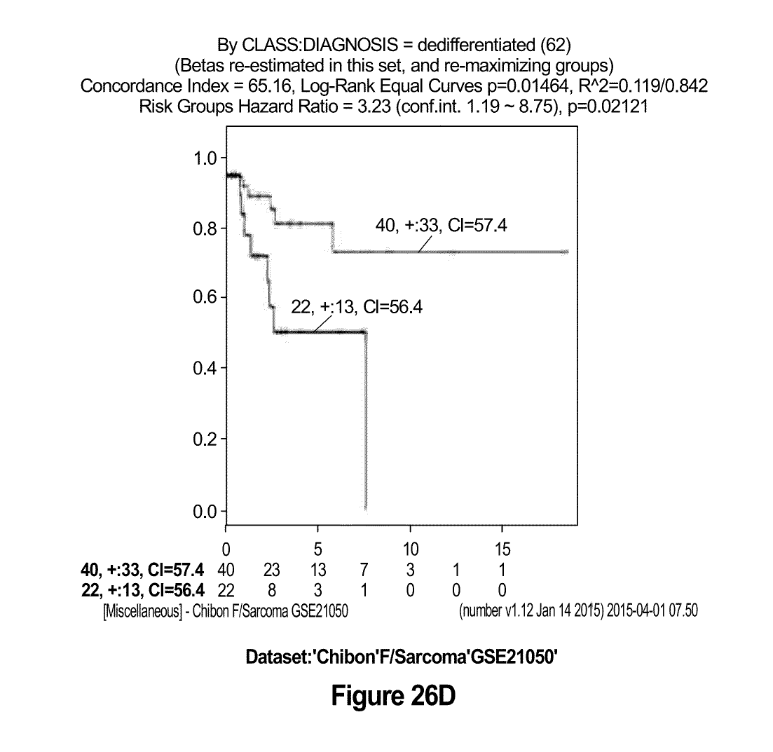

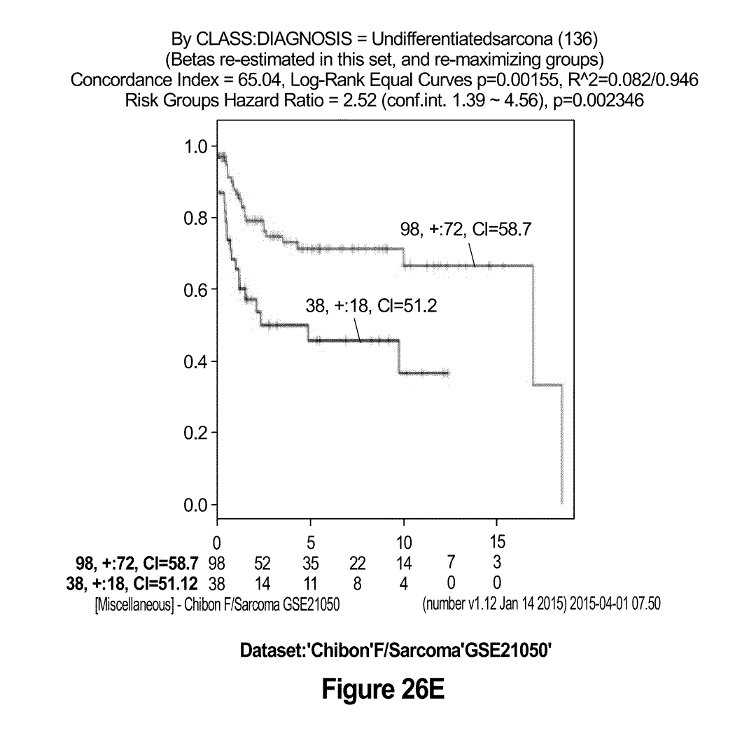

Our method and 6g-TAG assay could be used for classification and prognosis other (non-breast) cancers including (FIG. 24A to FIG. 26E). Survival prediction analysis was performed for multiple myeloma (GSE2658), kidney renal clear cell carcinoma (TCGA), sarcoma (GSE21050). This data analysis supports our results obtained for BC. In general, our finding strongly support the view that 6g-TAG signature could be used for development of high-uninformative quantitative indicator method of tumour aggressiveness, diagnostic and as the prognostic factor, which could be used in a regular clinical practice and clinical trials of many tumours.

### Example 50

### Discussion

Herein, we present the 6g-TAGs gene subset (module) as (i) the proliferative multi-gene low- and high-grades tumour classifier, (ii) early detection genetic signature of breast cancers and (iii) disease outcome predictor. This signature includes transcription factor E2F1 regulating other 5 periodic cell cycle genes of this structural and functional genetic module of the breast cancers and perhaps many other cancers.

### Example 51

### Discussion: 6g-TAGs Genes as Protein Inter-Connecting Network Hubs and Tumour-Related Functional Module of Chromosomal Aberrations, Mutations and Genomic Instability

Many gene signatures studied previously lack underlying functional mechanism attributing to breast cancer [46, 47]. In this current study, we represented 6g-TAGs genes as potential interacting network hubs with various components co-expressing in breast cancer datasets (FIG. 12A), validated further by qPCR (FIG. 12C). We explored 6g-TAGs genes as strong interacting network hubs playing critical role in G1/S, G2/M cell cycle phases in breast cancer. Indeed, gene ontology (GO) functions of 6g-TAGs genes and its interconnection network components implicate functional role in cell cycle progression, G1/S transition, and mitotic check points (Table EE2). Our co-localization studies on breast cancer cell line showed that PRR11, BRRN1 and MELK strongly co-localize (FIG. 13A(a-p)) and also interact as protein complexes (FIG. 13B(a-d)). These novel interactions observed suggest close interaction of the 6g-TAG genes between each other and many dozen other cell cycle genes and should be elucidated in details further in characterizing the functional role of the specific cell cycle/mitotic genes in breast cancers initiation, variation and progression. As we expected, an extensive literature mining has shown that overexpression of a significant number of the 6g-TAG interconnection network proteins and suppression of tumour suppresser-related genes is associated with abnormal G2-mitotic transition, mitosis phases, and post-mitotic events that lead to abnormal cell division, clonal diversity and consequently an increased rate of chromosomal aberrations, mutations and genomic instability.

### Example 52

### Discussion: 6g-TAGs Genes as Key Regulators at Various Cell Cycle Phases and as Proliferative Biomarkers in Aggressive Breast Cancer Cells

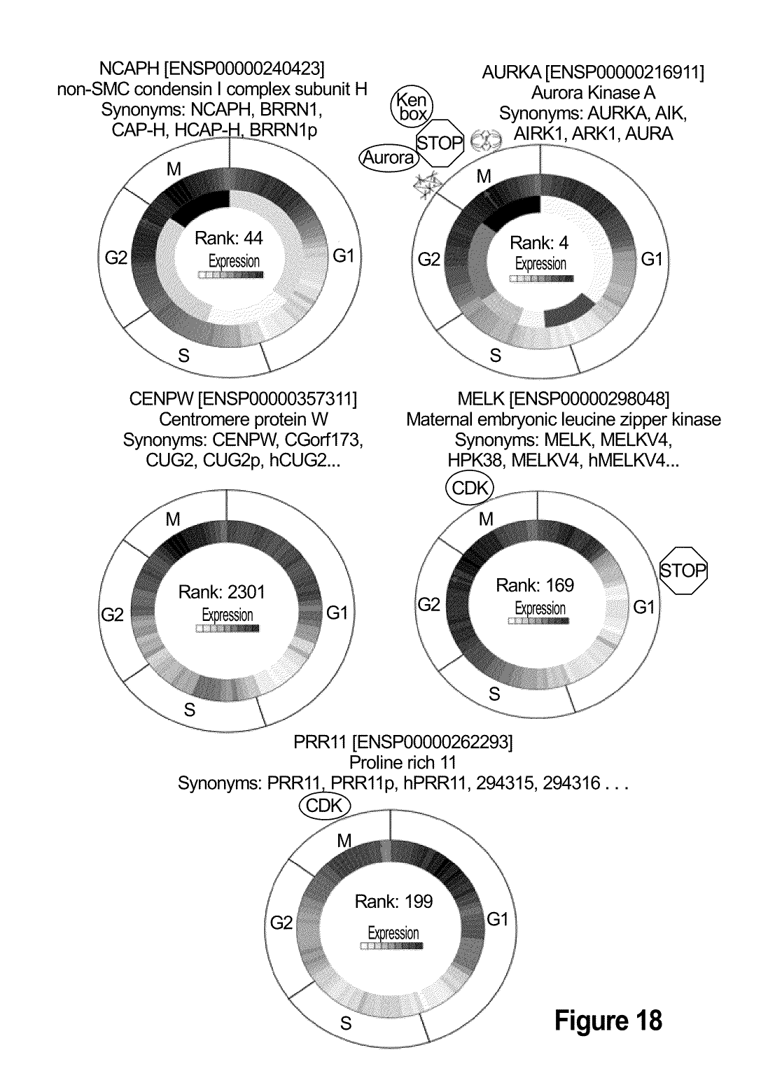

The predicted cell cycle regulatory role of 6g-TAGs genes was experimentally validated using RT-PCR studies on MDA-MB 436 cells sorted at various cell cycle (G1, S and G2/M) phases. AURKA-A, E2F1 showed high expression at G2/M check point as evident from its key role during mitotic chromosomal segregation [48]. E2F1 also showed high expression in G1/S [49-51]. BRRN1, CENPW are relatively higher in G2/M compared to other cell cycle phases (FIG. 14A). PRR11 expression is relatively higher in G2/M and in G1 cell cycle phases. This observation was corroborated further by conducting independent siRNA silencing experiments on all our TAGs genes to check at which phases of cell cycle the MDA-MB436 cells are arresting. Silencing of AURKA and CENPW showed cells arresting at G2/M transition and silencing of E2F1 showed cells arresting at S phase of cell cycle while independent silencing of BRRN1 and MELK showed cells arresting at G1 phase of cell cycle. However, PRR11 after silencing, showed 13.7% accumulation of cells in sub-G fraction, assuming tumour cells undergoing apoptosis (FIG. 14B, FIG. 18). This was further confirmed at flow cytometry studies using Annexin V apoptosis kit providing the assessment of the proportion of cells undergoing apoptosis after silencing of PRR11 in MDA-MB-436 cells (FIG. 18).

The 6g-TAGs genes functional role at cell cycle check points was further corroborated by CycleBase 3.0 web tool studied on Hela cancer cells. FIGS. 19A-19H show higher expression of AURKA and CENPW at G2/M check point, consistent with our RT-PCR and siRNA studies conducted on MDA-MB-436 breast cancer aggressive cells. Further NCAPH, MELK and PRR11 also showed higher levels of RNA expression at G1 and G2/M check points using cyclebase tool (FIGS. 19A-19H), which was supported further using our RT-PCR and siRNA studies conducted in breast cancer cell lines.

We further assessed the proliferation potential of 6g-TAGs genes by independently silencing 6g-TAGs genes in MDA-MB-436 cells at various time points (12, 24, 36, 48, 60 and 72 hrs). FIG. 12C shows 6g-TAGs genes inability to proliferate when relatively compared to control siRNA treated cells indicating 6g-TAGs genes capability to potentially induce proliferation in breast cancer (FIG. 14C).

### Example 53

### Discussion: Prognostic Significance of 6g-TAGs Genes in Breast Cancers

The results consisted of our previous finding (Ivshina et al, 2006; Kuznetsov et al, 2006) that the TAGs genes can be prognostic markers of breast cancer. FIGS. 15A(1)-15A(7) and 15B(1)-15B(7) clearly show 6g-TAGs genes as potential recurrence free survival biomarkers in Uppsala and BII-US breast cancer microarray cohorts. These observations are consistent with various other breast cancer microarray datasets analysed in microarray and qPCR study (FIGS. 15A(1)-15F; Table EE7).

### Example 54

### Discussion: Reproducibility of Prognostic Significance of the TAGs Gene Prediction Across Different Cohorts and within Tumour Subgroups of Breast Cancer Patients

FIG. 19A to FIG. 24B demonstrate that reproducibility of prognostic significance of 6g-TAGs gene prediction across different cohorts and within tumour subgroups of breast cancer patients. It includes multiple data sets which reproduce the low- and high-aggressive patterns of the tumours across different cohort and within very specific clinical and molecular sub-classes. These results were generated using Express Survival web application. In general these finding strongly support the view that our signature could be used even for phase I and II clinical trials in which usually the patients with high-aggressive tumours, higher grades, later stages and distant metastases are enrolled.

### Example 55

### Discussion: 6g-TAGs are Critical Regulators of Cancer Progression and could be Potential Targets for Cancer Treatment

PRR11

Our previous microarray studies strongly suggested that the products of poorly-annotated gene, PRR11 can be strictly associated cell cycle, breast cancer aggressiveness and patient' treatment outcome. Specifically FLJ11029 (detected by Affymetrix probsets 228273_at), RNA transcript of PRR11 gene could play important pro-oncogenic and prognostic role in BC (Ivshina et al, 2006; Kuznetsov et al, 2006). We have observed that the transcribed locus FLJ11029 was strongly expressed in BC and positively correlated with expression of other genes 232g-TAGs. These findings suggest that transcriptional regulation of FLJ11029 could be related to cells cycle/mitosis. Additionally, FIG. 14B and FIG. 18 show that PRR11 silencing experiments provide associations with apoptosis. Other studies have supported these findings [52-54]. In their studies, Zhou at al. [53] observed that over-expression of PRR11 associated with poor prognosis of breast cancer patients. They demonstrated a significance involvement of the PRR11 in the regulation of EMT pathway in breast cancer cells and its involvement in metastatic process [53]. It was shown, that PRR11 could regulate from late-S to G2/M phase progression and induces premature chromatin condensation, implicating in both cell cycle progression and lung cancer cells growth [52, 54]. Further structural, functional and clinical characterization of PRR1 and its products have to be carried out.

BRRN1/NCAPH/Condensin I

This gene encodes a member of the barr gene family and a regulatory subunit of the condensin complex. This complex is required for the conversion of interphase chromatin into condensed chromosomes. [55-58] BRRN1/NCAPH Condensin I defects could be associated with genome instability—the inherent feature of the most cancers and is the basis for selective killing of cancer cells by genotoxic therapeutics (Taxol, Vinblastine). Our current studies indicated that NCAPH interacts with PRR11 and further based on RT-PCR and siRNA silencing experiments it was shown that NCAPH could play critical regulatory role in cell cycle (G1/S phase) in breast cancer cells (FIGS. 14A-14B).

AURKA

This gene is one of the relatively well characterised members of our 6g-TAGs. AURKA protein is well known for its role in spindle assembly [59] and deregulation of this gene is known to have profound affect in chromosomal abnormalities in colorectal carcinoma progression [60]. In our current study it is shown to have critical role in breast cancer progression by regulating G2/M check point and further silencing of AURKA in breast cancer cell lines proved to be detrimental to cancer cells, indicating potential target for cancer therapy (FIG. 14B).

Further it was reported that genetic polymorphisms in AURKA and BRACA1 are associated with breast cancer susceptibility in Chinese Han population. [61]. It is a key regulator of chromosome segregation and cytokinesis and is currently undergoing clinical trials. Alisertib is an investigational, oral, selective inhibitor of AURKA used with several others specific Aurora A kinase inhibitors (e.g. MLN8237) and studied in clinical trials [62, 63]. These inhibitors could stop the growth of tumour cells by blocking some of the specific enzymes needed for cell proliferation and could be used starting from phase I and II of clinical trials as the common proliferative and tumour aggressiveness markers. Aurora A kinase inhibitors work in treating patients with high aggressive (triple-negative) tumours and/or at late stages/high-grade of BC and other cancers. Moreover, down regulation of AURKA can also reverse estrogen-mediated growth in breast cancer cells [84]. These findings also suggest that AURKA and their products could be used efficient therapeutic targets for different subtypes BCs (see above). Our 6-gene TAGs qPCR assay (including AURKA) should be useful in estimating the degree of clinical benefit based on objective clinical responses with AURKA inhibitor in breast and other cancer patients.

MELK

The maternal embryonic leucine zipper kinase (MELK) is the upregulated gene in high-grade prostate cancer [64], brain tumours [65], colorectal cancer [66], and also in breast cancer. [67, 68] MELK is part of our 6g-TAGs gene signature, which together or separately with its products could be used as early diagnostic, prognostic and periodic cell cycle marker, playing critical role in quantification of cell proliferation and tumour aggressiveness (FIGS. 8A(1)-8C, 14A-14C, 15A(1)-15F). In our current studies, we showed that MELK can interact with PRR11 and play important role in breast cancer diagnostics, prognosis and prediction. MELK is a normally non-essential kinase, but is critical for basal breast cancer and thus represents a promising selective therapeutic target for the most aggressive subtypes of breast cancer. Phase 1 Study of OTS167 in Patients with solid tumours. OTS167 is MELK inhibitor which demonstrated antitumour properties in laboratory tests. OTS167 has been being developed as anti-proliferative anti-cancer drug. In this first-in-human study OTS167 will be administered to patients with solid tumours which have not responded to treatment [69].

CENPW

CENPW is a centromere protein coding gene [70, 71]. It has been initially called C6orf173 orCUG2, cancer upregulated gene 2 [72-77] and was computationally selected as a part of our 6g-TAGs. In this work we showed its early diagnostic capacity and also proliferative capability and survival prognostic potential in breast cancer patients (FIGS. 8A(1)-8C, 14A-14C, 15A(1)-15F). Silencing of CENPW could alter proliferative capacity of MDA-MB-436 breast cancer cell line, indicating a potential target for cancer treatment in breast cancers.

### Example 56

### Discussion: Role of E2F1 in Coordination of 6g-TAGs Gene Expression in Breast Cancer Cells

It is well documented that Retinoblastoma protein (Rb, tumour suppressor gene) regulates cell cycle by forming protein complex with E2F1 [78, 79]. Based on previous studies, it was shown that loss of Rb leads to genomic instability and disruption of kinetochore complex with underlying mechanism unclear [49, 80]. In our current study, we showed that 6-g TAGs genes act as targets of E2F1 (FIG. 8C), with diverse functions include G1/S (PRR11), G2/M (BRRN1), kinetochore (CENPW), chromosomal segregation (AURKA) and chromosomal instability (MELK) (FIGS. 14A-C). Based on our current studies, we propose that E2F1 plays critical role in breast cancer by regulating various genes. Being targets of E2F1, TAGs genes with their diverse functions at various phases of cell cycle could play a role not only in breast cancer but may have impact in other cancer types. We present 6g-TAGs genes as comprehensive gene signature set having diagnostic, prognostic and predictive significance in breast cancer.

### Example 57

### Discussion: 6g-TAGs Genes as Genetic Grading System and Potential Early Diagnostic Markers in BC

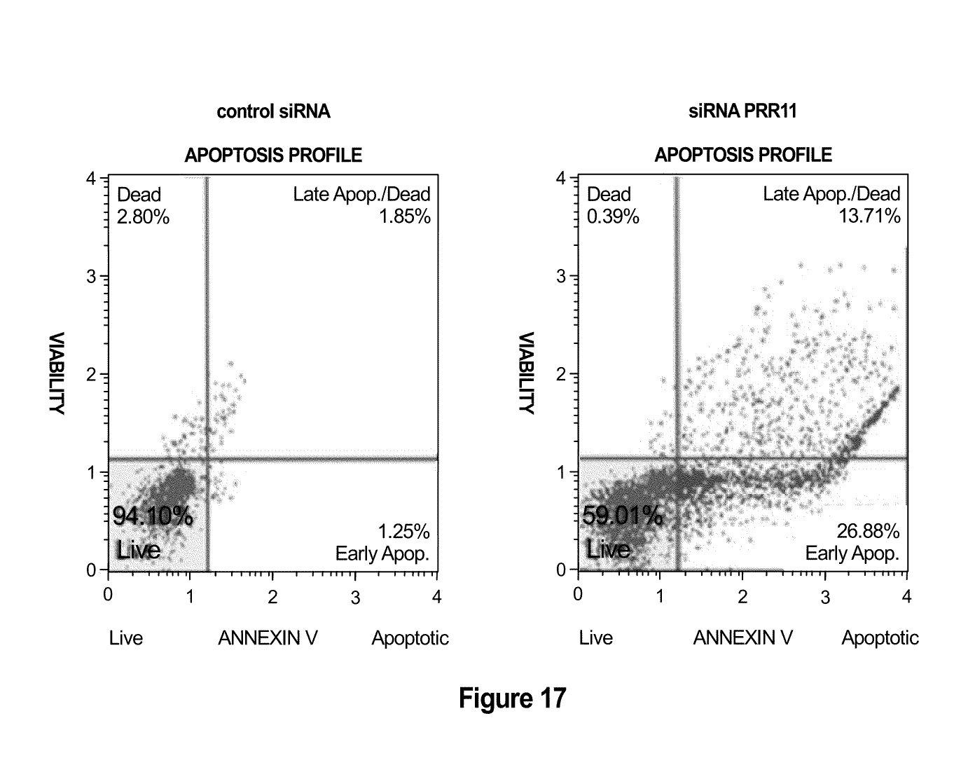

One of the major draw backs of various previously predicted biomarkers of breast cancer is lack of analysis at multi-cohort microarray datasets and the biomarkers predicted were not supported by experimental data. [81, 82]. To investigate 6g-TAGs genes grade signature potential, we analysed multi-cohort datasets (Singapore, Uppsala and Stockholm cohorts) and also in-house dataset (BII-US cohort) and further validated by breast cancer cell lines and by qPCR experiments (FIGS. 10A(1)-10C(7)). We further investigated at protein level the grade signature potential of 6g-TAGs genes (FIG. 10D) by relatively comparing MCF10A (G1 like) and MDA-MB-436 (G3 like) breast cancer cell lines. The 6g-TAGs genes show robust grade signature potential in breast cancer both at RNA and protein level. One of important features of 6g-TAGs is its ability to delineate histological grade 2 patients into HG1 like (low-grade) and HG3 like (high-grade) sub-classes [42]. The efficiency of G2 subclass in to GLG and to GHG is more than 95%, which is consistent in all diversified cohorts tested. This observation was validated by qPCR in BII-US cohort (FIGS. 11A(1)-11B(6)) and tested efficiently in other cohorts p<0.01 (FIG. 17). This subclass of G2 tumours will assist clinicians in effective treatment decision.

Further, we could show 6 TAGs genes as potential early diagnostic markers of cancer. FIGS. 8A(1)-8B(2) show clear discrimination between normal and breast tumour samples for all 6 TAGs genes in various stages of breast cancer. The robustness of 6-g TAGs as early diagnostic biomarkers was tested on two different datasets having matched pair dataset from TCGA and GSE10780 dataset. The modified Wilcoxon test statistics on the matched pair dataset strongly shows 6g-TAGs genes ability as early diagnostic markers (FIGS. 8A(1)-8B(2)). The 6g-TAGs was further tested successfully for prognostic potential in at least 3 cohorts (FIGS. 15A(1)-15B(7)). The disease free survival capability of 6g-TAGs genes in various microarray breast cancer cohort datasets was further validated using qPCR experiments (FIGS. 15C(1)-15C(7)). This observation was further supported with analysis at univariate and multivariate analysis, indicating 6g-TAGs as clinical factor with higher risk hazard ratio compared to all other clinical factors tested, in at least 4 different cohorts (Table EE3).

### Example 58

### Conclusions

This study provides a quantification of patho-biological and clinical significance of the six cell-cycle genes (BRRN1 (NM_015341), AURKA (NM_003600), MELK (NM_014791), PRR11 (NM_018304), CENPW (NM_001012507) and E2F1 (NM_005225)), representing the tumour aggressiveness grading (TAGs) signature (232 genes reported previously). We demonstrate that all of our TAGs genes are under regulation of E2F1 TF, these genes act as an inter-connecting gene network hubs, with regulatory role in G1/S, G2/M transition in BC. 6g-TAGs provides a dichotomization of proliferative capacity of the tumour cells in the low- and high-aggressive grades of BC with strong early cancer diagnostic, tumours classification, prognostic and therapeutic value. Each of these six genes can act as (i) a reproducible cell cycle-based clinical classifier of the low- and high-grade aggressive tumours and (ii) the early diagnostic multi-gene biomarker (iii) having the disease free survival and treatment outcome significances.

Based on finding we developed and validated a prototype of a qPCR-based method for early diagnostics, low- and high-aggressiveness grading classification and risk of recurrence prediction of BC. The method well reflects quantitatively the cancer cell cycle/mitosis rate, transcriptome over-expression and tumour aggressiveness of the different tumour types, subtypes and subclasses. Our TAG signature detection method could be implemented as uniform and objective prognostic factor, because it i) reflects and improves a measure of tumour aggressiveness previously based on clinical classification of tumours on low- and high-grade tumour classes and ii) it predicts outcome of BC patients without patient's preselection for assay conduction; our method could be apply for any cohorts regardless nuclear receptor status; tumour mass, tumour stages and subtypes. Therefore, we assume that our method could be useful on any phase of clinical trials and regular clinical practice for personalization of diagnosis and clinical outcome of many tumours, tumour' classes and subtypes.

Overall, our results could improve current clinical breast cancer classification (e.g. histologic grade, cancer recurrence risk assessment, management and counseling), and further provide a solution for the easily detection, outcome prognosis, and optimization of personalized medicine strategy of treating breast cancers in a clinical setting.

### REFERENCES FOR EXAMPLES 1 TO 24

1. Alizadeh, A. A., Eisen, M. B., Davis, R. E., Ma, C., Lossos, I. S., Rosenwald, A., Boldrick, J. C., Sabet, H., Tran, T., Yu, X., et al. 2000. Distinct types of diffuse large B-cell lymphoma identified by gene expression profiling. Nature 403:503-511.
2. Sorlie, T., Perou, C. M., Tibshirani, R., Aas, T., Geisler, S., Johnsen, H., Hastie, T., Eisen, M. B., van de Rijn, M., Jeffrey, S. S., et al. 2001. Gene expression patterns of breast carcinomas distinguish tumour subclasses with clinical implications. Proc Natl Acad Sci USA 98:10869-10874.
3. van't Veer, L. J., Dai, H., van de Vijver, M. J., He, Y. D., Hart, A. A., Mao, M., Peterse, H. L., van der Kooy, K., Marton, M. J., Witteveen, A. T., et al. 2002. Gene expression profiling predicts clinical outcome of breast cancer. Nature 415:530-536.
4. Bullinger, L., Dohner, K., Bair, E., Frohling, S., Schlenk, R. F., Tibshirani, R., Dohner, H., and Pollack, J. R. 2004. Use of gene-expression profiling to identify prognostic subclasses in adult acute myeloid leukemia. N Engl J Med 350:1605-1616.
5. Bloom, H. J., and Richardson, W. W. 1957. Histological grading and prognosis in breast cancer; a study of 1409 cases of which 359 have been followed for 15 years. Br J Cancer 11:359-377.
6. Elston, C. W., and Ellis, I. O. 1991. Pathological prognostic factors in breast cancer. I. The value of histological grade in breast cancer: experience from a large study with long-term follow-up. Histopathology 19:403-410.
7. Schumacher, M., Schmoor, C., Sauerbrei, W., Schauer, A., Ummenhofer, L., Gatzemeier, W., and Rauschecker, H. 1993. The prognostic effect of histological tumour grade in node-negative breast cancer patients. Breast Cancer Res Treat 25:235-245.
8. Roberti, N. E. 1997. The role of histologic grading in the prognosis of patients with carcinoma of the breast: is this a neglected opportunity? Cancer 80:1708-1716.
9. Lundin, J., Lundin, M., Holli, K., Kataja, V., Elomaa, L., Pylkkanen, L., Turpeenniemi-Huj anen, T., and Joensuu, H. 2001. Omission of histologic grading from clinical decision making may result in overuse of adjuvant therapies in breast cancer: results from a nationwide study. J Clin Oncol 19:28-36.
10. Harvey, J. M., de Klerk, N. H., and Sterrett, G. F. 1992. Histological grading in breast cancer: interobserver agreement, and relation to other prognostic factors including ploidy. Pathology 24:63-68.
11. Frierson, H. F., Jr., Wolber, R. A., Berean, K. W., Franquemont, D. W., Gaffey, M. J., Boyd, J. C., and Wilbur, D. C. 1995. Interobserver reproducibility of the Nottingham modification of the Bloom and Richardson histologic grading scheme for infiltrating ductal carcinoma. Am J Clin Pathol 103:195-198.
12. Robbins, P., Pinder, S., de Klerk, N., Dawkins, H., Harvey, J., Sterrett, G., Ellis, I., and Elston, C. 1995. Histological grading of breast carcinomas: a study of interobserver agreement. Hum Pathol 26:873-879.
13. Dalton, L. W., Pinder, S. E., Elston, C. E., Ellis, I. O., Page, D. L., Dupont, W. D., and Blamey, R. W. 2000. Histologic grading of breast cancer: linkage of patient outcome with level of pathologist agreement. Mod Pathol 13:730-735.
14. Younes, M., and Laucirica, R. 1997. Lack of prognostic significance of histological grade in node-negative invasive breast carcinoma. Clin Cancer Res 3:601-604.
15. Hayes, D. F., Isaacs, C., and Stearns, V. 2001. Prognostic factors in breast cancer: current and new predictors of metastasis. J Mammary Gland Biol Neoplasia 6:375-392.
16. Trudeau, M. E., Pritchard, K. I., Chapman, J. A., Hanna, W. M., Kahn, H. J., Murray, D., Sawka, C. A., Mobbs, B. G., Andrulis, I., McCready, D. R., et al. 2005. Prognostic factors affecting the natural history of node-negative breast cancer. Breast Cancer Res Treat 89:35-45.
17. Tibshirani, R., Hastie, T., Narasimhan, B., and Chu, G. 2002. Diagnosis of multiple cancer types by shrunken centroids of gene expression. Proc Natl Acad Sci USA 99:6567-6572.
18. Kuznetsov, V. A., Ivshina, A. V., Sen'ko, O. V., Kuznetsova, A. V. 1996. Syndrome approach for computer recognition of fuzzy systems and its application to immunological diagnostics and prognosis of human cancer. Math. Comput. Modeling 23:92-112.
19. Jackson, A. M., Ivshina, A. V., Senko, O., Kuznetsova, A., Sundan, A., O'Donnell, M. A., Clinton, S., Alexandroff, A. B., Selby, P. J., James, K., et al. 1998. Prognosis of intravesical bacillus Calmette-Guerin therapy for superficial bladder cancer by immunological urinary measurements: statistically weighted syndrome analysis. J Urol 159:1054-1063.
20. Lukas, J., Herzinger, T., Hansen, K., Moroni, M. C., Resnitzky, D., Helin, K., Reed, S. I., and Bartek, J. 1997. Cyclin E-induced S phase without activation of the pRb/E2F pathway. Genes Dev 11:1479-1492.
21. Spruck, C. H., Won, K. A., and Reed, S. I. 1999. Deregulated cyclin E induces chromosome instability. Nature 401:297-300.
22. Minella, A. C., Swanger, J., Bryant, E., Welcker, M., Hwang, H., and Clurman, B. E. 2002. p53 and p21 form an inducible barrier that protects cells against cyclin E-cdk2 deregulation. Curr Biol 12:1817-1827.
23. van Diest, P. J., van der Wall, E., and Baak, J. P. 2004. Prognostic value of proliferation in invasive breast cancer: a review. J Clin Pathol 57:675-681.
24. Haybittle, J. L., Blamey, R. W., Elston, C. W., Johnson, J., Doyle, P. J., Campbell, F. C., Nicholson, R. I., and Griffiths, K. 1982. A prognostic index in primary breast cancer. Br J Cancer 45:361-366.
25. Blarney, R. W. 1996. The design and clinical use of the Nottingham Prognostic Index in breast cancer. Breast 5:156-157.
26. Stotter, A. 1999. A prognostic table to guide practitioners advising patients on adjuvant systemic therapy in early breast cancer. Eur J Surg Oncol 25:341-343.
27. Feldman, M., Stanford, R., Catcheside, A., and Stotter, A. 2002. The use of a prognostic table to aid decision making on adjuvant therapy for women with early breast cancer. Eur J Surg Oncol 28:615-619.
28. Lacroix, M., Toillon, R. A., and Leclercq, G. 2004. Stable ‘portrait’ of breast tumours during progression: data from biology, pathology and genetics. Endocr Relat Cancer 11:497-522.
29. Ma, X. J., Salunga, R., Tuggle, J. T., Gaudet, J., Enright, E., McQuary, P., Payette, T., Pistone, M., Stecker, K., Zhang, B. M., et al. 2003. Gene expression profiles of human breast cancer progression. Proc Natl Acad Sci USA 100:5974-5979.
30. Lakhani, S. R. 1999. The transition from hyperplasia to invasive carcinoma of the breast. J Pathol 187:272-278.
31. Shackney, S. E., and Silverman, J. F. 2003. Molecular evolutionary patterns in breast cancer. Adv Anat Pathol 10:278-290.
32. Simpson, P. T., Reis-Filho, J. S., Gale, T., and Lakhani, S. R. 2005. Molecular evolution of breast cancer. J Pathol 205:248-254.
33. Tubiana, M., and Koscielny, S. 1991. Natural history of human breast cancer: recent data and clinical implications. Breast Cancer Res Treat 18:125-140.
34. Tabar, L., Fagerberg, G., Chen, H. H., Duffy, S. W., and Gad, A. 1996. Tumour development, histology and grade of breast cancers: prognosis and progression. Int J Cancer 66:413-419.
35. Millis, R. R., Barnes, D. M., Lampejo, O. T., Egan, M. K., and Smith, P. 1998. Tumour grade does not change between primary and recurrent mammary carcinoma. Eur J Cancer 34:548-553.
36. Roylance, R., Gorman, P., Harris, W., Liebmann, R., Barnes, D., Hanby, A., and Sheer, D. 1999. Comparative genomic hybridization of breast tumours stratified by histological grade reveals new insights into the biological progression of breast cancer. Cancer Res 59:1433-1436.
37. Buerger, H., Otterbach, F., Simon, R., Schafer, K. L., Poremba, C., Diallo, R., Brinkschmidt, C., Dockhorn-Dworniczak, B., and Boecker, W. 1999. Different genetic pathways in the evolution of invasive breast cancer are associated with distinct morphological subtypes. J Pathol 189:521-526.
38. Cserni, G. 2002. Tumour histological grade may progress between primary and recurrent invasive mammary carcinoma. J Clin Pathol 55:293-297.
39. Hitchcock, A., Ellis, I. O., Robertson, J. F., Gilmour, A., Bell, J., Elston, C. W., and Blamey, R. W. 1989. An observation of DNA ploidy, histological grade, and immunoreactivity for tumour-related antigens in primary and metastatic breast carcinoma. J Pathol 159:129-134.
40. Buerger, H., Mommers, E. C., Littmann, R., Simon, R., Diallo, R., Poremba, C., Dockhorn-Dworniczak, B., van Diest, P. J., and Boecker, W. 2001. Ductal invasive G2 and G3 carcinomas of the breast are the end stages of at least two different lines of genetic evolution. J Pathol 194:165-170.
41. Bergh, J., Norberg, T., Sjogren, S., Lindgren, A., and Holmberg, L. 1995. Complete sequencing of the p53 gene provides prognostic information in breast cancer patients, particularly in relation to adjuvant systemic therapy and radiotherapy. Nat Med 1:1029-1034.
42. Linderholm, B. K., Lindahl, T., Holmberg, L., Klaar, S., Lennerstrand, J., Henriksson, R., and Bergh, J. 2001. The expression of vascular endothelial growth factor correlates with mutant p53 and poor prognosis in human breast cancer. Cancer Res 61:2256-2260.
43. Lindahl, T., Landberg, G., Ahlgren, J., Nordgren, H., Norberg, T., Klaar, S., Holmberg, L., and Bergh, J. 2004. Overexpression of cyclin E protein is associated with specific mutation types in the p53 gene and poor survival in human breast cancer. Carcinogenesis 25:375-380.
44. Miller, L. D., Smeds, J., George, J., Vega, V. B., Vergara, L., Ploner, A., Pawitan, Y., Hall, P., Klaar, S., Liu, E. T., et al. 2005. An expression signature for p53 status in human breast cancer predicts mutation status, transcriptional effects, and patient survival. Proc Natl Acad Sci USA.
45. Kuznetsov, V. A., Knott, G. D., Ivshina, A. V. 1998. Artificial immune system based on syndromes-response approach: Theory and their application to recognition of the patterns of immune response and prognosis of therapy outcome. In Proc. of IEEE Intern. Conf. on Systems, Man, and Cybernetics. San Diego, Calif., USA. 3804-3809.
46. Mi, H., Lazareva-Ulitsky, B., Loo, R., Kejariwal, A., Vandergriff, J., Rabkin, S., Guo, N., Muruganuj an, A., Doremieux, O., Campbell, M. J., et al. 2005. The PANTHER database of protein families, subfamilies, functions and pathways. Nucleic Acids Res 33:D284-288.
47. 1996. Randomized trial of two versus five years of adjuvant tamoxifen for postmenopausal early stage breast cancer. Swedish Breast Cancer Cooperative Group. J Natl Cancer Inst 88:1543-1549.
Sotiriou, T., Perou, C. M., Tibshirani, R., Aas, T., Geisler, S., Johnsen, H., Hastie, T., Eisen, M. B., van de Rijn, M., Jeffrey, S. S., et al. 2001. Gene expression patterns of breast carcinomas distinguish tumour subclasses with clinical implications. Proc Natl Acad Sci USA 98:10869-10874.
van't Veer, L. J., Dai, H., van de Vijver, M. J., He, Y. D., Hart, A. A., Mao, M., Peterse, H. L., van der Kooy, K., Marton, M. J., Witteveen, A. T., et al. 2002. Gene expression profiling predicts clinical outcome of breast cancer. Nature 415:530-536.
Ma, X. J., Salunga, R., Tuggle, J. T., Gaudet, J., Enright, E., McQuary, P., Payette, T., Pistone, M., Stecker, K., Zhang, B. M., et al. 2003. Gene expression profiles of human breast cancer progression. Proc Natl Acad Sci USA 100:5974-5979.
Miller, L. D., Smeds, J., George, J., Vega, V. B., Vergara, L., Ploner, A., Pawitan, Y., Hall, P., Klaar, S., Liu, E. T., et al. 2005. An expression signature for p53 status in human breast cancer predicts mutation status, transcriptional effects, and patient survival. Proc Natl Acad Sci USA.
Kuznetsov, V. A., Ivshina, A. V., Sen'ko, O. V., Kuznetsova, A. V. 1996. Syndrome approach for computer recognition of fuzzy systems and its application to immunological diagnostics and prognosis of human cancer. Math. Comput. Modeling 23:92-112.
Kuznetsov, V. A., Knott, G. D., Ivshina, A. V. 1998. Artificial immune system based on syndromes-response approach: Theory and their application to recognition of the patterns of immune response and prognosis of therapy outcome. In Proc. of IEEE Intern. Conf. on Systems, Man, and Cybernetics. San Diego, Calif., USA. 3804-3809.
Jackson, A. M., Ivshina, A. V., Senko, O., Kuznetsova, A., Sundan, A., O'Donnell, M. A., Clinton, S., Alexandroff, A. B., Selby, P. J., James, K., Kuznetsov, V. A. 1998. Prognosis of intravesical bacillus Calmette-Guerin therapy for superficial bladder cancer by immunological urinary measurements: statistically weighted syndrome analysis. J Urol 159:1054-1063.
Mueller, B. U., Zeichner, S. L., Kuznetsov, V. A., Heath-Chiozzi, M., Pizzo P. A., and Dimitrov, D. S. Individual prognoses of long-term responses to antiretroviral treatment based on virological, immunological and pharmacological parameters measured during the first week under therapy. AIDS, 13, 1998, pp. f191-f196.
Tibshirani, R., Hastie, T., Narasimhan, B., and Chu, G. 2002. Diagnosis of multiple cancer types by shrunken centroids of gene expression. Proc Natl Acad Sci USA 99:6567-6572.
Haybittle J L, Blamey R W, Elston C W, Johnson J, Doyle P J, Campbell F C, Nicholson R I, Griffiths K. et al. A prognostic index in primary breast cancer. Br J Cancer 1982; 45 (3):361-6

### REFERENCES FOR EXAMPLE 25 TO EXAMPLE 58

1. Dupont V N, Gentien D, Oberkampf M, De Rycke Y, Blin N: A gene expression signature associated with metastatic cells in effusions of breast carcinoma patients. Int J Cancer 2007, 121:1036-1046.
2. Imyanitov E N, Hanson K P: Molecular pathogenesis of bilateral breast cancer. Cancer Lett 2003, 191:1-7.
3. Dressman H K, Hans C, Bild A, Olson J A, Rosen E, Marcom P K, Liotcheva V B, Jones E L, Vujaskovic Z, Marks J, et al: Gene expression profiles of multiple breast cancer phenotypes and response to neoadjuvant chemotherapy. Clin Cancer Res 2006, 12:819-826.
4. Nicolini A, Ferrari P, Cavazzana A, Carpi A, Berti P, Miccoli P: Conventional and new emerging prognostic factors in breast cancer: an update. Biomark Med 2007, 1:525-540.
5. Albain K S, Paik S, van't Veer L: Prediction of adjuvant chemotherapy benefit in endocrine responsive, early breast cancer using multigene assays. Breast 2009, 18 Suppl 3:S141-145.
6. Desmedt C, Ruiz-Garcia E, Andre F: Gene expression predictors in breast cancer: current status, limitations and perspectives. Eur J Cancer 2008, 44:2714-2720.
7. Dunn L, Demichele A: Genomic predictors of outcome and treatment response in breast cancer. Mol Diagn Ther 2009, 13:73-90.
8. Gokmen-Polar Y, Badve S: Molecular profiling assays in breast cancer: are we ready for prime time? Oncology (Williston Park) 2012, 26:350-357, 361.
9. Espinosa E, Gamez-Pozo A, Sanchez-Navarro I, Pinto A, Castaneda C A, Ciruelos E, Feliu J, Vara J A: The present and future of gene profiling in breast cancer. Cancer Metastasis Rev 2012, 31:41-46.
10. Espinosa E, Vara J A, Navarro I S, Gamez-Pozo A, Pinto A, Zamora P, Redondo A, Feliu J: Gene profiling in breast cancer: time to move forward. Cancer Treat Rev 2011, 37:416-421.
11. Sotiriou C, Piccart M J: Taking gene-expression profiling to the clinic: when will molecular signatures become relevant to patient care? Nat Rev Cancer 2007, 7:545-553.
12. Dinan M A, Mi X, Reed S D, Hirsch B R, Lyman G H, Curtis L H: Initial trends in the use of the 21-gene recurrence score assay for patients with breast cancer in the medicare population, 2005-2009. JAMA Oncology 2015, 1:158-166.
13. Ala U, Piro R M, Grassi E, Damasco C, Silengo L, Oti M, Provero P, Di Cunto F: Prediction of human disease genes by human-mouse conserved coexpression analysis. PLoS Comput Biol 2008, 4:e1000043.
14. Chen L, Xuan J, Riggins R B, Clarke R, Wang Y: Identifying cancer biomarkers by network-constrained support vector machines. BMC Syst Biol 2011, 5:161.
15. Djebbari A, Liu Z, Phan S, Famili F: An ensemble machine learning approach to predict survival in breast cancer. Int J Comput Biol Drug Des 2008, 1:275-294.
16. Gusev Y, Schmittgen T D, Lerner M, Postier R, Brackett D: Computational analysis of biological functions and pathways collectively targeted by co-expressed microRNAs in cancer. BMC Bioinformatics 2007, 8 Suppl 7:S16.
17. Gypas F, Bei E S, Zervakis M, Sfakianakis S: A disease annotation study of gene signatures in a breast cancer microarray dataset. Conf Proc IEEE Eng Med Biol Soc 2011, 2011:5551-5554.
18. Ivshina A V, George J, Senko O, Mow B, Putti T C, Smeds J, Lindahl T, Pawitan Y, Hall P, Nordgren H: Genetic reclassification of histologic grade delineates new clinical subtypes of breast cancer. Cancer research 2006, 66:10292-10301.
19. Hanahan D, Weinberg R A: The hallmarks of cancer. Cell 2000, 100:57-70.
20. Hanahan D, Weinberg R A: Hallmarks of cancer: the next generation. Cell 2011, 144:646-674.
21. Gevensleben H, Gohring U J, Buttner R, Heukamp L C, Kunz G, Dimpfl T, Jackisch C, Ortmann O, Albert U S, Bender R, et al: Comparison of MammaPrint and TargetPrint results with clinical parameters in German patients with early stage breast cancer. Int J Mol Med 2010, 26:837-843.
22. Gunven P, Randen M, Elmberger G, Bozoky B, Floore A: Gene expression profiling guiding diagnosis and therapy of rare mammary-like anogenital gland carcinomas. Med Oncol 2012, 29:127-132.
23. Nguyen B, Cusumano P G, Deck K, Kerlin D, Garcia A A, Barone J L, Rivera E, Yao K, de Snoo F A, van den Akker J, et al: Comparison of molecular subtyping with BluePrint, MammaPrint, and TargetPrint to local clinical subtyping in breast cancer patients. Ann Surg Oncol 2012, 19:3257-3263.
24. Roepman P, Horlings H M, Krijgsman O, Kok M, Bueno-de-Mesquita J M, Bender R, Linn S C, Glas A M, van de Vijver M J: Microarray-based determination of estrogen receptor, progesterone receptor, and HER2 receptor status in breast cancer. Clin Cancer Res 2009, 15:7003-7011.
25. Livak K J, Schmittgen T D: Analysis of relative gene expression data using real-time quantitative PCR and the 2(-Delta Delta C(T)) Method. Methods 2001, 25:402-408.
26. Schmittgen T D: Real-time quantitative PCR. Methods 2001, 25:383-385.
27. O'Connell J: The basics of R T-PCR. Some practical considerations. Methods Mol Biol 2002, 193:19-25.
28. Jensen E C: The basics of western blotting. Anat Rec (Hoboken) 2012, 295:369-371.
29. Blancher C, Jones A: SDS-PAGE and Western Blotting Techniques. Methods Mol Med 2001, 57:145-162.
30. Egger D, Bienz K: Protein (western) blotting. Mol Biotechnol 1994, 1:289-305.
31. Mattsson K Fau-Pokrovskaja K, Pokrovskaja K Fau-Kiss C, Kiss C Fau-Klein G, Klein G Fau-Szekely L, Szekely L: Proteins associated with the promyelocytic leukemia gene product (PML)-containing nuclear body move to the nucleolus upon inhibition of proteasome-dependent protein degradation.
32. Motakis E, Ivshina A V, Kuznetsov V A: Data-driven approach to predict survival of cancer patients: estimation of microarray genes' prediction significance by Cox proportional hazard regression model. IEEE Eng Med Biol Mag 2009, 28:58-66.
33. Tang Z, Ow G S, Thiery J P, Ivshina A V, Kuznetsov V A: Meta-analysis of transcriptome reveals let-7b as an unfavorable prognostic biomarker and predicts molecular and clinical subclasses in high-grade serous ovarian carcinoma. Int J Cancer 2013, . July 3. doi: 10.1002/ijc.28371.
34. Kuznetsov V A, Senko O V, Miller L D, Ivshina A V: Statistically Weighted Voting Analysis of Microarrays for Molecular Pattern Selection and Discovery Cancer Genotypes. IJCSNS Int J of Comput Sci Netw Secur 2006, 6:73-83.
35. Huang da W, Sherman B T, Lempicki R A: Bioinformatics enrichment tools: paths toward the comprehensive functional analysis of large gene lists. Nucleic Acids Res 2009, 37:1-13.
36. Huang da W, Sherman B T, Lempicki R A: Systematic and integrative analysis of large gene lists using DAVID bioinformatics resources. Nat Protoc 2009, 4:44-57.
37. Santos A, Wernersson R, Jensen L J: Cyclebase 3.0: a multi-organism database on cell-cycle regulation and phenotypes. Nucleic Acids Res 2015, 43:D1140-1144.
38. Gauthier N P, Jensen L J, Wernersson R, Brunak S, Jensen T S: Cyclebase.org: version 2.0, an updated comprehensive, multi-species repository of cell cycle experiments and derived analysis results. Nucleic Acids Res 2010, 38:D699-702.
39. Gauthier N P, Larsen M E, Wernersson R, de Lichtenberg U, Jensen L J, Brunak S, Jensen T S: Cyclebase.org—a comprehensive multi-organism online database of cell-cycle experiments. Nucleic Acids Res 2008, 36:D854-859.
40. Chen D T, Nasir A, Culhane A, Venkataramu C, Fulp W, Rubio R, Wang T, Agrawal D, McCarthy S M, Gruidl M, et al: Proliferative genes dominate malignancy-risk gene signature in histologically-normal breast tissue. Breast Cancer Res Treat 2010, 119:335-346.
41. Toh S H, Prathipati P, Motakis E, Kwoh C K, Yenamandra S P, Kuznetsov V A: A robust tool for discriminative analysis and feature selection in paired samples impacts the identification of the genes essential for reprogramming lung tissue to adenocarcinoma. BMC Genomics 2011, 12 Suppl 3:S24.
42. Ivshina A V, George J, Senko O, Mow B, Putti T C, Smeds J, Lindahl T, Pawitan Y, Hall P, Nordgren H, et al: Genetic reclassification of histologic grade delineates new clinical subtypes of breast cancer. Cancer Res 2006, 66:10292-10301.
43. Shi Z, Derow C K, Zhang B: Co-expression module analysis reveals biological processes, genomic gain, and regulatory mechanisms associated with breast cancer progression. BMC Syst Biol 2010, 4:74.
44. Tegge A N, Caldwell C W, Xu D: Pathway correlation profile of gene-gene co-expression for identifying pathway perturbation. PLoS One 2012, 7:e52127.
45. van den Akker E B, Verbruggen B, Heijmans B T, Beekman M, Kok J N, Slagboom P E, Reinders M J: Integrating protein-protein interaction networks with gene-gene co-expression networks improves gene signatures for classifying breast cancer metastasis. J Integr Bioinform 2011, 8:188.
46. Bertucci F, Finetti P, Cervera N, Maraninchi D, Viens P, Birnbaum D: Gene expression profiling and clinical outcome in breast cancer. OMICS 2006, 10:429-443.
47. Cheang M C, van de Rijn M, Nielsen T O: Gene expression profiling of breast cancer. Annu Rev Pathol 2008, 3:67-97.
48. Plotnikova O V, Nikonova A S, Loskutov Y V, Kozyulina P Y, Pugacheva E N, Golemis E A: Calmodulin activation of Aurora-A kinase (AURKA) is required during ciliary disassembly and in mitosis. Mol Biol Cell 2012, 23:2658-2670.
49. Amato A, Lentini L, Schillaci T, Iovino F, Di Leonardo A: RNAi mediated acute depletion of retinoblastoma protein (pRb) promotes aneuploidy in human primary cells via micronuclei formation. BMC Cell Biol 2009, 10:79.
50. Siddiqui H, Fox S R, Gunawardena R W, Knudsen E S: Loss of R B compromises specific heterochromatin modifications and modulates HP1alpha dynamics. J Cell Physiol 2007, 211:131-137.
51. Srinivasan S V, Mayhew C N, Schwemberger S, Zagorski W, Knudsen E S: R B loss promotes aberrant ploidy by deregulating levels and activity of DNA replication factors. J Biol Chem 2007, 282:23867-23877.
52. Zhang C, Zhang Y, Li Y, Zhu H, Wang Y, Cai W, Zhu J, Ozaki T, Bu Y: PRR11 regulates late-S to G2/M phase progression and induces premature chromatin condensation (PCC). Biochem Biophys Res Commun 2015, 458:501-508.
53. Zhou F, Liu H, Zhang X, Shen Y, Zheng D, Zhang A, Lai Y, Li H: Proline-rich protein 11 regulates epithelial-to-mesenchymal transition to promote breast cancer cell invasion. Int J Clin Exp Pathol 2014, 7:8692-8699.
54. Ji Y, Xie M, Lan H, Zhang Y, Long Y, Weng H, Li D, Cai W, Zhu H, Niu Y, et al: PRR11 is a novel gene implicated in cell cycle progression and lung cancer. The international journal of biochemistry & cell biology 2013, 45:645-656.
55. Cabello O A, Eliseeva E, He W G, Youssoufian H, Plon S E, Brinkley B R, Belmont J W: Cell cycle-dependent expression and nucleolar localization of hCAP-H. Mol Biol Cell 2001, 12:3527-3537.
56. Lai S K, Wong C H, Lee Y P, Li H Y: Caspase-3-mediated degradation of condensin Cap-H regulates mitotic cell death. Cell Death Differ 2011, 18:996-1004.
57. Heale J T, Ball A R, Jr., Schmiesing J A, Kim J S, Kong X, Zhou S, Hudson D F, Earnshaw W C, Yokomori K: Condensin I interacts with the PARP-1-XRCC1 complex and functions in DNA single-strand break repair. Mol Cell 2006, 21:837-848.
58. Tada K, Susumu H, Sakuno T, Watanabe Y: Condensin association with histone H2A shapes mitotic chromosomes. Nature 2011, 474:477-483.
59. Plotnikova O V, Nikonova A S, Loskutov Y V, Kozyulina P Y, Pugacheva E N, Golemis E A: Calmodulin activation of Aurora-A kinase (AURKA) is required during ciliary disassembly and in mitosis. Mol Biol Cell 2012, 23:2658-2670.
60. Sillars-Hardebol A H, Carvalho B, Tijssen M, Belien J A, de Wit M, Delis-van Diemen P M, Ponten F, van de Wiel M A, Fijneman R J, Meijer G A: TPX2 and AURKA promote 20q amplicon-driven colorectal adenoma to carcinoma progression. Gut 2012, 61:1568-1575.
61. Ruan Y, Song A P, Wang H, Xie Y T, Han J Y, Sajdik C, Tian X X, Fang W G: Genetic polymorphisms in AURKA and BRCA1 are associated with breast cancer susceptibility in a Chinese Han population. The Journal of pathology 2011, 225:535-543.
62. Caputo E, Miceli R, Motti M L, Tate R, Fratangelo F, Botti G, Mozzillo N, Carriero M V, Cavalcanti E, Palmieri G, et al: AurkA inhibitors enhance the effects of B-RAF and MEK inhibitors in melanoma treatment. J Transl Med 2015, 13:2069.
63. Sehdev V, Peng D, Soutto M, Washington M K, Revetta F, Ecsedy J, Zaika A, Rau T T, Schneider-Stock R, Belkhiri A, El-Rifai W: The aurora kinase A inhibitor MLN8237 enhances cisplatin-induced cell death in esophageal adenocarcinoma cells. Mol Cancer Ther 2012, 11:763-774.
64. Kuner R, Faith M, Pressinotti N C, Brase J C, Puig S B, Metzger J, Gade S, Schafer G, Bartsch G, Steiner E, et al: The maternal embryonic leucine zipper kinase (MELK) is upregulated in high-grade prostate cancer. J Mol Med 2013, 91:237-248.
65. Nakano I, Joshi K, Visnyei K, Hu B, Watanabe M, Lam D, Wexler E, Saigusa K, Nakamura Y, Laks D R, et al: Siomycin A targets brain tumour stem cells partially through a MELK-mediated pathway. Neuro Oncol 2011, 13:622-634.
66. Choi S, Ku J L: Resistance of colorectal cancer cells to radiation and 5-FU is associated with MELK expression. Biochem Biophys Res Commun 2011, 412:207-213.
67. Pickard M R, Green A R, Ellis T O, Caldas C, Hedge V L, Mourtada-Maarabouni M, Williams G T: Dysregulated expression of Fau and MELK is associated with poor prognosis in breast cancer. Breast cancer research: BCR 2009, 11:R60.
68. Lin M L, Park J H, Nishidate T, Nakamura Y, Katagiri T: Involvement of maternal embryonic leucine zipper kinase (MELK) in mammary carcinogenesis through interaction with Bcl-G, a pro-apoptotic member of the Bcl-2 family. Breast cancer research: BCR 2007, 9:R17.
69. Ganguly R, Mohyeldin A, Thiel J, Kornblum H I, Beullens M, Nakano I: MELK-a conserved kinase: functions, signaling, cancer, and controversy. Clinical and translational medicine 2015, 4:11.
70. Kaczmarczyk A, Sullivan K F: CENP-W plays a role in maintaining bipolar spindle structure. PLoS One 2014, 9:e106464.
71. Chun Y, Lee M, Park B, Lee S: CSN5/JAB1 interacts with the centromeric components CENP-T and CENP-W and regulates their proteasome-mediated degradation. The Journal of biological chemistry 2013, 288:27208-27219.
72. Malilas W, Koh S S, Lee S, Srisuttee R, Cho I R, Moon J, Kaowinn S, Johnston R N, Chung Y H: Suppression of autophagic genes sensitizes CUG2-overexpressing A549 human lung cancer cells to oncolytic vesicular stomatitis virus-induced apoptosis. Int J Oncol 2014, 44:1177-1184.
73. Kim H T, So J H, Jung S H, Ahn D G, Koh W, Kim N S, Kim S H, Lee S, Kim C H: Cug2 is essential for normal mitotic control and CNS development in zebrafish. BMC Dev Biol 2011, 11:49.
74. Lee S, Koh W, Kim H T, Kim C H: Cancer-upregulated gene 2 (CUG2) overexpression induces apoptosis in SKOV-3 cells. Cell Biochem Funct 2010, 28:461-468.
75. Park E H, Cho I R, Srisuttee R, Min H J, Oh M J, Jeong Y J, Jhun B H, Johnston R N, Lee S, Koh S S, Chung Y H: CUG2, a novel oncogene confers reoviral replication through Ras and p38 signaling pathway. Cancer Gene Ther 2010, 17:307-314.
76. Kim H, Lee M, Lee S, Park B, Koh W, Lee D J, Lim D S: Cancer-upregulated gene 2 (CUG2), a new component of centromere complex, is required for kinetochore function. Mol Cells 2009, 27:697-701.
77. Lee S, Gang J, Jeon S B, Choo S H, Lee B, Kim Y G, Lee Y S, Jung J, Song S Y, Koh S S: Molecular cloning and functional analysis of a novel oncogene, cancer-upregulated gene 2 (CUG2). Biochem Biophys Res Commun 2007, 360:633-639.
78. Dei Tos A P, Piccinin S, Doglioni C, Vukosavljevic T, Mentzel T, Boiocchi M, Fletcher C D: Molecular aberrations of the G1-S checkpoint in myxoid and round cell liposarcoma. Am J Pathol 1997, 151:1531-1539.
79. Lehman T A, Reddel R, Peiifer A M, Spillare E, Kaighn M E, Weston A, Gerwin B I, Harris C C: Oncogenes and tumour-suppressor genes. Environ Health Perspect 1991, 93:133-144.
80. Iovino F, Lentini L, Amato A, Di Leonardo A: R B acute loss induces centrosome amplification and aneuploidy in murine primary fibroblasts. Mol Cancer 2006, 5:38.
81. Yao J, Zhao Q, Yuan Y, Zhang L, Liu X, Yung W K, Weinstein J N: Identification of common prognostic gene expression signatures with biological meanings from microarray gene expression datasets. PLoS One 2012, 7:e45894.
82. Wu D, Smyth G K: Camera: a competitive gene set test accounting for inter-gene correlation. Nucleic Acids Res 2012, 40:e133.
83. Lee H H, Zhu Y, Govindasamy K M, Gopalan G. Downregulation of Aurora-A overrides estrogen-mediated growth and chemoresistance in breast cancer cells. Endocr Relat Cancer. 2008; 15(3):765-75
84. Loi S, Haibe-Kains B, Desmedt C, Lallemand F, Tutt A M, Gillet C, Ellis P, Harris A, Bergh J, Foekens J A, Klijn J G, Larsimont D, Buyse M, Bontempi G, Delorenzi M, Piccart M J, Sotiriou C. Definition of clinically distinct molecular subtypes in estrogen receptor-positive breast carcinomas through genomic grade. J Clin Oncol. 2007 Apr. 1; 25(10):1239-46. Erratum in: J Clin Oncol. 2007 Aug. 20; 25(24):3790.
85. Cheang M C, Martin M, Nielsen T O, Prat A, Voduc D, Rodriguez-Lescure A, Ruiz A, Chia S, Shepherd L, Ruiz-Borrego M, Calvo L, Alba E, Carrasco E, Caballero R, Tu D, Pritchard K I, Levine M N, Bramwell V H, Parker J, Bernard P S, Ellis M J, Perou C M, D1 Leo A, Carey L A. Defining breast cancer intrinsic subtypes by quantitative receptor expression. Oncologist. 2015 May; 20(5):474-82. doi:10.1634/theoncologist.2014-0372. Epub 2015 Apr. 23.

Further Aspects

Further aspects and embodiments of the invention are now set out in the following numbered Paragraphs; it is to be understood that the invention encompasses these aspects:

Paragraph 1. A method of assigning a grade to a breast tumour, which grade is indicative of the aggressiveness of the tumour, the method comprising detecting the expression of a gene selected from the genes set out in Table D1 (SWS Classifier 0).
Paragraph 2. A method according to Paragraph 1, in which the method comprises detecting a high level of expression of the gene and assigning the grade set out in Column 7 (“Grade with Higher Expression”) of Table D1 to the breast tumour or detecting a low level of expression of the gene and assigning the grade set out in Column 8 (“Grade with Lower Expression”) of Table D1 to the breast tumour.
Paragraph 3. A method according to Paragraph 1 or 2, in which a high level of expression is detected if the expression level of the gene is above the expression level set out in Column 9 (“Cut-Off”) of Table D1, and a low level of expression is detected if the expression level of the gene is below that level.
Paragraph 4. A method according to Paragraph 1, 2 or 3, in which the expression of a plurality of genes is detected, for example in the form of an expression profile of the plurality of genes.
Paragraph 5. A method according to any preceding Paragraph, in which the gene expression data or profile is derived from microarray hybridisation such as hybridisation to an Affymetrix microarray, or by real time polymerase chain reaction (RT-PCR).
Paragraph 6. A method according to any preceding Paragraph, in which the expression level of the gene or genes is detected using microarray analysis with a probe set consisting of a probe or probes having Affymetrix ID numbers as set out in Column 6 (“Affi ID”) of Table D1.
Paragraph 7. A method according to any preceding Paragraph, in which the method is capable of classifying a breast tumour to an accuracy of at least 85%, at least 90% accuracy, or at least 95% accuracy, with reference to the grade obtained of the breast tumour by histological grading.
Paragraph 8. A method according to any preceding Paragraph, in which the expression level of 5 or more genes is detected.
Paragraph 9. A method according to Paragraph 8, in which the 5 or more genes comprises the genes set out in Table D2 (SWS Classifier 1), viz: Barren homolog (Drosophila) (BRRN1, GenBank Accession No. D38553); Hypothetical protein F1111029 (F1111029, GenBank Accession No. BG165011); cDNA clone IMAGE:4452583, partial cds (GenBank Accession No. BG492359); Serine/threonine-protein kinase 6 (STK6); and Maternal embryonic leucine zipper kinase (MELK, GenBank Accession No. NM_014791).
Paragraph 10. A method according Paragraph 8 or 9, in which the expression level of the genes is detected using microarray analysis with a probe set consisting of probes having Affymetrix ID numbers as set out in Column 6 (“Affi ID”) of Table D2 (SWS Classifier 1), viz: B.228273_at, A.208079_s_at, B.226936_at, A.212949_at, A.204825_at, A.204092_s_at.
Paragraph 11. A method according to Paragraph 8, in which the 5 or more genes comprises the genes set out in Table D4 (SWS Classifier 3), viz: TPX2, microtubule-associated protein homolog (Xenopus laevis) (TPX2, GenBank Accession No. AF098158), Protein regulator of cytokinesis 1 (PRC1, GenBank Accession No. NM_003981), Neuro-oncological ventral antigen 1 (NOVA1, GenBank Accession No. NM_002515), Stanniocalcin 2 (STC2, GenBank Accession No. AI435828), Cold inducible RNA binding protein (CIRBP, GenBank Accession No. AL565767), Chemokine (C-X-C motif) ligand 14 (CXCL14, GenBank Accession No. NM_004887), Signal peptide, CUB domain, EGF-like 2 (SCUBE2, GenBank Accession No. AI424243).
Paragraph 12. A method according Paragraph 8 or 11, in which the expression level of the genes is detected using microarray analysis with a probe set consisting of probes having Affymetrix ID numbers as set out in Column 6 (“Affi ID”) of Table D4 (SWS Classifier 3), viz: A.210052_s_at, A.218009_s_at, A.205794_s_at, A.203438_at, B.225191_at, A.218002_s_at, A.219197_s_at.
Paragraph 13. A method according to Paragraph 8, in which the 5 or more genes comprises the genes set out in Table D5 (SWS Classifier 4), viz: cell division cycle associated 8 (CDCA8, GenBank Accession No. BC001651), centromere protein E, 312 kDa (CENPE, GenBank Accession No. NM_001813), steroid-5-alpha-reductase, alpha polypeptide 1 (3-oxo-5 alpha-steroid delta 4-dehydrogenase alpha 1) (SRD5A1, GenBank Accession No. BC006373), microtubule-associated protein tau (MAPT, GenBank Accession No. NM_016835), leucine zipper protein (FKSG14, GenBank Accession No. FKSG14), BC005400 (GenBank Accession No. R38110), EH-domain containing 2 (EHD2, GenBank Accession No. AI417917).
Paragraph 14. A method according Paragraph 8 or 13, in which the expression level of the genes is detected using microarray analysis with a probe set consisting of probes having Affymetrix ID numbers as set out in Column 6 (“Affi ID”) of Table D5 (SWS Classifier 4), viz: A.221520_s_at, A.205046_at, A.211056_s_at, A.203929_s_at, B.222848_at, B.240112_at, A.221870_at.
Paragraph 15. A method according to any of Paragraphs 1 to 7, in which the expression level of 17 or more genes in Table D1 is detected.
Paragraph 16. A method according to Paragraph 15, in which the 17 or more genes comprises the genes set out in Table D3 (SWS Classifier 2), viz: Barren homolog (Drosophila) (BRRN1, GenBank Accession No. D38553); Cell division cycle associated 8 (CDCA8, GenBank Accession No. BC001651); V-myb myeloblastosis viral oncogene homolog (avian)-like 2 (MYBL2, GenBank Accession No. NM_002466); Hypothetical protein F1111029 (F1111029, GenBank Accession No. BG165011); FBJ murine osteosarcoma viral oncogene homolog B (FOSB, GenBank Accession No. NM_006732); CDNA clone IMAGE:4452583, partial cds (GenBank Accession No. BG492359); Serine/threonine-protein kinase 6 (STK6, GenBank Accession No. BC027464); Anillin, actin binding protein (scraps homolog, Drosophila) (ANLN, GenBank Accession No. AK023208); Centromere protein E, 312 kDa (CENPE, GenBank Accession No. NM_001813); TTK protein kinase (TTK, GenBank Accession No. NM_003318); Signal peptide, CUB domain, EGF-like 2 (SCUBE2, GenBank Accession No. AI424243); V-fos FBJ murine osteosarcoma viral oncogene homolog (FOS, GenBank Accession No. BC004490); TPX2, microtubule-associated protein homolog (Xenopus laevis) (TPX2, GenBank Accession No. AF098158); Kinetochore protein Spc24 (Spc24, GenBank Accession No. AI469788); Forkhead box M1 (FOXM1, GenBank Accession No. NM_021953); Maternal embryonic leucine zipper kinase (MELK, GenBank Accession No. NM_014791); Cell division cycle associated 5 (CDCA5, GenBank Accession No. BE614410); and Cell division cycle associated 3 (CDCA3, GenBank Accession No. NM_031299).
Paragraph 17. A method according Paragraph 15 or 16, in which the expression level of the genes is detected using microarray analysis with a probe set consisting of probes having Affymetrix ID numbers as set out in Column 6 (“Affi ID”) of Table D3, viz: A.212949_at; A.221520_s_at; A.201710_at; B.228273_at; A.202768_at; B.226936_at; A.208079_s_at; B.222608_s_at; A.205046_at; A.204822_at; A.219197_s_at; A.209189_at; A.210052_s_at; B.235572_at; A.202580_x_at; A.204825_at; B.224753_at; and A.221436 s_at.
Paragraph 18. A method according to any of Paragraphs 8 to 17, in which the method comprises detecting a high level of expression of the gene, and assigning the grade set out in Column 7 (“Grade with Higher Expression”) of Table D2 (SWS Classifier 1), Table D3 (SWS Classifier 2), Table D4 (SWS Classifier 3) or Table D5 (SWS Classifier 4) to the breast tumour.
Paragraph 19. A method according to any of Paragraphs 8 to 17, in which the method comprises detecting a low level of expression of the gene, and assigning the grade set out in Column 8 (“Grade with Lower Expression”) of Table D2 (SWS Classifier 1), Table D3 (SWS Classifier 2), Table D4 (SWS Classifier 3) or Table D5 (SWS Classifier 4) to the breast tumour.
Paragraph 20. A method according to any of Paragraphs 8 to 18, in which a high level of expression is detected if the expression level of the gene is above the expression level set out in Column 9 (“Cut-Off”) of Table D2 (SWS Classifier 1), Table D3 (SWS Classifier 2), Table D4 (SWS Classifier 3) or Table D5 (SWS Classifier 4), and a low level of expression is detected if the expression level of the gene is below that level.
Paragraph 21. A method according to any preceding Paragraph, in which the expression level of all of the genes in Table D1 is detected.
Paragraph 22. A method according to any preceding Paragraph, in which the grade is assigned by applying a class prediction algorithm comprising a nearest shrunken centroid method (Tibshirani, et al., 2002, Proc Natl Acad Sci USA. 99(10): 6567-6572) to the expression data of the plurality of genes.
Paragraph 23. A method according to Paragraph 22, in which the class prediction algorithm comprises Prediction Analysis of Microarrays (PAM).
Paragraph 24. A method according to any preceding Paragraph, in which the grade is assigned by applying a class prediction algorithm comprising the steps of: (a) obtaining a set of predictor parameters; (b) re-coding the parameters to obtain discrete-valued variables; (c) selecting statistically robust discrete-valued variables and combinations thereof; (d) obtaining a sum of the statistically weighted discrete-valued variables and combinations thereof; and (e) obtaining a predictive outcome of breast cancer subtype based on the sum.
Paragraph 25. A method according to any preceding Paragraph, in which the grade is assigned by applying a class prediction algorithm comprising Statistically Weighted Syndromes (SWS) to the gene expression data.
Paragraph 26. A method according to any preceding Paragraph, in which the breast tumour comprises a histological Grade 2 breast tumour.
Paragraph 27. A method of classifying a histological Grade 2 tumour into a low aggressiveness tumour or a high aggressiveness tumour, the method comprising assigning a grade to the histological Grade 2 tumour according to any preceding Paragraph.
Paragraph 28. A method according to Paragraph 27, in which a histological Grade 2 breast tumour assigned a low aggressiveness grade has at least one feature of a histological Grade 1 breast tumour.
Paragraph 29. A method according to Paragraph 27, in which a breast tumour assigned a high aggressiveness grade has at least one feature of a histological Grade 3 breast tumour.
Paragraph 30. A method according to Paragraph 28 or 29, in which the feature comprises likelihood of tumour recurrence post-surgery or survival rate, such as disease free survival rate.
Paragraph 31. A method according to Paragraph 28 or 29, in which the feature comprises susceptibility to treatment.
Paragraph 32. A method according to any preceding Paragraph, in which the method is capable of classifying histological Grade 1 and histological Grade 3 tumours with an accuracy of 70% or above, 80% or above, or 90% or above.
Paragraph 33. A method of predicting a survival rate for an individual with a histological Grade 2 breast tumour, the method comprising assigning a grade to the breast tumour by a method according to any preceding Paragraph.
Paragraph 34. A method according to Paragraph 33, in which a low aggressiveness grade indicates a high probability of survival and a high aggressiveness grade indicates a low probability of survival.
Paragraph 35. A method of prognosis of an individual with a breast tumour, the method comprising assigning a grade to the breast tumour by a method according to any of Paragraphs 1 to 32.
Paragraph 36. A method of diagnosis of aggressive breast cancer in an individual, the method comprising assigning a grade indicative of high aggressiveness to a breast tumour of the individual by a method according to Paragraphs 1 to 32.
Paragraph 37. A method of choosing a therapy for an individual with breast cancer, the method comprising assigning a grade to the breast tumour by a method according to any of Paragraphs 1 to 32, and choosing an appropriate therapy based on the aggressiveness of the breast tumour.
Paragraph 38. A method of treatment of an individual with breast cancer, the method comprising assigning a grade to the breast tumour by a method according to any of Paragraphs 1 to 32, and administering an appropriate therapy to the individual based on the aggressiveness of the breast tumour.
Paragraph 39. A method according to Paragraph 36, 37 or 38, in which the diagnosis or choice of therapy is determined by further assessing the size of the tumour, or the lymph node stage or both, optionally together or in combination with other risk factors.
Paragraph 40. A method according to any of Paragraphs 36 to 39, in which the choice of therapy is determined by assessing the Nottingham Prognostic Index (Haybittle, et al., 1982).
Paragraph 41. A method according to any of Paragraphs 36 to 40, in which the choice of therapy is determined by further assessing the oestrogen receptor (ER) status of the breast tumour.
Paragraph 42. A method according to any preceding Paragraph, in which the histological grading comprises the Nottingham Grading System (NGS) or the Elston-Ellis Modified Scarff, Bloom, Richardson Grading System.
Paragraph 43. A method of determining the likelihood of success of a particular therapy on an individual with a breast tumour, the method comprising comparing the therapy with the therapy determined by a method according to Paragraph 37.
Paragraph 44. A method of assigning a breast tumour patient into a prognostic group, the method comprising applying the Nottingham Prognostic Index to a breast tumour, in which the histologic grade score of the breast tumour is replaced by a grade obtained by a method according to any of Paragraphs 2 to 32.
Paragraph 45. A method of assigning a breast tumour patient into a prognostic group, the method comprising deriving a score which is the sum of the following: (a) (0.2× tumour size in cm); (b) tumour grade in which the tumour grade is assigned by a method according to any of Paragraphs 2 to 32; and (c) lymph node stage; in which the tumour size and the lymph node stage are determined according to the Nottingham Prognostic Index, in which a patient with a score of 2.4 or less is categorised to a EPG (excellent prognostic group), a patient with a score of less than 3.4 is categorised to a GPG (good prognostic group), a patient with a score of between 3.4 and 5.4 is categorised to a MPG (moderate prognostic group), a patient with a score of greater than 5.4 is categorised to a PPG (poor prognostic group).
Paragraph 46. A method of determining whether a breast tumour is a metastatic breast tumour, the method comprising assigning a grade to the breast tumour by a method according to any of Paragraphs 1 to 32.
Paragraph 47. A method of identifying a molecule capable of treating or preventing breast cancer, the method comprising: (a) grading a breast tumour; (b) exposing the breast tumour to a candidate molecule; and (c) detecting a change in tumour grade; in which the grade or change thereof, or both, is assigned by a method according to any of Paragraphs 1 to 32.
Paragraph 48. A molecule identified by a method according to Paragraph 47.
Paragraph 49. Use of a molecule according to Paragraph 48 in a method of treatment or prevention of cancer in an individual.
Paragraph 50. A method of treatment or prevention of breast cancer in an individual, the method comprising modulating the expression of a gene set out in Table D1 (SWS Classifier 0).
Paragraph 51. A method of determining the proliferative state of a cell, the method comprising detecting the expression of a gene selected from the genes set out in Table D1 (SWS Classifier 0), in which: (a) a high level of expression of a gene which is annotated “3” in Column 7 (“Grade with Higher Expression”) indicates a highly proliferative cell; (b) a high level of expression of a gene which is annotated “1” in Column 7 (“Grade with Higher Expression”) indicates a non-proliferating cell or a slow-growing cell; (c) a low level of expression of a gene which is annotated “3” in Column 8 (“Grade with Lower Expression”) indicates a highly proliferative cell; and (d) a low level of expression of a gene which is annotated “1” in Column 8 (“Grade with Lower Expression”) indicates a non-proliferating cell or a slow-growing cell.
Paragraph 52. A method according to Paragraph 51, which comprises the features of any of Paragraphs 5 to 32.
Paragraph 53. A combination comprising the genes set out in Table D1 (SWS Classifier 0).
Paragraph 54. A combination comprising the probesets set out in Table D1 (SWS Classifier 0).
Paragraph 55. A combination comprising the genes set out in Paragraph 9, 11, 13 or 16.
Paragraph 56. A combination comprising the probesets set out in Paragraph 10, 12, 14 or 17.
Paragraph 57. A combination according to any of Paragraphs 53, 54, 55 or 56 in the form of an array.
Paragraph 58. A combination according to any of Paragraphs 53, 54, 55 or 56 in the form of a microarray.
Paragraph 59. A kit comprising a combination, array or microarray according to any of Paragraphs 53 to 58, together with instructions for use in a method according to any of Paragraphs 1 to 47 and 50 to 52.
Paragraph 60. Use of a combination, array or a microarray according to any of Paragraphs 53 to 58 or a kit according to Paragraph 59 in a method according to any of Paragraphs 1 to 47 and 50 to 52.
Paragraph 61. Use according to Paragraph 60, in which the method comprises a method of assigning a grade to a breast tumour according to any of Paragraphs 1 to 32.
Paragraph 62. A computer implemented method of assigning a grade to a breast tumour, the method comprising processing expression data for one or more genes set out in Table D1 (SWS Classifier 0) and obtaining a grade indicative of aggressiveness of the breast tumour.
Paragraph 63. A program storage device readable by a machine, tangibly embodying a program of instructions executable by the machine to perform method of assigning a grade to a breast tumour, the method comprising: processing expression data for one or more genes set out in Table D1 (SWS Classifier 0); and obtaining a grade indicative of aggressiveness of the breast tumour.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments and that many modifications and additions thereto may be made within the scope of the invention. Indeed, various modifications of the described modes for carrying out the invention which are obvious to those skilled in molecular biology or related fields are intended to be within the scope of the claims. Furthermore, various combinations of the features of the following dependent claims can be made with the features of the independent claims without departing from the scope of the present invention.

APPENDIX 1
SWS Classifier 0

| Or- | UGID(build | | Gene | Genbank | | Cut- | SWS: | Instability |
|---|---|---|---|---|---|---|---|---|
| der | #177) | UnigeneName | Symbol | Acc | Affi ID | off | Chi-2 | indices |
| 1 | Hs.528654 | Hypothetical protein FLJ11029 | FLJ11029 | BG165011 | B.228273_at | 7.7063 | 96.0 | 0.01139 |
| 2 | acc_NM_ | | | NM_003158 | A.208079_s_at | 6.6526 | 95.6 | 0.002087 |
| | 003158.1 | | | | | | | |
| 3 | Hs.308045 | Barren homolog (Drosophila) | BRRN1 | D38553 | A.212949_at | 5.9167 | 92.6 | 0.005697 |
| 4 | Hs.35962 | CDNA clone IMAGE: 4452583, partial cds | | BG492359 | B.226936_at | 7.5619 | 92.6 | 0.003179 |
| 5 | Hs.184339 | Maternal embryonic leucine zipper kinase | MELK | NM_014791 | A.204825_at | 7.1073 | 90.1 | 0.002296 |
| 6 | Hs.250822 | Serine/threonine kinase 6 | STK6 | NM_003600 | A.204092_s_at | 6.7266 | 88.6 | 0.003041 |
| 7 | Hs.9329 | TPX2, microtubule-associated protein | TPX2 | AF098158 | A.210052_s_at | 7.4051 | 86.2 | 0.000788 |
| | | homolog (Xenopus laevis) | | | | | | |
| 8 | Hs.1594 | Centromere protein A, 17 kDa | CENPA | NM_001809 | A.204962_s_at | 6.344 | 85.3 | 0.037328 |
| 9 | Hs.198363 | MCM10 minichromosome maintenance | MCM10 | AB042719 | B.222962_s_at | 6.1328 | 85.2 | 0.001132 |
| | | deficient 10 (S. cerevisiae) | | | | | | |
| 10 | Hs.48855 | Cell division cycle associated 8 | CDCA8 | BC001651 | A.221520_s_at | 5.2189 | 85.2 | 0.018247 |
| 11 | Hs.169840 | TTK protein kinase | TTK | NM_003318 | A.204822_at | 6.2397 | 82.2 | 0.017014 |
| 12 | Hs.69360 | Kinesin family member 2C | KIF2C | U63743 | A.209408_at | 7.3717 | 82.1 | 0.006487 |
| 13 | Hs.55028 | CDNA clone IMAGE: 6043059, partial cds | | BF111626 | B.228559_at | 7.2212 | 82.1 | 0.000785 |
| 14 | Hs.511941 | Forkhead box M1 | FOXM1 | NM_021953 | A.202580_x_at | 6.5827 | 81.9 | 0.001279 |
| 15 | Hs.3104 | Kinesin family member 14 | KIF14 | AW183154 | B.236641_at | 6.4175 | 81.9 | 0.02267 |
| 16 | Hs.179718 | V-myb myeloblastosis viral oncogene | MYBL2 | NM_002466 | A.201710_at | 6.0661 | 79.2 | 0.017019 |
| | | homolog (avian)-like 2 | | | | | | |
| 17 | Hs.93002 | Ubiquitin-conjugating enzyme E2C | UBE2C | NM_007019 | A.202954_at | 7.8431 | 79.2 | 0.06442 |
| 18 | Hs.344037 | Protein regulator of cytokinesis 1 | PRC1 | NM_003981 | A.218009_s_at | 7.3376 | 79.2 | 0.002774 |
| 19 | Hs.436187 | Thyroid hormone receptor interactor 13 | TRIP13 | NM_004237 | A.204033_at | 7.1768 | 79.0 | 0.090947 |
| 20 | Hs.408658 | Cyclin E2 | CCNE2 | NM_004702 | A.205034_at | 6.2055 | 78.6 | 0.018747 |
| 21 | Hs.30114 | Cell division cycle associated 3 | CDCA3 | BC002551 | B.223307_at | 7.8418 | 78.6 | 0.083659 |
| 22 | Hs.84113 | Cyclin-dependent kinase inhibitor 3 | CDKN3 | AF213033 | A.209714_s_at | 6.8414 | 78.6 | 0.005037 |
| | | (CDK2-associated dual specificity | | | | | | |
| | | phosphatase) | | | | | | |
| 23 | Hs.279766 | Kinesin family member 4A | KIF4A | NM_012310 | A.218355_at | 6.6174 | 78.2 | 0.013173 |
| 24 | Hs.104859 | Hypothetical protein DKFZp762E1312 | DKFZp762E | NM_018410 | A.218726_at | 6.3781 | 75.5 | 0.035806 |
| | | | 1312 | | | | | |
| 25 | Hs.444118 | MCM6 minichromosome maintenance | MCM6 | NM_005915 | A.201930_at | 7.9353 | 75.4 | 0.013732 |
| | | deficient 6 (MISS homolog, S. pombe) | | | | | | |
| | | (S. cerevisiae) | | | | | | |
| 26 | acc_NM_ | | | NM_018123 | A.219918_s_at | 6.5958 | 75.4 | 0.001536 |
| | 018123.1 | | | | | | | |
| 27 | Hs.287472 | BUB1 budding uninhibited by | BUB1 | AF043294 | A.209642_at | 6.0118 | 74.1 | 0.057721 |
| | | benzimidazoles 1 homolog (yeast) | | | | | | |
| 28 | Hs.36708 | BUB1 budding uninhibited by | BUB1B | NM_001211 | A.203755_at | 6.68 | 73.5 | 0.006753 |
| | | benzimidazoles 1 homolog beta (yeast) | | | | | | |
| 29 | Hs.77783 | Membrane-associated tyrosine- and | PKMYT1 | NM_004203 | A.204267_x_at | 6.9229 | 73.4 | 0.001777 |
| | | threonine-specific cdc2-inhibitory kinase | | | | | | |
| 30 | Hs.446554 | RAD51 homolog (RecA homolog, | RAD51 | NM_002875 | A.205024_s_at | 6.3524 | 73.4 | 0.016246 |
| | | E. coli) (S. cerevisiae) | | | | | | |
| 31 | Hs.82906 | CDC20 cell division cycle 20 homolog | CDC20 | NM_001255 | A.202870_s_at | 7.1291 | 73.0 | 0.108453 |
| | | (S. cerevisiae) | | | | | | |
| 32 | Hs.252712 | Karyopherin alpha 2 (RAG cohort 1, | KPNA2 | NM_002266 | A.201088_at | 8.4964 | 72.6 | 0.025069 |
| | | importin alpha 1) | | | | | | |
| 33 | Hs.3104 | | KIF14 | NM_014875 | A.206364_at | 6.1518 | 72.6 | 0.066755 |
| 34 | Hs.103305 | Chromobox homolog 2 (Pc class | | BE514414 | B.226473_at | 7.5588 | 72.6 | 0.013762 |
| | | homolog, Drosophila) | | | | | | |
| 35 | Hs.152759 | Activator of S phase kinase | ASK | NM_006716 | A.204244_s_at | 5.9825 | 72.3 | 0.018258 |
| 36 | acc_AL138828 | | | AL138828 | B.228069_at | 7.0119 | 72.3 | 0.084119 |
| 37 | Hs.226390 | Ribonucleotide reductase | RRM2 | NM_001034 | A.201890_at | 7.1014 | 71.0 | 0.00223 |
| | | M2 polypeptide | | | | | | |
| 38 | Hs.445890 | HSPC163 protein | HSPC163 | NM_014184 | A.218728_s_at | 7.6481 | 70.8 | 0.003156 |
| 39 | Hs.194698 | Cyclin B2 | CCNB2 | NM_004701 | A.202705_at | 7.0096 | 70.7 | 0.000753 |
| 40 | Hs.234545 | Cell division cycle associated 1 | CDCAI | AF326731 | B.223381_at | 6.4921 | 70.7 | 0.008259 |
| 41 | Hs.16244 | Sperm associated antigen 5 | SPAG5 | NM_006461 | A.203145_at | 6.4627 | 70.1 | 0.000806 |
| 42 | Hs.62180 | Anillin, actin binding protein (scraps | ANLN | AK023208 | B.222608_s_at | 6.9556 | 69.6 | 0.012886 |
| | | homolog, Drosophila) | | | | | | |
| 43 | Hs.14559 | Chromosome 10 open reading frame 3 | C10orf3 | NM_018131 | A.218542_at | 6.4965 | 69.3 | 0.048726 |
| 44 | Hs.122908 | DNA replication factor | CDT1 | AW075105 | B.228868_x_at | 7.0543 | 69.3 | 0.001059 |
| 45 | Hs.8878 | Kinesin family member 11 | KIF11 | NM_004523 | A.204444_at | 6.4655 | 69.3 | 0.005297 |
| 46 | Hs.83758 | CDC28 protein kinase regulatory | CKS2 | NM_001827 | A.204170_s_at | 7.8353 | 69.2 | 0.027378 |
| | | subunit 2 | | | | | | |
| 47 | Hs.112160 | Chromosome 15 open reading frame 20 | PIF1 | AF108138 | B.228252_at | 6.6518 | 69.2 | 0.038767 |
| 48 | Hs.79078 | MAD2 mitotic arrest deficient-like | MAD2L1 | NM_002358 | A.203362_s_at | 6.4606 | 68.0 | 0.038039 |
| | | 1 (yeast) | | | | | | |
| 49 | Hs.226390 | Ribonucleotide reductase | RRM2 | BC001886 | A.209773_s_at | 7.2979 | 67.4 | 0.135043 |
| | | M2 polypeptide | | | | | | |
| 50 | Hs.462306 | Ubiquitin-conjugating enzyme E2S | UBE2S | NM_014501 | A.202779_s_at | 6.9165 | 67.4 | 0.01343 |
| 51 | Hs.70704 | Chromosome 20 open reading | C20orf129 | BC001068 | B.225687_at | 7.2322 | 67.4 | 0.038884 |
| | | frame 129 | | | | | | |
| 52 | Hs.294088 | GAJ protein | GAJ | AY028916 | B.223700_at | 5.8432 | 67.3 | 0.00478 |
| 53 | Hs.381225 | Kinetochore protein Spc24 | Spc24 | AI469788 | B.235572_at | 6.7839 | 67.3 | 0.002404 |
| 54 | Hs.334562 | Cell division cycle 2, G1 to S and | CDC2 | AL524035 | A.203213_at | 7.0152 | 66.9 | 0.024298 |
| | | G2 to M | | | | | | |
| 55 | Hs.109706 | Hematological and neurological | HN1 | NM_016185 | A.217755_at | 7.9118 | 66.8 | 0.008041 |
| | | expressed 1 | | | | | | |
| 56 | Hs.23900 | Rac GTPase activating protein 1 | RACGAP1 | AU153848 | A.222077_s_at | 7.1207 | 66.5 | 0.042338 |
| 57 | Hs.77695 | Discs, large homolog 7 (Drosophila) | DLG7 | NM_014750 | A.203764_at | 6.3122 | 66.4 | 0.001011 |
| 58 | Hs.46423 | Histone 1, H4c | HIST1H4F | NM_ 003542 | A.205967_at | 8.3796 | 66.4 | 0.00462 |
| 59 | Hs.20830 | Kinesin family member C1 | KIFC1 | BC000712 | A.209680_s_at | 6.9746 | 66.4 | 0.041639 |
| 60 | Hs.339665 | Similar to Gastric cancer up-regulated-2 | AL135396 | B.225834_at | | 7.2467 | 66.4 | 0.019861 |
| 61 | Hs.94292 | FLJ23311 protein | FLJ23311 | NM_024680 | A.219990_at | 5.0277 | 66.3 | 0.006891 |
| 62 | Hs.73625 | Kinesin family member 20A | KIF20A | NM_005733 | A.218755_at | 7.2115 | 66.3 | 0.000671 |
| 63 | Hs.315167 | Defective in sister chromatid cohesion | MGC5528 | NM_024094 | A.219000_s_at | 6.2835 | 66.3 | 0.001518 |
| | | homolog 1 (S. cerevisiae) | | | | | | |
| 64 | Hs.85137 | Cyclin A2 | CCNA2 | NM_001237 | A.203418_at | 6.194 | 66.2 | 0.00117 |
| 65 | Hs.528669 | Chromosome condensation protein G | HCAP-G | NM_022346 | A.218662_s_at | 6.0594 | 66.2 | 0.01287 |
| 66 | Hs.75573 | Centromere protein E, 312 kDa | CENPE | NM_001813 | A.205046_at | 5.1972 | 65.5 | 0.002372 |
| 67 | acc_BE966146 | RAD51 associated protein 1 | BE966146 | A.204146_at | | 6.3049 | 65.3 | 0.006989 |
| 68 | Hs.334562 | Cell division cycle 2, G1 to S and | CDC2 | D88357 | A.210559_s_at | 7.0395 | 64.8 | 0.000887 |
| | | G2 to M | | | | | | |
| 69 | Hs.108106 | Ubiquitin-like, containing PHD and | UHRF1 | AK025578 | B.225655_at | 7.7335 | 64.8 | 0.024133 |
| | | RING finger domains, 1 | | | | | | |
| 70 | Hs.1578 | Baculoviral IAP repeat-containing 5 | BIRCS | NM_001168 | A.202095_s_at | 6.8907 | 64.6 | 0.090038 |
| | | (survivin) | | | | | | |
| 71 | acc_NM_ | | | NM_021067 | A.206102_at | 6.714 | 64.6 | 0.01255 |
| | 021067.1 | | | | | | | |
| 72 | Hs.244723 | Cyclin E1 | CCNE1 | AI671049 | A.213523_at | 6.082 | 64.6 | 0.000547 |
| 73 | Hs.198363 | MCM10 minichromosome maintenance | MCM10 | NM_018518 | A.220651_s_at | 5.6784 | 64.2 | 0.080997 |
| | | deficient 10 (S. cerevisiae) | | | | | | |
| 74 | Hs.155223 | Stanniocalcin 2 | STC2 | AI435828 | A.203438_at | 7.5388 | 64.0 | 0.011227 |
| 75 | Hs.25647 | V-fos FBJ murine osteosarcoma viral | FOS | BC004490 | A.209189_at | 8.9921 | 63.9 | 0.162153 |
| | | oncogene homolog | | | | | | |
| 76 | Hs.184601 | Solute carrier family 7 (cationic amino | SLC7A5 | AB018009 | A.201195_s_at | 7.4931 | 63.6 | 0.010677 |
| | | acid transporter, y+ system), member 5 | | | | | | |
| 77 | Hs.528669 | Chromosome condensation protein G | HCAP-G | NM_022346 | A.218663_at | 5.7831 | 63.6 | 0.0072 |
| 78 | Hs.30114 | Cell division cycle associated 3 | CDCA3 | NM_031299 | A.221436_s_at | 6.1898 | 63.6 | 0.001853 |
| 79 | Hs.296398 | Lysosomal associated protein | LAPTM4B | T15777 | A.214039_s_at | 9.3209 | 63.3 | 0.001249 |
| | | transmembrane 4 beta | | | | | | |
| 80 | Hs.442658 | Aurora kinase B | AURKB | AB011446 | A.209464_at | 5.9611 | 63.3 | 0.005453 |
| 81 | Hs.6879 | DC13 protein | DC13 | NM_020188 | A.218447_at | 7.436 | 63.3 | 0.027988 |
| 82 | Hs.78913 | Chemokine (C-X3-C motif) receptor 1 | CX3CR1 | U20350 | A.205898_at | 6.7764 | 63.2 | 0.014155 |
| 83 | Hs.406684 | Sodium channel, voltage-gated, | SCN7A | AI828648 | B.228504_at | 5.8248 | 63.2 | 0.003803 |
| | | type VII, alpha | | | | | | |
| 84 | Hs.80976 | Antigen identified by monoclonal | MKI67 | BF001806 | A.212022_s_at | 6.7255 | 62.4 | 0.124758 |
| | | antibody Ki-67 | | | | | | |
| 85 | Hs.406639 | Hypothetical protein LOC146909 | LOC146909 | AA292789 | A.222039_at | 6.4591 | 62.2 | 0.017876 |
| 86 | Hs.334562 | Cell division cycle 2, G1 to S and | CDC2 | NM_001786 | A.203214_x_at | 6.588 | 61.5 | 0.001897 |
| | | G2 to M | | | | | | |
| 87 | Hs.23960 | Cyclin B1 | CCNB1 | BE407516 | A.214710_s_at | 7.1555 | 60.8 | 0.01353 |
| 88 | Hs.445098 | DEP domain containing 1 | SDP35 | AK000490 | B.222958_s_at | 6.8747 | 60.8 | 0.003156 |
| 89 | Hs.58241 | Serine/threonine kinase 32B | HSA250839 | NM_018401 | A.219686_at | 4.5663 | 60.4 | 0.005019 |
| 90 | Hs.5199 | HSPC150 protein similar to ubiquitin- | HSPC150 | AB032931 | B.223229_at | 7.3947 | 60.4 | 0.010211 |
| | | conjugating enzyme | | | | | | |
| 91 | acc_T58044 | | | T58044 | B.227232_at | 8.5021 | 60.4 | 0.00327 |
| 92 | Hs.421337 | DEP domain containing 1B | XTP1 | AK001166 | B.226980_at | 5.4977 | 60.4 | 0.033734 |
| 93 | Hs.238205 | Chromosome 6 open reading frame 115 | C6orf115 | AF116682 | B.223361_at | 8.7555 | 60.1 | 0.003347 |
| 94 | Hs.27860 | Prostaglandin E receptor 3 | | AW242315 | A.213933_at | 7.3561 | 59.8 | 0.256699 |
| | | (subtype EP3) | | | | | | |
| 95 | Hs.292511 | Neuro-oncological ventral antigen 1 | NOVAI | NM_002515 | A.205794_s_at | 6.7682 | 59.5 | 0.010617 |
| 96 | Hs.276466 | Hypothetical protein FLJ21062 | FLJ21062 | NM_024788 | A.219455_at | 5.5257 | 59.3 | 0.003021 |
| 97 | Hs.270845 | Kinesin family member 23 | KIF23 | NM_004856 | A.204709_s_at | 5.1731 | 59.3 | 0.15391 |
| 98 | Hs.293257 | Epithelial cell transforming sequence 2 | ECT2 | NM_018098 | A.219787_s_at | 6.8052 | 59.3 | 0.000246 |
| | | oncogene | | | | | | |
| 99 | Hs.156346 | Topoisomerase (DNA) II alpha | TOP2A | NM_001067 | A.201292_at | 7.2468 | 59.1 | 0.011073 |
| | | 170 kDa | | | | | | |
| 100 | Hs.31297 | Cytochrome b reductase 1 | CYBRD1 | AL136693 | B.222453_at | 9.3991 | 59.1 | 0.001036 |
| 101 | Hs.414407 | Kinetochore associated 2 | KNTC2 | NM_006101 | A.204162_at | 6.017 | 58.7 | 0.076227 |
| 102 | Hs.445098 | DEP domain containing 1 | SDP35 | AI810054 | B.235545_at | 6.2495 | 58.7 | 0.133208 |
| 103 | Hs.301052 | Kinesin family member 18A | DKFZP434 | NM_031217 | A.221258_s_at | 5.3649 | 58.2 | 0.157731 |
| | | | G2226 | | | | | |
| 104 | Hs.431762 | Tetratricopeptide repeat domain 18 | LOC118491 | AW024437 | B.229170_s_at | 6.2298 | 58.2 | 0.065188 |
| 105 | Hs.24529 | CHK1 checkpoint homolog (S. pombe) | CHEK1 | NM_001274 | A.205394_at | 5.6217 | 58.1 | 0.016515 |
| 106 | Hs.87507 | BRCAI interacting protein C-terminal | BRIP1 | BF056791 | B.235609_at | 7.1489 | 58.1 | 0.010814 |
| | | helicase 1 | | | | | | |
| 107 | Hs.348920 | FSH primary response (LRPR1 | FSHPRH1 | BF793446 | A.214804_at | 5.0105 | 57.8 | 0.056646 |
| | | homolog, rat) 1 | | | | | | |
| 108 | Hs.127797 | CDNA FLJ11381 fis, clone | | AI807356 | B.227350_at | 6.8658 | 57.8 | 0.014086 |
| | | HEMBAI000501 | | | | | | |
| 109 | Hs.92458 | G protein-coupled receptor 19 | GPR19 | NM_006143 | A.207183_at | 5.2568 | 57.6 | 0.001708 |
| 110 | Hs.552 | Steroid-5-alpha-reductase, alpha | SRD5AI | BC006373 | A.211056_s_at | 6.7605 | 57.6 | 0.00075 |
| | | polypeptide 1 (3-oxo-5 alpha-steroid | | | | | | |
| | | delta 4-dehydrogenase alpha 1) | | | | | | |
| 111 | Hs.435733 | Cell division cycle associated 7 | CDCA7 | AY029179 | B.224428_s_at | 7.6746 | 57.6 | 0.020822 |
| 112 | Hs.101174 | Microtubule-associated protein tau | MAPT | NM_016835 | A.203929_s_at | 7.7914 | 57.6 | 0.003067 |
| 113 | Hs.436376 | Synaptotagmin binding, cytoplasmic | SYNCRIP | NM_006372 | A.217834_s_at | 6.8123 | 57.6 | 0.000586 |
| | | RNA interacting protein | | | | | | |
| 114 | Hs.122552 | G-2 and S-phase expressed 1 | GTSE1 | NM_016426 | A.204315_s_at | 6.4166 | 57.5 | 0.036289 |
| 115 | Hs.153704 | NIMA (never in mitosis gene a)-related | NEK2 | NM_002497 | A.204641_at | 7.0017 | 57.5 | 0.03551 |
| | | kinase 2 | | | | | | |
| 116 | Hs.208912 | Chromosome 22 open reading frame 18 | C22orf18 | NM_024053 | A.218741_at | 6.3488 | 56.8 | 0.006304 |
| 117 | Hs.81892 | KIAA0101 | KIAA0101 | NM_014736 | A.202503_s_at | 8.2054 | 56.6 | 0.029102 |
| 118 | Hs.279905 | Nucleolar and spindle associated | NUSAP1 | NM_016359 | A.218039_at | 7.542 | 56.6 | 0.005918 |
| | | protein 1 | | | | | | |
| 119 | Hs.170915 | Hypothetical protein FLJ10948 | FLJ10948 | NM_018281 | A.218552_at | 7.9778 | 56.0 | 0.00983 |
| 120 | Hs.144151 | Transcribed locus | | AI668620 | B.237339_at | 9.6693 | 56.0 | 0.028527 |
| 121 | Hs.433180 | DNA replication complex GINS protein | Pfs2 | BC003186 | A.221521_s_at | 6.3201 | 56.0 | 0.058903 |
| | | PSF2 | | | | | | |
| 122 | Hs.47504 | Exonuclease 1 | EXO1 | NM_003686 | A.204603_at | 5.927 | 56.0 | 0.001031 |
| 123 | Hs.293257 | Epithelial cell transforming sequence 2 | ECT2 | BG170335 | B.234992_x_at | 5.1653 | 55.6 | 0.001881 |
| | | oncogene | | | | | | |
| 124 | Hs.385913 | Acidic (leucine-rich) nuclear | ANP32E | NM_030920 | A.208103_s_at | 6.2989 | 55.6 | 0.001331 |
| | | phosphoprotein 32 family, member E | | | | | | |
| 125 | Hs.44380 | Transcribed locus, weakly similar to NP_060312.1 | | AA938184 | B.236312_at | 5.7016 | 55.6 | 0.007219 |
| | | hypothetical protein FLJ20489 [Homo sapiens] | | | | | | |
| 126 | Hs.19322 | Chromosome 9 open reading frame 140 | LOC89958 | AW250904 | B.225777_at | 7.8877 | 55.2 | 0.003266 |
| 127 | Hs.188173 | Lymphoid nuclear protein related to AF4 | | AA572675 | B.232286_at | 7.169 | 55.2 | 0.008402 |
| 128 | Hs.28264 | Chromosome 10 open reading frame 56 | FLJ90798 | AL049949 | A.212419_at | 7.6504 | 55.2 | 0.017182 |
| 129 | Hs.387057 | Hypothetical protein FLJ13710 | FLJ13710 | AK024132 | B.232944_at | 6.1947 | 55.2 | 0.03374 |
| 130 | acc_AL031658 | | | AL031658 | B.232357_at | 5.9761 | 54.9 | 0.032742 |
| 131 | Hs.286049 | Phosphoserine aminotransferase 1 | PSAT1 | BC004863 | B.223062_s_at | 6.1035 | 54.9 | 0.003426 |
| 132 | Hs.19173 | Nucleoporin 88kDa | | AI806781 | B.235786_at | 7.2856 | 54.9 | 0.036867 |
| 133 | Hs.155223 | Stanniocalcin 2 | STC2 | BC000658 | A.203439_s_at | 7.6806 | 54.8 | 0.039627 |
| 134 | acc_NM_ | | | NM_030896 | A.221275_s_at | 3.9611 | 54.8 | 0.001787 |
| | 030896.1 | | | | | | | |
| 135 | Hs.101174 | Microtubule-associated protein tau | MAPT | AAI99717 | B.225379_at | 7.8574 | 54.8 | 0.021421 |
| 136 | Hs.446680 | Retinoic acid induced 2 | RAI2 | NM_021785 | A.219440_at | 6.6594 | 54.3 | 0.057037 |
| 137 | Hs.431762 | Tetratricopeptide repeat domain 18 | LOC118491 | AW024437 | B.229169_at | 5.8266 | 53.6 | 0.002367 |
| 138 | acc_NM_ | | | NM_005196 | A.207828_s_at | 7.237 | 53.1 | 0.007336 |
| | 005196.1 | | | | | | | |
| 139 | acc_T90295 | Arsenic transactivated protein 1 | | T90295 | B.226661_at | 6.6825 | 52.8 | 0.001873 |
| 140 | Hs.42650 | ZW10 interactor | ZWINT | NM_007057 | A.204026_s_at | 7.5055 | 52.7 | 0.033812 |
| 141 | Hs.6641 | | KIF5C | NM_004522 | A.203130_s_at | 7.3214 | 52.7 | 0.012878 |
| 142 | Hs.23960 | Cyclin B1 | CCNB1 | N90191 | B.228729_at | 6.8018 | 52.6 | 0.031361 |
| 143 | Hs.72550 | Hyaluronan-mediated motility receptor | HMMR | NM_012485 | A.207165_at | 6.5885 | 52.4 | 0.065936 |
| | | (RHAMM) | | | | | | |
| 144 | Hs.73239 | Hypothetical protein FLJ10901 | FLJ10901 | NM_018265 | A.219010_at | 6.9429 | 52.3 | 0.020279 |
| 145 | Hs.163533 | V-erb-a erythroblastic leukemia viral | | AK024204 | B.233498_at | 7.5435 | 52.2 | 0.002319 |
| | | oncogene homolog 4 (avian) | | | | | | |
| 146 | Hs.109706 | Hematological and | HN1 | AF060925 | B.222396_at | 8.4225 | 52.2 | 0.000387 |
| | | neurological expressed 1 | | | | | | |
| 147 | Hs.165258 | Nuclear receptor subfamily 4, group A, | | AA523939 | B.235739_at | 7.1874 | 52.0 | 0.000444 |
| | | member 2 | | | | | | |
| 148 | Hs.20575 | Growth arrest-specific 2 like 3 | LOC283431 | H37811 | B.235709_at | 6.7278 | 51.9 | 0.009763 |
| 149 | Hs.75678 | FBJ murine osteosarcoma viral | FOSB | NM_006732 | A.202768_at | 6.1922 | 51.9 | 0.059132 |
| | | oncogene homolog B | | | | | | |
| 150 | Hs.437351 | Cold inducible RNA binding protein | CIRBP | AL565767 | B.225191_at | 8.033 | 51.9 | 0.00158 |
| 151 | Hs.57101 | MCM2 minichromosome maintenance | MCM2 | NM_004526 | A.202107_s_at | 7.861 | 51.7 | 0.27277 |
| | | deficient 2, mitotin (S. cerevisiae) | | | | | | |
| 152 | Hs.326736 | Ankyrin repeat domain 30A | NY-BR-1 | AF269087 | B.223864_at | 9.4144 | 51.3 | 0.042111 |
| 153 | Hs.298646 | ATPase family, AAA domain | PRO2000 | AI925583 | B.222740_at | 6.8416 | 50.8 | 0.130085 |
| | | containing 2 | | | | | | |
| 154 | Hs.119192 | H2A histone family, member Z | H2AFZ | NM_002106 | A.200853_at | 8.5896 | 50.1 | 0.007836 |
| 155 | Hs.119960 | PHD finger protein 19 | PHF19 | BE544837 | B.227211_at | 6.3487 | 50.1 | 0.084007 |
| 156 | Hs.78619 | Gamma-glutamyl hydrolase (conjugase, | GGH | NM_003878 | A.203560_at | 6.7708 | 49.9 | 0.006283 |
| | | folylpolygammaglutamyl hydrolase) | | | A.219555_s_at | 4.1739 | 49.9 | 0.13406 |
| 157 | Hs.283532 | Uncharacterized bone marrow protein | BM039 | NM_018455 | | | | |
| | | BM039 | | | | | | |
| 158 | Hs.221941 | Cytochrome b reductase 1 | | AI669804 | B.232459_at | 7.1171 | 49.9 | 0.01473 |
| 159 | Hs.104019 | Transforming, acidic coiled-coil | TACC3 | NM_006342 | A.218308_at | 6.1303 | 49.8 | 0.022905 |
| | | containing protein 3 | | | | | | |
| 160 | acc_ | | | AK002203 | B.226992_at | 7.9091 | 49.7 | 0.036845 |
| | AK002203.1 | | | | | | | |
| 161 | Hs.28625 | Transcribed locus | | AI693516 | B.228750_at | 7.1249 | 49.6 | 0.055282 |
| 162 | Hs.206868 | B-cell CLL/lymphoma 2 | | AU146384 | B.232210_at | 8.0948 | 49.6 | 0.002178 |
| 163 | Hs.75528 | Dynein, axonemal, light intermediate | HUMAUAN | AW299538 | B.227081_at | 7.0851 | 49.5 | 0.003326 |
| | | polypeptide 1 | TIG | | | | | |
| 164 | acc_AW271106 | | | AW271106 | B.229490_s_at | 6.2222 | 49.5 | 0.017341 |
| 165 | Hs.298646 | ATPase family, AAA domain | PRO2000 | AI139629 | B.235266_at | 6.1913 | 49.5 | 0.009434 |
| | | containing 2 | | | | | | |
| 166 | Hs.303090 | Protein phosphatase 1, regulatory | PPP1R3C | N26005 | A.204284_at | 7.0275 | 49.5 | 0.011239 |
| | | (inhibitor) subunit 3C | | | | | | |
| 167 | Hs.83169 | Matrix metalloproteinase 1 (interstitial | MMP1 | NM_002421 | A.204475_at | 7.1705 | 49.4 | 0.027959 |
| | | collagenase) | | | | | | |
| 168 | Hs.441708 | Leucine-rich repeat kinase 1 | MGC45866 | AI638593 | B.230021_at | 6.424 | 49.4 | 0.005067 |
| 169 | acc_AV733950 | | | AV733950 | A.201693_s_at | 7.9061 | 48.8 | 0.004773 |
| 170 | Hs.171695 | Dual specificity phosphatase 1 | DUSP1 | NM_004417 | A.201041_s_at | 9.7481 | 48.7 | 0.002971 |
| 171 | Hs.87491 | Thymidylate synthetase | TYMS | NM_001071 | A.202589_at | 7.8242 | 48.7 | 0.040774 |
| 172 | Hs.434886 | Cell division cycle associated 5 | CDCA5 | BE614410 | B.224753_at | 4.9821 | 48.5 | 0.106362 |
| 173 | Hs.24395 | Chemokine (C-X-C motif) ligand 14 | CXCL14 | NM_004887 | A.218002_s_at | 8.2513 | 48.2 | 0.002571 |
| 174 | Hs.104741 | T-LAK cell-originated protein kinase | TOPK | NM_018492 | A.219148_at | 6.4626 | 48.2 | 0.001405 |
| 175 | Hs.272027 | F-box protein 5 | FBXO5 | AK026197 | B.234863_x_at | 6.935 | 48.2 | 0.036746 |
| 176 | Hs.101174 | Microtubule-associated protein tau | MAPT | J03778 | A.206401_s_at | 6.4557 | 48.2 | 0.020545 |
| 177 | Hs.7888 | V-erb-a erythroblastic leukemia viral oncogene homolog | | AW772192 | A.214053_at | 7.0744 | 48.2 | 0.028848 |
| | | 4 (avian) | | | | | | |
| 178 | Hs.372254 | Lymphoid nuclear protein related to AF4 | | AI033582 | B.244696_at | 7.4158 | 48.2 | 0.001898 |
| 179 | Hs.435861 | Signal peptide, CUB domain, EGF-like 2 | SCUBE2 | AI424243 | A.219197_s_at | 8.3819 | 48.0 | 0.037351 |
| 180 | Hs.385998 | WD repeat and HMG-box DNA binding | WDHD1 | AK001538 | A.216228_s_at | 4.541 | 47.7 | 0.000561 |
| | | protein 1 | | | | | | |
| 181 | Hs.306322 | Neuron navigator 3 | NAV3 | NM_014903 | A.204823_at | 5.8235 | 47.7 | 0.003778 |
| 182 | Hs.21380 | CDNA F1136725 fis, clone | | AV709727 | B.225996_at | 7.5715 | 47.6 | 0.038219 |
| | | UTERU2012230 | | | | | | |
| 183 | Hs.89497 | Lamin B1 | LMNB1 | NM_005573 | A.203276_at | 7.11 | 47.3 | 0.003693 |
| 184 | acc_NM_ | | | NM_017669 | A.219650_at | 5.0422 | 47.3 | 0.003906 |
| | 017669.1 | | | | | | | |
| 185 | Hs.12532 | Chromosome 1 open reading frame 21 | C1orf21 | NM_030806 | A.221272_s_at | 5.6228 | 47.1 | 0.06632 |
| 186 | Hs.399966 | Calcium channel, voltage-dependent, L | CACNAID | BE550599 | A.210108_at | 6.2612 | 47.0 | 0.063467 |
| | | type, alpha 1D subunit | | | | | | |
| 187 | Hs.159264 | Clone 23948 mRNA sequence | | U79293 | A.215304_at | 6.9317 | 47.0 | 0.066157 |
| 188 | Hs.212787 | KIAA0303 protein | KIAA0303 | AW971134 | A.222348_at | 4.964 | 47.0 | 0.002269 |
| 189 | Hs.325650 | EH-domain containing 2 | EHD2 | AI417917 | A.221870_at | 6.4774 | 46.0 | 0.001916 |
| 190 | Hs.388347 | Hypothetical protein LOC143381 | | AW242720 | B.227550_at | 7.657 | 45.3 | 0.001238 |
| 191 | Hs.283853 | MRNA full length insert cDNA clone | | AL360204 | B.232855_at | 4.6288 | 45.3 | 0.00605 |
| | | EUROIMAGE 980547 | | | | | | |
| 192 | Hs.57301 | High mobility group AT-hook 1 | HMGAI | NM_002131 | A.206074_s_at | 7.6723 | 44.9 | 0.001416 |
| 193 | Hs.529285 | Solute carrier family 40 (iron-regulated | | AA588092 | B.239723_at | 6.9222 | 44.8 | 0.051707 |
| | | transporter), member 1 | | | | | | |
| 194 | Hs.252938 | Low density lipoprotein-related protein 2 | LRP2 | R73030 | B.230863_at | 7.4648 | 44.7 | 0.003167 |
| 195 | Hs.552 | Steroid-5-alpha-reductase, alpha | SRD5AI | NM_001047 | A.204675_at | 7.1002 | 44.7 | 0.000327 |
| | | polypeptide 1 (3-oxo-5 alpha-steroid | | | | | | |
| | | delta 4-dehydrogenase alpha 1) | | | | | | |
| 196 | Hs.156346 | Topoisomerase (DNA) II alpha 170 kDa | TOP2A | NM_001067 | A.201291_s_at | 7.3566 | 44.6 | 0.110228 |
| 197 | Hs.413924 | Chemokine (C-X-C motif) ligand 10 | CXCL10 | NM_001565 | A.204533_at | 7.9131 | 44.6 | 0.06956 |
| 198 | Hs.287466 | CDNA FLJ11928 fis, clone | | AK021990 | B.232699_at | 5.8675 | 44.6 | 0.001646 |
| | | HEMBB1000420 | | | | | | |
| 199 | acc_X07868 | | | X07868 | A.202409_at | 7.9917 | 44.5 | 0.001984 |
| 200 | Hs.101174 | Microtubule-associated protein tau | MAPT | NM_016835 | A.203928_x_at | 6.9103 | 44.5 | 0.005431 |
| 201 | Hs.334828 | Hypothetical protein FLJ10719 | FLJ10719 | BG478677 | A.213008_at | 6.4461 | 44.5 | 0.009488 |
| 202 | Hs.326035 | Early growth response 1 | EGR1 | NM_001964 | A.201694_s_at | 8.6202 | 44.2 | 0.024935 |
| 203 | Hs.122552 | G-2 and S-phase expressed 1 | GTSE1 | BF973178 | A.215942_s_at | 5.4688 | 44.2 | 0.041015 |
| 204 | Hs.24395 | Chemokine (C-X-C motif) ligand 14 | CXCL14 | AF144103 | B.222484_s_at | 9.3366 | 44.2 | 0.005525 |
| 205 | Hs.102406 | Melanophilin | | AI810764 | B.229150_at | 8.078 | 44.2 | 0.030939 |
| 206 | Hs.164018 | Leucine zipper protein FKSG14 | FKSG14 | BC005400 | B.222848_at | 6.6517 | 43.8 | 0.001146 |
| 207 | Hs.19114 | High-mobility group box 3 | HMGB3 | NM_005342 | A.203744_at | 7.5502 | 43.7 | 0.007416 |
| 208 | Hs.103982 | Chemokine (C-X-C motif) ligand 11 | CXCL11 | AF002985 | A.211122_s_at | 6.1001 | 43.0 | 0.003299 |
| 209 | Hs.356349 | Transcribed locus | ZNF145 | AI492388 | B.228854_at | 6.8198 | 43.0 | 0.001352 |
| 210 | Hs.1657 | Estrogen receptor 1 | ESR1 | NM_000125 | A.205225_at | 7.4943 | 43.0 | 0.188092 |
| 211 | Hs.144479 | Transcribed locus | | BF433570 | B.237301_at | 6.3171 | 42.8 | 0.003359 |
| 212 | acc_BF508074 | | | BF508074 | B.240465_at | 6.0041 | 42.7 | 0.001555 |
| 213 | Hs.326391 | Phytanoyl-CoA dioxygenase domain | PHYHD1 | AL545998 | B.226846_at | 7.2214 | 42.4 | 0.100092 |
| | | containing 1 | | | | | | |
| 214 | Hs.338851 | FLJ41238 protein | FLJ41238 | AW629527 | B.229764_at | 6.5319 | 42.3 | 0.032903 |
| 215 | Hs.65239 | Sodium channel, voltage-gated, type IV, | SCN4B | AW026241 | B.236359_at | 5.5526 | 42.1 | 0.106317 |
| | | beta | | | | | | |
| 216 | Hs.88417 | Sushi domain containing 3 | SUSD3 | AW966474 | B.227182_at | 8.195 | 41.8 | 0.015261 |
| 217 | Hs.16530 | Chemokine (C-C motif) ligand 18 | CCL18 | Y13710 | A.32128_at | 6.2442 | 41.3 | 0.003608 |
| | | (pulmonary and activation-regulated) | | | | | | |
| 218 | Hs.384944 | Superoxide dismutase 2, mitochondrial | SOD2 | X15132 | A.216841_s_at | 6.0027 | 41.3 | 0.115014 |
| 219 | Hs.406050 | Dynein, axonemal, light intermediate | DNALI1 | NM_003462 | A.205186_at | 4.2997 | 40.9 | 0.008737 |
| | | polypeptide 1 | | | | | | |
| 220 | Hs.458430 | N-acetyltransferase 1 (arylamine N- | NAT1 | NM_000662 | A.214440_at | 7.7423 | 40.8 | 0.001176 |
| | | acetyltransferase) | | | | | | |
| 221 | Hs.437023 | Nucleoporin 62 kDa | IL4I1 | AI859620 | B.230966_at | 6.4289 | 40.6 | 0.041224 |
| 222 | Hs.279905 | Nucleolar and spindle associated | NUSAP1 | NM_018454 | A.219978_s_at | 6.3357 | 40.1 | 0.011365 |
| | | protein 1 | | | | | | |
| 223 | Hs.505337 | Claudin 5 (transmembrane protein | CLDN5 | NM_003277 | A.204482_at | 6.1516 | 40.1 | 0.00138 |
| | | deleted in velocardiofacial syndrome) | | | | | | |
| 224 | Hs.44227 | Heparanase | HPSE | NM_00666 | A.219403_s_at | 5.2989 | 40.0 | 0.252507 |
| 225 | Hs.512555 | Collagen, type XIV, alpha 1 (undulin) | COL14AI | BF449063 | A.212865_s_at | 7.2876 | 40.0 | 0.00117 |
| 226 | Hs.511950 | Sirtuin (silent mating type information | SIRT3 | AF083108 | A.221562_s_at | 5.9645 | 40.0 | 0.018847 |
| | | regulation 2 homolog) 3 (S. cerevisiae) | | | | | | |
| 227 | Hs.371357 | RNA binding motif, single stranded | | AW338699 | B.241789_at | 6.3656 | 40.0 | 0.009148 |
| | | interacting protein | | | | | | |
| 228 | Hs.81131 | Guanidinoacetate N-methyltransferase | GAMT | NM_000156 | A.205354_at | 5.9474 | 39.9 | 0.005094 |
| 229 | Hs.158992 | FLJ45983 protein | | AI631850 | B.240192_at | 5.2898 | 39.9 | 0.344219 |
| 230 | Hs.104624 | Aquaporin 9 | AQP9 | NM_020980 | A.205568_at | 4.9519 | 39.8 | 0.010084 |
| 231 | Hs.437867 | Homo sapiens, clone IMAGE: 5759947, | | AW970881 | A.222314_x_at | 5.2505 | 39.8 | 0.042065 |
| | | mRNA | | | | | | |
| 232 | Hs.296049 | Microfibrillar-associated protein 4 | MFAP4 | R72286 | A.212713_at | 6.5149 | 39.7 | 0.001482 |
| 233 | Hs.109439 | Osteoglycin (osteoinductive factor, | OGN | NM_014057 | A.218730_s_at | 4.9325 | 39.7 | 0.014665 |
| | | mimecan) | | | | | | |
| 234 | Hs.29190 | Hypothetical protein MGC24047 | MGC24047 | AI732488 | B.229381_at | 7.2281 | 39.7 | 0.068574 |
| 235 | Hs.252418 | Elastin (supravalvular aortic stenosis, | ELN | AA479278 | A.212670_at | 6.8951 | 39.5 | 0.148698 |
| | | Williams-Beuren syndrome) | | | | | | |
| 236 | Hs.252938 | Low density lipoprotein-related protein 2 | LRP2 | NM_004525 | A.205710_at | 5.9845 | 39.2 | 0.003389 |
| 237 | Hs.32405 | MRNA; cDNA DKFZp586G0321 | | AL137566 | B.228554_at | 7.1124 | 38.6 | 0.014875 |
| | | (from clone DKFZp586G0321) | | | | | | |
| 238 | Hs.288720 | Leucine rich repeat containing 17 | LRRC17 | NM_005824_ | A.205381_at | 7.217 | 38.5 | 0.278881 |
| 239 | Hs.203963 | Helicase, lymphoid-specific | HELLS | NM_018063_ | A.220085_at | 5.2886 | 38.5 | 0.001189 |
| 240 | Hs.361171 | Placenta-specific 9 | PLAC9 | AW964972 | B.227419_x_at | 6.689 | 38.2 | 0.000231 |
| 241 | Hs.396595 | Flavin containing monooxygenase 5 | FMO5 | AK022172 | A.215300_s_at | 4.1433 | 37.5 | 0.00184 |
| 242 | Hs.105434 | Interferon stimulated gene 20 kDa | ISG20 | NM_002201 | A.204698_at | 6.2999 | 37.4 | 0.002793 |
| 243 | Hs.460184 | MCM4 minichromosome maintenance | MCM4 | X74794 | A.212141_at | 6.7292 | 36.6 | 0.175849 |
| | | deficient 4 (S. cerevisiae) | | | | | | |
| 244 | Hs.169266 | Neuropeptide Y receptor Y1 | NPY1R | NM_000909 | A.205440_s_at | 5.8305 | 36.0 | 0.011114 |
| 245 | acc_R38110 | | | R38110 | B.240112_at | 5.1631 | 35.4 | 0.020648 |
| 246 | Hs.63931 | Dachshund homolog 1 (Drosophila) | DACH | AI650353 | B.228915_at | 7.6716 | 35.3 | 0.318902 |
| 247 | Hs.102541 | Netrin 4 | NTN4 | AF278532 | B.223315_at | 8.2693 | 35.2 | 0.132405 |
| 248 | Hs.418367 | Neuromedin U | NMU | NM_006681 | A.206023_at | 5.1017 | 34.6 | 0.03508 |
| 249 | Hs.232127 | MRNA; cDNA DKFZp547P042 (from | | AL512727 | A.215014_at | 4.8334 | 34.6 | 0.035434 |
| | | clone DKFZp547P042) | | | | | | |
| 250 | Hs.212088 | Epoxide hydrolase 2, cytoplasmic | EPHX2 | AF233336 | A.209368_at | 6.4031 | 34.5 | 0.153812 |
| 251 | Hs.439760 | Cytochrome P450, family 4, subfamily | CYP4X1 | AA557324 | B.227702_at | 8.5972 | 34.5 | 0.015323 |
| | | X, polypeptide 1 | | | | | | |
| 252 | acc_BF513468 | | | BF513468 | B.241505_at | 7.1517 | 34.1 | 0.001404 |
| 253 | Hs.413078 | Nudix (nucleoside diphosphate linked | NUDT1 | NM_002452 | A.204766_s_at | 5.6705 | 34.0 | 0.069005 |
| | | moiety X)-type motif 1 | | | | | | |
| 254 | acc_AI492376 | | | AI492376 | B.231195_at | 5.1967 | 33.6 | 0.029021 |
| 255 | acc_AW512787 | | | AW512787 | B.238481_at | 8.5117 | 33.6 | 0.004714 |
| 256 | Hs.74369 | Integrin, alpha 7 | ITGA7 | AK022548 | A.216331_at | 5.1535 | 33.3 | 0.003271 |
| 257 | Hs.63931 | Dachshund homolog 1 (Drosophila) | DACH | NM_004392 | A.205472_s_at | 3.9246 | 33.2 | 0.001985 |
| 258 | Hs.225952 | Protein tyrosine phosphatase, receptor | PTPRT | NM_007050 | A.205948_at | 6.7634 | 32.2 | 0.190046 |
| | | type, T | | | | | | |
| 259 | acc_BF793701 | Musculoskeletal, embryonic | | BF793701 | B.226856_at | 5.5626 | 31.8 | 0.002068 |
| | | nuclear protein 1 | | | | | | |
| 260 | Hs.283417 | Transcribed locus | | AI826437 | B.229975_at | 6.381 | 31.3 | 0.008528 |
| 261 | Hs.21948 | Zinc finger protein 533 | | H15261 | B.243929_at | 4.7165 | 30.3 | 0.14416 |
| 262 | Hs.31297 | Cytochrome b reductase 1 | CYBRD1 | NM_024843 | A.217889_s_at | 5.6427 | 27.6 | 0.055739 |
| 263 | Hs.180142 | Calmodulin-like 5 | CALML5 | NM_017422 | A.220414_at | 5.994 | 27.4 | 0.008616 |
| 264 | Hs.176588 | Cytochrome P450, family 4, | CYP4Z1 | AV700083 | B.237395_at | 8.7505 | 24.4 | 0.399969 |
| | | subfamily Z, polypeptide 1 | | | | | | |

APPENDIX 1A
SWS Classifier 0 Accuracy G1 vs G3
Accuracy: G1 vs
G3
G1 = 63/68 (92.6%)
G3 = 51/55 (92.7%)

| | Patient | Histolgic | Probability | Probability | Predicted |
|---|---|---|---|---|---|
| Number | ID | grade | for G1 | for G3 | grade |
| 1 | X100B08 | 1 | 0.956 | 0.044 | 1 |
| 2 | X209C10 | 1 | 0.930 | 0.070 | 1 |
| 3 | X21C28 | 1 | 0.941 | 0.059 | 1 |
| 4 | X220C70 | 1 | 0.941 | 0.059 | 1 |
| 5 | X224C93 | 1 | 0.834 | 0.166 | 1 |
| 6 | X227C50 | 1 | 0.950 | 0.050 | 1 |
| 7 | X229C44 | 1 | 0.917 | 0.083 | 1 |
| 8 | X231C80 | 1 | 0.860 | 0.140 | 1 |
| 9 | X233C91 | 1 | 0.958 | 0.042 | 1 |
| 10 | X235C20 | 1 | 0.231 | 0.769 | 3 |
| 11 | X236C55 | 1 | 0.955 | 0.045 | 1 |
| 12 | X114B68 | 1 | 0.502 | 0.498 | 1 |
| 13 | X243C70 | 1 | 0.951 | 0.049 | 1 |
| 14 | X246C75 | 1 | 0.950 | 0.050 | 1 |
| 15 | X248C91 | 1 | 0.956 | 0.044 | 1 |
| 16 | X253C20 | 1 | 0.948 | 0.052 | 1 |
| 17 | X259C74 | 1 | 0.949 | 0.051 | 1 |
| 18 | X261C94 | 1 | 0.952 | 0.048 | 1 |
| 19 | X262C85 | 1 | 0.924 | 0.076 | 1 |
| 20 | X263C82 | 1 | 0.955 | 0.045 | 1 |
| 21 | X266C51 | 1 | 0.950 | 0.050 | 1 |
| 22 | X267C04 | 1 | 0.628 | 0.372 | 1 |
| 23 | X282C51 | 1 | 0.942 | 0.058 | 1 |
| 24 | X284C63 | 1 | 0.923 | 0.077 | 1 |
| 25 | X289C75 | 1 | 0.958 | 0.042 | 1 |
| 26 | X28C76 | 1 | 0.927 | 0.073 | 1 |
| 27 | X294C04 | 1 | 0.310 | 0.690 | 3 |
| 28 | X309C49 | 1 | 0.013 | 0.987 | 3 |
| 29 | X316C65 | 1 | 0.952 | 0.048 | 1 |
| 30 | X128B48 | 1 | 0.962 | 0.038 | 1 |
| 31 | X33C30 | 1 | 0.945 | 0.055 | 1 |
| 32 | X39C24 | 1 | 0.935 | 0.065 | 1 |
| 33 | X42C57 | 1 | 0.912 | 0.088 | 1 |
| 34 | X45A96 | 1 | 0.844 | 0.156 | 1 |
| 35 | X48A46 | 1 | 0.942 | 0.058 | 1 |
| 36 | X49A07 | 1 | 0.886 | 0.114 | 1 |
| 37 | X52A90 | 1 | 0.954 | 0.046 | 1 |
| 38 | X61A53 | 1 | 0.878 | 0.122 | 1 |
| 39 | X65A68 | 1 | 0.888 | 0.112 | 1 |
| 40 | X6B85 | 1 | 0.212 | 0.788 | 3 |
| 41 | X72A92 | 1 | 0.867 | 0.133 | 1 |
| 42 | X135B40 | 1 | 0.901 | 0.099 | 1 |
| 43 | X74A63 | 1 | 0.635 | 0.365 | 1 |
| 44 | X83A37 | 1 | 0.779 | 0.221 | 1 |
| 45 | X8B87 | 1 | 0.949 | 0.051 | 1 |
| 46 | X99A50 | 1 | 0.767 | 0.233 | 1 |
| 47 | X138B34 | 1 | 0.956 | 0.044 | 1 |
| 48 | X155B52 | 1 | 0.961 | 0.039 | 1 |
| 49 | X156B01 | 1 | 0.962 | 0.038 | 1 |
| 50 | X160B16 | 1 | 0.956 | 0.044 | 1 |
| 51 | X163B27 | 1 | 0.945 | 0.055 | 1 |
| 52 | X105B13 | 1 | 0.877 | 0.123 | 1 |
| 53 | X173B43 | 1 | 0.959 | 0.041 | 1 |
| 54 | X174B41 | 1 | 0.910 | 0.090 | 1 |
| 55 | X177B67 | 1 | 0.958 | 0.042 | 1 |
| 56 | X106B55 | 1 | 0.940 | 0.060 | 1 |
| 57 | X180B38 | 1 | 0.948 | 0.052 | 1 |
| 58 | X181B70 | 1 | 0.834 | 0.166 | 1 |
| 59 | X184B38 | 1 | 0.936 | 0.064 | 1 |
| 60 | X185B44 | 1 | 0.943 | 0.057 | 1 |
| 61 | X10B88 | 1 | 0.444 | 0.556 | 3 |
| 62 | X192B69 | 1 | 0.960 | 0.040 | 1 |
| 63 | X195B75 | 1 | 0.916 | 0.084 | 1 |
| 64 | X196B81 | 1 | 0.868 | 0.132 | 1 |
| 65 | X19C33 | 1 | 0.690 | 0.310 | 1 |
| 66 | X204B85 | 1 | 0.948 | 0.052 | 1 |
| 67 | X205B99 | 1 | 0.570 | 0.430 | 1 |
| 68 | X207C08 | 1 | 0.921 | 0.079 | 1 |
| 69 | X111B51 | 3 | 0.043 | 0.957 | 3 |
| 70 | X222C26 | 3 | 0.680 | 0.320 | 1 |
| 71 | X226C06 | 3 | 0.013 | 0.987 | 3 |
| 72 | X113B11 | 3 | 0.077 | 0.923 | 3 |
| 73 | X232C58 | 3 | 0.040 | 0.960 | 3 |
| 74 | X234C15 | 3 | 0.086 | 0.914 | 3 |
| 75 | X238C87 | 3 | 0.153 | 0.847 | 3 |
| 76 | X241C01 | 3 | 0.035 | 0.965 | 3 |
| 77 | X249C42 | 3 | 0.036 | 0.964 | 3 |
| 78 | X250C78 | 3 | 0.039 | 0.961 | 3 |
| 79 | X252C64 | 3 | 0.033 | 0.967 | 3 |
| 80 | X269C68 | 3 | 0.015 | 0.985 | 3 |
| 81 | X26C23 | 3 | 0.250 | 0.750 | 3 |
| 82 | X270C93 | 3 | 0.028 | 0.972 | 3 |
| 83 | X271C71 | 3 | 0.065 | 0.935 | 3 |
| 84 | X279C61 | 3 | 0.024 | 0.976 | 3 |
| 85 | X287C67 | 3 | 0.045 | 0.955 | 3 |
| 86 | X291C17 | 3 | 0.015 | 0.985 | 3 |
| 87 | X127B00 | 3 | 0.026 | 0.974 | 3 |
| 88 | X303C36 | 3 | 0.017 | 0.983 | 3 |
| 89 | X304C89 | 3 | 0.961 | 0.039 | 1 |
| 90 | X311A27 | 3 | 0.041 | 0.959 | 3 |
| 91 | X313A87 | 3 | 0.024 | 0.976 | 3 |
| 92 | X314B55 | 3 | 0.016 | 0.984 | 3 |
| 93 | X101B88 | 3 | 0.014 | 0.986 | 3 |
| 94 | X37C06 | 3 | 0.030 | 0.970 | 3 |
| 95 | X46A25 | 3 | 0.044 | 0.956 | 3 |
| 96 | X131B79 | 3 | 0.151 | 0.849 | 3 |
| 97 | X54A09 | 3 | 0.013 | 0.987 | 3 |
| 98 | X55A79 | 3 | 0.075 | 0.925 | 3 |
| 99 | X62A02 | 3 | 0.018 | 0.982 | 3 |
| 100 | X66A84 | 3 | 0.019 | 0.981 | 3 |
| 101 | X67A43 | 3 | 0.020 | 0.980 | 3 |
| 102 | X69A93 | 3 | 0.084 | 0.916 | 3 |
| 103 | X70A79 | 3 | 0.016 | 0.984 | 3 |
| 104 | X73A01 | 3 | 0.324 | 0.676 | 3 |
| 105 | X76A44 | 3 | 0.123 | 0.877 | 3 |
| 106 | X79A35 | 3 | 0.048 | 0.952 | 3 |
| 107 | X82A83 | 3 | 0.235 | 0.765 | 3 |
| 108 | X89A64 | 3 | 0.015 | 0.985 | 3 |
| 109 | X90A63 | 3 | 0.031 | 0.969 | 3 |
| 110 | X139B03 | 3 | 0.133 | 0.867 | 3 |
| 111 | X102B06 | 3 | 0.034 | 0.966 | 3 |
| 112 | X142B05 | 3 | 0.037 | 0.963 | 3 |
| 113 | X143B81 | 3 | 0.073 | 0.927 | 3 |
| 114 | X146B39 | 3 | 0.015 | 0.985 | 3 |
| 115 | X147B19 | 3 | 0.037 | 0.963 | 3 |
| 116 | X103B41 | 3 | 0.016 | 0.984 | 3 |
| 117 | X153B09 | 3 | 0.023 | 0.977 | 3 |
| 118 | X104B91 | 3 | 0.104 | 0.896 | 3 |
| 119 | X162B98 | 3 | 0.503 | 0.497 | 1 |
| 120 | X172B19 | 3 | 0.079 | 0.921 | 3 |
| 121 | X182B43 | 3 | 0.014 | 0.986 | 3 |
| 122 | X194B60 | 3 | 0.030 | 0.970 | 3 |
| 123 | X200B47 | 3 | 0.951 | 0.049 | 1 |

APPENDIX 2
SWS Classifier 0: Prediction of genetic G2a and G2b tumour
sub-types based on 264 gene classifier

| | Patient | Histologic | Probability | Probability | Predicted |
|---|---|---|---|---|---|
| Order | ID | grade | for G2a | for G2b | grade |
| 1 | X210C72 | 2 | 0.404 | 0.596 | 2b |
| 2 | X211C88 | 2 | 0.445 | 0.555 | 2b |
| 3 | X212C21 | 2 | 0.959 | 0.041 | 2a |
| 4 | X213C36 | 2 | 0.333 | 0.667 | 2b |
| 5 | X216C61 | 2 | 0.856 | 0.144 | 2a |
| 6 | X217C79 | 2 | 0.943 | 0.057 | 2a |
| 7 | X218C29 | 2 | 0.805 | 0.195 | 2a |
| 8 | X112B55 | 2 | 0.337 | 0.663 | 2b |
| 9 | X221C14 | 2 | 0.612 | 0.388 | 2a |
| 10 | X223C51 | 2 | 0.818 | 0.182 | 2a |
| 11 | X225C52 | 2 | 0.055 | 0.945 | 2b |
| 12 | X22C62 | 2 | 0.82 | 0.18 | 2a |
| 13 | X230C47 | 2 | 0.042 | 0.958 | 2b |
| 14 | X237C56 | 2 | 0.046 | 0.954 | 2b |
| 15 | X23C52 | 2 | 0.095 | 0.905 | 2b |
| 16 | X240C54 | 2 | 0.157 | 0.843 | 2b |
| 17 | X242C21 | 2 | 0.287 | 0.713 | 2b |
| 18 | X244C89 | 2 | 0.104 | 0.896 | 2b |
| 19 | X245C22 | 2 | 0.142 | 0.858 | 2b |
| 20 | X247C76 | 2 | 0.501 | 0.499 | 2a |
| 21 | X11B47 | 2 | 0.941 | 0.059 | 2a |
| 22 | X24C30 | 2 | 0.924 | 0.076 | 2a |
| 23 | X251C14 | 2 | 0.95 | 0.05 | 2a |
| 24 | X254C80 | 2 | 0.949 | 0.051 | 2a |
| 25 | X255C06 | 2 | 0.905 | 0.095 | 2a |
| 26 | X256C45 | 2 | 0.025 | 0.975 | 2b |
| 27 | X120B73 | 2 | 0.032 | 0.968 | 2b |
| 28 | X257C87 | 2 | 0.931 | 0.069 | 2a |
| 29 | X258C21 | 2 | 0.958 | 0.042 | 2a |
| 30 | X260C91 | 2 | 0.643 | 0.357 | 2a |
| 31 | X265C40 | 2 | 0.253 | 0.747 | 2b |
| 32 | X122B81 | 2 | 0.933 | 0.067 | 2a |
| 33 | X268C87 | 2 | 0.013 | 0.987 | 2b |
| 34 | X272C88 | 2 | 0.939 | 0.061 | 2a |
| 35 | X274C81 | 2 | 0.918 | 0.082 | 2a |
| 36 | X275C70 | 2 | 0.933 | 0.067 | 2a |
| 37 | X277C64 | 2 | 0.957 | 0.043 | 2a |
| 38 | X124B25 | 2 | 0.921 | 0.079 | 2a |
| 39 | X278C80 | 2 | 0.219 | 0.781 | 2b |
| 40 | X27C82 | 2 | 0.892 | 0.108 | 2a |
| 41 | X280C43 | 2 | 0.957 | 0.043 | 2a |
| 42 | X286C91 | 2 | 0.959 | 0.041 | 2a |
| 43 | X288C57 | 2 | 0.943 | 0.057 | 2a |
| 44 | X290C91 | 2 | 0.945 | 0.055 | 2a |
| 45 | X292C66 | 2 | 0.914 | 0.086 | 2a |
| 46 | X296C95 | 2 | 0.932 | 0.068 | 2a |
| 47 | X297C26 | 2 | 0.945 | 0.055 | 2a |
| 48 | X298C47 | 2 | 0.609 | 0.391 | 2a |
| 49 | X301C66 | 2 | 0.372 | 0.628 | 2b |
| 50 | X307C50 | 2 | 0.752 | 0.248 | 2a |
| 51 | X308C93 | 2 | 0.044 | 0.956 | 2b |
| 52 | X34C80 | 2 | 0.931 | 0.069 | 2a |
| 53 | X35C29 | 2 | 0.872 | 0.128 | 2a |
| 54 | X36C17 | 2 | 0.933 | 0.067 | 2a |
| 55 | X40C57 | 2 | 0.814 | 0.186 | 2a |
| 56 | X41B65 | 2 | 0.859 | 0.141 | 2a |
| 57 | X130B92 | 2 | 0.954 | 0.046 | 2a |
| 58 | X43C47 | 2 | 0.564 | 0.436 | 2a |
| 59 | X44A53 | 2 | 0.696 | 0.304 | 2a |
| 60 | X47A87 | 2 | 0.025 | 0.975 | 2b |
| 61 | X50A91 | 2 | 0.779 | 0.221 | 2a |
| 62 | X51A98 | 2 | 0.386 | 0.614 | 2b |
| 63 | X53A06 | 2 | 0.336 | 0.664 | 2b |
| 64 | X56A94 | 2 | 0.853 | 0.147 | 2a |
| 65 | X58A50 | 2 | 0.017 | 0.983 | 2b |
| 66 | X5B97 | 2 | 0.049 | 0.951 | 2b |
| 67 | X60A05 | 2 | 0.9 | 0.1 | 2a |
| 68 | X134B33 | 2 | 0.197 | 0.803 | 2b |
| 69 | X63A62 | 2 | 0.919 | 0.081 | 2a |
| 70 | X64A59 | 2 | 0.186 | 0.814 | 2b |
| 71 | X75A01 | 2 | 0.506 | 0.494 | 2a |
| 72 | X77A50 | 2 | 0.593 | 0.407 | 2a |
| 73 | X7B96 | 2 | 0.461 | 0.539 | 2b |
| 74 | X84A44 | 2 | 0.127 | 0.873 | 2b |
| 75 | X136B04 | 2 | 0.74 | 0.26 | 2a |
| 76 | X85A03 | 2 | 0.364 | 0.636 | 2b |
| 77 | X86A40 | 2 | 0.02 | 0.98 | 2b |
| 78 | X87A79 | 2 | 0.817 | 0.183 | 2a |
| 79 | X88A67 | 2 | 0.262 | 0.738 | 2b |
| 80 | X94A16 | 2 | 0.957 | 0.043 | 2a |
| 81 | X96A21 | 2 | 0.817 | 0.183 | 2a |
| 82 | X137B88 | 2 | 0.579 | 0.421 | 2a |
| 83 | X9B52 | 2 | 0.712 | 0.288 | 2a |
| 84 | X13B79 | 2 | 0.955 | 0.045 | 2a |
| 85 | X140B91 | 2 | 0.958 | 0.042 | 2a |
| 86 | X144B49 | 2 | 0.87 | 0.13 | 2a |
| 87 | X145B10 | 2 | 0.056 | 0.944 | 2b |
| 88 | X14B98 | 2 | 0.754 | 0.246 | 2a |
| 89 | X150B81 | 2 | 0.914 | 0.086 | 2a |
| 90 | X151B84 | 2 | 0.926 | 0.074 | 2a |
| 91 | X152B99 | 2 | 0.934 | 0.066 | 2a |
| 92 | X154B42 | 2 | 0.07 | 0.93 | 2b |
| 93 | X158B84 | 2 | 0.922 | 0.078 | 2a |
| 94 | X159B47 | 2 | 0.14 | 0.86 | 2b |
| 95 | X15C94 | 2 | 0.944 | 0.056 | 2a |
| 96 | X161B31 | 2 | 0.949 | 0.051 | 2a |
| 97 | X164B81 | 2 | 0.024 | 0.976 | 2b |
| 98 | X165B72 | 2 | 0.384 | 0.616 | 2b |
| 99 | X166B79 | 2 | 0.399 | 0.601 | 2b |
| 100 | X168B51 | 2 | 0.889 | 0.111 | 2a |
| 101 | X169B79 | 2 | 0.751 | 0.249 | 2a |
| 102 | X16C97 | 2 | 0.946 | 0.054 | 2a |
| 103 | X170B15 | 2 | 0.867 | 0.133 | 2a |
| 104 | X171B77 | 2 | 0.05 | 0.95 | 2b |
| 105 | X175B72 | 2 | 0.762 | 0.238 | 2a |
| 106 | X176B74 | 2 | 0.955 | 0.045 | 2a |
| 107 | X178B74 | 2 | 0.814 | 0.186 | 2a |
| 108 | X179B28 | 2 | 0.793 | 0.207 | 2a |
| 109 | X17C40 | 2 | 0.909 | 0.091 | 2a |
| 110 | X183B75 | 2 | 0.834 | 0.166 | 2a |
| 111 | X186B22 | 2 | 0.216 | 0.784 | 2b |
| 112 | X187B36 | 2 | 0.017 | 0.983 | 2b |
| 113 | X188B13 | 2 | 0.384 | 0.616 | 2b |
| 114 | X189B83 | 2 | 0.035 | 0.965 | 2b |
| 115 | X18C56 | 2 | 0.747 | 0.253 | 2a |
| 116 | X191B79 | 2 | 0.038 | 0.962 | 2b |
| 117 | X193B72 | 2 | 0.218 | 0.782 | 2b |
| 118 | X197B95 | 2 | 0.247 | 0.753 | 2b |
| 119 | X198B90 | 2 | 0.943 | 0.057 | 2a |
| 120 | X199B55 | 2 | 0.668 | 0.332 | 2a |
| 121 | X110B34 | 2 | 0.016 | 0.984 | 2b |
| 122 | X201B68 | 2 | 0.884 | 0.116 | 2a |
| 123 | X202B44 | 2 | 0.944 | 0.056 | 2a |
| 124 | X203B49 | 2 | 0.961 | 0.039 | 2a |
| 125 | X206C05 | 2 | 0.675 | 0.325 | 2a |
| 126 | X208C06 | 2 | 0.07 | 0.93 | 2b |

APPENDIX 3
SWS Classifier 0:Tests of differences G2a and G2b by 264 gene classifier

| | | | | | G2a-G2b: U- | G2a-G2b:t- | | |
|---|---|---|---|---|---|---|---|---|
| Nn | GeneSymbol | Genbank AccNo | Affy ID | SWS Cut-off | test, p-value | test, p value | hazard ratio | survival p value |
| 11 | CENPW/ | BG492359 | B.226936_at | 7.561905 | 8.79E−17 | 1.69E−16 | 1.134468229 | 0.003878804 |
| | c6orf173 | | | | | | | |
| 77 | FLJ11029 | BG165011 | B.228273_at | 7.706303 | 1.00E−16 | 8.50E−17 | 0.670107381 | 0.076512816 |
| 108 | KIF2C | U63743 | A.209408_at | 7.371746 | 2.81E−16 | 1.78E−16 | 0.567505306 | 0.139342988 |
| 59 | CDC20 | NM_001255 | A.202870_s_at | 7.129081 | 3.34E−16 | 1.12E−16 | 0.763919106 | 0.050953165 |
| 19 | BRRN1 | D38553 | A.212949_at | 5.916703 | 3.07E−15 | 3.10E−19 | 0.979515664 | 0.009067157 |
| 30 | LOCI 46909 | AA292789 | A.222039_at | 6.459052 | 5.69E−15 | 3.50E−16 | 0.883296839 | 0.019272796 |
| 70 | BIRC5 | NM_001168 | A.202095_s_at | 6.890672 | 5.69E−15 | 4.44E−17 | 0.708102857 | 0.064775046 |
| 36 | TRIP13 | NM_004237 | A.204033_at | 7.176822 | 6.43E−15 | 3.00E−15 | 0.850126178 | 0.022566954 |
| 129 | KNTC2 | NM_006101 | A.204162_at | 6.017032 | 7.57E−15 | 1.06E−18 | 0.490312356 | 0.194120238 |
| 110 | TPX2 | AF098158 | A.210052_s_at | 7.405101 | 1.05E−14 | 1.42E−17 | 0.573617337 | 0.142253402 |
| 79 | CDCA8 | BC001651 | A.221520_s_at | 5.218868 | 1.09E−14 | 1.33E−14 | 0.658701923 | 0.078806541 |
| 204 | MCM10 | NM_018518 | A.220651_s_at | 5.678376 | 1.09E−14 | 6.18E−17 | 0.241978243 | 0.517878593 |
| 123 | MELK | NM_014791 | A.204825_at | 7.107259 | 1.13E−14 | 5.94E−12 | 0.564480902 | 0.182369797 |
| 181 | UBE2C | NM_007019 | A.202954_at | 7.84307 | 1.18E−14 | 1.13E−15 | 0.330908338 | 0.37930096 |
| 71 | DLG7 | NM_014750 | A.203764_at | 6.312237 | 1.91E−14 | 1.00E−16 | 0.690085276 | 0.067402271 |
| 189 | BUB1 | AF043294 | A.209642_at | 6.011844 | 2.16E−14 | 1.92E−16 | 0.307317212 | 0.412526477 |
| 45 | KIF11 | NM_004523 | A.204444_at | 6.4655 | 2.85E−14 | 9.97E−17 | 0.79912997 | 0.033774349 |
| 92 | NUSAP1 | NM_016359 | A.218039_at | 7.542048 | 2.85E−14 | 2.99E−17 | 0.637335706 | 0.097019049 |
| 81 | CCNB2 | NM_004701 | A.202705_at | 7.009613 | 3.77E−14 | 3.42E−14 | 0.657262238 | 0.080389152 |
| 65 | CENPA | NM_001809 | A.204962_s_at | 6.344048 | 4.08E−14 | 3.68E−15 | 0.704184519 | 0.059400118 |
| 153 | TACC3 | NM_006342 | A.218308_at | 6.130286 | 7.36E−14 | 3.10E−18 | 0.412032354 | 0.281085866 |
| 149 | C10orf3 | NM_018131 | A.218542_at | 6.496495 | 1.17E−13 | 9.06E−14 | 0.420069588 | 0.270728962 |
| 1 | TTK | NM_003318 | A.204822_at | 6.239673 | 1.22E−13 | 2.13E−11 | 1.171238059 | 0.001762406 |
| 121 | BUB1B | NM_001211 | A.203755_at | 6.680032 | 1.22E−13 | 1.02E−15 | 0.516583867 | 0.174775842 |
| 87 | KIFC1 | BC000712 | A.209680_s_at | 6.974641 | 1.27E−13 | 1.21E−17 | 0.666825849 | 0.082783088 |
| 57 | PRC1 | NM_003981 | A.218009_s_at | 7.337561 | 1.37E−13 | 2.10E−15 | 0.739645377 | 0.049096515 |
| 113 | RRM2 | NM_001034 | A.201890_at | 7.101362 | 1.43E−13 | 8.41E−17 | 0.546002522 | 0.149339996 |
| 80 | | AI807356 | B.227350_at | 6.865844 | 1.48E−13 | 2.74E−15 | 0.67252443 | 0.080294447 |
| 98 | CENPE | NM_001813 | A.205046_at | 5.197169 | 1.60E−13 | 1.08E−17 | 0.599151091 | 0.113911695 |
| 72 | | AL138828 | B.228069_at | 7.011902 | 1.94E−13 | 5.85E−13 | 0.688832061 | 0.067813492 |
| 35 | RRM2 | BC001886 | A.209773_s_at | 7.297867 | 2.35E−13 | 1.38E−14 | 0.872228422 | 0.021541003 |
| 88 | MCM10 | AB042719 | B.222962_s_at | 6.132775 | 2.35E−13 | 6.93E−13 | 0.654527529 | 0.082868968 |
| 131 | FOXM1 | NM_021953 | A.202580_x_at | 6.582712 | 3.20E−13 | 1.17E−11 | 0.474709695 | 0.205857802 |
| 48 | HMMR | NM_012485 | A.207165_at | 6.588466 | 3.87E−13 | 1.27E−15 | 0.779819603 | 0.035980677 |
| 135 | C15orf20 | AF108138 | B.228252_at | 6.651787 | 5.24E−13 | 1.78E−14 | 0.46154664 | 0.218296542 |
| 224 | | NM_018123 | A.219918_s_at | 6.595823 | 5.65E−13 | 1.99E−14 | 0.187818441 | 0.619909548 |
| 120 | CDKN3 | AF213033 | A.209714_s_at | 6.841428 | 6.09E−13 | 8.44E−14 | 0.515982924 | 0.173720857 |
| 147 | KIAA0101 | NM_014736 | A.202503_s_at | 8.205376 | 7.09E−13 | 2.32E−15 | 0.419483056 | 0.265960679 |
| 103 | TOP2A | NM_001067 | A.201292_at | 7.246792 | 7.93E−13 | 1.91E−12 | 0.580536011 | 0.127083337 |
| 244 | CCNA2 | NM_001237 | A.203418_at | 6.194046 | 9.57E−13 | 7.68E−13 | 0.145722581 | 0.709744105 |
| 260 | MCM6 | NM_005915 | A.201930_at | 7.935338 | 1.07E−12 | 1.16E−11 | 0.052604412 | 0.888772119 |
| 144 | | NM_003158 | A.208079_s_at | 6.652593 | 1.11E−12 | 3.25E−12 | 0.433825107 | 0.249984002 |
| 228 | CDCA3 | BC002551 | B.223307_at | 7.841831 | 1.20E−12 | 5.50E−12 | 0.179511584 | 0.640656915 |
| 32 | RACGAP1 | AU153848 | A.222077_s_at | 7.120661 | 1.24E−12 | 2.34E−14 | 0.913401129 | 0.020315096 |
| 63 | CDC2 | AL524035 | A.203213_at | 7.015218 | 1.34E−12 | 9.22E−15 | 0.735324772 | 0.055865092 |
| 200 | TYMS | NM_001071 | A.202589_at | 7.824209 | 1.39E−12 | 1.51E−13 | 0.263662339 | 0.502055144 |
| 107 | SPAG5 | NM_006461 | A.203145_at | 6.462682 | 1.44E−12 | 2.15E−10 | 0.558587857 | 0.135557314 |
| 105 | | AL135396 | B.225834_at | 7.24667 | 1.67E−12 | 8.09E−13 | 0.564178077 | 0.129238859 |
| 82 | HCAP-G | NM_022346 | A.218663_at | 5.783124 | 1.94E−12 | 1.35E−13 | 0.656424847 | 0.080453666 |
| 28 | KIF20A | NM_005733 | A.218755_at | 7.211537 | 2.33E−12 | 3.05E−12 | 1.045743941 | 0.01613823 |
| 21 | FLJ10719 | BG478677 | A.213008_at | 6.446077 | 2.42E−12 | 3.00E−13 | 0.965117941 | 0.01033584 |
| 245 | LMNB1 | NM_005573 | A.203276_at | 7.110038 | 3.36E−12 | 1.50E−12 | −0.13973266 | 0.719231757 |
| 215 | AURKB | AB011446 | A.209464_at | 5.961137 | 4.18E−12 | 1.56E−12 | 0.221725785 | 0.555668954 |
| 138 | STK6 | NM_003600 | A.204092_s_at | 6.726571 | 4.84E−12 | 5.72E−12 | 0.442828835 | 0.235837295 |
| 33 | CCNB1 | BE407516 | A.214710_s_at | 7.155461 | 5.20E−12 | 5.40E−12 | 0.864913582 | 0.021007755 |
| 119 | ZWINT | NM_007057 | A.204026_s_at | 7.505467 | 6.01 E−12 | 1.15E−12 | 0.55129017 | 0.171799897 |
| 226 | HSPC150 | AB032931 | B.223229_at | 7.394742 | 6.95E−12 | 3.63E−13 | 0.183095635 | 0.629346456 |
| 50 | DKFZp762E1312 | NM_018410 | A.218726_at | 6.378121 | 9.59E−12 | 1.09E−12 | 0.773287213 | 0.038624799 |
| 199 | KIF14 | AW183154 | B.236641_at | 6.417492 | 1.07E−11 | 2.06E−13 | 0.255996821 | 0.501112791 |
| 139 | CDC2 | NM_001786 | A.203214_x_at | 6.588012 | 1.19E−11 | 6.90E−12 | 0.474661236 | 0.239070992 |
| 66 | CDC2 | D88357 | A.210559_s_at | 7.039539 | 1.42E−11 | 4.29E−13 | 0.738161604 | 0.059693607 |
| 173 | MAD2L1 | NM_002358 | A.203362_s_at | 6.460559 | 1.42E−11 | 1.47E−12 | 0.351480911 | 0.351833246 |
| 46 | HCAP-G | NM_022346 | A.218662_s_at | 6.059402 | 1.47E−11 | 1.20E−11 | 0.794011776 | 0.033909771 |
| 180 | | NM_005196 | A.207828_s_at | 7.236993 | 1.52E−11 | 1.01E−11 | 0.331918842 | 0.374266884 |
| 208 | KIF4A | NM_012310 | A.218355_at | 6.617376 | 1.64E−11 | 3.36E−10 | 0.249364706 | 0.538318296 |
| 95 | C6orf115 | AF116682 | B.223361_at | 8.755507 | 1.70E−11 | 1.02E−12 | 0.681679019 | 0.104802269 |
| 104 | DEPDC1 | AK000490 | B.222958_s_at | 6.874692 | 1.82E−11 | 1.69E−11 | 0.589562887 | 0.127107203 |
| 38 | FKSG14 | BC005400 | B.222848_at | 6.651721 | 1.88E−11 | 1.30E−12 | 0.884636483 | 0.024726016 |
| 89 | CKS2 | NM_001827 | A.204170_s_at | 7.835274 | 1.88E−11 | 2.57E−13 | 0.663167842 | 0.083465644 |
| 155 | CDCAI | AF326731 | B.223381_at | 6.49209 | 3.80E−11 | 5.13E−13 | 0.388889256 | 0.296165769 |
| 94 | DEPDC1 | AI810054 | B.235545_at | 6.249524 | 3.93E−11 | 5.99E−11 | 0.627093597 | 0.104698657 |
| 220 | ANLN | AK023208 | B.222608_s_at | 6.955614 | 4.68E−11 | 1.12E−11 | 0.198482286 | 0.602004883 |
| 213 | HN1 | AF060925 | B.222396_at | 8.422507 | 4.84E−11 | 6.28E−11 | −0.230083835 | 0.550728055 |
| 85 | NEK2 | NM_002497 | A.204641_at | 7.001719 | 5.19E−11 | 1.15E−12 | 0.647731608 | 0.081742332 |
| 150 | PKMYT1 | NM_004203 | A.204267_x_at | 6.922908 | 5.37E−11 | 1.32E−10 | 0.411866565 | 0.277601663 |
| 231 | BRIP1 | BF056791 | B.235609_at | 7.148933 | 5.75E−11 | 9.99E−12 | 0.16413055 | 0.666683251 |
| 263 | DEPDC1B | AK001166 | B.226980_at | 5.497689 | 5.75E−11 | 2.26E−09 | −0.024099105 | 0.95106539 |
| 17 | Spc24 | AI469788 | B.235572_at | 6.783946 | 6.38E−11 | 6.44E−12 | 0.992685847 | 0.007915906 |
| 115 | CCNB1 | N90191 | B.228729_at | 6.801847 | 6.38E−11 | 1.14E−11 | 0.528115076 | 0.166575624 |
| 61 | GAJ | AY028916 | B.223700_at | 5.843192 | 6.60E−11 | 6.67E−12 | 0.741255524 | 0.055223051 |
| 91 | C9orf140 | AW250904 | B.225777_at | 7.887661 | 6.83E−11 | 4.69E−10 | 0.679522784 | 0.08594282 |
| 125 | KPNA2 | NM_002266 | A.201088_at | 8.496449 | 7.07E−11 | 7.68E−11 | 0.519228058 | 0.185275145 |
| 86 | | NM_021067 | A.206102_at | 6.71395 | 7.57E−11 | 2.09E−11 | 0.646830568 | 0.081940926 |
| 165 | TOPK | NM_018492 | A.219148_at | 6.462595 | 7.84E−11 | 4.16E−11 | 0.3730149 | 0.327935025 |
| 15 | GAS2L3 | H37811 | B.235709_at | 6.727849 | 8.11E−11 | 3.33E−12 | 1.034666753 | 0.00553654 |
| 20 | C22orf18 | NM_024053 | A.218741_at | 6.348817 | 8.11E−11 | 2.63E−10 | 0.960849718 | 0.010156324 |
| 163 | MK167 | BF001806 | A.212022_s_at | 6.725468 | 8.11E−11 | 4.78E−11 | 0.429593931 | 0.323243491 |
| 111 | MYBL2 | NM_002466 | A.201710_at | 6.06614 | 8.98E−11 | 5.62E−11 | 0.550044743 | 0.143391526 |
| 214 | UHRF1 | AK025578 | B.225655_at | 7.733479 | 9.62E−11 | 5.34E−12 | 0.224764258 | 0.552775395 |
| 248 | ANP32E | NM_030920 | A.208103_s_at | 6.298887 | 1.07E−10 | 1.11E−08 | 0.103382105 | 0.797118551 |
| 236 | GTSE1 | BF973178 | A.215942_s_at | 5.468846 | 1.22E−10 | 3.81E−12 | 0.162445666 | 0.691025332 |
| 13 | RAD51 | NM_002875 | A.205024_s_at | 6.352379 | 1.26E−10 | 1.00E−12 | 1.114959663 | 0.004430713 |
| 178 | UBE2S | NM_014501 | A.202779_s_at | 6.916494 | 1.31E−10 | 3.36E−10 | 0.363864456 | 0.368883213 |
| 74 | GTSE1 | NM_016426 | A.204315_s_at | 6.416579 | 1.65E−10 | 7.20E−12 | 0.678223538 | 0.069012359 |
| 101 | TOP2A | NM_001067 | A.201291_s_at | 7.356644 | 2.24E−10 | 5.61E−11 | 0.578232509 | 0.125387811 |
| 172 | CDCA7 | AY029179 | 8.224428_s_at | 7.674613 | 3.56E−10 | 1.86E−08 | 0.429731941 | 0.350206624 |
| 122 | CDCA3 | NM_031299 | A.221436_s_at | 6.189773 | 3.93E−10 | 1.33E−09 | 0.511019556 | 0.176038534 |
| 93 | | NM_014875 | A.206364_at | 6.151827 | 5.11E−10 | 7.01E−11 | 0.614988939 | 0.103135349 |
| 183 | | T90295 | B.226661_at | 6.682487 | 6.64E−10 | 5.62E−09 | 0.346703445 | 0.401640846 |
| 166 | MGC45866 | AI638593 | B.230021_at | 6.42395 | 7.32E−10 | 2.47E−11 | 0.446135442 | 0.332297655 |
| 205 | MCM2 | NM_004526 | A.202107_s_at | 7.860975 | 8.89E−10 | 8.26E−10 | 0.274006856 | 0.528409926 |
| 78 | | AW271106 | 8.229490_s_at | 6.222193 | 9.18E−10 | 3.24E−10 | 0.677333915 | 0.077888591 |
| 198 | C20orf129 | BC001068 | B.225687_at | 7.232237 | 1.08E−09 | 5.23E−10 | 0.257721719 | 0.500092255 |
| 40 | RAD51AP1 | BE966146 | A.204146_at | 6.304944 | 1.11E−09 | 3.76E−08 | 0.865618275 | 0.026849949 |
| 207 | CCNE2 | NM_004702 | A.205034_at | 6.205506 | 1.64E−09 | 1.51E−08 | 0.231488922 | 0.536273359 |
| 185 | NUDT1 | NM_002452 | A.204766_s_at | 5.670523 | 2.04E−09 | 3.43E−11 | 0.336279873 | 0.404064878 |
| 34 | GPR19 | NM_006143 | A.207183_at | 5.256843 | 3.83E−09 | 1.26E−08 | 0.929389932 | 0.021115848 |
| 247 | | NM_017669 | A.219650_at | 5.042153 | 3.95E−09 | 1.21E−08 | 0.116316954 | 0.762199631 |
| 140 | HN1 | NM_016185 | A.217755_at | 7.911819 | 5.22E−09 | 4.13E−08 | 0.44433103 | 0.239189026 |
| 237 | HIST1H4C | NM_003542 | A.205967_at | 8.379597 | 5.55E−09 | 3.41E−08 | 0.155454713 | 0.692380424 |
| 102 | HMGAI | NM_002131 | A.206074_s_at | 7.672253 | 6.68E−09 | 2.90E−08 | 0.57340264 | 0.126796719 |
| 141 | H2AFZ | NM_002106 | A.200853_at | 8.589569 | 6.68E−09 | 1.57E−09 | 0.438942866 | 0.241203655 |
| 168 | WDHD1 | AK001538 | A.216228_s_at | 4.541043 | 6.68E−09 | 3.23E−09 | 0.362835144 | 0.336253542 |
| 2 | KIF18A | NM_031217 | A.221258_s_at | 5.364945 | 6.89E−09 | 7.41E−10 | 1.170250756 | 0.001940291 |
| 39 | | X07868 | A.202409_at | 7.991737 | 8.27E−09 | 1.54E−08 | −0.856276422 | 0.025419272 |
| 174 | ATAD2 | AI925583 | B.222740_at | 6.841603 | 8.53E−09 | 2.90E−08 | 0.349834975 | 0.351965862 |
| 37 | CENPN | BF111626 | B.228559_at | 7.221195 | 1.16E−08 | 1.63E−07 | 0.89220144 | 0.022622085 |
| 22 | E2F8/FLJ23311 | NM_024680 | A.219990_at | 5.027727 | 1.81E−08 | 7.53E−10 | 1.11499904 | 0.010526137 |
| 212 | ASK | NM_006716 | A.204244_s_at | 5.982485 | 2.04E−08 | 8.87E−08 | 0.22517382 | 0.547726962 |
| 127 | DC13 | NM_020188 | A.218447_at | 7.435987 | 2.59E−08 | 3.28E−08 | 0.49836629 | 0.192923434 |
| 146 | FLJ10948 | NM_018281 | A.218552_at | 7.977808 | 2.59E−08 | 7.07E−08 | −0.420287158 | 0.265366947 |
| 187 | CHEK1 | NM_001274 | A.205394_at | 5.621699 | 2.67E−08 | 1.47E−07 | 0.313136396 | 0.408533969 |
| 84 | FBXO5 | AK026197 | B.234863_x_at | 6.934979 | 3.37E−08 | 8.76E−09 | 0.655530277 | 0.08133619 |
| 221 | NUP62 | AI859620 | B.230966_at | 6.428907 | 5.37E−08 | 9.30E−08 | 0.194175581 | 0.602079507 |
| 191 | CDCA5 | BE614410 | B.224753_at | 4.982139 | 5.85E−08 | 1.79E−07 | 0.29495453 | 0.433517741 |
| 56 | DCC1 | NM_024094 | A.219000_s_at | 6.283528 | 8.74E−08 | 6.85E−06 | 0.768011092 | 0.045286733 |
| 69 | HELLS | NM_018063 | A.220085_at | 5.288593 | 8.74E−08 | 3.52E−07 | 0.713632416 | 0.06333745 |
| 83 | CDT1 | AW075105 | B.228868_x_at | 7.054331 | 9.79E−08 | 5.55E−07 | 0.648477951 | 0.081174122 |
| 203 | Pfs2 | BC003186 | A.221521 sat | 6.320114 | 1.45E−07 | 9.94E−08 | 0.246497936 | 0.516223881 |
| 255 | | AA938184 | B.236312_at | 5.701626 | 1.62E−07 | 2.80E−08 | −0.07481093 | 0.857385605 |
| 192 | | T58044 | B.227232_at | 8.502082 | 1.67E−07 | 8.48E−08 | −0.297539293 | 0.446463222 |
| 229 | FLJ13710 | AK024132 | B.232944_at | 6.19474 | 1.67E−07 | 2.60E−07 | −0.186238076 | 0.642824579 |
| 223 | PHF19 | BE544837 | 8.227211_at | 6.348665 | 2.03E−07 | 3.74E−07 | −0.223589203 | 0.606554898 |
| 206 | KIF23 | NM_004856 | A.204709_s_at | 5.173124 | 2.74E−07 | 2.81E−08 | 0.274556227 | 0.529893874 |
| 243 | EXO1 | NM_003686 | A.204603_at | 5.927018 | 3.60E−07 | 1.00E−07 | 0.141097415 | 0.709073685 |
| 170 | CXCL10 | NM_001565 | A.204533_at | 7.91312 | 6.01E−07 | 1.24E−06 | 0.354258493 | 0.340498438 |
| 256 | MLPH | AI810764 | B.229150_at | 8.078007 | 7.23E−07 | 1.23E−05 | −0.076268477 | 0.86056146 |
| 29 | LAPTM4B | T15777 | A.214039_s_at | 9.320913 | 7.83E−07 | 5.35E−06 | 0.889471325 | 0.016767645 |
| 42 | NUSAP1 | NM_018454 | A.219978_s_at | 6.335678 | 1.07E−06 | 2.59E−06 | 0.903401222 | 0.029991418 |
| 44 | EHD2 | AI417917 | A.221870_at | 6.477374 | 1.22E−06 | 3.36E−06 | −0.893991178 | 0.032844532 |
| 148 | C10orf56 | AL049949 | A.212419_at | 7.650367 | 1.25E−06 | 2.36E−06 | −0.426019275 | 0.266863651 |
| 145 | FSHPRH1 | BF793446 | A.214804_at | 5.010521 | 1.32E−06 | 1.57E−05 | 0.422634781 | 0.264823066 |
| 134 | ECT2 | NM_018098 | A.219787_s_at | 6.80516 | 1.43E−06 | 1.69E−06 | 0.486045036 | 0.213892061 |
| 116 | SLC7A5 | AB018009 | A.201195_s_at | 7.493131 | 1.46E−06 | 7.74E−06 | 0.540964485 | 0.166626703 |
| 26 | NUP88 | AI806781 | B.235786_at | 7.285647 | 1.62E−06 | 6.36E−07 | −0.911250522 | 0.014788954 |
| 136 | SCN7A | AI828648 | B.228504_at | 5.824759 | 1.89E−06 | 1.44E−06 | −0.453703343 | 0.222310621 |
| 171 | HPSE | NM_006665 | A.219403_s_at | 5.298862 | 1.99E−06 | 1.28E−06 | 0.394569194 | 0.343049791 |
| 25 | FLJ21062 | NM_024788 | A.219455_at | 5.525652 | 2.15E−06 | 5.20E−06 | −0.941228426 | 0.014095722 |
| 259 | CLDN5 | NM_003277 | A.204482_at | 6.151636 | 2.32E−06 | 6.44E−06 | −0.055268705 | 0.883493297 |
| 218 | SRD5AI | NM_001047 | A.204675_at | 7.100171 | 2.70E−06 | 4.78E−05 | 0.219783486 | 0.596970945 |
| 142 | SOD2 | X15132 | A.216841_s_at | 6.002653 | 3.14E−06 | 3.73E−06 | 0.444778653 | 0.246622419 |
| 210 | | AI668620 | B.237339_at | 9.669306 | 3.22E−06 | 1.88E−05 | −0.226029013 | 0.54306855 |
| 157 | ANKRD30A | AF269087 | B.223864_at | 9.414368 | 3.30E−06 | 9.96E−05 | −0.387746621 | 0.299216824 |
| 58 | COL14AI | BF449063 | A.212865_s_at | 7.287585 | 4.02E−06 | 1.36E−05 | −0.749700525 | 0.05022335 |
| 230 | C1orf21 | NM_030806 | A.221272_s_at | 5.622823 | 4.55E−06 | 1.14E−05 | −0.1682899 | 0.656466607 |
| 55 | CX3CR1 | U20350 | A.205898_at | 6.776389 | 5.27E−06 | 1.23E−04 | −0.749645527 | 0.043720315 |
| 151 | EGR1 | NM_001964 | A.201694_s_at | 8.620234 | 5.81E−06 | 3.60E−06 | −0.423112634 | 0.279987351 |
| 222 | | U79293 | A.215304_at | 6.931746 | 5.96E−06 | 2.81E−05 | −0.201281803 | 0.606462487 |
| 3 | CCL18 | Y13710 | A.32128_at | 6.244174 | 6.41E−06 | 2.75E−05 | 1.14221045 | 0.002597504 |
| 12 | CBX2 | BE514414 | B.226473_at | 7.558812 | 6.41E−06 | 1.13E−04 | 1.07504449 | 0.004054863 |
| 109 | ISG20 | NM_002201 | A.204698_at | 6.299944 | 6.73E−06 | 4.62E−06 | 0.5459336 | 0.14211529 |
| 118 | | AL360204 | B.232855_at | 4.628799 | 6.89E−06 | 9.05E−06 | −0.535303385 | 0.171221041 |
| 219 | DACH1 | NM_004392 | A.205472_s_at | 3.924559 | 6.89E−06 | 1.02E−05 | −0.212822165 | 0.597050977 |
| 132 | HSPC163 | NM_014184 | A.218728_s_at | 7.648067 | 7.41E−06 | 3.15E−06 | 0.507545115 | 0.210023483 |
| 152 | CIRBP | AL565767 | B.225191_at | 8.032986 | 8.16E−06 | 2.52E−06 | −0.469803635 | 0.280312337 |
| 158 | CYBRD1 | AI669804 | B.232459_at | 7.117116 | 8.36E−06 | 3.94E−05 | −0.388568867 | 0.310287696 |
| 160 | MCM4 | X74794 | A.212141_at | 6.729237 | 8.36E−06 | 1.02E−05 | 0.406623286 | 0.316436679 |
| 49 | FOS | BC004490 | A.209189_at | 8.992075 | 8.98E−06 | 4.05E−05 | −0.911746653 | 0.036012408 |
| 143 | CCNE1 | AI671049 | A.213523_at | 6.08195 | 1.04E−05 | 5.87E−05 | 0.463724353 | 0.248407611 |
| 137 | RBMS3 | AW338699 | 8.241789_at | 6.365561 | 1.14E−05 | 2.42E−04 | −0.454436208 | 0.224664187 |
| 112 | ITGA7 | AK022548 | A.216331_at | 5.153545 | 1.62E−05 | 1.32E−05 | −0.541433612 | 0.145348566 |
| 232 | CXCL11 | AF002985 | A.211122_s_at | 6.1001 | 1.66E−05 | 1.05E−05 | −0.1728883 | 0.666951268 |
| 76 | BM039 | NM_018455 | A.219555_s_at | 4.173851 | 2.14E−05 | 9.20E−06 | 0.673164666 | 0.074344562 |
| 62 | ATAD2 | AI139629 | B.235266_at | 6.191308 | 2.34E−05 | 1.39E−04 | 0.748127999 | 0.055689556 |
| 193 | GGH | NM_003878 | A.203560_at | 6.77081 | 2.75E−05 | 2.09E−05 | −0.293893248 | 0.453096633 |
| 14 | | AI693516 | B.228750_at | 7.124873 | 2.94E−05 | 2.85E−04 | −1.073910408 | 0.00444517 |
| 179 | ELN | AA479278 | A.212670_at | 6.895109 | 3.08E−05 | 1.86E−04 | −0.334047514 | 0.369570896 |
| 133 | NOVAI | NM_002515 | A.205794_s_at | 6.768152 | 3.68E−05 | 3.98E−04 | −0.489575159 | 0.211015726 |
| 90 | CACNAID | BE550599 | A.210108_at | 6.26118 | 4.21E−05 | 5.08E−05 | −0.642967417 | 0.084876377 |
| 234 | | AK002203 | B.226992_at | 7.90914 | 5.25E−05 | 2.56E−04 | −0.154632796 | 0.678987899 |
| 67 | NR4A2 | AA523939 | B.235739_at | 7.187449 | 5.73E−05 | 3.92E−06 | −0.731391224 | 0.062004634 |
| 190 | | AL512727 | A.215014_at | 4.833426 | 5.99E−05 | 1.73E−04 | −0.295736426 | 0.432032039 |
| 73 | DUSP1 | NM_004417 | A.201041_s_at | 9.748091 | 6.12E−05 | 4.18E−05 | −0.758479385 | 0.068505145 |
| 262 | | R38110 | B.240112_at | 5.163128 | 6.53E−05 | 2.48E−04 | −0.036764785 | 0.921615167 |
| 7 | STC2 | BC000658 | A.203439_s_at | 7.680632 | 6.82E−05 | 2.05E−04 | −1.191837167 | 0.003010392 |
| 52 | PLAC9 | AW964972 | 8.227419_x_at | 6.688968 | 7.76E−05 | 2.06E−04 | −0.786290483 | 0.040004936 |
| 211 | | BF508074 | B.240465_at | 6.004131 | 8.10E−05 | 5.21E−05 | 0.233504153 | 0.545194111 |
| 254 | KIAA0303 | AW971134 | A.222348_at | 4.963999 | 8.10E−05 | 3.04E−04 | −0.080778342 | 0.833005228 |
| 97 | PSAT1 | BC004863 | B.223062_s_at | 6.103481 | 9.21E−05 | 5.37E−05 | 0.595123345 | 0.109627082 |
| 68 | LRP2 | R73030 | B.230863_at | 7.464817 | 1.00E−04 | 6.57E−05 | −0.69766747 | 0.062336219 |
| 161 | | AL137566 | B.228554_at | 7.112413 | 1.05E−04 | 1.18E−04 | −0.40109127 | 0.318261339 |
| 162 | | BF513468 | B.241505_at | 7.15166 | 1.05E−04 | 1.53E−04 | 0.374700717 | 0.32253637 |
| 252 | MGC24047 | AI732488 | 8.229381_at | 7.228131 | 1.07E−04 | 1.17E−04 | −0.082087159 | 0.83034645 |
| 195 | NPY1R | NM_000909 | A.205440_s_at | 5.830472 | 1.11E−04 | 4.10E−04 | 0.337889908 | 0.461696619 |
| 27 | SIRT3 | AF083108 | A.221562_s_at | 5.964518 | 1.16E−04 | 6.45E−04 | −0.927132823 | 0.01545353 |
| 128 | LRP2 | NM_004525 | A.205710_at | 5.984454 | 1.19E−04 | 1.06E−04 | −0.492675347 | 0.193865955 |
| 235 | | AI492376 | B.231195_at | 5.196657 | 1.21E−04 | 2.67E−04 | −0.161302941 | 0.680051165 |
| 246 | NTN4 | AF278532 | B.223315_at | 8.269299 | 1.24E−04 | 1.70E−04 | −0.132354027 | 0.725835139 |
| 43 | STC2 | AI435828 | A.203438_at | 7.538814 | 1.32E−04 | 1.57E−04 | −0.797860709 | 0.031924561 |
| 175 | | AV733950 | A.201693_s_at | 7.906065 | 1.37E−04 | 9.86E−06 | −0.347314523 | 0.355018177 |
| 8 | RAI2 | NM_021785 | A.219440_at | 6.659438 | 1.99E−04 | 2.01E−04 | −1.108174776 | 0.003077111 |
| 196 | NMU | NM_006681 | A.206023_at | 5.10173 | 2.49E−04 | 1.99E−04 | 0.298272606 | 0.461878171 |
| 24 | | AI492388 | B.228854_at | 6.819756 | 2.70E−04 | 7.97E−04 | −0.950969041 | 0.013149939 |
| 5 | PTGER3 | AW242315 | A.213933_at | 7.356099 | 2.98E−04 | 1.25E−03 | −1.295337189 | 0.002908446 |
| 117 | FLJ10901 | NM_018265 | A.219010_at | 6.942924 | 3.29E−04 | 5.19E−04 | 0.519806366 | 0.168424663 |
| 41 | FOSB | NM_006732 | A.202768_at | 6.19218 | 3.35E−04 | 1.36E−04 | −0.815647159 | 0.028388157 |
| 177 | ERBB4 | AK024204 | B.233498_at | 7.543523 | 3.77E−04 | 6.61E−04 | −0.336800577 | 0.367457847 |
| 106 | LAF4 | AI033582 | B.244696_at | 7.41577 | 4.24E−04 | 4.62E−04 | −0.572783549 | 0.134590614 |
| 6 | MAPT | NM_016835 | A.203928_x_at | 6.910278 | 4.41E−04 | 1.10E−03 | −1.114016712 | 0.002947734 |
| 124 | | AW970881 | A.222314_x_at | 5.250506 | 4.67E−04 | 3.33E−04 | −0.49598679 | 0.183685062 |
| 240 | SRD5AI | BC006373 | A.211056_s_at | 6.760491 | 4.95E−04 | 1.14E−03 | −0.177760256 | 0.69950506 |
| 176 | FMO5 | AK022172 | A.215300_s_at | 4.143345 | 5.24E−04 | 2.15E−04 | −0.338873235 | 0.365924454 |
| 186 | ZNF533 | H15261 | B.243929_at | 4.716503 | 5.77E−04 | 7.17E−05 | −0.312801434 | 0.408005813 |
| 169 | TTC18 | AW024437 | 8.229170_s_at | 6.229818 | 6.11E−04 | 1.99E−03 | −0.373029504 | 0.339898261 |
| 54 | BCL2 | AU146384 | B.232210_at | 8.094828 | 6.71E−04 | 1.23E−03 | −0.760752368 | 0.043704125 |
| 47 | CYBRD1 | NM_024843 | A.217889_s_at | 5.642724 | 6.97E−04 | 6.69E−04 | −0.79117731 | 0.035959897 |
| 201 | SLC40AI | AA588092 | B.239723_at | 6.922208 | 6.97E−04 | 2.68E−04 | 0.246250082 | 0.508863506 |
| 253 | MUSTN1 | BF793701 | B.226856_at | 5.562608 | 7.51E−04 | 1.01E−03 | 0.096986779 | 0.832624185 |
| 9 | MFAP4 | R72286 | A.212713_at | 6.51492 | 8.09E−04 | 1.76E−03 | −1.113082042 | 0.003213842 |
| 99 | LRRC17 | NM_005824 | A.205381_at | 7.216997 | 8.24E−04 | 1.34E−03 | −0.571472856 | 0.124800311 |
| 239 | STK32B | NM_018401 | A.219686_at | 4.566312 | 8.88E−04 | 1.45E−03 | −0.157335553 | 0.695207523 |
| 164 | | BF433570 | B.237301_at | 6.317098 | 1.09E−03 | 1.13E−03 | −0.408773136 | 0.325727984 |
| 114 | | AW512787 | B.238481_at | 8.511705 | 1.17E−03 | 1.56E−03 | −0.558216466 | 0.164426983 |
| 242 | NAT1 | NM_000662 | A.214440_at | 7.742309 | 1.19E−03 | 1.86E−03 | 0.171562865 | 0.708554521 |
| 60 | EPHX2 | AF233336 | A.209368_at | 6.403114 | 1.21E−03 | 1.87E−04 | −0.760554602 | 0.052355087 |
| 167 | PHYHD1 | AL545998 | B.226846_at | 7.221441 | 1.25E−03 | 1.72E−03 | −0.359283155 | 0.333823065 |
| 159 | | NM_030896 | A.221275_s_at | 3.961128 | 1.28E−03 | 5.94E−04 | −0.376502717 | 0.314482067 |
| 130 | CYBRD1 | AL136693 | B.222453_at | 9.399092 | 1.30E−03 | 1.48E−03 | −0.48333705 | 0.195588312 |
| 238 | NAV3 | NM_014903 | A.204823_at | 5.823519 | 1.47E−03 | 1.61E−03 | −0.158076864 | 0.693777462 |
| 53 | OGN | NM_014057 | A.218730_s_at | 4.932506 | 1.64E−03 | 5.08E−03 | −0.757516472 | 0.042394291 |
| 100 | SYNCRIP | NM_006372 | A.217834_s_at | 6.812321 | 1.85E−03 | 1.62E−03 | 0.587077047 | 0.125280752 |
| 154 | | AK021990 | B.232699_at | 5.867527 | 1.95E−03 | 1.28E−03 | −0.393427065 | 0.289892653 |
| 184 | ERBB4 | AW772192 | A.214053_at | 7.07437 | 2.09E−03 | 8.91E−04 | −0.336719007 | 0.401781194 |
| 216 | | NM_004522 | A.203130_s_at | 7.321429 | 2.28E−03 | 1.57E−02 | 0.231698726 | 0.564239609 |
| 4 | MAPT | J03778 | A.206401_s_at | 6.455705 | 2.36E−03 | 5.08E−03 | −1.13042772 | 0.002820509 |
| 64 | HMGB3 | NM_005342 | A.203744_at | 7.550192 | 3.25E−03 | 4.04E−03 | 0.738482321 | 0.05884424 |
| 251 | LAF4 | AA572675 | B.232286_at | 7.169029 | 3.25E−03 | 3.10E−03 | 0.108992215 | 0.812211511 |
| 31 | AQP9 | NM_020980 | A.205568_at | 4.951949 | 3.53E−03 | 1.59E−03 | 0.895190406 | 0.019505478 |
| 188 | DACH1 | AI650353 | B.228915_at | 7.671623 | 3.53E−03 | 1.64E−03 | −0.311012322 | 0.411423928 |
| 75 | SCN4B | AW026241 | B.236359_at | 5.552642 | 3.89E−03 | 6.07E−03 | −0.677852485 | 0.073197783 |
| 233 | FLJ41238 | AW629527 | B.229764_at | 6.531923 | 4.16E−03 | 5.68E−03 | −0.176712594 | 0.671030052 |
| 156 | SCUBE2 | AI424243 | A.219197_s_at | 8.381941 | 5.04E−03 | 5.90E−03 | −0.386317867 | 0.298631944 |
| 227 | CYP4Z1 | AV700083 | B.237395_at | 8.750525 | 5.04E−03 | 3.96E−03 | 0.18037008 | 0.631134131 |
| 217 | ESR1 | NM_000125 | A.205225_at | 7.494275 | 5.12E−03 | 4.39E−04 | 0.416453493 | 0.570106612 |
| 225 | CYP4X1 | AA557324 | B.227702_at | 8.597239 | 5.29E−03 | 5.71E−03 | −0.187667891 | 0.625691687 |
| 202 | TTC18 | AW024437 | B.229169_at | 5.826554 | 5.55E−03 | 6.63E−03 | −0.242354326 | 0.51485792 |
| 16 | MAPT | NM_016835 | A.203929_s_at | 7.791403 | 5.73E−03 | 3.01E−03 | −1.029153262 | 0.00579453 |
| 182 | ECT2 | BG170335 | 8.234992_x_at | 5.165319 | 6.91E−03 | 9.85E−03 | 0.329010815 | 0.379594706 |
| 261 | | AV709727 | B.225996_at | 7.571507 | 7.58E−03 | 5.58E−04 | 0.044547315 | 0.905089266 |
| 250 | PTPRT | NM_007050 | A.205948_at | 6.763414 | 8.18E−03 | 7.66E−03 | −0.089691431 | 0.810363802 |
| 209 | CALML5 | NM_017422 | A.220414_at | 5.994003 | 8.56E−03 | 3.32E−03 | 0.267191443 | 0.540775453 |
| 18 | SUSD3 | AW966474 | 8.227182_at | 8.195015 | 1.04E−02 | 8.78E−03 | −1.297832347 | 0.008305284 |
| 10 | STH | AAI99717 | B.225379_at | 7.857365 | 2.30E−02 | 7.91E−03 | −1.097446295 | 0.003735657 |
| 197 | FLJ45983 | AI631850 | B.240192_at | 5.289779 | 4.94E−02 | 4.10E−02 | 0.314861713 | 0.468985395 |
| 241 | | AL031658 | B.232357_at | 5.976136 | 5.06E−02 | 4.81E−02 | −0.145562103 | 0.700546776 |
| 96 | | AI826437 | B.229975_at | 6.381037 | 5.90E−02 | 5.75E−02 | 0.78769613 | 0.109281577 |
| 249 | LOC143381 | AW242720 | B.227550_at | 7.656959 | 9.35E−02 | 2.86E−02 | −0.106502567 | 0.798016237 |
| 258 | DNALI1 | AW299538 | B.227081_at | 7.085104 | 1.03E−01 | 5.27E−03 | −0.068369896 | 0.881511542 |
| 194 | GAMT | NM_000156 | A.205354_at | 5.947354 | 1.53E−01 | 2.91E−02 | −0.284372326 | 0.457600609 |
| 257 | DNALI1 | NM_003462 | A.205186_at | 4.299739 | 1.54E−01 | 2.58E−02 | −0.08851533 | 0.869483818 |
| 23 | MMP1 | NM_002421 | A.204475_at | 7.170495 | 2.04E−01 | 2.26E−01 | 1.047070923 | 0.01186788 |
| 264 | PPP1R3C | N26005 | A.204284_at | 7.027458 | 2.85E−01 | 6.40E−01 | −0.006752502 | 0.987063337 |
| 126 | CXCL14 | NM_004887 | A.218002_s_at | 8.251287 | 4.49E−01 | 5.03E−01 | −0.502169588 | 0.190758302 |
| 51 | CXCL14 | AF144103 | 6.222484_s_at | 9.336584 | 6.54E−01 | 5.00E−01 | −0.777835445 | 0.03993233 |

APPENDIX 4
SWS Classifier 0: Clinical validation (survival analysis) of G2a and
G2b tumour subtypes (264 classifier).
# Cox PH test summary (Baseline
group 1)
coef exp(coef) se(coef) z p

| | | | | |
|---|---|---|---|---|
| group2b | 0.795 | 2.21 | 0.292 | 2.72 |
| 0.0066 | | | | |

Likelihood ratio test = 7.25 on 1 df, p = 0.00711

n = 126

n events rmean se(rmean) median 0.95LCL

| 0.95UCL | | | | | |
|---|---|---|---|---|---|
| group 2a = 79 | 23 9.97 | 0.507 | Inf | Inf | Inf |
| group 2b = 47 | 24 7.35 | 0.793 | 8.5 | 2.58 | |
| Inf | | | | | |

APPENDIX 5A
SWS Classifier 1

| | UGID(build | Unigen | | Genbank | | |
|---|---|---|---|---|---|---|
| Order | #183) | eName | GeneSymbol | Acc | Affi ID | Cut-off |
| 1 | Hs.528654 | Hypothetical | FLJ1102911 | BG165011 | B.228273_at | 7.706303 |
| | | protein | | | | |
| | | FLJ11029 | | | | |
| 2 | acc_NM_003158.1 | Serine/threonine | STK6 | NM_003158 | A.208079_s_at | 6.652593 |
| | | kinase 6. | | | | |
| | | transcript 1 | | | | |
| 3 | Hs.35962 | CDNA clone | | BG492359 | B.226936_at | 7.561905 |
| | | IMAGE: 4452583, | | | | |
| | | partial cds | | | | |
| 4 | Hs.308045 | Barren | BRRN1 | D38553 | A.212949_at | 5.916703 |
| | | homolog | | | | |
| | | (Drosophila) | | | | |
| 5 | Hs.184339 | Maternal | MELK | NM_014791 | A.204825_at | 7.107259 |
| | | embryonic | | | | |
| | | leucine | | | | |
| | | zipper | | | | |
| | | kinase | | | | |
| 6 | Hs.250822 | Serine/threonine | STK6 | NM_003600 | A.204092_s_at | 6.726571 |
| | | kinase 6, | | | | |
| | | transcript 2 | | | | |

APPENDIX 5B
SWS Classifier 1: Classifier Accuracy
Accuracy
G1 = 65/68
(95.6%)
G3 = 51/55 ?
(94.5%)

| | Patient | Histologic | Probability | Probability | Predicted |
|---|---|---|---|---|---|
| Number | ID | grade | for G 1 | for G3 | grade |
| 1 | X100B08 | 1 | 0.959 | 0.041 | 1 |
| 2 | X209C10 | 1 | 0.959 | 0.041 | 1 |
| 3 | X21C28 | 1 | 0.959 | 0.041 | 1 |
| 4 | X220C70 | 1 | 0.959 | 0.041 | 1 |
| 5 | X224C93 | 1 | 0.959 | 0.041 | 1 |
| 6 | X227C50 | 1 | 0.959 | 0.041 | 1 |
| 7 | X229C44 | 1 | 0.959 | 0.041 | 1 |
| 8 | X231C80 | 1 | 0.959 | 0.041 | 1 |
| 9 | X233C91 | 1 | 0.959 | 0.041 | 1 |
| 10 | X235C20 | 1 | 0.287 | 0.713 | 3 |
| 11 | X236C55 | 1 | 0.959 | 0.041 | 1 |
| 12 | X114B68 | 1 | 0.782 | 0.218 | 1 |
| 13 | X243C70 | 1 | 0.959 | 0.041 | 1 |
| 14 | X246C75 | 1 | 0.959 | 0.041 | 1 |
| 15 | X248C91 | 1 | 0.959 | 0.041 | 1 |
| 16 | X253C20 | 1 | 0.959 | 0.041 | 1 |
| 17 | X259C74 | 1 | 0.959 | 0.041 | 1 |
| 18 | X261C94 | 1 | 0.959 | 0.041 | 1 |
| 19 | X262C85 | 1 | 0.959 | 0.041 | 1 |
| 20 | X263C82 | 1 | 0.959 | 0.041 | 1 |
| 21 | X266C51 | 1 | 0.959 | 0.041 | 1 |
| 22 | X267C04 | 1 | 0.959 | 0.041 | 1 |
| 23 | X282C51 | 1 | 0.959 | 0.041 | 1 |
| 24 | X284C63 | 1 | 0.959 | 0.041 | 1 |
| 25 | X289C75 | 1 | 0.959 | 0.041 | 1 |
| 26 | X28C76 | 1 | 0.959 | 0.041 | 1 |
| 27 | X294C04 | 1 | 0.887 | 0.113 | 1 |
| 28 | X309C49 | 1 | 0.01 | 0.99 | 3 |
| 29 | X316C65 | 1 | 0.959 | 0.041 | 1 |
| 30 | X128B48 | 1 | 0.959 | 0.041 | 1 |
| 31 | X33C30 | 1 | 0.959 | 0.041 | 1 |
| 32 | X39C24 | 1 | 0.959 | 0.041 | 1 |
| 33 | X42C57 | 1 | 0.959 | 0.041 | 1 |
| 34 | X45A96 | 1 | 0.959 | 0.041 | 1 |
| 35 | X48A46 | 1 | 0.959 | 0.041 | 1 |
| 36 | X49A07 | 1 | 0.959 | 0.041 | 1 |
| 37 | X52A90 | 1 | 0.959 | 0.041 | 1 |
| 38 | X61A53 | 1 | 0.959 | 0.041 | 1 |
| 39 | X65A68 | 1 | 0.959 | 0.041 | 1 |
| 40 | X6B85 | 1 | 0.733 | 0.267 | 1 |
| 41 | X72A92 | 1 | 0.489 | 0.511 | 3 |
| 42 | X135B40 | 1 | 0.959 | 0.041 | 1 |
| 43 | X74A63 | 1 | 0.894 | 0.106 | 1 |
| 44 | X83A37 | 1 | 0.733 | 0.267 | 1 |
| 45 | X8B87 | 1 | 0.959 | 0.041 | 1 |
| 46 | X99A50 | 1 | 0.959 | 0.041 | 1 |
| 47 | X138B34 | 1 | 0.959 | 0.041 | 1 |
| 48 | X155B52 | 1 | 0.959 | 0.041 | 1 |
| 49 | X156B01 | 1 | 0.959 | 0.041 | 1 |
| 50 | X160B16 | 1 | 0.959 | 0.041 | 1 |
| 51 | X163B27 | 1 | 0.959 | 0.041 | 1 |
| 52 | X105B13 | 1 | 0.959 | 0.041 | 1 |
| 53 | X173B43 | 1 | 0.959 | 0.041 | 1 |
| 54 | X174B41 | 1 | 0.959 | 0.041 | 1 |
| 55 | X177B67 | 1 | 0.959 | 0.041 | 1 |
| 56 | X106B55 | 1 | 0.959 | 0.041 | 1 |
| 57 | X180B38 | 1 | 0.959 | 0.041 | 1 |
| 58 | X181B70 | 1 | 0.887 | 0.113 | 1 |
| 59 | X184B38 | 1 | 0.959 | 0.041 | 1 |
| 60 | X185B44 | 1 | 0.959 | 0.041 | 1 |
| 61 | X10B88 | 1 | 0.678 | 0.322 | 1 |
| 62 | X192B69 | 1 | 0.959 | 0.041 | 1 |
| 63 | X195B75 | 1 | 0.959 | 0.041 | 1 |
| 64 | X196B81 | 1 | 0.887 | 0.113 | 1 |
| 65 | X19C33 | 1 | 0.959 | 0.041 | 1 |
| 66 | X204B85 | 1 | 0.959 | 0.041 | 1 |
| 67 | X205B99 | 1 | 0.915 | 0.085 | 1 |
| 68 | X207C08 | 1 | 0.959 | 0.041 | 1 |
| 69 | X111B51 | 3 | 0.001 | 0.999 | 3 |
| 70 | X222C26 | 3 | 0.036 | 0.974 | 3 |
| 71 | X226C06 | 3 | 0.001 | 0.999 | 3 |
| 72 | X113B11 | 3 | 0.001 | 0.999 | 3 |
| 73 | X232C58 | 3 | 0.001 | 0.999 | 3 |
| 74 | X234C15 | 3 | 0.003 | 0.997 | 3 |
| 75 | X238C87 | 3 | 0.163 | 0.837 | 3 |
| 76 | X241C01 | 3 | 0.001 | 0.999 | 3 |
| 77 | X249C42 | 3 | 0.001 | 0.999 | 3 |
| 78 | X250C78 | 3 | 0.001 | 0.999 | 3 |
| 79 | X252C64 | 3 | 0.001 | 0.999 | 3 |
| 80 | X269C68 | 3 | 0.001 | 0.999 | 3 |
| 81 | X26C23 | 3 | 0.047 | 0.953 | 3 |
| 82 | X270C93 | 3 | 0.001 | 0.999 | 3 |
| 83 | X271C71 | 3 | 0.001 | 0.999 | 3 |
| 84 | X279C61 | 3 | 0.001 | 0.999 | 3 |
| 85 | X287C67 | 3 | 0.001 | 0.999 | 3 |
| 86 | X291C17 | 3 | 0.001 | 0.999 | 3 |
| 87 | X127B00 | 3 | 0.001 | 0.999 | 3 |
| 88 | X303C36 | 3 | 0.001 | 0.999 | 3 |
| 89 | X304C89 | 3 | 0.996 | 0.004 | 1 |
| 90 | X311A27 | 3 | 0.001 | 0.999 | 3 |
| 91 | X313A87 | 3 | 0.001 | 0.999 | 3 |
| 92 | X314B55 | 3 | 0.001 | 0.999 | 3 |
| 93 | X101B88 | 3 | 0.001 | 0.999 | 3 |
| 94 | X37C06 | 3 | 0.001 | 0.999 | 3 |
| 95 | X46A25 | 3 | 0.001 | 0.999 | 3 |
| 96 | X131B79 | 3 | 0.597 | 0.403 | 1 |
| 97 | X54A09 | 3 | 0.001 | 0.999 | 3 |
| 98 | X55A79 | 3 | 0.001 | 0.999 | 3 |
| 99 | X62A02 | 3 | 0.001 | 0.999 | 3 |
| 100 | X66A84 | 3 | 0.001 | 0.999 | 3 |
| 101 | X67A43 | 3 | 0.001 | 0.999 | 3 |
| 102 | X69A93 | 3 | 0.001 | 0.999 | 3 |
| 103 | X70A79 | 3 | 0.001 | 0.999 | 3 |
| 104 | X73A01 | 3 | 0.034 | 0.966 | 3 |
| 105 | X76A44 | 3 | 0.005 | 0.995 | 3 |
| 106 | X79A35 | 3 | 0.005 | 0.995 | 3 |
| 107 | X82A83 | 3 | 0.005 | 0.995 | 3 |
| 108 | X89A64 | 3 | 0.001 | 0.999 | 3 |
| 109 | X90A63 | 3 | 0.001 | 0.999 | 3 |
| 110 | X139B03 | 3 | 0.001 | 0.999 | 3 |
| 111 | X102B06 | 3 | 0.001 | 0.999 | 3 |
| 112 | X142B05 | 3 | 0.003 | 0.998 | 3 |
| 113 | X143B81 | 3 | 0.016 | 0.984 | 3 |
| 114 | X146B39 | 3 | 0.001 | 0.999 | 3 |
| 115 | X147B19 | 3 | 0.001 | 0.999 | 3 |
| 116 | X103B41 | 3 | 0.001 | 0.999 | 3 |
| 117 | X153B09 | 3 | 0.001 | 0.999 | 3 |
| 118 | X104B91 | 3 | 0.001 | 0.999 | 3 |
| 119 | X162B98 | 3 | 0.033 | 0.977 | 3 |
| 120 | X172B19 | 3 | 0.004 | 0.996 | 3 |
| 121 | X182B43 | 3 | 0.001 | 0.999 | 3 |
| 122 | X194B60 | 3 | 0.005 | 0.995 | 3 |
| 123 | X200B47 | 3 | 0.931 | 0.069 | 1 |

APPENDIX 5C
SWS Classifier 1: Prediction validation
# Cox PH test summary (Baseline
group 1)
coef exp(coef) se(coef) z p

| | | | | |
|---|---|---|---|---|
| group3 | 0.921 | 2.51 | 0.292 | 3.15 |
| 0.0016 | | | | |

Likelihood ratio test = 9.66 on 1 df, p = 0.00189

n = 126

n events rmean se(rmean) median 0.95LCL

| 0.95UCL | | | | | |
|---|---|---|---|---|---|
| group 2a = 83 | 23 | 10.0 | 0.489 | Inf | Inf |
| Inf | | | | | |
| group 2b = 43 | 24 | 7.0 | 0.820 | 6.5 | 2.58 |
| Inf | | | | | |

*DFS Event defined as any type of recurrence or death because
of breast cancer, whichever comes first

| | | Prob- | Prob- | Pre- | | DFS |
|---|---|---|---|---|---|---|
| | Patient | ability | ability | dicted | DFS | E- |
| Number | ID | for G2a | for G2b | grade | TIME | VENT* |
| 1 | X210C72 | 0.894 | 0.106 | 2a | 0.5 | 1 |
| 2 | X211C88 | 0.777 | 0.223 | 2a | 1.5 | 0 |
| 3 | X212C21 | 0.959 | 0.041 | 2a | 3.75 | 1 |
| 4 | X213C36 | 0.005 | 0.995 | 2b | 10.08 | 0 |
| 5 | X216C61 | 0.959 | 0.041 | 2a | 10.75 | 0 |
| 6 | X217C79 | 0.959 | 0.041 | 2a | 10.75 | 0 |
| 7 | X218C29 | 0.894 | 0.106 | 2a | 10.75 | 0 |
| 8 | X112B55 | 0.007 | 0.993 | 2b | 0.92 | 1 |
| 9 | X221C14 | 0.143 | 0.857 | 2b | 3 | 1 |
| 10 | X223C51 | 0.894 | 0.106 | 2a | 8.42 | 0 |
| 11 | X225C52 | 0.001 | 0.999 | 2b | 10.75 | 0 |
| 12 | X22C62 | 0.959 | 0.041 | 2a | 4.83 | 0 |
| 13 | X230C47 | 0.001 | 0.999 | 2b | 0.5 | 1 |
| 14 | X237C56 | 0.143 | 0.857 | 2b | 10.67 | 0 |
| 15 | X23C52 | 0.005 | 0.995 | 2b | 8.5 | 1 |
| 16 | X240C54 | 0.005 | 0.995 | 2b | 2.42 | 1 |
| 17 | X242C21 | 0.209 | 0.791 | 2b | 2.17 | 1 |
| 18 | X244C89 | 0.777 | 0.223 | 2a | 7.25 | 1 |
| 19 | X245C22 | 0.143 | 0.857 | 2b | 0 | 1 |
| 20 | X247C76 | 0.959 | 0.041 | 2a | 10.5 | 0 |
| 21 | X11B47 | 0.959 | 0.041 | 2a | 7.42 | 0 |
| 22 | X24C30 | 0.959 | 0.041 | 2a | 10.67 | 0 |
| 23 | X251C14 | 0.959 | 0.041 | 2a | 10.5 | 0 |
| 24 | X254C80 | 0.959 | 0.041 | 2a | 10.5 | 0 |
| 25 | X255C06 | 0.959 | 0.041 | 2a | 10.5 | 0 |
| 26 | X256C45 | 0.001 | 0.999 | 2b | 1.25 | 1 |
| 27 | X120B73 | 0.001 | 0.999 | 2b | 11.58 | 0 |
| 28 | X257C87 | 0.959 | 0.041 | 2a | 10.5 | 0 |
| 29 | X258C21 | 0.959 | 0.041 | 2a | 5.75 | 1 |
| 30 | X260C91 | 0.09 | 0.91 | 2b | 10.42 | 0 |
| 31 | X265C40 | 0.777 | 0.223 | 2a | 10.42 | 0 |
| 32 | X122B81 | 0.959 | 0.041 | 2a | 11.17 | 0 |
| 33 | X268C87 | 0.001 | 0.999 | 2b | 10.33 | 0 |
| 34 | X272C88 | 0.959 | 0.041 | 2a | 10.33 | 0 |
| 35 | X274C81 | 0.959 | 0.041 | 2a | 10.33 | 0 |
| 36 | X275C70 | 0.959 | 0.041 | 2a | 10.25 | 0 |
| 37 | X277C64 | 0.959 | 0.041 | 2a | 8.58 | 0 |
| 38 | X124B25 | 0.959 | 0.041 | 2a | 5 | 1 |
| 39 | X278C80 | 0.351 | 0.649 | 2b | 10.25 | 0 |
| 40 | X27C82 | 0.959 | 0.041 | 2a | 6.83 | 0 |
| 41 | X280C43 | 0.959 | 0.041 | 2a | 1 | 1 |
| 42 | X286C91 | 0.959 | 0.041 | 2a | 10 | 0 |
| 43 | X288C57 | 0.959 | 0.041 | 2a | 10 | 0 |
| 44 | X290C91 | 0.959 | 0.041 | 2a | 10 | 0 |
| 45 | X292C66 | 0.959 | 0.041 | 2a | 10 | 0 |
| 46 | X296C95 | 0.959 | 0.041 | 2a | 9.92 | 0 |
| 47 | X297C26 | 0.959 | 0.041 | 2a | 9.92 | 0 |
| 48 | X298C47 | 0.959 | 0.041 | 2a | 6.5 | 1 |
| 49 | X301C66 | 0.959 | 0.041 | 2a | 9.92 | 0 |
| 50 | X307C50 | 0.777 | 0.223 | 2a | 9.83 | 0 |
| 51 | X308C93 | 0.005 | 0.995 | 2b | 2.25 | 1 |
| 52 | X34C80 | 0.959 | 0.041 | 2a | 10.17 | 0 |
| 53 | X35C29 | 0.202 | 0.798 | 2b | 2.42 | 1 |
| 54 | X36C17 | 0.959 | 0.041 | 2a | 10.08 | 0 |
| 55 | X40C57 | 0.877 | 0.123 | 2a | 10 | 0 |
| 56 | X41C65 | 0.959 | 0.041 | 2a | 9.92 | 0 |
| 57 | X130B92 | 0.959 | 0.041 | 2a | 4.42 | 1 |
| 58 | X43C47 | 0.877 | 0.123 | 2a | 9.92 | 0 |
| 59 | X44A53 | 0.123 | 0.877 | 2b | 12.75 | 0 |
| 60 | X47A87 | 0.001 | 0.999 | 2b | 9.58 | 1 |
| 61 | X50A91 | 0.777 | 0.223 | 2a | 9.08 | 1 |
| 62 | X51A98 | 0.959 | 0.041 | 2a | 12.67 | 0 |
| 63 | X53A06 | 0.202 | 0.798 | 2b | 2.58 | 1 |
| 64 | X56A94 | 0.959 | 0.041 | 2a | 1.08 | 1 |
| 65 | X58A50 | 0.001 | 0.999 | 2b | 0.42 | 1 |
| 66 | X5B97 | 0.001 | 0.999 | 2b | 0.75 | 1 |
| 67 | X60A05 | 0.959 | 0.041 | 2a | 0.67 | 1 |
| 68 | X134B33 | 0.015 | 0.985 | 2b | 2 | 1 |
| 69 | X63A62 | 0.959 | 0.041 | 2a | 0.17 | 1 |
| 70 | X64A59 | 0.046 | 0.954 | 2b | 12.42 | 0 |
| 71 | X75A01 | 0.202 | 0.798 | 2b | 3.58 | 1 |
| 72 | X77A50 | 0.662 | 0.338 | 2a | 1.08 | 1 |
| 73 | X7B96 | 0.959 | 0.041 | 2a | 2.42 | 1 |
| 74 | X84A44 | 0.017 | 0.983 | 2b | 12.17 | 0 |
| 75 | X136B04 | 0.959 | 0.041 | 2a | 2.42 | 1 |
| 76 | X85A03 | 0.777 | 0.223 | 2a | 2.08 | 0 |
| 77 | X86A40 | 0.001 | 0.999 | 2b | 12.17 | 0 |
| 78 | X87A79 | 0.662 | 0.338 | 2a | 12.08 | 0 |
| 79 | X88A67 | 0.029 | 0.971 | 2b | 4.25 | 1 |
| 80 | X94A16 | 0.959 | 0.041 | 2a | 11.08 | 0 |
| 81 | X96A21 | 0.959 | 0.041 | 2a | 0.08 | 1 |
| 82 | X137B88 | 0.894 | 0.106 | 2a | 10.5 | 1 |
| 83 | X9B52 | 0.877 | 0.123 | 2a | 11.33 | 0 |
| 84 | X13B79 | 0.959 | 0.041 | 2a | 10.83 | 0 |
| 85 | X140B91 | 0.959 | 0.041 | 2a | 11.5 | 0 |
| 86 | X144B49 | 0.959 | 0.041 | 2a | 11.5 | 0 |
| 87 | X145B10 | 0.003 | 0.997 | 2b | 11.42 | 0 |
| 88 | X14B98 | 0.924 | 0.076 | 2a | 10.83 | 0 |
| 89 | X150B81 | 0.777 | 0.223 | 2a | 11.42 | 0 |
| 90 | X151B84 | 0.894 | 0.106 | 2a | 11.42 | 0 |
| 91 | X152B99 | 0.959 | 0.041 | 2a | 2.08 | 0 |
| 92 | X154B42 | 0.005 | 0.995 | 2b | 3.42 | 1 |
| 93 | X158B84 | 0.959 | 0.041 | 2a | 4.67 | 1 |
| 94 | X159B47 | 0.001 | 0.999 | 2b | 6.5 | 1 |
| 95 | X15C94 | 0.959 | 0.041 | 2a | 4.42 | 0 |
| 96 | X161B31 | 0.959 | 0.041 | 2a | 11.42 | 0 |
| 97 | X164B81 | 0.001 | 0.999 | 2b | 11.33 | 0 |
| 98 | X165B72 | 0.046 | 0.954 | 2b | 1.5 | 1 |
| 99 | X166B79 | 0.025 | 0.975 | 2b | 11.33 | 0 |
| 100 | X168B51 | 0.959 | 0.041 | 2a | 5.33 | 0 |
| 101 | X169B79 | 0.959 | 0.041 | 2a | 11.33 | 0 |
| 102 | X16C97 | 0.877 | 0.123 | 2a | 3.58 | 1 |
| 103 | X170B15 | 0.894 | 0.106 | 2a | 4.08 | 1 |
| 104 | X171B77 | 0.005 | 0.995 | 2b | 1.75 | 1 |
| 105 | X175B72 | 0.894 | 0.106 | 2a | 0 | 1 |
| 106 | X176B74 | 0.959 | 0.041 | 2a | 6 | 0 |
| 107 | X178B74 | 0.761 | 0.239 | 2a | 7.42 | 0 |
| 108 | X179B28 | 0.959 | 0.041 | 2a | 2.33 | 1 |
| 109 | X17C40 | 0.959 | 0.041 | 2a | 1.92 | 0 |
| 110 | X183B75 | 0.894 | 0.106 | 2a | 7 | 1 |
| 111 | X186B22 | 0.029 | 0.971 | 2b | 0.17 | 1 |
| 112 | X187B36 | 0.001 | 0.999 | 2b | 0 | 1 |
| 113 | X188B13 | 0.469 | 0.531 | 2b | 11 | 0 |
| 114 | X189B83 | 0.005 | 0.995 | 2b | 11 | 0 |
| 115 | X18C56 | 0.777 | 0.223 | 2a | 10.75 | 0 |
| 116 | X191B79 | 0.001 | 0.999 | 2b | 4.42 | 1 |
| 117 | X193B72 | 0.469 | 0.531 | 2b | 10.92 | 0 |
| 118 | X197B95 | 0.777 | 0.223 | 2a | 10.92 | 0 |
| 119 | X198B90 | 0.959 | 0.041 | 2a | 10.92 | 0 |
| 120 | X199B55 | 0.894 | 0.106 | 2a | 10.92 | 0 |
| 121 | X110B34 | 0.001 | 0.999 | 2b | 11.67 | 0 |
| 122 | X201B68 | 0.959 | 0.041 | 2a | 10.92 | 0 |
| 123 | X202B44 | 0.959 | 0.041 | 2a | 10.83 | 0 |
| 124 | X203B49 | 0.959 | 0.041 | 2a | 10.83 | 0 |
| 125 | X206C05 | 0.924 | 0.076 | 2a | 6.42 | 0 |
| 126 | X208C06 | 0.001 | 0.999 | 2b | 0.08 | 0 |

APPENDIX 6A
SWS Classifier 2

| | UGID | | | | | |
|---|---|---|---|---|---|---|
| | (build | | Gene | Genbank | | |
| Order | #177) | Unigene Name | Symbol | Acc | AffyID | cut-off |
| 1 | Hs.184339 | Maternal embryonic | MELK | NM_014791 | A.204825_at | 5.43711 |
| | | leucine zipper kinase | | | | |
| 2 | Hs.308045 | Barren homolog | BRRN1 | D38553 | A.212949_at | 5.50455 |
| | | (Drosophila) | | | | |
| 3 | Hs.244580 | TPX2, microtubule- | TPX2 | AF098158 | A.210052_s_at | 5.87219 |
| | | associated protein | | | | |
| | | homolog (Xenopus | | | | |
| | | laevis) | | | | |
| 4 | Hs.486401 | CDNA clone IMAGE: 4452583, | | BG492359 | B.226936_at | 7.56993 |
| | | partial cds | | | | |
| 5 | Hs.75573 | Centromere protein E, | CENPE | NM_001813 | A.205046_at | 6.94342 |
| | | 312 kDa | | | | |
| 6 | Hs.528654 | Hypothetical protein | FLJ11029 | BG165011 | B.228273_at | 7.71114 |
| | | FLJ11029 | | | | |
| 7 | acc_NM_003158 | | | NM_003158 | A.208079_s_at | 6.57103 |
| 8 | Hs.524571 | Cell division cycle | CDCA8 | BC001651 | A.221520_s_at | 6.8942 |
| | | associated 8 | | | | |
| 9 | Hs.239 | Forkhead box M1 | FOXM1 | NM_021953 | A.202580_x_at | 5.21151 |
| 10 | Hs.179718 | V-myb myeloblastosis | MYBL2 | NM_002466 | A.201710_at | 6.26908 |
| | | viral oncogene homolog | | | | |
| | | (avian)-like 2 | | | | |
| 11 | Hs.169840 | TTK protein kinase | TTK | NM_003318 | A.204822_at | 8.2308 |
| 12 | Hs.75678 | FBJ murine | FOSB | NM_006732 | A.202768_at | 8.76158 |
| | | osteosarcoma viral | | | | |
| | | oncogene homolog B | | | | |
| 13 | Hs.25647 | V-fos FBJ murine | FOS | BC004490 | A.209189_at | 7.08598 |
| | | osteosarcoma viral | | | | |
| | | oncogene homolog | | | | |
| 14 | Hs.524216 | Cell division cycle | CDCA3 | NM_031299 | A.221436_s_at | 6.29283 |
| | | associated 3 | | | | |
| 15 | Hs.381225 | Kinetochore protein | Spc24 | AI469788 | B.235572_at | 6.3405 |
| | | Spc24 | | | | |
| 16 | Hs.62180 | Anillin, actin binding | ANLN | AK023208 | B.222608_s_at | 6.84578 |
| | | protein (scraps homolog, | | | | |
| | | Drosophila) | | | | |
| 17 | Hs.434886 | Cell division cycle | CDCA5 | BE614410 | B.224753_at | 5.29067 |
| | | associated 5 | | | | |
| 18 | Hs.523468 | Signal peptide, CUB | SCUBE2 | AI424243 | A.219197_s_at | 5.79216 |
| | | domain, EGF-like 2 | | | | |

APPENDIX 6B
SWS Classifier 2: Accuracy
Accuracy
G1 = 65/68
(95.6%)
G3 = 53/55
(96.4%)

| | | | | | Predicted |
|---|---|---|---|---|---|
| | Patients | Histologic | Probability | Probability | grade |
| Number | ID | grade | for G1 | for G3 | G1 or G3 |
| 1 | X100B08 | 1 | 0.993 | 0.007 | 1 |
| 2 | X209C10 | 1 | 0.982 | 0.018 | 1 |
| 3 | X21C28 | 1 | 0.993 | 0.007 | 1 |
| 4 | X220C70 | 1 | 0.993 | 0.007 | 1 |
| 5 | X224C93 | 1 | 0.991 | 0.009 | 1 |
| 6 | X227C50 | 1 | 0.995 | 0.005 | 1 |
| 7 | X229C44 | 1 | 0.987 | 0.013 | 1 |
| 8 | X231C80 | 1 | 0.978 | 0.022 | 1 |
| 9 | X233C91 | 1 | 0.993 | 0.007 | 1 |
| 10 | X235C20 | 1 | 0.120 | 0.880 | 3 |
| 11 | X236C55 | 1 | 0.995 | 0.005 | 1 |
| 12 | X114B68 | 1 | 0.684 | 0.316 | 1 |
| 13 | X243C70 | 1 | 0.993 | 0.007 | 1 |
| 14 | X246C75 | 1 | 0.993 | 0.007 | 1 |
| 15 | X248C91 | 1 | 0.995 | 0.005 | 1 |
| 16 | X253C20 | 1 | 0.995 | 0.005 | 1 |
| 17 | X259C74 | 1 | 0.991 | 0.009 | 1 |
| 18 | X261C94 | 1 | 0.995 | 0.005 | 1 |
| 19 | X262C85 | 1 | 0.995 | 0.005 | 1 |
| 20 | X263C82 | 1 | 0.995 | 0.005 | 1 |
| 21 | X266C51 | 1 | 0.976 | 0.024 | 1 |
| 22 | X267C04 | 1 | 0.812 | 0.188 | 1 |
| 23 | X282C51 | 1 | 0.995 | 0.005 | 1 |
| 24 | X284C63 | 1 | 0.989 | 0.011 | 1 |
| 25 | X289C75 | 1 | 0.995 | 0.005 | 1 |
| 26 | X28C76 | 1 | 0.995 | 0.005 | 1 |
| 27 | X294C04 | 1 | 0.859 | 0.141 | 1 |
| 28 | X309C49 | 1 | 0.086 | 0.914 | 3 |
| 29 | X316C65 | 1 | 0.993 | 0.007 | 1 |
| 30 | X128B48 | 1 | 0.995 | 0.005 | 1 |
| 31 | X33C30 | 1 | 0.995 | 0.005 | 1 |
| 32 | X39C24 | 1 | 0.989 | 0.011 | 1 |
| 33 | X42C57 | 1 | 0.995 | 0.005 | 1 |
| 34 | X45A96 | 1 | 0.995 | 0.005 | 1 |
| 35 | X48A46 | 1 | 0.995 | 0.005 | 1 |
| 36 | X49A07 | 1 | 0.993 | 0.007 | 1 |
| 37 | X52A90 | 1 | 0.985 | 0.015 | 1 |
| 38 | X61A53 | 1 | 0.968 | 0.032 | 1 |
| 39 | X65A68 | 1 | 0.991 | 0.009 | 1 |
| 40 | X6B85 | 1 | 0.035 | 0.965 | 3 |
| 41 | X72A92 | 1 | 0.855 | 0.145 | 1 |
| 42 | X135B40 | 1 | 0.995 | 0.005 | 1 |
| 43 | X74A63 | 1 | 0.927 | 0.073 | 1 |
| 44 | X83A37 | 1 | 0.833 | 0.167 | 1 |
| 45 | X8B87 | 1 | 0.995 | 0.005 | 1 |
| 46 | X99A50 | 1 | 0.759 | 0.241 | 1 |
| 47 | X138B34 | 1 | 0.995 | 0.005 | 1 |
| 48 | X155B52 | 1 | 0.995 | 0.005 | 1 |
| 49 | X156B01 | 1 | 0.995 | 0.005 | 1 |
| 50 | X160B16 | 1 | 0.993 | 0.007 | 1 |
| 51 | X163B27 | 1 | 0.995 | 0.005 | 1 |
| 52 | X105B13 | 1 | 0.870 | 0.130 | 1 |
| 53 | X173B43 | 1 | 0.995 | 0.005 | 1 |
| 54 | X174B41 | 1 | 0.990 | 0.010 | 1 |
| 55 | X177B67 | 1 | 0.993 | 0.007 | 1 |
| 56 | X106B55 | 1 | 0.993 | 0.007 | 1 |
| 57 | X180B38 | 1 | 0.993 | 0.007 | 1 |
| 58 | X181B70 | 1 | 0.969 | 0.031 | 1 |
| 59 | X184B38 | 1 | 0.983 | 0.017 | 1 |
| 60 | X185B44 | 1 | 0.995 | 0.005 | 1 |
| 61 | X10B88 | 1 | 0.892 | 0.108 | 1 |
| 62 | X192B69 | 1 | 0.995 | 0.005 | 1 |
| 63 | X195B75 | 1 | 0.993 | 0.007 | 1 |
| 64 | X196B81 | 1 | 0.644 | 0.356 | 1 |
| 65 | X19C33 | 1 | 0.986 | 0.014 | 1 |
| 66 | X204B85 | 1 | 0.995 | 0.005 | 1 |
| 67 | X205B99 | 1 | 0.837 | 0.163 | 1 |
| 68 | X207C08 | 1 | 0.993 | 0.007 | 1 |
| 69 | X111B51 | 3 | 0.001 | 0.999 | 3 |
| 70 | X222C26 | 3 | 0.240 | 0.760 | 3 |
| 71 | X226C06 | 3 | 0.001 | 0.999 | 3 |
| 72 | X113B11 | 3 | 0.005 | 0.995 | 3 |
| 73 | X232C58 | 3 | 0.001 | 0.999 | 3 |
| 74 | X234C15 | 3 | 0.014 | 0.986 | 3 |
| 75 | X238C87 | 3 | 0.293 | 0.707 | 3 |
| 76 | X241C01 | 3 | 0.001 | 0.999 | 3 |
| 77 | X249C42 | 3 | 0.002 | 0.998 | 3 |
| 78 | X250C78 | 3 | 0.004 | 0.996 | 3 |
| 79 | X252C64 | 3 | 0.002 | 0.998 | 3 |
| 80 | X269C68 | 3 | 0.001 | 0.999 | 3 |
| 81 | X26C23 | 3 | 0.444 | 0.556 | 3 |
| 82 | X270C93 | 3 | 0.018 | 0.982 | 3 |
| 83 | X271C71 | 3 | 0.005 | 0.995 | 3 |
| 84 | X279C61 | 3 | 0.001 | 0.999 | 3 |
| 85 | X287C67 | 3 | 0.005 | 0.995 | 3 |
| 86 | X291C17 | 3 | 0.001 | 0.999 | 3 |
| 87 | X127B00 | 3 | 0.001 | 0.999 | 3 |
| 88 | X303C36 | 3 | 0.001 | 0.999 | 3 |
| 89 | X304C89 | 3 | 0.999 | 0.001 | 1 |
| 90 | X311A27 | 3 | 0.004 | 0.996 | 3 |
| 91 | X313A87 | 3 | 0.001 | 0.999 | 3 |
| 92 | X314B55 | 3 | 0.002 | 0.998 | 3 |
| 93 | X101B88 | 3 | 0.001 | 0.999 | 3 |
| 94 | X37C06 | 3 | 0.003 | 0.997 | 3 |
| 95 | X46A25 | 3 | 0.002 | 0.998 | 3 |
| 96 | X131B79 | 3 | 0.241 | 0.759 | 3 |
| 97 | X54A09 | 3 | 0.001 | 0.999 | 3 |
| 98 | X55A79 | 3 | 0.002 | 0.998 | 3 |
| 99 | X62A02 | 3 | 0.001 | 0.999 | 3 |
| 100 | X66A84 | 3 | 0.001 | 0.999 | 3 |
| 101 | X67A43 | 3 | 0.001 | 0.999 | 3 |
| 102 | X69A93 | 3 | 0.043 | 0.957 | 3 |
| 103 | X70A79 | 3 | 0.001 | 0.999 | 3 |
| 104 | X73A01 | 3 | 0.145 | 0.855 | 3 |
| 105 | X76A44 | 3 | 0.018 | 0.982 | 3 |
| 106 | X79A35 | 3 | 0.004 | 0.996 | 3 |
| 107 | X82A83 | 3 | 0.012 | 0.988 | 3 |
| 108 | X89A64 | 3 | 0.000 | 1.000 | 3 |
| 109 | X90A63 | 3 | 0.001 | 0.999 | 3 |
| 110 | X139B03 | 3 | 0.003 | 0.997 | 3 |
| 111 | X102B06 | 3 | 0.001 | 0.999 | 3 |
| 112 | X142B05 | 3 | 0.006 | 0.994 | 3 |
| 113 | X143B81 | 3 | 0.009 | 0.991 | 3 |
| 114 | X146B39 | 3 | 0.001 | 0.999 | 3 |
| 115 | X147B19 | 3 | 0.003 | 0.997 | 3 |
| 116 | X103B41 | 3 | 0.001 | 0.999 | 3 |
| 117 | X153B09 | 3 | 0.001 | 0.999 | 3 |
| 118 | X104B91 | 3 | 0.023 | 0.977 | 3 |
| 119 | X162B98 | 3 | 0.134 | 0.866 | 3 |
| 120 | X172B19 | 3 | 0.051 | 0.949 | 3 |
| 121 | X182B43 | 3 | 0.001 | 0.999 | 3 |
| 122 | X194B60 | 3 | 0.004 | 0.996 | 3 |
| 123 | X200B47 | 3 | 1.000 | 0.000 | 1 |

APPENDIX 6C
SWS Classifier 2: G2a-G2b Prediction and Survival
# Cox PH test summary (Baseline
group 1)
coef exp(coef) se(coef) z p

| | | | | |
|---|---|---|---|---|
| group2b | 1.06 | 2.87 | 0.298 | 3.54 |
| 4e−04 | | | | |

Likelihood ratio test = 12.8 on 1 df, p = 0.000341

n = 126

n events rmean se(rmean) median 0.95LCL

| 0.95UCL | | | | | |
|---|---|---|---|---|---|
| group 2a = 77 | 19 | 10.33 | 0.499 | Inf | Inf |
| Inf | | | | | |
| group 2b = 49 | 28 | 6.98 | 0.750 | 7 | 3 |
| Inf | | | | | |

*DFS Event defined as any type of recurrence or death because
of breast cancer, whichever comes first

| | | | | | Pre- | | |
|---|---|---|---|---|---|---|---|
| | | | | | dicted | | |
| | | | | | grade | | |
| | | | Prob- | Prob- | (2a- | | |
| | | Histo- | ability | ability | G2a, | | DFS |
| | Patient | logic | for | for | 2b- | DFS | E- |
| Number | ID | grade | G2a | G2b | G2b) | TIME | VENT* |
| 1 | X210C72 | 2 | 0.017 | 0.983 | 2b | 0.5 | 1 |
| 2 | X211C88 | 2 | 0.673 | 0.327 | 2a | 1.5 | 0 |
| 3 | X212C21 | 2 | 1.000 | 0.000 | 2a | 3.75 | 1 |
| 4 | X216C61 | 2 | 0.999 | 0.001 | 2a | 10.75 | 0 |
| 5 | X217C79 | 2 | 0.999 | 0.001 | 2a | 10.75 | 0 |
| 6 | X218C29 | 2 | 0.999 | 0.001 | 2a | 10.75 | 0 |
| 7 | X223C51 | 2 | 0.997 | 0.003 | 2a | 8.42 | 0 |
| 8 | X22C62 | 2 | 0.999 | 0.001 | 2a | 4.83 | 0 |
| 9 | X244C89 | 2 | 0.059 | 0.941 | 2b | 7.25 | 1 |
| 10 | X247C76 | 2 | 0.894 | 0.106 | 2a | 10.5 | 0 |
| 11 | X11B47 | 2 | 0.999 | 0.001 | 2a | 7.42 | 0 |
| 12 | X24C30 | 2 | 1.000 | 0.000 | 2a | 10.67 | 0 |
| 13 | X251C14 | 2 | 1.000 | 0.000 | 2a | 10.5 | 0 |
| 14 | X254C80 | 2 | 1.000 | 0.000 | 2a | 10.5 | 0 |
| 15 | X255C06 | 2 | 0.999 | 0.001 | 2a | 10.5 | 0 |
| 16 | X257C87 | 2 | 1.000 | 0.000 | 2a | 10.5 | 0 |
| 17 | X258C21 | 2 | 1.000 | 0.000 | 2a | 5.75 | 1 |
| 18 | X265C40 | 2 | 0.934 | 0.066 | 2a | 10.42 | 0 |
| 19 | X122B81 | 2 | 0.999 | 0.001 | 2a | 11.17 | 0 |
| 20 | X272C88 | 2 | 1.000 | 0.000 | 2a | 10.33 | 0 |
| 21 | X274C81 | 2 | 1.000 | 0.000 | 2a | 10.33 | 0 |
| 22 | X275C70 | 2 | 0.999 | 0.001 | 2a | 10.25 | 0 |
| 23 | X277C64 | 2 | 1.000 | 0.000 | 2a | 8.58 | 0 |
| 24 | X124B25 | 2 | 0.999 | 0.001 | 2a | 5 | 1 |
| 25 | X27C82 | 2 | 1.000 | 0.000 | 2a | 6.83 | 0 |
| 26 | X280C43 | 2 | 1.000 | 0.000 | 2a | 1 | 1 |
| 27 | X286C91 | 2 | 1.000 | 0.000 | 2a | 10 | 0 |
| 28 | X288C57 | 2 | 0.999 | 0.001 | 2a | 10 | 0 |
| 29 | X290C91 | 2 | 1.000 | 0.000 | 2a | 10 | 0 |
| 30 | X292C66 | 2 | 0.961 | 0.039 | 2a | 10 | 0 |
| 31 | X296C95 | 2 | 1.000 | 0.000 | 2a | 9.92 | 0 |
| 32 | X297C26 | 2 | 1.000 | 0.000 | 2a | 9.92 | 0 |
| 33 | X298C47 | 2 | 0.998 | 0.002 | 2a | 6.5 | 1 |
| 34 | X301C66 | 2 | 0.902 | 0.098 | 2a | 9.92 | 0 |
| 35 | X307C50 | 2 | 0.406 | 0.594 | 2b | 9.83 | 0 |
| 36 | X34C80 | 2 | 0.999 | 0.001 | 2a | 10.17 | 0 |
| 37 | X36C17 | 2 | 1.000 | 0.000 | 2a | 10.08 | 0 |
| 38 | X40C57 | 2 | 0.805 | 0.195 | 2a | 10 | 0 |
| 39 | X41C65 | 2 | 0.999 | 0.001 | 2a | 9.92 | 0 |
| 40 | X130B92 | 2 | 1.000 | 0.000 | 2a | 4.42 | 1 |
| 41 | X43C47 | 2 | 0.539 | 0.461 | 2a | 9.92 | 0 |
| 42 | X50A91 | 2 | 0.998 | 0.002 | 2a | 9.08 | 1 |
| 43 | X51A98 | 2 | 0.155 | 0.845 | 2b | 12.67 | 0 |
| 44 | X56A94 | 2 | 0.999 | 0.001 | 2a | 1.08 | 1 |
| 45 | X60A05 | 2 | 0.999 | 0.001 | 2a | 0.67 | 1 |
| 46 | X63A62 | 2 | 0.999 | 0.001 | 2a | 0.17 | 1 |
| 47 | X7B96 | 2 | 0.081 | 0.919 | 2b | 2.42 | 1 |
| 48 | X136B04 | 2 | 0.999 | 0.001 | 2a | 2.42 | 1 |
| 49 | X85A03 | 2 | 0.939 | 0.061 | 2a | 2.08 | 0 |
| 50 | X94A16 | 2 | 1.000 | 0.000 | 2a | 11.08 | 0 |
| 51 | X96A21 | 2 | 0.999 | 0.001 | 2a | 0.08 | 1 |
| 52 | X137B88 | 2 | 0.992 | 0.008 | 2a | 10.5 | 1 |
| 53 | X9B52 | 2 | 0.134 | 0.866 | 2b | 11.33 | 0 |
| 54 | X13B79 | 2 | 1.000 | 0.000 | 2a | 10.83 | 0 |
| 55 | X140B91 | 2 | 1.000 | 0.000 | 2a | 11.5 | 0 |
| 56 | X144B49 | 2 | 1.000 | 0.000 | 2a | 11.5 | 0 |
| 57 | X14B98 | 2 | 0.997 | 0.003 | 2a | 10.83 | 0 |
| 58 | X150B81 | 2 | 0.995 | 0.005 | 2a | 11.42 | 0 |
| 59 | X151B84 | 2 | 0.998 | 0.002 | 2a | 11.42 | 0 |
| 60 | X152B99 | 2 | 1.000 | 0.000 | 2a | 2.08 | 0 |
| 61 | X158B84 | 2 | 1.000 | 0.000 | 2a | 4.67 | 1 |
| 62 | X15C94 | 2 | 1.000 | 0.000 | 2a | 4.42 | 0 |
| 63 | X161B31 | 2 | 0.999 | 0.001 | 2a | 11.42 | 0 |
| 64 | X168B51 | 2 | 1.000 | 0.000 | 2a | 5.33 | 0 |
| 65 | X169B79 | 2 | 0.996 | 0.004 | 2a | 11.33 | 0 |
| 66 | X16C97 | 2 | 0.997 | 0.003 | 2a | 3.58 | 1 |
| 67 | X170B15 | 2 | 0.913 | 0.087 | 2a | 4.08 | 1 |
| 68 | X175B72 | 2 | 0.760 | 0.240 | 2a | 0 | 1 |
| 69 | X176B74 | 2 | 1.000 | 0.000 | 2a | 6 | 0 |
| 70 | X178B74 | 2 | 0.996 | 0.004 | 2a | 7.42 | 0 |
| 71 | X179B28 | 2 | 0.999 | 0.001 | 2a | 2.33 | 1 |
| 72 | X17C40 | 2 | 0.999 | 0.001 | 2a | 1.92 | 0 |
| 73 | X183B75 | 2 | 0.045 | 0.955 | 2b | 7 | 1 |
| 74 | X18C56 | 2 | 0.997 | 0.003 | 2a | 10.75 | 0 |
| 75 | X197B95 | 2 | 0.072 | 0.928 | 2b | 10.92 | 0 |
| 76 | X198B90 | 2 | 0.999 | 0.001 | 2a | 10.92 | 0 |
| 77 | X199B55 | 2 | 0.074 | 0.926 | 2b | 10.92 | 0 |
| 78 | X201B68 | 2 | 0.998 | 0.002 | 2a | 10.92 | 0 |
| 79 | X202B44 | 2 | 1.000 | 0.000 | 2a | 10.83 | 0 |
| 80 | X203B49 | 2 | 1.000 | 0.000 | 2a | 10.83 | 0 |
| 81 | X206C05 | 2 | 0.994 | 0.006 | 2a | 6.42 | 0 |
| 82 | X278C80 | 2 | 0.990 | 0.010 | 2a | 10.25 | 0 |
| 83 | X77A50 | 2 | 0.989 | 0.011 | 2a | 1.08 | 1 |
| 84 | X87A79 | 2 | 0.927 | 0.073 | 2a | 12.08 | 0 |
| 85 | X188B13 | 2 | 0.934 | 0.066 | 2a | 11 | 0 |
| 86 | X193B72 | 2 | 0.400 | 0.600 | 2b | 10.92 | 0 |
| 87 | X213C36 | 2 | 0.041 | 0.959 | 2b | 10.08 | 0 |
| 88 | X112B55 | 2 | 0.000 | 1.000 | 2b | 0.92 | 1 |
| 89 | X221C14 | 2 | 0.363 | 0.637 | 2b | 3 | 1 |
| 90 | X225C52 | 2 | 0.000 | 1.000 | 2b | 10.75 | 0 |
| 91 | X230C47 | 2 | 0.000 | 1.000 | 2b | 0.5 | 1 |
| 92 | X237C56 | 2 | 0.001 | 0.999 | 2b | 10.67 | 0 |
| 93 | X23C52 | 2 | 0.000 | 1.000 | 2b | 8.5 | 1 |
| 94 | X240C54 | 2 | 0.050 | 0.950 | 2b | 2.42 | 1 |
| 95 | X242C21 | 2 | 0.099 | 0.901 | 2b | 2.17 | 1 |
| 96 | X245C22 | 2 | 0.005 | 0.995 | 2b | 0 | 1 |
| 97 | X256C45 | 2 | 0.000 | 1.000 | 2b | 1.25 | 1 |
| 98 | X120B73 | 2 | 0.000 | 1.000 | 2b | 11.58 | 0 |
| 99 | X260C91 | 2 | 0.005 | 0.995 | 2b | 10.42 | 0 |
| 100 | X268C87 | 2 | 0.000 | 1.000 | 2b | 10.33 | 0 |
| 101 | X308C93 | 2 | 0.000 | 1.000 | 2b | 2.25 | 1 |
| 102 | X35C29 | 2 | 0.003 | 0.997 | 2b | 2.42 | 1 |
| 103 | X44A53 | 2 | 0.996 | 0.004 | 2a | 12.75 | 0 |
| 104 | X47A87 | 2 | 0.000 | 1.000 | 2b | 9.58 | 1 |
| 105 | X53A06 | 2 | 0.038 | 0.962 | 2b | 2.58 | 1 |
| 106 | X58A50 | 2 | 0.000 | 1.000 | 2b | 0.42 | 1 |
| 107 | X5B97 | 2 | 0.000 | 1.000 | 2b | 0.75 | 1 |
| 108 | X134B33 | 2 | 0.000 | 1.000 | 2b | 2 | 1 |
| 109 | X64A59 | 2 | 0.001 | 0.999 | 2b | 12.42 | 0 |
| 110 | X75A01 | 2 | 0.001 | 0.999 | 2b | 3.58 | 1 |
| 111 | X84A44 | 2 | 0.000 | 1.000 | 2b | 12.17 | 0 |
| 112 | X86A40 | 2 | 0.000 | 1.000 | 2b | 12.17 | 0 |
| 113 | X88A67 | 2 | 0.000 | 1.000 | 2b | 4.25 | 1 |
| 114 | X145B10 | 2 | 0.000 | 1.000 | 2b | 11.42 | 0 |
| 115 | X154B42 | 2 | 0.000 | 1.000 | 2b | 3.42 | 1 |
| 116 | X159B47 | 2 | 0.010 | 0.990 | 2b | 6.5 | 1 |
| 117 | X164B81 | 2 | 0.000 | 1.000 | 2b | 11.33 | 0 |
| 118 | X165B72 | 2 | 0.304 | 0.696 | 2b | 1.5 | 1 |
| 119 | X166B79 | 2 | 0.064 | 0.936 | 2b | 11.33 | 0 |
| 120 | X171B77 | 2 | 0.000 | 1.000 | 2b | 1.75 | 1 |
| 121 | X186B22 | 2 | 0.002 | 0.998 | 2b | 0.17 | 1 |
| 122 | X187B36 | 2 | 0.000 | 1.000 | 2b | 0 | 1 |
| 123 | X189B83 | 2 | 0.000 | 1.000 | 2b | 11 | 0 |
| 124 | X191B79 | 2 | 0.000 | 1.000 | 2b | 4.42 | 1 |
| 125 | X110B34 | 2 | 0.000 | 1.000 | 2b | 11.67 | 0 |
| 126 | X208C06 | 2 | 0.000 | 1.000 | 2b | 0.08 | 0 |

APPENDIX 7A
SWS Classifier 3

| | UGID(build | | | | | |
|---|---|---|---|---|---|---|
| Order | #183) | UnigeneName | GeneSymbol | GenbankAcc | Affi ID | Cut-off |
| 1 | Hs.9329 | TPX2, microtubule- | TPX2 | AF098158 | A.210052_s_at | 8.7748 |
| | | associated protein | | | | |
| | | homolog (Xenopus | | | | |
| | | laevis) | | | | |
| 2 | Hs.344037 | Protein regulator of | PRC1 | NM_003981 | A.218009_s_at | 8.2222 |
| | | cytokinesis 1 | | | | |
| 3 | Hs.292511 | Neuro-oncological | NOVA1 | NM_002515 | A.205794_s_at | 6.7387 |
| | | ventral antigen 1 | | | | |
| 4 | Hs.155223 | Stanniocalcin 2 | STC2 | AI435828 | A.203438_at | 8.0766 |
| 5 | Hs.437351 | Cold inducible RNA | CIRBP | AL565767 | 8.225191_at | 8.2308 |
| | | binding protein | | | | |
| 6 | Hs.24395 | Chemokine (C-X-C | CXCL14 | NM_004887 | A.218002_s_at | 7.086 |
| | | motif) ligand 14 | | | | |
| 7 | Hs.435861 | Signal peptide, CUB | SCUBE2 | AI424243 | A.219197_s_at | 7.2545 |
| | | domain, EGF-like 2 | | | | |

APPENDIX 7B
SWS Classifier 3: Classifier Accuracy
Accuracy
G1 = 67/68 (98.5%)
G3 = 51/55 (92.7%)

| | | Histo- | | | |
|---|---|---|---|---|---|
| | Patients | logic | Probability | Probability | Predicted |
| Number | ID | grade | for G1 | for G3 | grade |
| 1 | X100B08 | 1 | 0.990 | 0.010 | 1 |
| 2 | X209C10 | 1 | 0.818 | 0.182 | 1 |
| 3 | X21C28 | 1 | 0.964 | 0.036 | 1 |
| 4 | X220C70 | 1 | 0.990 | 0.010 | 1 |
| 5 | X224C93 | 1 | 0.587 | 0.413 | 1 |
| 6 | X227C50 | 1 | 1.000 | 0.000 | 1 |
| 7 | X229C44 | 1 | 0.981 | 0.019 | 1 |
| 8 | X231C80 | 1 | 1.000 | 0.000 | 1 |
| 9 | X233C91 | 1 | 0.990 | 0.010 | 1 |
| 10 | X235C20 | 1 | 0.976 | 0.024 | 1 |
| 11 | X236C55 | 1 | 1.000 | 0.000 | 1 |
| 12 | X114B68 | 1 | 0.990 | 0.010 | 1 |
| 13 | X243C70 | 1 | 0.818 | 0.182 | 1 |
| 14 | X246C75 | 1 | 0.990 | 0.010 | 1 |
| 15 | X248C91 | 1 | 0.907 | 0.093 | 1 |
| 16 | X253C20 | 1 | 1.000 | 0.000 | 1 |
| 17 | X259C74 | 1 | 0.990 | 0.010 | 1 |
| 18 | X261C94 | 1 | 1.000 | 0.000 | 1 |
| 19 | X262C85 | 1 | 1.000 | 0.000 | 1 |
| 20 | X263C82 | 1 | 1.000 | 0.000 | 1 |
| 21 | X266C51 | 1 | 1.000 | 0.000 | 1 |
| 22 | X267C04 | 1 | 0.907 | 0.093 | 1 |
| 23 | X282C51 | 1 | 0.907 | 0.093 | 1 |
| 24 | X284C63 | 1 | 1.000 | 0.000 | 1 |
| 25 | X289C75 | 1 | 1.000 | 0.000 | 1 |
| 26 | X28C76 | 1 | 1.000 | 0.000 | 1 |
| 27 | X294C04 | 1 | 0.587 | 0.413 | 1 |
| 28 | X309C49 | 1 | 0.015 | 0.985 | 3 |
| 29 | X316C65 | 1 | 0.990 | 0.010 | 1 |
| 30 | X128B48 | 1 | 1.000 | 0.000 | 1 |
| 31 | X33C30 | 1 | 1.000 | 0.000 | 1 |
| 32 | X39C24 | 1 | 0.907 | 0.093 | 1 |
| 33 | X42C57 | 1 | 0.983 | 0.017 | 1 |
| 34 | X45A96 | 1 | 0.765 | 0.235 | 1 |
| 35 | X48A46 | 1 | 1.000 | 0.000 | 1 |
| 36 | X49A07 | 1 | 0.990 | 0.010 | 1 |
| 37 | X52A90 | 1 | 0.990 | 0.010 | 1 |
| 38 | X61A53 | 1 | 1.000 | 0.000 | 1 |
| 39 | X65A68 | 1 | 0.827 | 0.173 | 1 |
| 40 | X6B85 | 1 | 0.529 | 0.471 | 1 |
| 41 | X72A92 | 1 | 0.907 | 0.093 | 1 |
| 42 | X135B40 | 1 | 0.907 | 0.093 | 1 |
| 43 | X74A63 | 1 | 0.529 | 0.471 | 1 |
| 44 | X83A37 | 1 | 0.976 | 0.024 | 1 |
| 45 | X8B87 | 1 | 0.910 | 0.090 | 1 |
| 46 | X99A50 | 1 | 0.531 | 0.469 | 1 |
| 47 | X138B34 | 1 | 1.000 | 0.000 | 1 |
| 48 | X155B52 | 1 | 1.000 | 0.000 | 1 |
| 49 | X156B01 | 1 | 1.000 | 0.000 | 1 |
| 50 | X160B16 | 1 | 1.000 | 0.000 | 1 |
| 51 | X163B27 | 1 | 1.000 | 0.000 | 1 |
| 52 | X105B13 | 1 | 0.907 | 0.093 | 1 |
| 53 | X173B43 | 1 | 0.910 | 0.090 | 1 |
| 54 | X174B41 | 1 | 1.000 | 0.000 | 1 |
| 55 | X177B67 | 1 | 0.990 | 0.010 | 1 |
| 56 | X106B55 | 1 | 0.990 | 0.010 | 1 |
| 57 | X180B38 | 1 | 0.990 | 0.010 | 1 |
| 58 | X181B70 | 1 | 0.990 | 0.010 | 1 |
| 59 | X184B38 | 1 | 0.907 | 0.093 | 1 |
| 60 | X185B44 | 1 | 1.000 | 0.000 | 1 |
| 61 | X10B88 | 1 | 0.739 | 0.261 | 1 |
| 62 | X192B69 | 1 | 1.000 | 0.000 | 1 |
| 63 | X195B75 | 1 | 1.000 | 0.000 | 1 |
| 64 | X196B81 | 1 | 1.000 | 0.000 | 1 |
| 65 | X19C33 | 1 | 0.587 | 0.413 | 1 |
| 66 | X204B85 | 1 | 1.000 | 0.000 | 1 |
| 67 | X205B99 | 1 | 0.827 | 0.173 | 1 |
| 68 | X207C08 | 1 | 1.000 | 0.000 | 1 |
| 69 | X111B51 | 3 | 0.006 | 0.994 | 3 |
| 70 | X222C26 | 3 | 0.623 | 0.377 | 1 |
| 71 | X226C06 | 3 | 0.005 | 0.995 | 3 |
| 72 | X113B11 | 3 | 0.093 | 0.907 | 3 |
| 73 | X232C58 | 3 | 0.016 | 0.984 | 3 |
| 74 | X234C15 | 3 | 0.005 | 0.995 | 3 |
| 75 | X238C87 | 3 | 0.205 | 0.795 | 3 |
| 76 | X241C01 | 3 | 0.009 | 0.991 | 3 |
| 77 | X249C42 | 3 | 0.002 | 0.998 | 3 |
| 78 | X250C78 | 3 | 0.016 | 0.984 | 3 |
| 79 | X252C64 | 3 | 0.016 | 0.984 | 3 |
| 80 | X269C68 | 3 | 0.002 | 0.998 | 3 |
| 81 | X26C23 | 3 | 0.129 | 0.871 | 3 |
| 82 | X270C93 | 3 | 0.000 | 1.000 | 3 |
| 83 | X271C71 | 3 | 0.002 | 0.998 | 3 |
| 84 | X279C61 | 3 | 0.002 | 0.998 | 3 |
| 85 | X287C67 | 3 | 0.005 | 0.995 | 3 |
| 86 | X291C17 | 3 | 0.006 | 0.994 | 3 |
| 87 | X127B00 | 3 | 0.016 | 0.984 | 3 |
| 88 | X303C36 | 3 | 0.005 | 0.995 | 3 |
| 89 | X304C89 | 3 | 0.899 | 0.101 | 1 |
| 90 | X311A27 | 3 | 0.045 | 0.955 | 3 |
| 91 | X313A87 | 3 | 0.002 | 0.998 | 3 |
| 92 | X314B55 | 3 | 0.002 | 0.998 | 3 |
| 93 | X101B88 | 3 | 0.009 | 0.991 | 3 |
| 94 | X37C06 | 3 | 0.006 | 0.994 | 3 |
| 95 | X46A25 | 3 | 0.057 | 0.943 | 3 |
| 96 | X131B79 | 3 | 0.075 | 0.925 | 3 |
| 97 | X54A09 | 3 | 0.000 | 1.000 | 3 |
| 98 | X55A79 | 3 | 0.028 | 0.972 | 3 |
| 99 | X62A02 | 3 | 0.006 | 0.994 | 3 |
| 100 | X66A84 | 3 | 0.002 | 0.998 | 3 |
| 101 | X67A43 | 3 | 0.002 | 0.998 | 3 |
| 102 | X69A93 | 3 | 0.136 | 0.864 | 3 |
| 103 | X70A79 | 3 | 0.005 | 0.995 | 3 |
| 104 | X73A01 | 3 | 0.194 | 0.806 | 3 |
| 105 | X76A44 | 3 | 0.022 | 0.978 | 3 |
| 106 | X79A35 | 3 | 0.006 | 0.994 | 3 |
| 107 | X82A83 | 3 | 0.062 | 0.938 | 3 |
| 108 | X89A64 | 3 | 0.005 | 0.995 | 3 |
| 109 | X90A63 | 3 | 0.002 | 0.998 | 3 |
| 110 | X139B03 | 3 | 0.022 | 0.978 | 3 |
| 111 | X102B06 | 3 | 0.006 | 0.994 | 3 |
| 112 | X142B05 | 3 | 0.005 | 0.995 | 3 |
| 113 | X143B81 | 3 | 0.002 | 0.998 | 3 |
| 114 | X146B39 | 3 | 0.002 | 0.998 | 3 |
| 115 | X147B19 | 3 | 0.016 | 0.984 | 3 |
| 116 | X103B41 | 3 | 0.002 | 0.998 | 3 |
| 117 | X153B09 | 3 | 0.002 | 0.998 | 3 |
| 118 | X104B91 | 3 | 0.119 | 0.881 | 3 |
| 119 | X162B98 | 3 | 0.623 | 0.377 | 1 |
| 120 | X172B19 | 3 | 0.055 | 0.945 | 3 |
| 121 | X182B43 | 3 | 0.002 | 0.998 | 3 |
| 122 | X194B60 | 3 | 0.002 | 0.998 | 3 |
| 123 | X200B47 | 3 | 0.979 | 0.021 | 1 |

APPENDIX 7C
SWS Classifier 3: G2a-G2b Prediction Validation
# Cox PH test summary (Baseline group 1)
coef exp(coef) se(coef)
z p

| | | | | |
|---|---|---|---|---|
| group2b | 1.05 | 2.85 | 0.292 | 3.58 |
| 0.00035 | | | | |

Likelihood ratio test = 12.2 on 1 df, p = 0.000485 n = 126

# Survival fit
summaries

n events rmean se(rmean) median 0.95LCL

| | | | | |
|---|---|---|---|---|
| 0.95UCL | | | | |
| group2a = 87 | 24 | 10.05 | 0.482 | Inf |
| Inf | Inf | | | |
| group2b = 39 | 23 | 6.61 | 0.844 | 6.5 |
| 2.42 | Inf | | | |

* DFS Event defined as any type of recurrence or death because
of breast cancer, whichever comes first

| | | | | | Pre- | | |
|---|---|---|---|---|---|---|---|
| | | | | | dicted | | |
| | | | Prob- | Prob- | grade | | |
| | | Histo- | ability | ability | (2a- | | DFS |
| | Patient | logic | for | for | G2a, 2b- | DFS | E- |
| Number | ID | grade | G2a | G2b | G2b) | TIME | vent |
| 1 | X210C72 | 2 | 0.012 | 0.988 | 2b | 0.5 | 1 |
| 2 | X211C88 | 2 | 0.999 | 0.001 | 2a | 1.5 | 0 |
| 3 | X212C21 | 2 | 1.000 | 0.000 | 2a | 3.75 | 1 |
| 4 | X213C36 | 2 | 0.001 | 0.999 | 2b | 10.08 | 0 |
| 5 | X216C61 | 2 | 0.820 | 0.180 | 2a | 10.75 | 0 |
| 6 | X217C79 | 2 | 0.999 | 0.001 | 2a | 10.75 | 0 |
| 7 | X218C29 | 2 | 0.996 | 0.004 | 2a | 10.75 | 0 |
| 8 | X112B55 | 2 | 0.418 | 0.582 | 2b | 0.92 | 1 |
| 9 | X221C14 | 2 | 0.901 | 0.099 | 2a | 3 | 1 |
| 10 | X223C51 | 2 | 0.999 | 0.001 | 2a | 8.42 | 0 |
| 11 | X225C52 | 2 | 0.001 | 0.999 | 2b | 10.75 | 0 |
| 12 | X22C62 | 2 | 0.901 | 0.099 | 2a | 4.83 | 0 |
| 13 | X230C47 | 2 | 0.000 | 1.000 | 2b | 0.5 | 1 |
| 14 | X237C56 | 2 | 0.000 | 1.000 | 2b | 10.67 | 0 |
| 15 | X23C52 | 2 | 0.001 | 0.999 | 2b | 8.5 | 1 |
| 16 | X240C54 | 2 | 0.001 | 0.999 | 2b | 2.42 | 1 |
| 17 | X242C21 | 2 | 0.634 | 0.366 | 2a | 2.17 | 1 |
| 18 | X244C89 | 2 | 0.001 | 0.999 | 2b | 7.25 | 1 |
| 19 | X245C22 | 2 | 0.004 | 0.996 | 2b | 0 | 1 |
| 20 | X247C76 | 2 | 0.996 | 0.004 | 2a | 10.5 | 0 |
| 21 | X11B47 | 2 | 0.640 | 0.360 | 2a | 7.42 | 0 |
| 22 | X24C30 | 2 | 0.999 | 0.001 | 2a | 10.67 | 0 |
| 23 | X251C14 | 2 | 0.999 | 0.001 | 2a | 10.5 | 0 |
| 24 | X254C80 | 2 | 0.999 | 0.001 | 2a | 10.5 | 0 |
| 25 | X255C06 | 2 | 0.744 | 0.256 | 2a | 10.5 | 0 |
| 26 | X256C45 | 2 | 0.000 | 1.000 | 2b | 1.25 | 1 |
| 27 | X120B73 | 2 | 0.000 | 1.000 | 2b | 11.58 | 0 |
| 28 | X257C87 | 2 | 0.901 | 0.099 | 2a | 10.5 | 0 |
| 29 | X258C21 | 2 | 0.999 | 0.001 | 2a | 5.75 | 1 |
| 30 | X260C91 | 2 | 0.640 | 0.360 | 2a | 10.42 | 0 |
| 31 | X265C40 | 2 | 0.578 | 0.422 | 2a | 10.42 | 0 |
| 32 | X122B81 | 2 | 0.999 | 0.001 | 2a | 11.17 | 0 |
| 33 | X268C87 | 2 | 0.000 | 1.000 | 2b | 10.33 | 0 |
| 34 | X272C88 | 2 | 0.998 | 0.002 | 2a | 10.33 | 0 |
| 35 | X274C81 | 2 | 0.820 | 0.180 | 2a | 10.33 | 0 |
| 36 | X275C70 | 2 | 0.999 | 0.001 | 2a | 10.25 | 0 |
| 37 | X277C64 | 2 | 0.999 | 0.001 | 2a | 8.58 | 0 |
| 38 | X124B25 | 2 | 0.640 | 0.360 | 2a | 5 | 1 |
| 39 | X278C80 | 2 | 0.002 | 0.998 | 2b | 10.25 | 0 |
| 40 | X27C82 | 2 | 0.550 | 0.450 | 2a | 6.83 | 0 |
| 41 | X280C43 | 2 | 1.000 | 0.000 | 2a | 1 | 1 |
| 42 | X286C91 | 2 | 1.000 | 0.000 | 2a | 10 | 0 |
| 43 | X288C57 | 2 | 0.820 | 0.180 | 2a | 10 | 0 |
| 44 | X290C91 | 2 | 1.000 | 0.000 | 2a | 10 | 0 |
| 45 | X292C66 | 2 | 0.999 | 0.001 | 2a | 10 | 0 |
| 46 | X296C95 | 2 | 1.000 | 0.000 | 2a | 9.92 | 0 |
| 47 | X297C26 | 2 | 0.820 | 0.180 | 2a | 9.92 | 0 |
| 48 | X298C47 | 2 | 0.999 | 0.001 | 2a | 6.5 | 1 |
| 49 | X301C66 | 2 | 0.640 | 0.360 | 2a | 9.92 | 0 |
| 50 | X307C50 | 2 | 0.744 | 0.256 | 2a | 9.83 | 0 |
| 51 | X308C93 | 2 | 0.000 | 1.000 | 2b | 2.25 | 1 |
| 52 | X34C80 | 2 | 0.820 | 0.180 | 2a | 10.17 | 0 |
| 53 | X35C29 | 2 | 0.999 | 0.001 | 2a | 2.42 | 1 |
| 54 | X36C17 | 2 | 0.901 | 0.099 | 2a | 10.08 | 0 |
| 55 | X40C57 | 2 | 0.999 | 0.001 | 2a | 10 | 0 |
| 56 | X41C65 | 2 | 1.000 | 0.000 | 2a | 9.92 | 0 |
| 57 | X130B92 | 2 | 1.000 | 0.000 | 2a | 4.42 | 1 |
| 58 | X43C47 | 2 | 0.574 | 0.426 | 2a | 9.92 | 0 |
| 59 | X44A53 | 2 | 1.000 | 0.000 | 2a | 12.75 | 0 |
| 60 | X47A87 | 2 | 0.000 | 1.000 | 2b | 9.58 | 1 |
| 61 | X50A91 | 2 | 0.012 | 0.988 | 2b | 9.08 | 1 |
| 62 | X51A98 | 2 | 0.998 | 0.002 | 2a | 12.67 | 0 |
| 63 | X53A06 | 2 | 1.000 | 0.000 | 2a | 2.58 | 1 |
| 64 | X56A94 | 2 | 0.998 | 0.002 | 2a | 1.08 | 1 |
| 65 | X58A50 | 2 | 0.000 | 1.000 | 2b | 0.42 | 1 |
| 66 | X5B97 | 2 | 0.000 | 1.000 | 2b | 0.75 | 1 |
| 67 | X60A05 | 2 | 1.000 | 0.000 | 2a | 0.67 | 1 |
| 68 | X134B33 | 2 | 0.001 | 0.999 | 2b | 2 | 1 |
| 69 | X63A62 | 2 | 0.999 | 0.001 | 2a | 0.17 | 1 |
| 70 | X64A59 | 2 | 0.001 | 0.999 | 2b | 12.42 | 0 |
| 71 | X75A01 | 2 | 0.999 | 0.001 | 2a | 3.58 | 1 |
| 72 | X77A50 | 2 | 0.391 | 0.609 | 2b | 1.08 | 1 |
| 73 | X7B96 | 2 | 0.391 | 0.609 | 2b | 2.42 | 1 |
| 74 | X84A44 | 2 | 0.002 | 0.998 | 2b | 12.17 | 0 |
| 75 | X136B04 | 2 | 0.012 | 0.988 | 2b | 2.42 | 1 |
| 76 | X85A03 | 2 | 0.012 | 0.988 | 2b | 2.08 | 0 |
| 77 | X86A40 | 2 | 0.000 | 1.000 | 2b | 12.17 | 0 |
| 78 | X87A79 | 2 | 0.820 | 0.180 | 2a | 12.08 | 0 |
| 79 | X88A67 | 2 | 0.574 | 0.426 | 2a | 4.25 | 1 |
| 80 | X94A16 | 2 | 0.999 | 0.001 | 2a | 11.08 | 0 |
| 81 | X96A21 | 2 | 0.020 | 0.980 | 2b | 0.08 | 1 |
| 82 | X137B88 | 2 | 0.640 | 0.360 | 2a | 10.5 | 1 |
| 83 | X9B52 | 2 | 0.999 | 0.001 | 2a | 11.33 | 0 |
| 84 | X13B79 | 2 | 0.999 | 0.001 | 2a | 10.83 | 0 |
| 85 | X140B91 | 2 | 0.901 | 0.099 | 2a | 11.5 | 0 |
| 86 | X144B49 | 2 | 0.796 | 0.204 | 2a | 11.5 | 0 |
| 87 | X145B10 | 2 | 0.000 | 1.000 | 2b | 11.42 | 0 |
| 88 | X14B98 | 2 | 0.999 | 0.001 | 2a | 10.83 | 0 |
| 89 | X150B81 | 2 | 1.000 | 0.000 | 2a | 11.42 | 0 |
| 90 | X151B84 | 2 | 1.000 | 0.000 | 2a | 11.42 | 0 |
| 91 | X152B99 | 2 | 1.000 | 0.000 | 2a | 2.08 | 0 |
| 92 | X154B42 | 2 | 0.099 | 0.901 | 2b | 3.42 | 1 |
| 93 | X158B84 | 2 | 0.999 | 0.001 | 2a | 4.67 | 1 |
| 94 | X159B47 | 2 | 0.002 | 0.998 | 2b | 6.5 | 1 |
| 95 | X15C94 | 2 | 1.000 | 0.000 | 2a | 4.42 | 0 |
| 96 | X161B31 | 2 | 1.000 | 0.000 | 2a | 11.42 | 0 |
| 97 | X164B81 | 2 | 0.000 | 1.000 | 2b | 11.33 | 0 |
| 98 | X165B72 | 2 | 0.944 | 0.056 | 2a | 1.5 | 1 |
| 99 | X166B79 | 2 | 0.980 | 0.020 | 2a | 11.33 | 0 |
| 100 | X168B51 | 2 | 0.800 | 0.200 | 2a | 5.33 | 0 |
| 101 | X169B79 | 2 | 0.995 | 0.005 | 2a | 11.33 | 0 |
| 102 | X16C97 | 2 | 1.000 | 0.000 | 2a | 3.58 | 1 |
| 103 | X170B15 | 2 | 0.999 | 0.001 | 2a | 4.08 | 1 |
| 104 | X171B77 | 2 | 0.000 | 1.000 | 2b | 1.75 | 1 |
| 105 | X175B72 | 2 | 0.901 | 0.099 | 2a | 0 | 1 |
| 106 | X176B74 | 2 | 1.000 | 0.000 | 2a | 6 | 0 |
| 107 | X178B74 | 2 | 1.000 | 0.000 | 2a | 7.42 | 0 |
| 108 | X179B28 | 2 | 0.999 | 0.001 | 2a | 2.33 | 1 |
| 109 | X17C40 | 2 | 0.999 | 0.001 | 2a | 1.92 | 0 |
| 110 | X183B75 | 2 | 0.820 | 0.180 | 2a | 7 | 1 |
| 111 | X186B22 | 2 | 0.786 | 0.214 | 2a | 0.17 | 1 |
| 112 | X187B36 | 2 | 0.000 | 1.000 | 2b | 0 | 1 |
| 113 | X188B13 | 2 | 0.999 | 0.001 | 2a | 11 | 0 |
| 114 | X189B83 | 2 | 0.000 | 1.000 | 2b | 11 | 0 |
| 115 | X18C56 | 2 | 1.000 | 0.000 | 2a | 10.75 | 0 |
| 116 | X191B79 | 2 | 0.099 | 0.901 | 2b | 4.42 | 1 |
| 117 | X193B72 | 2 | 0.640 | 0.360 | 2a | 10.92 | 0 |
| 118 | X197B95 | 2 | 0.297 | 0.703 | 2b | 10.92 | 0 |
| 119 | X198B90 | 2 | 0.901 | 0.099 | 2a | 10.92 | 0 |
| 120 | X199B55 | 2 | 0.820 | 0.180 | 2a | 10.92 | 0 |
| 121 | X110B34 | 2 | 0.000 | 1.000 | 2b | 11.67 | 0 |
| 122 | X201B68 | 2 | 0.999 | 0.001 | 2a | 10.92 | 0 |
| 123 | X202B44 | 2 | 0.999 | 0.001 | 2a | 10.83 | 0 |
| 124 | X203B49 | 2 | 1.000 | 0.000 | 2a | 10.83 | 0 |
| 125 | X206C05 | 2 | 1.000 | 0.000 | 2a | 6.42 | 0 |
| 126 | X208C06 | 2 | 0.136 | 0.864 | 2b | 0.08 | 0 |

APPENDIX 8A
SWS Classifier 4

| | UGID(build | | | | | |
|---|---|---|---|---|---|---|
| Order | #183) | UnigeneName | GeneSymbol | GenbankAcc | Affi ID | Cut-off |
| 1 | Hs.48855 | cell division cycle | CDCA8 | BC001651 | A.221520_s_at | 5.5046 |
| | | associated 8 | | | | |
| 2 | Hs.75573 | centromere protein | CENPE | NM_001813 | A.205046_at | 5.2115 |
| | | E, 312 kDa | | | | |
| 3 | Hs.552 | steroid-5-alpha- | SRD5A1 | BC006373 | A.211056_s_at | 6.9192 |
| | | reductase, alpha | | | | |
| | | polypeptide 1 (3- | | | | |
| | | oxo-5 alpha-steroid | | | | |
| | | delta 4- | | | | |
| | | dehydrogenase | | | | |
| | | alpha 1) | | | | |
| 4 | Hs.101174 | microtubule- | MAPT | NM_016835 | A.203929_s_at | 4.8246 |
| | | associated protein | | | | |
| | | tau | | | | |
| 5 | Hs.164018 | leucine zipper | FKSG14 | BC005400 | B.222848_at | 6.1846 |
| | | protein FKSG14 | | | | |
| 6 | acc_R38110 | N.A. | R38110 | B.240112_at | 6.2557 | |
| 7 | Hs.325650 | EH-domain | EHD2 | AI417917 | A.221870_at | 7.6677 |
| | | containing 2 | | | | |

APPENDIX 8B
SWS Classifier 4: Classifier Accuracy
Accuracy
G1 = 67/68
(98.5%)
G3 = 52/55
(94.5%)

| | | | | | Predicted |
|---|---|---|---|---|---|
| Num- | Patients | Histologic | Probability | Probability | grade (G1 |
| ber | ID | grade | for G 1 | for G3 | or G3) |
| 1 | X100B08 | 1 | 1.000 | 0 | 1 |
| 2 | X209C10 | 1 | 0.992 | 0.008 | 1 |
| 3 | X21C28 | 1 | 0.992 | 0.008 | 1 |
| 4 | X220C70 | 1 | 1.000 | 0.000 | 1 |
| 5 | X224C93 | 1 | 0.962 | 0.038 | 1 |
| 6 | X227C50 | 1 | 1.000 | 0.000 | 1 |
| 7 | X229C44 | 1 | 0.962 | 0.038 | 1 |
| 8 | X231C80 | 1 | 0.742 | 0.258 | 1 |
| 9 | X233C91 | 1 | 1.000 | 0.000 | 1 |
| 10 | X235C20 | 1 | 0.633 | 0.367 | 1 |
| 11 | X236C55 | 1 | 0.986 | 0.014 | 1 |
| 12 | X114B68 | 1 | 0.852 | 0.148 | 1 |
| 13 | X243C70 | 1 | 1.000 | 0.000 | 1 |
| 14 | X246C75 | 1 | 1.000 | 0.000 | 1 |
| 15 | X248C91 | 1 | 1.000 | 0.000 | 1 |
| 16 | X253C20 | 1 | 1.000 | 0.000 | 1 |
| 17 | X259C74 | 1 | 1.000 | 0.000 | 1 |
| 18 | X261C94 | 1 | 1.000 | 0.000 | 1 |
| 19 | X262C85 | 1 | 0.992 | 0.008 | 1 |
| 20 | X263C82 | 1 | 1.000 | 0.000 | 1 |
| 21 | X266C51 | 1 | 1.000 | 0.000 | 1 |
| 22 | X267C04 | 1 | 0.633 | 0.367 | 1 |
| 23 | X282C51 | 1 | 0.962 | 0.038 | 1 |
| 24 | X284C63 | 1 | 0.992 | 0.008 | 1 |
| 25 | X289C75 | 1 | 0.969 | 0.031 | 1 |
| 26 | X28C76 | 1 | 0.992 | 0.008 | 1 |
| 27 | X294C04 | 1 | 0.667 | 0.333 | 1 |
| 28 | X309C49 | 1 | 0.531 | 0.469 | 1 |
| 29 | X316C65 | 1 | 1.000 | 0.000 | 1 |
| 30 | X128B48 | 1 | 1.000 | 0.000 | 1 |
| 31 | X33C30 | 1 | 0.992 | 0.008 | 1 |
| 32 | X39C24 | 1 | 0.992 | 0.008 | 1 |
| 33 | X42C57 | 1 | 1.000 | 0.000 | 1 |
| 34 | X45A96 | 1 | 0.703 | 0.297 | 1 |
| 35 | X48A46 | 1 | 1.000 | 0.000 | 1 |
| 36 | X49A07 | 1 | 0.992 | 0.008 | 1 |
| 37 | X52A90 | 1 | 0.992 | 0.008 | 1 |
| 38 | X61A53 | 1 | 0.742 | 0.258 | 1 |
| 39 | X65A68 | 1 | 0.975 | 0.025 | 1 |
| 40 | X6B85 | 1 | 0.633 | 0.367 | 1 |
| 41 | X72A92 | 1 | 0.992 | 0.008 | 1 |
| 42 | X135B40 | 1 | 1.000 | 0.000 | 1 |
| 43 | X74A63 | 1 | 0.852 | 0.148 | 1 |
| 44 | X83A37 | 1 | 0.852 | 0.148 | 1 |
| 45 | X8B87 | 1 | 1.000 | 0.000 | 1 |
| 46 | X99A50 | 1 | 0.738 | 0.262 | 1 |
| 47 | X138B34 | 1 | 0.992 | 0.008 | 1 |
| 48 | X155B52 | 1 | 1.000 | 0.000 | 1 |
| 49 | X156B01 | 1 | 1.000 | 0.000 | 1 |
| 50 | X160B16 | 1 | 0.992 | 0.008 | 1 |
| 51 | X163B27 | 1 | 0.992 | 0.008 | 1 |
| 52 | X105B13 | 1 | 0.939 | 0.061 | 1 |
| 53 | X173B43 | 1 | 1.000 | 0.000 | 1 |
| 54 | X174B41 | 1 | 0.986 | 0.014 | 1 |
| 55 | X177B67 | 1 | 1.000 | 0.000 | 1 |
| 56 | X106B55 | 1 | 1.000 | 0.000 | 1 |
| 57 | X180B38 | 1 | 1.000 | 0.000 | 1 |
| 58 | X181B70 | 1 | 0.947 | 0.053 | 1 |
| 59 | X184B38 | 1 | 0.852 | 0.148 | 1 |
| 60 | X185B44 | 1 | 0.992 | 0.008 | 1 |
| 61 | X10B88 | 1 | 0.463 | 0.537 | 3 |
| 62 | X192B69 | 1 | 0.992 | 0.008 | 1 |
| 63 | X195B75 | 1 | 1.000 | 0.000 | 1 |
| 64 | X196B81 | 1 | 0.742 | 0.258 | 1 |
| 65 | X19C33 | 1 | 0.962 | 0.038 | 1 |
| 66 | X204B85 | 1 | 1.000 | 0.000 | 1 |
| 67 | X205B99 | 1 | 0.633 | 0.367 | 1 |
| 68 | X207C08 | 1 | 1.000 | 0.000 | 1 |
| 69 | X111B51 | 3 | 0.027 | 0.973 | 3 |
| 70 | X222C26 | 3 | 0.105 | 0.895 | 3 |
| 71 | X226C06 | 3 | 0.003 | 0.997 | 3 |
| 72 | X113B11 | 3 | 0.320 | 0.680 | 3 |
| 73 | X232C58 | 3 | 0.020 | 0.980 | 3 |
| 74 | X234C15 | 3 | 0.028 | 0.972 | 3 |
| 75 | X238C87 | 3 | 0.062 | 0.938 | 3 |
| 76 | X241C01 | 3 | 0.009 | 0.991 | 3 |
| 77 | X249C42 | 3 | 0.003 | 0.997 | 3 |
| 78 | X250C78 | 3 | 0.007 | 0.993 | 3 |
| 79 | X252C64 | 3 | 0.020 | 0.980 | 3 |
| 80 | X269C68 | 3 | 0.003 | 0.997 | 3 |
| 81 | X26C23 | 3 | 0.078 | 0.922 | 3 |
| 82 | X270C93 | 3 | 0.105 | 0.895 | 3 |
| 83 | X271C71 | 3 | 0.009 | 0.991 | 3 |
| 84 | X279C61 | 3 | 0.009 | 0.991 | 3 |
| 85 | X287C67 | 3 | 0.079 | 0.921 | 3 |
| 86 | X291C17 | 3 | 0.008 | 0.992 | 3 |
| 87 | X127B00 | 3 | 0.003 | 0.997 | 3 |
| 88 | X303C36 | 3 | 0.003 | 0.997 | 3 |
| 89 | X304C89 | 3 | 0.888 | 0.112 | 1 |
| 90 | X311A27 | 3 | 0.010 | 0.990 | 3 |
| 91 | X313A87 | 3 | 0.059 | 0.941 | 3 |
| 92 | X314B55 | 3 | 0.010 | 0.990 | 3 |
| 93 | X101B88 | 3 | 0.007 | 0.993 | 3 |
| 94 | X37C06 | 3 | 0.003 | 0.997 | 3 |
| 95 | X46A25 | 3 | 0.064 | 0.936 | 3 |
| 96 | X131B79 | 3 | 0.078 | 0.922 | 3 |
| 97 | X54A09 | 3 | 0.007 | 0.993 | 3 |
| 98 | X55A79 | 3 | 0.322 | 0.678 | 3 |
| 99 | X62A02 | 3 | 0.007 | 0.993 | 3 |
| 100 | X66A84 | 3 | 0.003 | 0.997 | 3 |
| 101 | X67A43 | 3 | 0.003 | 0.997 | 3 |
| 102 | X69A93 | 3 | 0.007 | 0.993 | 3 |
| 103 | X70A79 | 3 | 0.003 | 0.997 | 3 |
| 104 | X73A01 | 3 | 0.643 | 0.357 | 1 |
| 105 | X76A44 | 3 | 0.064 | 0.936 | 3 |
| 106 | X79A35 | 3 | 0.007 | 0.993 | 3 |
| 107 | X82A83 | 3 | 0.147 | 0.853 | 3 |
| 108 | X89A64 | 3 | 0.003 | 0.997 | 3 |
| 109 | X90A63 | 3 | 0.009 | 0.991 | 3 |
| 110 | X139B03 | 3 | 0.067 | 0.933 | 3 |
| 111 | X102B06 | 3 | 0.003 | 0.997 | 3 |
| 112 | X142B05 | 3 | 0.010 | 0.990 | 3 |
| 113 | X143B81 | 3 | 0.020 | 0.980 | 3 |
| 114 | X146B39 | 3 | 0.007 | 0.993 | 3 |
| 115 | X147B19 | 3 | 0.020 | 0.980 | 3 |
| 116 | X103B41 | 3 | 0.009 | 0.991 | 3 |
| 117 | X153B09 | 3 | 0.007 | 0.993 | 3 |
| 118 | X104B91 | 3 | 0.052 | 0.948 | 3 |
| 119 | X162B98 | 3 | 0.439 | 0.561 | 3 |
| 120 | X172B19 | 3 | 0.007 | 0.993 | 3 |
| 121 | X182B43 | 3 | 0.003 | 0.997 | 3 |
| 122 | X194B60 | 3 | 0.009 | 0.991 | 3 |
| 123 | X200B47 | 3 | 0.795 | 0.205 | 1 |

APPENDIX 8C
SWS Classifier 4: G2a-G2b Prediction Validation
# Cox PH test summary (Baseline group 1)
coef exp(coef) se(coef) z p

| | | | | | |
|---|---|---|---|---|---|
| group2b | 0.789 | 2.2 | 0.293 | 2.69 | 0.007 |

Likelihood ratio test = 7.2 on 1 df, p = 0.0073 n = 126
n events rmean se(rmean) median 0.95LCL

| 0.95UCL | | | | | | |
|---|---|---|---|---|---|---|
| Grade 2a = 77 | 22 | 10.0 | 0.508 | Inf | Inf | Inf |
| Grade 2b = 49 | 25 | 7.4 | 0.777 | 8.5 | 3 | Inf |

* DFS Event defined as any type of recurrence or death because
of breast cancer, whichever comes first

| | Prob- | Predicted | | |
|---|---|---|---|---|
| Probability | ability | grade | | |
| for | for | (2a-G2a, | DFS | DFS |
| G2a | G2b | 2b-G2b) | TIME | Event * |
| 0.001 | 0.999 | 2b | 0.5 | 1 |
| 0.001 | 0.999 | 2b | 1.5 | 0 |
| 0.999 | 0.001 | 2a | 3.75 | 1 |
| 0.003 | 0.997 | 2b | 10.08 | 0 |
| 0.999 | 0.001 | 2a | 10.75 | 0 |
| 1.000 | 0.000 | 2a | 10.75 | 0 |
| 1.000 | 0.000 | 2a | 10.75 | 0 |
| 0.024 | 0.976 | 2b | 0.92 | 1 |
| 0.024 | 0.976 | 2b | 3 | 1 |
| 0.998 | 0.002 | 2a | 8.42 | 0 |
| 0.001 | 0.999 | 2b | 10.75 | 0 |
| 1.000 | 0.000 | 2a | 4.83 | 0 |
| 0.001 | 0.999 | 2b | 0.5 | 1 |
| 0.000 | 1.000 | 2b | 10.67 | 0 |
| 0.001 | 0.999 | 2b | 8.5 | 1 |
| 0.002 | 0.998 | 2b | 2.42 | 1 |
| 0.670 | 0.330 | 2a | 2.17 | 1 |
| 0.007 | 0.993 | 2b | 7.25 | 1 |
| 0.002 | 0.998 | 2b | 0 | 1 |
| 0.525 | 0.475 | 2a | 10.5 | 0 |
| 1.000 | 0.000 | 2a | 7.42 | 0 |
| 1.000 | 0.000 | 2a | 10.67 | 0 |
| 0.999 | 0.001 | 2a | 10.5 | 0 |
| 1.000 | 0.000 | 2a | 10.5 | 0 |
| 1.000 | 0.000 | 2a | 10.5 | 0 |
| 0.000 | 1.000 | 2b | 1.25 | 1 |
| 0.000 | 1.000 | 2b | 11.58 | 0 |
| 1.000 | 0.000 | 2a | 10.5 | 0 |
| 1.000 | 0.000 | 2a | 5.75 | 1 |
| 0.025 | 0.975 | 2b | 10.42 | 0 |
| 0.008 | 0.992 | 2b | 10.42 | 0 |
| 1.000 | 0.000 | 2a | 11.17 | 0 |
| 0.000 | 1.000 | 2b | 10.33 | 0 |
| 1.000 | 0.000 | 2a | 10.33 | 0 |
| 1.000 | 0.000 | 2a | 10.33 | 0 |
| 0.999 | 0.001 | 2a | 10.25 | 0 |
| 1.000 | 0.000 | 2a | 8.58 | 0 |
| 0.999 | 0.001 | 2a | 5 | 1 |
| 0.997 | 0.003 | 2a | 10.25 | 0 |
| 1.000 | 0.000 | 2a | 6.83 | 0 |
| 0.999 | 0.001 | 2a | 1 | 1 |
| 1.000 | 0.000 | 2a | 10 | 0 |
| 1.000 | 0.000 | 2a | 10 | 0 |
| 1.000 | 0.000 | 2a | 10 | 0 |
| 1.000 | 0.000 | 2a | 10 | 0 |
| 1.000 | 0.000 | 2a | 9.92 | 0 |
| 1.000 | 0.000 | 2a | 9.92 | 0 |
| 1.000 | 0.000 | 2a | 6.5 | 1 |
| 0.007 | 0.993 | 2b | 9.92 | 0 |
| 0.754 | 0.246 | 2a | 9.83 | 0 |
| 0.001 | 0.999 | 2b | 2.25 | 1 |
| 1.000 | 0.000 | 2a | 10.17 | 0 |
| 0.003 | 0.997 | 2b | 2.42 | 1 |
| 1.000 | 0.000 | 2a | 10.08 | 0 |
| 1.000 | 0.000 | 2a | 10 | 0 |
| 0.999 | 0.001 | 2a | 9.92 | 0 |
| 1.000 | 0.000 | 2a | 4.42 | 1 |
| 0.727 | 0.273 | 2a | 9.92 | 0 |
| 0.525 | 0.475 | 2a | 12.75 | 0 |
| 0.000 | 1.000 | 2b | 9.58 | 1 |
| 0.999 | 0.001 | 2a | 9.08 | 1 |
| 0.007 | 0.993 | 2b | 12.67 | 0 |
| 0.001 | 0.999 | 2b | 2.58 | 1 |
| 1.000 | 0.000 | 2a | 1.08 | 1 |
| 0.000 | 1.000 | 2b | 0.42 | 1 |
| 0.001 | 0.999 | 2b | 0.75 | 1 |
| 0.999 | 0.001 | 2a | 0.67 | 1 |
| 0.007 | 0.993 | 2b | 2 | 1 |
| 1.000 | 0.000 | 2a | 0.17 | 1 |
| 0.001 | 0.999 | 2b | 12.42 | 0 |
| 0.848 | 0.152 | 2a | 3.58 | 1 |
| 0.719 | 0.281 | 2a | 1.08 | 1 |
| 0.719 | 0.281 | 2a | 2.42 | 1 |
| 0.001 | 0.999 | 2b | 12.17 | 0 |
| 0.693 | 0.307 | 2a | 2.42 | 1 |
| 0.999 | 0.001 | 2a | 2.08 | 0 |
| 0.001 | 0.999 | 2b | 12.17 | 0 |
| 1.000 | 0.000 | 2a | 12.08 | 0 |
| 0.001 | 0.999 | 2b | 4.25 | 1 |
| 1.000 | 0.000 | 2a | 11.08 | 0 |
| 0.999 | 0.001 | 2a | 0.08 | 1 |
| 0.999 | 0.001 | 2a | 10.5 | 1 |
| 0.754 | 0.246 | 2a | 11.33 | 0 |
| 1.000 | 0.000 | 2a | 10.83 | 0 |
| 1.000 | 0.000 | 2a | 11.5 | 0 |
| 1.000 | 0.000 | 2a | 11.5 | 0 |
| 0.000 | 1.000 | 2b | 11.42 | 0 |
| 0.848 | 0.152 | 2a | 10.83 | 0 |
| 1.000 | 0.000 | 2a | 11.42 | 0 |
| 1.000 | 0.000 | 2a | 11.42 | 0 |
| 0.999 | 0.001 | 2a | 2.08 | 0 |
| 0.002 | 0.998 | 2b | 3.42 | 1 |
| 1.000 | 0.000 | 2a | 4.67 | 1 |
| 0.001 | 0.999 | 2b | 6.5 | 1 |
| 1.000 | 0.000 | 2a | 4.42 | 0 |
| 1.000 | 0.000 | 2a | 11.42 | 0 |
| 0.000 | 1.000 | 2b | 11.33 | 0 |
| 0.001 | 0.999 | 2b | 1.5 | 1 |
| 0.001 | 0.999 | 2b | 11.33 | 0 |
| 1.000 | 0.000 | 2a | 5.33 | 0 |
| 0.525 | 0.475 | 2a | 11.33 | 0 |
| 1.000 | 0.000 | 2a | 3.58 | 1 |
| 1.000 | 0.000 | 2a | 4.08 | 1 |
| 0.001 | 0.999 | 2b | 1.75 | 1 |
| 0.003 | 0.997 | 2b | 0 | 1 |
| 0.999 | 0.001 | 2a | 6 | 0 |
| 0.999 | 0.001 | 2a | 7.42 | 0 |
| 0.999 | 0.001 | 2a | 2.33 | 1 |
| 1.000 | 0.000 | 2a | 1.92 | 0 |
| 0.592 | 0.408 | 2a | 7 | 1 |
| 0.001 | 0.999 | 2b | 0.17 | 1 |
| 0.000 | 1.000 | 2b | 0 | 1 |
| 0.005 | 0.995 | 2b | 11 | 0 |
| 0.000 | 1.000 | 2b | 11 | 0 |
| 0.030 | 0.970 | 2b | 10.75 | 0 |
| 0.001 | 0.999 | 2b | 4.42 | 1 |
| 0.000 | 1.000 | 2b | 10.92 | 0 |
| 0.001 | 0.999 | 2b | 10.92 | 0 |
| 1.000 | 0.000 | 2a | 10.92 | 0 |
| 0.001 | 0.999 | 2b | 10.92 | 0 |
| 0.000 | 1.000 | 2b | 11.67 | 0 |
| 1.000 | 0.000 | 2a | 10.92 | 0 |
| 1.000 | 0.000 | 2a | 10.83 | 0 |
| 1.000 | 0.000 | 2a | 10.83 | 0 |
| 0.754 | 0.246 | 2a | 6.42 | 0 |
| 0.001 | 0.999 | 2b | 0.08 | 0 |

1. A method of treating a patient having a high aggressiveness tumour, the method comprising:
(a) identifying the high aggressiveness tumour by:
(i) obtaining, from a sample of a histological Grade 2 tumour isolated from the patient, gene expression data of BRRN1, AURKA, MELK, PRR11, CENPW and E2F1;
(ii) assigning a grade to the tumour by applying a class prediction algorithm to the gene expression data, wherein a Grade 3 tumour is classified as a high aggressiveness tumour; and
(b) treating the patient by administering an agent selected from the group consisting of: an antiproliferative chemotherapeutic agent, a vinca alkaloid, a condensin inhibitor, vinblastine, vincristine, vindesine, vinorelbine, desoxyvincaminol, vincaminol, vinburnine, vincamajine, vineridine, vinburnine, vinpocetine, a taxane, paclitaxel (taxol), docetaxel (taxotere), cabazitaxel, an AURKA inhibitor, alisertib, a MELK inhibitor, OTS167, an anthracycline, doxorubicin, idarubicin, epirubicin, a CDK 4/6 inhibitor or palbociclib.

2. A method of treating a patient having a low aggressiveness tumour, the method comprising:
(a) identifying the low aggressiveness tumour by:
(i) obtaining, from a sample of a histological Grade 2 tumour isolated from the patient, gene expression data of BRRN1, AURKA, MELK, PRR11, CENPW and E2F1;
(ii) assigning a grade to the tumour by applying a class prediction algorithm to the gene expression data, wherein a Grade 1 tumour is classified as a low aggressiveness tumour; and
(b) treating the patient by administering an agent selected from the group consisting of: an mTOR inhibitor, rapamycin, a rapalog, sirolimus, everolimus, temsirolimus, bevacizumab, tamoxifen, anastrozole, letrozole, exemestane or goserelin.

3. A method of assigning a grade to a tumour, which grade is indicative of the aggressiveness of the tumour, the method comprising detecting the expression of one or more genes selected from the genes set out in Table D0 (6g-TAGs) or Table D1 (SWS Classifier 0).

4. A method according to claim 3, in which the method comprises detecting a high level of expression of the gene and assigning the grade set out in Column 7 (“Grade with Higher Expression”) of the Table to the tumour or detecting a low level of expression of the gene and assigning the grade set out in Column 8 (“Grade with Lower Expression”) of the Table to the tumour.

5. A method according to claim 3, in which a high level of expression is detected if the expression level of the gene is above the expression level set out in Column 9 (“Cut-Off”) of the Table, and a low level of expression is detected if the expression level of the gene is below that level.

6. A method according to claim 3, in which the method comprises detecting the expression of two, three, four, five or all of the genes set out in Table D0 (6g-TAGs), viz: BRRN1 (GenBank Accession No. NM_015341), AURKA (GenBank Accession No. NM_003600), MELK (GenBank Accession No. NM_014791), PRR11 (GenBank Accession No. NM_018304), CENPW (GenBank Accession No. NM_001012507) and E2F1 (GenBank Accession No. NM_005225).

7. A method according to claim 3, in which the method comprises detecting the expression of two, three, four, five or all of the genes set out in Table D2 (SWS Classifier 1), viz: Barren homolog (Drosophila) (BRRN1, GenBank Accession No. D38553); Hypothetical protein FLJ11029 (FLJ11029, GenBank Accession No. BG165011); cDNA clone IMAGE:4452583, partial cds (GenBank Accession No. BG492359); Serine/threonine-protein kinase 6 (STK6); and Maternal embryonic leucine zipper kinase (MELK, GenBank Accession No. NM_014791).

8. A method according to claim 3, in which the method comprises detecting the expression of two, three, four, five or all of the genes set out in Table D4 (SWS Classifier 3), viz: TPX2, microtubule-associated protein homolog (Xenopus laevis) (TPX2, GenBank Accession No. AF098158), Protein regulator of cytokinesis 1 (PRC1, GenBank Accession No. NM_003981), Neuro-oncological ventral antigen 1 (NOVA1, GenBank Accession No. NM_002515), Stanniocalcin 2 (STC2, GenBank Accession No. AI435828), Cold inducible RNA binding protein (CIRBP, GenBank Accession No. AL565767), Chemokine (C-X-C motif) ligand 14 (CXCL14, GenBank Accession No. NM_004887), Signal peptide, CUB domain, EGF-like 2 (SCUBE2, GenBank Accession No. AI424243).

9. A method according to claim 3, in which the method comprises detecting the expression of two, three, four, five or all of the genes set out in Table D5 (SWS Classifier 4), viz: cell division cycle associated 8 (CDCA8, GenBank Accession No. BC001651), centromere protein E, 312 kDa (CENPE, GenBank Accession No. NM_001813), steroid-5-alpha-reductase, alpha polypeptide 1 (3-oxo-5 alpha-steroid delta 4-dehydrogenase alpha 1) (SRD5A1, GenBank Accession No. BC006373), microtubule-associated protein tau (MAPT, GenBank Accession No. NM_016835), leucine zipper protein (FKSG14, GenBank Accession No. FKSG14), BC005400 (GenBank Accession No. R38110), EH-domain containing 2 (EHD2, GenBank Accession No. AI417917).

10. A method according to claim 3, in which the method comprises detecting the expression of two, three, four, five or all of the genes set out in Table D3 (SWS Classifier 2), viz: Barren homolog (Drosophila) (BRRN1, GenBank Accession No. D38553); Cell division cycle associated 8 (CDCA8, GenBank Accession No. BC001651); V-myb myeloblastosis viral oncogene homolog (avian)-like 2 (MYBL2, GenBank Accession No. NM_002466); Hypothetical protein FLJ11029 (FLJ11029, GenBank Accession No. BG165011); FBJ murine osteosarcoma viral oncogene homolog B (FOSB, GenBank Accession No. NM_006732); CDNA clone IMAGE:4452583, partial cds (GenBank Accession No. BG492359); Serine/threonine-protein kinase 6 (STK6, GenBank Accession No. BC027464); Anillin, actin binding protein (scraps homolog, Drosophila) (ANLN, GenBank Accession No. AK023208); Centromere protein E, 312 kDa (CENPE, GenBank Accession No. NM_001813); TTK protein kinase (TTK, GenBank Accession No. NM_003318); Signal peptide, CUB domain, EGF-like 2 (SCUBE2, GenBank Accession No. AI424243); V-fos FBJ murine osteosarcoma viral oncogene homolog (FOS, GenBank Accession No. BC004490); TPX2, microtubule-associated protein homolog (Xenopus laevis) (TPX2, GenBank Accession No. AF098158); Kinetochore protein Spc24 (Spc24, GenBank Accession No. AI469788); Forkhead box M1 (FOXM1, GenBank Accession No. NM_021953); Maternal embryonic leucine zipper kinase (MELK, GenBank Accession No. NM_014791); Cell division cycle associated 5 (CDCA5, GenBank Accession No. BE614410); and Cell division cycle associated 3 (CDCA3, GenBank Accession No. NM_031299).

11. A method according to claim 3, in which the tumour is selected from the group consisting of: a breast tumour, multiple myeloma (GSE2658), kidney renal clear cell carcinoma (TCGA) and sarcoma (GSE21050).

12. A method of classifying a histological Grade 2 tumour into a low aggressiveness tumour or a high aggressiveness tumour, the method comprising assigning a grade to the histological Grade 2 tumour according to claim 3.

13. A method of predicting a survival rate for an individual with a histological Grade 2 breast tumour, the method comprising assigning a grade to the breast tumour by a method according to claim 3, in which a low aggressiveness grade indicates a high probability of survival and a high aggressiveness grade indicates a low probability of survival.

14. A method of prognosis of an individual with a breast tumour, the method comprising assigning a grade to the breast tumour by a method according to claim 3.

15. A method of diagnosis of aggressive breast cancer in an individual, the method comprising assigning a grade indicative of high aggressiveness to a breast tumour of the individual by a method according to claim 3.

16. A method of choosing a therapy for an individual with breast cancer, the method comprising assigning a grade to the breast tumour by a method according to claim 3, and choosing an appropriate therapy based on the aggressiveness of the breast tumour, in which a high aggressiveness tumour is treated by administering an antiproliferative chemotherapeutic agent, a vinca alkaloid, a condensin inhibitor, vinblastine, vincristine, vindesine, vinorelbine, desoxyvincaminol, vincaminol, vinburnine, vincamajine, vineridine, vinburnine, vinpocetine, a taxane, paclitaxel (taxol), docetaxel (taxotere), cabazitaxel, an AURKA inhibitor, alisertib, a MELK inhibitor, OTS167, an anthracycline, doxorubicin, idarubicin, epirubicin, a CDK 4/6 inhibitor or palbociclib to the patient, and in which a low aggressiveness tumour is treated by administering an mTOR inhibitor, rapamycin, a rapalog, sirolimus, everolimus, temsirolimus, bevacizumab, tamoxifen, anastrozole, letrozole, exemestane or goserelin to the patient.

17. A method of treatment of an individual with breast cancer, the method comprising assigning a grade to the breast tumour by a method according to any of claim 3, and administering an appropriate therapy to the individual based on the aggressiveness of the breast tumour, in which a high aggressiveness tumour is treated by administering an antiproliferative chemotherapeutic agent, a vinca alkaloid, a condensin inhibitor, vinblastine, vincristine, vindesine, vinorelbine, desoxyvincaminol, vincaminol, vinburnine, vincamajine, vineridine, vinburnine, vinpocetine, a taxane, paclitaxel (taxol), docetaxel (taxotere), cabazitaxel, an AURKA inhibitor, alisertib, a MELK inhibitor, OTS167, an anthracycline, doxorubicin, idarubicin, epirubicin, a CDK 4/6 inhibitor or palbociclib to the patient, and in which a low aggressiveness tumour is treated by administering an mTOR inhibitor, rapamycin, a rapalog, sirolimus, everolimus, temsirolimus, bevacizumab, tamoxifen, anastrozole, letrozole, exemestane or goserelin to the patient.

18. A method of determining whether a breast tumour is a metastatic breast tumour, the method comprising assigning a grade to the breast tumour by a method according to claim 3.

19. A method of identifying a molecule capable of treating or preventing breast cancer, the method comprising: (a) grading a breast tumour; (b) exposing the breast tumour to a candidate molecule; and (c) detecting a change in tumour grade; in which the grade or change thereof, or both, is assigned by a method according to any of claim 3.

20. A method of treatment or prevention of breast cancer in an individual, the method comprising modulating the expression of a gene set out in Table D0 (6g-TAGs) or Table D1 (SWS Classifier 0).

21. A method of determining the proliferative state of a cell, the method comprising detecting the expression of a gene selected from the genes set out in Table D0 (6g-TAGs) or Table D1 (SWS Classifier 0), in which:
(a) a high level of expression of a gene which is annotated “3” in Column 7 (“Grade with Higher Expression”) indicates a highly proliferative cell;
(b) a high level of expression of a gene which is annotated “1” in Column 7 (“Grade with Higher Expression”) indicates a non-proliferating cell or a slow-growing cell;
(c) a low level of expression of a gene which is annotated “3” in Column 8 (“Grade with Lower Expression”) indicates a highly proliferative cell; and
(d) a low level of expression of a gene which is annotated “1” in Column 8 (“Grade with Lower Expression”) indicates a non-proliferating cell or a slow-growing cell.

22. A combination comprising the genes or probesets set out in Table D0 (6-TAGs) or in Table D1 (SWS Classifier 0).

23. A primer pair selected from the group consisting of:
(a) a primer pair suitable for amplification of CENPW comprising CGTCATACGGACCGGATTGT and GGAGACTATGGTCGACAGCG;
(b) a primer pair suitable for amplification of PRR11 comprising CAAAGCTGCTACTGCCATTG and CTGGTTGCCA TTCAGTCTCA;
(c) a primer pair suitable for amplification of MELK comprising CAAACTTGCCTGCCATATCCT and GGCTGTCTCTAGCACATGGTA;
(d) a primer pair suitable for amplification of AURKA comprising AGCTAGAGGCATCATGGACCG and GCTCAGCTGGAGAAAGCCGGA;
(e) a primer pair suitable for amplification of BRRN1 comprising TGCCAAAAAGATGGACATGA and CCGCTAAGCATCTTCTCGTC; and
(f) a primer pair suitable for amplification of E2F1 comprising GCTGTTCTTCTGCCCCATAC and GAAGGCCCATCTCATATCCA.

24. A computer implemented method of assigning a grade to a breast tumour, the method comprising processing expression data for one or more genes set out in Table D0 (6g-TAGs) or Table D1 (SWS Classifier 0) and obtaining a grade indicative of aggressiveness of the breast tumour.

25. A program storage device readable by a machine, tangibly embodying a program of instructions executable by the machine to perform method of assigning a grade to a breast tumour, the method comprising: processing expression data for one or more genes set out in Table D0 (6g-TAGs) or Table D1 (SWS Classifier 0); and obtaining a grade indicative of aggressiveness of the breast tumour.


SEQUENCE LISTING (12 sequences)

SEQ ID NO: 1 - DNA, 20 bp, Artificial Sequence
  Description of Artificial Sequence Synthetic primer
cgtcatacggaccggattgt

SEQ ID NO: 2 - DNA, 20 bp, Artificial Sequence
  Description of Artificial Sequence Synthetic primer
ggagactatggtcgacagcg

SEQ ID NO: 3 - DNA, 20 bp, Artificial Sequence
  Description of Artificial Sequence Synthetic primer
caaagctgctactgccattg

SEQ ID NO: 4 - DNA, 20 bp, Artificial Sequence
  Description of Artificial Sequence Synthetic primer
ctggttgccattcagtctca

SEQ ID NO: 5 - DNA, 21 bp, Artificial Sequence
  Description of Artificial Sequence Synthetic primer
caaacttgcctgccatatcct

SEQ ID NO: 6 - DNA, 21 bp, Artificial Sequence
  Description of Artificial Sequence Synthetic primer
ggctgtctctagcacatggta

SEQ ID NO: 7 - DNA, 21 bp, Artificial Sequence
  Description of Artificial Sequence Synthetic primer
agctagaggcatcatggaccg

SEQ ID NO: 8 - DNA, 21 bp, Artificial Sequence
  Description of Artificial Sequence Synthetic primer
gctcagctggagaaagccgga

SEQ ID NO: 9 - DNA, 20 bp, Artificial Sequence
  Description of Artificial Sequence Synthetic primer
tgccaaaaagatggacatga

SEQ ID NO: 10 - DNA, 20 bp, Artificial Sequence
  Description of Artificial Sequence Synthetic primer
ccgctaagcatcttctcgtc

SEQ ID NO: 11 - DNA, 20 bp, Artificial Sequence
  Description of Artificial Sequence Synthetic primer
gctgttcttctgccccatac

SEQ ID NO: 12 - DNA, 20 bp, Artificial Sequence
  Description of Artificial Sequence Synthetic primer
gaaggcccatctcatatcca